(19) United States
(12) Patent Application Publication
(10) Pub. No.: US 2025/0243471 A1
(43) Pub. Date: Jul. 31, 2025

(54) PROGRAMMABLE PATTERN RECOGNITION COMPOSITIONS

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Alex Gao, Cambridge, MA (US); Max Wilkinson, Cambridge, MA (US); Jonathan Strecker, Cambridge, MA (US)

(21) Appl. No.: 19/040,594

(22) Filed: Jan. 29, 2025

Related U.S. Application Data
(63) Continuation of application No. PCT/US2023/071227, filed on Jul. 28, 2023.
(60) Provisional application No. 63/393,403, filed on Jul. 29, 2022.

(51) Int. Cl. C12N 9/14; C12N 15/70; G01N 33/569

(52) U.S. Cl. CPC C12N 9/14; C12N 15/70; C12Y 306/01015; G01N 33/56983

(57) ABSTRACT

Described in several example embodiments herein are engineered programmable pattern recognition compositions and uses thereof. In an embodiment, the engineered protein contains an NTPase of a Signal Transduction ATPases with Numerous-associated Domains (STAND) superfamily (STAND NTPase), comprising a pathogen-associated molecular pattern (PAMP) recognition activity, wherein the STAND NTPase and the PAMP recognition activity are derived from the same or different prokaryotes.

### CROSS-REFERENCE TO RELATED APPLICATIONS

This application is a continuation of International Application No. PCT/US2023/071227 filed Jul. 28, 2023, which claims the benefit of and priority to U.S. Provisional Patent Application No. 63/393,403, filed on Jul. 29, 2022, the contents of each of which are incorporated by reference herein in their entirety.

### STATEMENT REGARDING FEDERALLY SPONSORED RESEARCH

This invention was made with government support under Grant No. HL141201 and HG009761-05 awarded by the National Institutes of Health. The government has certain rights in the invention.

### SEQUENCE LISTING

This application contains a sequence listing filed in electronic form as an xml file entitled “BROD-5585US_ST26.xml”, created on Jan. 29, 2025, and having a size of 109,276 bytes. The content of the sequence listing is incorporated herein in its entirety.

### TECHNICAL FIELD

The subject matter disclosed herein is generally directed to prokaryotic innate immunity via pattern recognition of conserved viral proteins.

### BACKGROUND

All organisms have evolved specialized immune proteins, including pattern recognition receptors consisting of nucleotide-binding oligomerization domain-like receptors (NLRs) of the STAND superfamily ubiquitous in eukaryotes. NLRs recognize conserved pathogen-associated molecular patterns, leading to activation of an effector domain and an inflammatory or apoptotic response. The roles of NLRs in eukaryotic immunity are well established, but it is unknown whether prokaryotes use similar defense mechanisms.

Citation or identification of any document in this application is not an admission that such a document is available as prior art to the present invention.

### SUMMARY

Described in an embodiment herein are engineered proteins comprising an effector domain, an effector activation domain, and a recognition domain, wherein binding of a target polypeptide to the recognition domain leads to activation of the effector domain via the effector activation domain, and wherein at least one of the effector, effector activation, and recognition domains is derived from a STAND NTPase protein.

In an embodiment, the STAND NTPase protein is an antiviral STAND (Avs). In an embodiment, the Avs is an Avs1, Avs2, Avs3, or Avs4.

In an embodiment, the effector domain is an endonuclease, a protease, a nucleosidase, hydrolase, or caspase-like domain.

In an embodiment, the effector activation domain is an NTPase.

In an embodiment, the recognition domain is engineered to recognize a target polypeptide other than a target polypeptide of a wild-type STAND NTPase protein. In an embodiment, the recognition domain comprises tetratricopeptide repeat (TPR) domains.

In an embodiment, wherein a microbe comprises the target polypeptide, optionally wherein the microbe is part of a microbiome.

In an embodiment, the target polypeptide is a phage polypeptide.

Described in an embodiment herein are oligomers comprising at least two of the engineered proteins of the present invention. In an embodiment, the oligomer is a tetramer, trimer, or dimer.

Described in an embodiment herein are detection compositions comprising (a) an engineered protein of any one of the preceding paragraphs and as described in greater detail elsewhere herein; (b) a detection construct, wherein binding of a target polypeptide to the recognition domain activates the effector domain and mediates effector domain modification of the detection construct resulting in generation of a detectable signal.

Described in an embodiment herein are polynucleotide(s) encoding the engineered protein of any one of the preceding paragraphs and as described in greater detail elsewhere herein.

Described in an embodiment herein are polynucleotide(s) encoding component (a), component (b), or both of the detection composition.

Described in an embodiment herein are vectors and vector systems comprising a polynucleotide encoding an engineered protein described herein and/or a detection composition described herein.

Described in an embodiment herein are cells or cell populations comprising an engineered protein of the present invention described herein, a detection composition of the present invention described herein, a polynucleotide encoding an engineered protein of the present invention and/or a detection composition of the present invention, a vector or vector system of the present invention, or any combination thereof.

Described in an embodiment herein are formulation comprising an engineered protein of the present invention described herein, a detection composition of the present invention described herein, a polynucleotide encoding an engineered protein of the present invention and/or a detection composition of the present invention, a vector or vector system of the present invention, a cell or cell population of the present invention, or any combination thereof; and optionally a pharmaceutically acceptable carrier.

Described in an embodiment herein are methods of modifying a target molecule and/or cell comprising delivering an engineered protein of the present invention described herein, a detection composition of the present invention described herein, a polynucleotide encoding an engineered protein of the present invention and/or a detection composition of the present invention, a vector or vector system of the present invention, or any combination thereof to the target molecule and/or cell, wherein the target molecule and/or cell is or comprises a target polypeptide; and activating an effector domain of the engineered protein by allowing binding of the target polypeptide to the recognition domain thereby activating the effector domain via the effector activation domain, wherein effector domain activity modifies the target molecule and/or cell. In an embodiment, delivering comprises in vitro, ex vivo, or in vivo delivery.

Described in an embodiment herein are methods of detecting a target molecule and/or cell, the method comprising combining a detection composition of the present invention or a formulation thereof and a sample or component thereof; and activating an effector domain of the engineered protein via binding of a target polypeptide in the sample to the recognition domain thereby mediating effector domain modification of the detection construct and generation of a detectable signal. In an embodiment, the method is performed in whole or in part in vitro, ex vivo, or in vivo.

Described in an embodiment herein are methods of modifying a microbiome structure comprising introducing an engineered protein of any one of the engineered proteins of the present invention capable of recognizing a target polypeptide of one or more microbes in a microbiome into a microbiome, wherein activation of the effector domain via binding of a target polypeptide of one or more microbes in the microbiome to the recognition domain results in modification of the one or more microbes thereby modifying the microbiome structure.

Described in an embodiment herein are methods of engineered phage-resistant bacteria comprising expressing an engineered protein of the present invention capable of recognizing a phage polypeptide in a bacterium or bacteria population.

Described in an embodiment herein are methods of cargo delivery comprising delivering, to a cell, (a) an engineered protein of the present invention; (b) a cargo, (c) a detection composition or (d) any combination thereof, wherein the engineered protein comprises the cargo or wherein the cargo comprises the target polypeptide, wherein the cell optionally comprises the target polypeptide, and wherein activation of the effector domain by binding of the target polypeptide to the recognition domain results in delivery of the cargo.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—Example of avs genes (FIG. 1A) present in defense islands and (FIG. 1B) clustered with other avs genes. Other defense genes are highlighted in gray.

FIG. 2—Heterologous reconstitution of Avs antiphage activity in E. coli. Plaque assay spots correspond to 10-fold dilutions of phages T7, PhiV-1, P1, Lambda, T4, T5, and ZL19 on E. coli containing Avs-expressing plasmids.

FIG. 3A-3F—Prokaryotic STAND NTPases recognize phage terminase and portal proteins. (FIG. 3A) Maximum likelihood tree of the ATPase domain of selected NLR-like STAND NTPases in four model organisms across kingdoms of life. (FIG. 3B) Domain architectures of representative NLR-like genes in FIG. 3A. LRR, leucine-rich repeat; TPR, tetratricopeptide repeat; WD40, WD40 repeat; ankyrin, ankyrin repeat; BIR, baculoviral inhibitor of apoptosis repeat; PYD, pyrin domain; FUND, function to find domain; CARD, caspase activation and recruitment domain; RX-CC, potato virus X resistance protein coiled-coil domain; PLP, patatin-like phospholipase; TIR, toll/interleukin-1 receptor homology domain. (FIG. 3C) Schematic of genetic screening approach to identify phage-encoded activators of Avs proteins that induce cell death. (FIG. 3D) Genetic screen results for phage-encoded activators. (FIG. 3E) Quantification of the phage DNA band intensity in a Southern blot of DNA isolated from phage-infected E. coli. (FIG. 3F) Photographs of E. coli co-transformation assays with Avs genes and phage activators identified in FIG. 3D.

FIG. 4A-4B—Four distinct clades of Avs proteins. (FIG. 4A) Phylogenetic tree of the ATPase domain of selected Avs proteins and other related ATPases identified by PSIBLAST. (FIG. 4B) UPGMA dendrogram including the ATPase domains in FIG. 4A and profiles of additional ATPases in pfam.

FIG. 5A-5B—Related to FIG. 3A-3F. (FIG. 5A) Schematic of PhiV-1 fragment screen (FIG. 1C). (FIG. 5B) Read coverage of PhiV-1 fragment screen, without normalizing to the empty vector control.

FIG. 6A-6E—Related to FIG. 3A-3F. (FIG. 6A) Schematic for PhiV-1 mutant construction via plasmid homology donors in E. coli. A trans complementation plasmid encoding gp8 or gp19 was maintained in the cells to support phage growth. (FIG. 6B) Plaque assay validation of PhiV-1 knockout phages across different complementation plasmids. Spots correspond to 10-fold phage dilutions from right to left. (FIG. 6C) Southern blot analysis of phage-infected E. coli cell lysates using a PhiV-1 specific probe. WT, Δgp8, and Δgp19 were used at an MOI of 1, and Δgp19CTD was used at an MOI of 0.25. END-seq analysis (74) of DNA double-strand breaks (DSBs) in (FIG. 6D) NotI-digested PhiV-1 DNA as a positive control and (FIG. 6E) E. coli cultures infected with wild type or mutant PhiV-1. Input DNA was normalized across samples.

FIG. 7A-7D—Avs proteins are pattern-recognition receptors for the terminase and portal of diverse tailed phages. (FIG. 7A) Schematic of plasmid depletion assay. (FIG. 7B) Heatmaps of plasmid depletion for the terminase and portal proteins of representative phages spanning nine major tailed phage families. The native Avs promoter was retained for all homologs except for those outside of the Enterobacteriaceae family (EpAvs1 and CcAvs4). Terminases and portals were induced with 0.002% arabinose. Horizontal black bars indicate groups of terminase proteins with at least 20% pairwise sequence identity. Asterisks indicate prophages. S. flava, Sphingopyxis flava R11H; D. archaeon, Desulfurococcales archaeon ex4484_217_2; E. coli-1, Escherichia coli NCTC9020; E. coli-2, Escherichia coli M885. (FIG. 7C) Pairwise amino acid sequence identity between the core folds of the terminases and portals in (FIG. 7B), excluding non-conserved regions. (FIG. 7D) Activity of four Avs proteins against the human herpesvirus 8 (HHV-8) terminase and portal.

FIG. 8—Related to FIG. 3A-3F. Photographs of E. coli co-transformation assays with Avs1-2 and activators from phage PhiV-1. The left spot on each image corresponds to a 10-fold dilution of the right spot.

FIG. 9A-9B—Robustness of the terminase and portal plasmid depletion assay in FIG. 7A-7D. (FIG. 9A) Specificity of Avs target recognition with avs genes expressed under the control of a lac promoter and weak induction of terminases and portals (0.002% arabinose). (FIG. 9B) Specificity of Avs target recognition with native avs promoters and strong induction of terminases and portals (0.2% arabinose). Terminases and portals were expressed under the control of a pBAD promoter. Gray boxes indicate pairwise combinations not assessed due to the toxicity of terminase overexpression.

FIG. 10A-10C—Avs1, Avs2, and Avs3 contain a structurally conserved C-terminal domain essential for defense activity. (FIG. 10A) Structures predicted by AlphaFold2 of the C-terminal domains (CTDs) of the seven Avs1-3 homologs investigated in this study. The N- and C-termini are colored blue and red, respectively and represented in greyscale. (FIG. 10B) Heatmap of Dali Z-scores of pairwise comparisons between the Avs1-3 CTDs in (A) (smallest Z-score=7). Z-scores above 2 indicate significant structural similarity (Holm, Methods Mol Biol 2112, 29-42 (2020)). (FIG. 10C) Effect of CTD deletion on EcAvs2 defense activity against T7 and PhiV-1. Spots correspond to 10-fold dilutions from right to left.

FIG. 11—Structures of portal proteins predicted by AlphaFold2. The core portal fold is shown in gray. The clip, crown, and other insertions are colored blue, red, and orange, respectively and as represented in greyscale. Asterisks indicate prophages.

FIG. 12—Structures of the N-terminal ATPase domains of large terminases predicted by AlphaFold2. The core ATPase fold is shown in gray.

FIG. 13—Structures of the C-terminal nuclease domains of large terminases predicted by AlphaFold2. The core nuclease fold is shown in gray.

FIG. 14A-14H—SeAvs3 and EcAvs4 are phage-activated DNA endonucleases. (FIG. 14A) Domain architecture of SeAvs3 and EcAvs4. (FIG. 14B) (SEQ ID NO: 1-6) Alignment of Avs D-QxK nuclease motifs with characterized Cap4 and Mrr representatives. Single-letter abbreviations for the amino acid residues are as follows: A, Ala; C, Cys; D, Asp; E, Glu; F, Phe; G, Gly; H., His; I, Ile; K, Lys; L, Leu; M, Met; N, Asn; P, Pro; Q, Gln; R, Arg; S, Ser; T, Thr; V, Val; W, Trp; and Y, Tyr. (FIG. 14C-14E) Agarose gel analysis of SeAvs3 nuclease activity in vitro with a linear dsDNA substrate [(FIG. 14C) and (FIG. 14D)] and cofactor requirements (FIG. 14E). (FIG. 14F-14H) Agarose gel analysis of EcAvs4 nuclease activity in vitro with a linear dsDNA substrate (FIG. 14F-14G] and cofactor requirements (FIG. 14H).

FIG. 15A-15B—Requirements for Avs3 and Avs4 defense activity. Effects of (FIG. 15A) Avs3 small ORF deletion and (FIG. 15B) Avs3-4 nuclease and ATPase Walker A/B mutations on activity against T7 and PhiV-1.

FIG. 16A-16C—In vitro reconstitution of Avs activity. (FIG. 16A) Coomassie stained SDS-PAGE gel of purified Avs proteins and phage triggers. (FIG. 16B, 16C) Agarose gel analysis of SeAvs3 nucleic acid substrate specificity. Related to FIG. 14A-14H.

FIG. 17A-17L—Bacterial two-hybrid analysis of EcAvs4-portal interactions. (FIG. 17A) Schematic of a bacterial two-hybrid system for detecting protein-protein interactions. (FIG. 17B) Two-hybrid analysis of pairwise interactions of EcAvs4 and PhiV-1 proteins grown on S-gal indicator plates. (FIG. 17C) Interactions between EcAvs4 and the portal and terminase genes from eight phages. (FIG. 17D) Schematic of EcAvs4 protein domains. (FIG. 17E) Two-hybrid analysis of EcAvs4 mutations and truncations. (FIG. 17F) Two-hybrid analysis of PhiV-1 portal deletions. (FIG. 17G) Effect of T7 portal deletions on the activation of Avs4 as assessed by plasmid depletion. Arrows represent lac promoters. (FIG. 17H) Locations of mutations in the T7 portal (PDB: 6R21) generated by error-prone PCR that abolish activation of Avs4 (Cuervo et al., Nat. Commun. 10, 3746 (2019). (FIG. 17I) Schematic of tandem affinity purification of the SeAvs3-terminase complex. (FIG. 17J) Size exclusion chromatography of SeAvs3, PhiV-1 terminase, and the SeAvs3-terminase complex. (FIG. 17K) Coomassie-stained SDS-PAGE protein gel of the SeAvs3-terminase complex. (FIG. 17L) Effect of terminase domain deletions on the activation of Avs1, Avs2, and Avs3. The structure of the T4 terminase (Sun et al., Cell. 135, 1251-1262 (2008) is shown as an example.

FIG. 18A-18C—Identification of single amino acid substitutions in the T7 portal protein that abrogate Avs4 activation. (FIG. 18A) (SEQ ID NO: 7) Design of a translation-reinitiation reporter system used to facilitate screening of Avs4 mutants. (FIG. 18B) Validation of reporter performance via mNeonGreen fluorescence from E. coli colonies. Scale bar: 1 cm. (FIG. 18C) Activity and location of the 29 identified portal mutants that abrogate Avs4 activation.

FIG. 19A-19G—Related to FIG. 17A-17L. (FIG. 19A) Two-hybrid analysis of pairwise interactions between SeAvs3 components and PhiV-1 triggers grown on S-gal indicator plates and (FIG. 19B) pairwise interactions between SeAvs3 and the portal and terminase genes from eight phages. (FIG. 19C) Schematic for Avs co-purification strategy. (FIG. 19D) SDS-PAGE analysis of SeAvs3 and EcAvs4 affinity purification in the presence of gp8 portal or gp19 terminase. Highlighted bands were excised and analyzed by mass spectrometry. (FIG. 19E) Total and unique mapped peptides from mass spectrometry analysis of gp19 and gp8 gel bands. (FIG. 19F) Size exclusion chromatography of protein standards (a: thyroglubulin, 670 kDa, b: γ-globulin, 158 kDa, c: ovalbumin, 44 Kda, d: myoglobin, 17 kDa, e: vitamin B12, 1.35 kDa). (FIG. 19G) Calibration curve of the Superose 6 Increase column.

FIG. 20A-20F—Taxonomic distribution and domain architectures of Avs families. (FIG. 20A) Distribution of avs genes across phyla. The values above the bars indicate the number and percentage of genomes containing each gene. PVC, Planctomycetota, Verrucomicrobiota, and Chlamydiota. The values above the bars indicate the number and percentage of genomes containing each gene. (FIG. 20B) Number of bacterial and archaeal phyla (minimum 100 sequenced isolates) with at least one detected instance of an avs gene. (FIG. 20C) Kernel density plots of the length distribution of Avs proteins, excluding the N-terminal domain. The red lines, as represented in greyscale, indicate medians. ****p<0.0001 (Mann-Whitney). (FIG. 20D-20E) Maximum likelihood tree of representatives of the ATPase+C-terminal domain of (FIG. 20D) Avs2 terminase sensors (n=1,255) and (FIG. 20E) Avs4 portal sensors (n=1,089) clustered at 95% sequence identity. See FIG. 21B-21C for the trees for Avs1 and Avs3. Stars on the outer ring indicate homologs investigated experimentally in this study. HTH, helix-turn-helix; MBL, metallo-b-lactamase; REase, restriction endonuclease. (FIG. 20F) Phage plaque assays showing antiphage defense activity of a chimeric Avs4 with transmembrane N-terminal helices from Sulfurospirillum sp. replacing the Mrr-like nuclease domain of EcAvs4. The X indicates a nuclease domain mutation.

FIG. 21A-21C—Related to FIG. 20A-20F. (FIG. 21A) Taxonomic distribution of Avs families, stratified by bacterial and archaeal phylum. The bar graphs show the number of genomes available for analysis. Maximum likelihood phylogenetic trees of the (FIG. 21B) Avs1 (n=843) and (FIG. 21C) Avs3 (n=630) families. Inner, middle, and outer rings indicate taxonomy, N-terminal effectors domains, and locus architecture, respectively.

FIG. 22A-22B—Examples of Avs proteins implicated in protein-protein signaling. (FIG. 22A) Predicted caspase recruitment by cyanobacterial Avs2 homologs via an N-terminal EAD10 protein recruitment domain that is also shared by proteins encoded in the vicinity. The tree was constructed from a multiple sequence alignment of the caspase. Protein accession numbers refer to the STAND NTPase. (FIG. 22B) An Avs3 homolog within a genomic locus from Sulfurovum sp. enriched in TIR domains related to those mediating second messenger signaling (Ofir et al. Nature 600, 116-120 (2021)).

FIG. 23A-23C—Related to FIG. 20A-20F. (FIG. 23A) (SEQ ID NO: 8-9) Amino acid sequence surrounding the EcAvs4 chimera break point. (FIG. 23B) Chimera activity against phage T7. (FIG. 23C) Plasmid depletion assay for the target recognition specificity of the EcAvs4 chimera in comparison with EcAvs4.

FIG. 24A-24E—Phage-encoded genes inhibit Avs activity. (FIG. 24A) Schematic of a pooled screen in E. coli for phage early genes that rescue Avs-mediated toxicity. CmR, chloramphenicol resistance gene. (FIG. 24B) Deep sequencing readout of anti-defense candidate genes co-expressed with SeAvs3, EcAvs4, or KpAvs4. (FIG. 24C) A hypervariable early gene locus within a closely related set of wastewater-isolated Autographiviridae phages contains abundant anti-defense genes. The tree was constructed from a concatenated alignment of conserved proteins present in all ten phages. Greyscale represents groups of proteins clustered at 40% sequence identity at 70% coverage. (FIG. 24D) Agarose gel analysis showing in vitro reconstitution of anti-SeAvs3 activity by three antidefense candidates. (FIG. 24E) Schematic of the mechanism of Avs proteins as antiphage pattern-recognition receptors.

FIG. 25A-25B—Related to FIG. 24A-24E. Antidefense genes inhibit Avs activity in bacterial cells. Plaque assays against (FIG. 25A) phage ZL19 and (FIG. 25B) phage T7 with E. coli strain C containing both an Avs plasmid and an antidefense plasmid. Antidefense genes were expressed under the control of a J23105 promoter. Spots correspond to 10-fold dilutions from right to left.

FIG. 26—Mechanism and structures of NLR-like defense proteins in prokaryotes. (Left) Comparison of the domain architectures of 11 representative NLR-like pattern-recognition receptors across four kingdoms of life. Selected structures of activated complexes are shown as examples. T3SS, type 3 secretion system. (Right) Defense mechanism of Avs proteins in bacteria and archaea (this study). Target binding triggers the formation of Avs tetramers, which activates an N-terminal effector that disrupts the viral life cycle.

FIG. 27A-27N—Cryo-EM structures of SeAvs3 and EcAvs4 in complex with their cognate triggers. (FIG. 27A-27B) Structure of the SeAvs3-terminase complex. (FIG. 27C-27D) Structure of the EcAvs4-portal complex. (FIG. 27E-27F) ATP molecule in the STAND ATPase active site of EcAvs4 and SeAvs3. The cryo-EM density is shown as a transparent surface. (FIG. 27G) SeAvs3 Cap4-like nuclease effector domain. (FIG. 27H-27I) Active sites for the inward- and outward-facing protomers of the SeAvs3 Cap4-like nuclease. (FIG. 27J) Equivalent view of the active site of HindIII bound to target DNA with two divalent metal ions [Protein Data Bank (PDB) ID 3A4K]. (FIG. 27K) Electrostatic surface potential for the SeAvs3 Cap4-like nuclease and the EcAvs4 Mrr-like nuclease. Active sites are indicated by purple circles. Ideal B-form DNA is modeled on both surfaces based on the crystal structure of Hind III bound to its target (PDB ID 3A4K). (FIG. 27L) EcAvs4 Mrr-like nuclease effector domain. (FIG. 27M-27N) Active sites for the inward- and outward-facing protomers of the EcAvs4 Mrr-like nuclease.

FIG. 28A-28I—Structural basis for viral-fold recognition by SeAvs3 and EcAvs4. (FIG. 28A) The interface between SeAvs3 and the PhiV-1 terminase. An SeAvs3 surface view is shown in transparency. SeAvs3 is colored from the N to C terminus according to the key. (FIG. 28B) AlphaFold or crystal structures of different terminases modeled into SeAvs3. The ATPase and nuclease domains were individually aligned to the PhiV-1 terminase domains. (FIG. 28C-28D) Recognition of the PhiV-1 terminase ATPase and nuclease active sites by the SeAvs3 TPR domain. (FIG. 28E) Sequence logos for terminase ATPase Walker A motifs and terminase nuclease active sites. A total of 11,000 terminase sequences were clustered at 30% sequence identity, and motifs were extracted from clusters containing terminases targeted or not targeted by SeAvs3 according to FIG. 7B (see also FIG. 34). (FIG. 28F) Plasmid depletion assay for SeAvs3 co-expressed in E. coli with a terminase ATPase or nuclease domain harboring active-site mutations. (FIG. 28G) The interface between EcAvs4 and the PhiV-1 portal. An EcAvs4 surface view is shown in transparency. EcAvs4 is colored from the N to C terminus according to the key. (FIG. 28H) b-sheet augmentation between EcAvs4 and the portal clip domain. (FIG. 28I) Comparison of the EcAvs4-bound state of the PhiV-1 portal, the cryo-EM structure of the highly homologous T7 portal in its native virion, and AlphaFold models of diverse portals. A top view of the assembled dodecamer of the T7 portal is also shown.

FIG. 29A-29F—Imaging Avs proteins by electron microscopy. (FIG. 29A) Example cryo-EM micrograph of assembled SeAvs3-gp19 complex. (FIG. 29B) Representative 2D class averages of SeAvs3-gp19 from 128,500 automatically picked particles. (FIG. 29C)2D averages from cryo-EM imaging of SeAvs3 alone. One class is shown magnified with the structure of SeAvs3 residues 655-2087 superimposed, based on the structure of the SeAvs3-gp19 complex. This dataset did not allow high resolution structure determination, potentially due to inherent flexibility in apo-SeAvs3. (FIG. 29D) Example cryo-EM micrograph of purified EcAvs4-gp8 complex. (FIG. 29E) Representative 2D class averages of the tetrameric and octameric species of EcAvs4-gp8 from 444,626 automatically picked particles. Also shown are 2D averages from a small screening cryo-EM dataset from the same sample diluted 2-fold, showing only the tetrameric species. (FIG. 29F) Avs samples imaged by negative-stain electron microscopy using an FEI Tecnai 12 microscope operated at 120 keV. Samples were applied to continuous carbon and stained using 2% uranyl formate. Avs3 and Avs4 do not assemble into tetramers in the absence of their cognate ligands.

FIG. 30A-30B—Cryo-EM data processing scheme. Flowchart outlining the data processing for (FIG. 30A) the SeAvs3-gp19 complex and (FIG. 30B) the EcAvs4-gp8 complex. Final maps deposited to the EMDB are highlighted.

FIG. 31A-31C—Cryo-EM data statistics. (FIG. 31A-31B) Orientation distributions for reconstructions of the SeAvs3-gp19 terminase complex and EcAvs4-gp8 complex. The range of the x-axis, corresponding to the RELION metadata parameter ‘rlnAngleRot,’ is set according to the symmetry of the reconstruction. (FIG. 31C) Gold-standard Fourier-Shell Correlation curves.

FIG. 32A-32C—Cryo-EM map quality and map-to-model fitting. (FIG. 32A) Cryo-EM densities colored by local resolution as calculated within RELION. The overall maps are shown filtered by local resolution, while the focus-refined maps are shown auto-sharpened. Sharpened maps are also shown just around the phage ligands. (FIG. 32B) Map-to-model Fourier-Shell Correlation as calculated in PHENIX, softly masking each map around the fitted model. (FIG. 32C) Example cryo-EM densities for different parts of the structures.

FIG. 33A-33B—Comparison of activated STAND structures. (FIG. 33A) STAND oligomers from different domains of life (29, 30, 47, 76-79), shaded by function. The ROQ1 resistosome structure is a composite by imposing C4 symmetry on PDB 7JLU and merging it with PDB 7JLV and 7JLX (Martin et al., Science 370, eabd9993 (2020)). The NAIP inflammasome structure is an alignment of the C11 symmetric NLRC4 oligomer (with four subunits hidden) (Zhang et al., Science 350, 404-409 (2015)) with the NAIP-NLRC4-flagellin filament structure (Tenthorey et al., Science 358, 888-893 (2017)). (FIG. 33B) Two adjacent STAND ATPase domains from these structures, aligned on the nucleotide-binding domain of one ATPase (blue, as represented in greyscale), showing different relative positions of the adjacent ATPase. NBD; nucleotide-binding domain. HD1; helical domain 1. WHD; winged-helix domain.

FIG. 34—Related to FIG. 28A-28I. Weblogos of the Walker A motifs of phage terminases. Each motif represents a cluster of terminases that contain at least one representative that was tested experimentally in this study. Terminase sequences (Esterman et al., Virus Evol. 7, veab015 (2021)) were supplemented with the 24 terminases in this study and clustered at 30% sequence identity. Clusters containing terminases that do not activate SeAvs3 are shown in red. The UPGMA tree was built using a procedure described previously (Makarova et al., Nat. Rev. Microbiol. 18, 67-83 (2020)).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Bacteria and archaea have evolved numerous defense mechanisms against viral infections involving a wide range of strategies and enzymatic activities (Makarova et al. J. Bacteriol. 193, 6039-6056 (2011); Makarova et al., Annu. Rev. Microbiol. 71, 233-261 (2017); Doron et al., Science. 359 (2018), doi:10.1126/science.aar4120; and Gao et al., Science. 369, 1077-1084 (2020)). Defense systems are activated by viral nucleic acids, in the case of restriction-modification and CRISPR-Cas systems; or by different types of infection-induced cellular stress, including DNA double-strand breaks (Klainman et al., Nucleic Acids Res. 42, 328-339 (2014)), inhibition of host transcription (Guegler et al., Mol. Cell. 81, 2361-2373.e9 (2021)), cytosolic nucleotide depletion (Cheng et al., Nucleic Acids Res. 49, 5216-5229 (2021)), and the disruption of translation elongation factor EF-Tu (Bingham et al., J. Biol. Chem. 275, 23219-23226 (2000)) or RecBCD repair nuclease (Millman et al., Y. Oppenheimer-Shaanan, R. Sorek, Bacterial Retrons Function In Anti-Phage Defense. Cell. 183, 1551-1561.e12 (2020)). Alternatively, some systems constitutively synthesize small molecules that interfere with phage replication (Kronheim et al., Nature. 564 (2018) 283-286; Bernheim et al., Nature. 589, 120-124 (2021)). However, for numerous defense systems, the mechanisms of activation remain uncharacterized, and it appears likely that distinct modes of activation exist within the diverse repertoire of recently discovered systems (Makarova et al. J. Bacteriol. 193, 6039-6056 (2011); Doron et al., Science. 359 (2018), doi:10.1126/science.aar4120; Gao et al., Science. 369, 1077-1084 (2020); Makarova et al., Nucleic Acids Res. 41, 4360-4377 (2013)). NLRs are among the key proteins involved in immunity, cell signaling, and particularly programmed cell death in eukaryotes (Koonin et al., Cell Death Differ. 9, 394-404 (2002); Leipe et al., J. Mol. Biol. 343, 1-28 (2004); Zhao et al., Nature. 477, 596-600, (2011); Kofoed et al., Nature. 477. 592-595 (2011); Caruso et al., Immunity. 41, 898-908 (2014); Jones et al., Science. 354 (2016), doi:10.112/science.aaf6395; Heller et al. Proc. Natl. Acad. Scie. U.S.A. 115, E2292-E2301 (2018); Bauernfried et al., Science. 371 (2021), doi:10.1126/science.abd0811). In animal and plant innate immunity, these proteins function by recognizing pathogen-associated molecular patterns (PAMPs). Animal NLRs consist of a central STAND NTPase domain, a C-terminal region containing leucine-rich repeats (LRRs) or WD40 repeats, and in many cases, an N-terminal pyrin domain or caspase activation and recruitment domain (CARD). Similarly, plant NLRs contain the STAND domain, a C-terminal LRR array and often an N-terminal TIR (Toll/interleukin-1 receptor) domain. The diverse eukaryotic NLRs recognize many different PAMPs; for example, animal NOD1 and NOD2 proteins recognize peptidoglycan fragments from the bacterial cell wall (Caruso et al., Immunity. 41, 898-908 (2014)), NLRP1 binds viral dsRNA (Bauernfried et al., Science. 371 (2021), doi:10.1126/science.abd0811), and NAIP detects bacterial flagellin and type 3 secretion systems (Zhao et al., Nature. 477, 596-600, (2011); Kofoed et al., Nature. 477. 592-595 (2011)). In all of these cases, recognition of the PAMP leads to oligomerization of the NLR and recruitment of effector proteins. Bacteria and archaea, especially those with complex signaling systems, also encode a diverse repertoire of STAND NTPases that are predicted to be involved in signal transduction and possibly in programmed cell death (Koonin et al., Cell Death Differ. 9, 394-404 (2002); Leipe et al., J. Mol. Biol. 343, 1-28 (2004)). However, the functions of these proteins are largely unknown, with the exception of several that have been characterized as transcription regulators (Danot et al., Proc. Natl. Acad. Sci. U.S.A 98, 435-440 (2001); Horinouchi et al. Gene. 95, 49-56 (1990); Ye et al., Microbiol. Mol. Biol. Rev. 84 (2020), doi:10.1128/MMBR.00061-19).

Applicant demonstrates herein that antiviral STAND (Avs) homologs in bacteria and archaea are pattern recognition receptors that detect conserved viral proteins and activate diverse N-terminal effectors, including DNA endonucleases. This work further reveals remarkable similarity between the defense strategies of prokaryotes and eukaryotes and extends the paradigm of pattern recognition of pathogen-specific proteins across all domains of life. Embodiments disclosed herein provide programmable pattern recognition proteins that are capable of recognizing and binding a molecular pattern. The programmable pattern recognition proteins can have one or more effector domains that can be activated upon pattern recognition. In this way, the programmable pattern recognition proteins can be engineered to specifically recognize a target pattern (i.e., programmed), which can lead to effector activity at or in proximity to the recognized target pattern. Combining different pattern recognition capabilities with different effector functions can provide, without limitation, a modular system with a myriad of utilities such as molecular pattern-based in vitro diagnostics, cargo delivery, therapeutic applications, and microbiome structure engineering. Other embodiments, applications, and uses are described herein and will be appreciated in view of the present exemplary embodiments and working examples herein.

### Programmable Pattern Recognition Proteins

Described in several example embodiments herein are engineered programmable pattern recognition proteins. The programmable pattern recognition proteins comprise an effector domain, an effector activation domain, and a pattern recognition domain, wherein binding of the recognition domain to a target molecule leads to activation of the effector domain, and wherein at least one of the effector domains, effector activation domain, or pattern recognition domain is derived from a Signal Transduction ATPases with Numerous-associated Domains (STAND) protein. In one example embodiment, the engineered protein comprises a STAND NTPase. In one example embodiment the STAND NTPase functions as the effector activation domain and further comprises an effector domain and a pattern recognition domain derived from the same STAND protein or from an ortholog or homolog thereof. The effector domain may also be a non-STAND effector domain.

Generally, and without being bound by theory, upon pattern recognition by the engineered protein, the engineered protein is activated. Activation can include activating the STAND NTPase and/or other effector domains of the engineered protein. In an embodiment, the activity of the engineered protein includes nuclease and/or protease activity. Thus, when activated in response to pattern recognition, such as a PAMP or other molecular pattern associated with a target cell or molecule (e.g, a target polypeptide), the engineered protein can have effector function (e.g., nuclease, protease, etc. activity) at the target molecule and/or cell. In an embodiment, such effector function can lead to cell death or cell or molecule modification. Other functions and activities will be appreciated in view of the description herein.

In an embodiment, the engineered protein has a central STAND NTPase that is flanked by an N-terminal region and/or a C-terminal region. In an embodiment, the N-terminal region has one or more effector domains. In an embodiment, the C-terminal domain comprises one or more structural and/or interaction motifs.

In an embodiment, the STAND NTPase, the N-terminal region, and/or the C-terminal region can be engineered such that the engineered protein recognizes a specific molecular pattern, has a specific desired effector function in addition to any effector function of the STAND NTPase, and/or has specific interaction capabilities beyond molecular pattern recognition and/or interaction. Without being bound by theory, the protein compositions of the present invention provide the ability to have a modular and programmable composition in which molecular pattern recognition, effector functionality and effector activation can be configured so as to target a particular cell or molecule comprising or otherwise associated with a target molecular pattern and provide a desired effector action at the targeted cell or molecule.

### Effector Domains

In an embodiment, the engineered protein composition of the present invention comprises one or more effector domains. In an embodiment one or more effector domains are derived from a STAND protein. In an embodiment one or more effector domains are derived from a STAND NTPase protein. In an embodiment one or more effector domains are derived from a prokaryotic STAND protein. In an embodiment, one or more effector domains are derived from a prokaryotic STAND NTPase. STAND proteins and STAND NTPase proteins are discussed and described in greater detail elsewhere herein.

In an embodiment, one or more effector domains are not derived from a STAND protein and/or STAND NTPase.

In an embodiment, the N-terminal region, the C-terminal region, or both the N- and the C-terminal regions of the engineered protein comprises the one or more effector domains. In an embodiment, one or more of the effector domains are contained between the N-terminal region and the C-terminal region of the engineered protein.

In an embodiment, the one or more effector domains are independently selected from a nuclease, a nickase, a protease or peptidase, nucleosidase, a helicase, a methylase, an acetylase, a demethylase, a deacetylase, a transcriptase, a hydrolase, a phosphatase, a phosphorylase, a caspase or caspase like domain, a glycosylase, a lipase, a transferase, any combination thereof, and/or the like.

### Exemplary Effector Domains

Exemplary nucleases include, without limitation, Cas proteins and systems (see e.g, Koonin and Makarova et al., Origins and evolution of CRISPR-Cas systems. Phil. Trans R. Soc. B3742018008720180087 and Makarova et al., CRISPR J. 2018 Oct. 1; 1(5): 325-336). In an embodiment, the Cas is a Cas having collateral nuclease activity (e.g, a Cas12 or a Cas13). In an embodiment, the Cas is a Cas nickase or dead Cas. In an embodiment, the nuclease is a single stranded DNA (ssDNA) nuclease. In an embodiment, the nuclease is a dsDNA nuclease. In an embodiment, the nuclease is an exonuclease. In an embodiment, the nuclease is an endonuclease. In an embodiment, the nuclease is a circular DNA nuclease. In an embodiment, the nuclease is a linear nuclease. In an embodiment, the nuclease is an RNA nuclease. In an embodiment, one or more effector domains comprise one or more PD-DExK-family nuclease domains. In an embodiment, the nuclease activity is organism and phage independent.

Exemplary proteases include without limitation, aspartic, glutamic, and metalloproteases, cysteine, serine, and threonine proteases. In an embodiment, the protease/peptidase comprises a TPR and/or CHAT domain. In an embodiment, the protease comprises or is a caspase or caspase like protein or functional domain thereof. In an embodiment, the protease is a bacterial protease or functional domain thereof (See e.g., Culp and Wright. J. Antibiotics. 70:366-377 (2017)). In an embodiment, the protease is a eukaryotic protease or a functional domain thereof (see e.g., Quesada et al. Nuc. Acid. Res. 2009 37:D239-D243).

In an embodiment, an effector domain comprises nuclease, protease, nucleosidase, sirtunins (SIR2), Toll/interleukin-1 receptor homology (TIR), cytidine monophosphate (CMP) hydrolase and/or caspase-like enzyme activities. In an embodiment, the effector domain comprises dsDNA nuclease activity. In an embodiment, the effector domain comprises circular DNA and/or linear DNA nuclease activity. In an embodiment, the effector is SIR2, TIR, or a CMP hydrolase.

In an embodiment, the one or more effector domains comprise one or more D-QxK and/or one or more E-Q-QxK catalytic motifs.

### Effector Domain Targets

The target of an effector domain of the engineered programmable pattern recognition protein composition of the present invention can be any target comprising, be fused to, linked to, tethered to, coupled to, or otherwise integrated or associated with a target polypeptide and/or target molecular pattern, optionally a PAMP, that is recognized by the engineered protein composition of the present invention. In an embodiment, the target is a cell. In an embodiment the target is a polypeptide or peptide. In an embodiment, the target is a nucleic acid (e.g., DNA or RNA). In an embodiment the target is a double stranded (ds) nucleic acid, such as dsRNA or dsDNA. In an embodiment the target is a circular DNA.

In an embodiment, an effector domain acts on the same molecule that contains or is fused to, linked to, tethered to, coupled to, or otherwise integrated or associated with the target polypeptide, target molecule, and/or target molecular pattern recognized and/or bound by the engineered protein composition of the present invention. In an embodiment, an effector domain acts on a molecule that does not contain or is not fused to, linked to, tethered to, coupled to, or otherwise integrated or associated with the target polypeptide, target molecule, and/or target molecular pattern recognized and/or bound by the engineered protein composition of the present invention. For example, the recognition/binding activity may be used to target the engineered protein composition of the present invention to a specific cell but that the effector function may be carried out on a component of that cell, such as a protein or nucleic acid within the targeted cell not directly containing the target polypeptide, target molecule, and/or target molecular pattern. In another example, the recognition activity may be used to target the engineered protein composition of the present invention to a specific region in an organism, on a device or substrate, such as a region on a microfluidic chip, lateral flow device, or region within an organism. In another example, the effector domains (s) of the engineered composition then may take effect on any substrate molecule (with or without a target polypeptide, target molecule, and/or target molecular pattern) that is within effective proximity of the engineered protein composition of the present invention.

### Effector Activation Domains

The engineered proteins can contain an effector activation domain. Without being bound by theory, the effector activation domain can interact with the recognition domain, target molecule, and/or the effector domain such that the effector domain is activated. In an embodiment, the effector activation domain is or is derived from a STAND protein. In an embodiment, the effector activation is or is derived from a STAND NTPase protein. In an embodiment, the effector activation is or is derived from a prokaryotic STAND protein. In an embodiment, effector activation is or is derived from a prokaryotic STAND NTPase. STAND proteins and STAND NTPAse proteins are discussed and described in greater detail elsewhere herein. In an embodiment, the effector activation domain

In an embodiment, the N-terminal region, the C-terminal region, or both the N- and the C-terminal regions of the engineered protein comprises the effector activation domain or component thereof. In an embodiment, an effector activation domain is contained between the N-terminal region and the C-terminal region of the engineered protein.

### Pattern Recognition Domains

The engineered proteins contain a pattern recognition domain, which is also referred to herein as a “recognition domain”. In an embodiment, the recognition domain is capable of recognizing and/or binding a target polypeptide, such as once comprising a specific molecular pattern. Exemplary molecular patterns include 2-D and 3D structures. A non-limiting example of a molecular pattern are pathogen-associated molecular patterns, which are described in further detail below. In an embodiment, the recognition domain contains one or more tetratricopeptide repeat (TPR) domains.

In an embodiment, the recognition domain or portion thereof is in the N terminal region, C-terminal region, or both of the engineered protein of the present invention. In an embodiment, the recognition domain is contained between the N-terminal region and the C-terminal region of the engineered protein.

In an embodiment, the recognition domain recognizes a native target polypeptide and/or molecular pattern of wild-type STAND protein. In an embodiment, the recognition domain recognizes a native target polypeptide and/or molecular pattern of wild-type prokaryotic STAND protein. In an embodiment, the recognition domain recognizes a native target polypeptide and/or molecular pattern of wild-type STAND NTPase protein. In an embodiment, the recognition domain recognizes a native target polypeptide and/or molecular pattern of wild-type prokaryotic STAND NTPase protein. In an embodiment, the recognition domain targets a PAMP recognized by a wild-type STAND protein. In an embodiment, the recognition domain targets a PAMP recognized by a wild-type STAND NTPase protein, optionally a prokaryotic wild-type STAND protein or STAND NTPase protein.

In an embodiment, the recognition domain is engineered to recognize a target polypeptide and/or molecular pattern other than a native target polypeptide or molecular pattern of a wild-type STAND protein or STAND NTPase protein. In other words, the recognition domain can be engineered to recognize a target polypeptide and/or molecular pattern that is not a native recognition partner (or target) to a wild-type STAND protein or wild-type STAND NTPase protein. In an embodiment, the recognition domain recognizes a target polypeptide and/or molecular pattern that is not a native recognition partner (or target) to a wild-type STAND protein or STAND NTPase. In an embodiment, the recognizes a PAMP that is not a native PAMP for a wild-type STAND protein or STAND NTPase. In an embodiment, the recognizes a PAMP that is not a native PAMP for a wild-type prokaryotic STAND protein or STAND NTPase.

In an embodiment, the recognition domain is derived from a STAND protein. In an embodiment, recognition domain is derived from a STAND NTPase protein. In an embodiment, the recognition domain is derived from a prokaryotic STAND protein. In an embodiment, the recognition domain is derived from a prokaryotic STAND NTPase. STAND proteins and STAND NTPase proteins are discussed and described in greater detail elsewhere herein.

### Pathogen-associated Molecular Pattern (PAMP)

PAMPs are known in the art as molecular motifs that form structural “patterns” whose structure is recognized by receptors and proteins. The term originated from the observation that classes of microbes, particularly pathogenic microbes, contained structural motifs that were recognized by cell receptors that stimulated the immune response. Although the term originated from the study of host-pathogen interaction, it will be appreciated that in the context of the present invention PAMPs are not limited to those relating to pathogenic cells or molecules. As previously mentioned, the engineered protein compositions have molecular pattern recognition activity. In an embodiment, the engineered protein compositions of the present invention have PAMP recognition activity. In other words, in an embodiment the engineered proteins of the present invention can recognize PAMPs. Without being bound by theory, by engineering the protein composition to recognize particular PAMPs, the targets of the protein can be specified. Further, by incorporating different PAMPs target molecule specificity of protein can be engineered to recognize different target molecules. Thus, in an embodiment, the PAMPs recognized by the engineered proteins of the present invention may be native to a target cell or molecule or may be exogenous to the target cell or molecule. Where the PAMPs are exogenous to a target cell or molecule, the PAMPs may be fused to, linked to, tethered to, coupled to, or otherwise integrated or associated with the target cell or molecule.

In an embodiment, the PAMPs are proteins, peptides, sugars or other carbohydrates, lipopolysaccharides, peptidoglycans, nucleic acids (particularly double stranded variants), and/or the like. It will be appreciated that although PAMPs are traditionally thought of as being associated with pathogens, that PAMPs may also be found or associated with non-pathogenic organisms or cells.

In an embodiment the PAMP recognized by the engineered protein of the present invention is a large terminase subunit. In an embodiment, the PAMP recognized by the engineered protein present invention is a large terminase subunit of a virus or phage. In an embodiment, the PAMP recognized by the engineered protein of the present invention is gp19 or a structural homologue thereof. In an embodiment, the PAMP recognized by the engineered protein of the present invention is a portal protein. In an embodiment, the portal protein is a viral or a phage portal protein. In an embodiment, the PAMP recognized by the engineered protein of the present invention is a gp8 portal protein or a structural homologue thereof. Exemplary terminase and portal proteins are shown in Table 1. In an embodiment, the PAMP recognized by the engineered protein is or comprises an ATPase domain or portion thereof or a 3-D structural feature thereof and/or a nuclease domain or a portion thereof or 3-D structural feature thereof of a large terminase subunit. In an embodiment, the PAMP recognized by the engineered protein is or comprises an ATPase domain or portion thereof, or a 3-D structural feature thereof and/or a nuclease domain or a portion thereof or 3-D structural feature thereof of a gp9 protein or a structural homologue thereof. In an embodiment, the PAMP recognized by the engineered protein is or comprises an portal protein or portion thereof, or a 3-D structural feature thereof. In an embodiment, the PAMP recognized by the engineered protein is or comprises a gp8 portal protein or structural homologue thereof, a portion thereof or a 3-D structural feature thereof.

TABLE 1
Phage terminases and portals tested.

| # | Phage | Family | Gene | Accession | Amino Acids | Codon | Note |
|---|---|---|---|---|---|---|---|
| 1 | T3 | Autographiviridae | Terminase | NP_523347.1 | 586 | Native | |
| 1 | T3 | Autographiviridae | Portal | YP_009792958.1 | 535 | Native | |
| 2 | T7 | Autographiviridae | Terminase | NP_042010.1 | 586 | E. coli | |
| 2 | T7 | Autographiviridae | Portal | NP_041995.1 | 536 | E. coli | |
| 3 | PhiV-1 | Autographiviridae | Terminase | QLF85798.1 | 586 | E. coli | |
| 3 | PhiV-1 | Autographiviridae | Portal | QLF85783.1 | 535 | E. coli | |
| 4 | Pisces | Autographiviridae | Terminase | QEG09602.1 | 587 | E. coli | |
| 4 | Pisces | Autographiviridae | Portal | QEG09587.1 | 522 | E. coli | |
| 5 | RsoP1EGY | Autographiviridae | Terminase | YP_009799710.1 | 561 | E. coli | Ralstonia phage |
| | | | | | | | RsoP1EGY |
| 5 | RsoP1EGY | Autographiviridae | Portal | YP_009799742.1 | 519 | E. coli | Ralstonia phage |
| | | | | | | | RsoP1EGY |
| 6 | Uncultured | Autographiviridae | Terminase | CAB4140543.1 | 551 | E. coli | Uncultured |
| | | | | | | | Caudovirales |
| | | | | | | | phage |
| 6 | Uncultured | Autographiviridae | Portal | CAB4140338.1 | 518 | E. coli | Uncultured |
| | | | | | | | Caudovirales |
| | | | | | | | phage |
| 7 | S. flava* | Autographiviridae | Terminase | WP_079638097.1 | 589 | E. coli | Sphingopyxis |
| | | | | | | | flava R11H |
| | | | | | | | (prophage) |
| 7 | S. flava* | Autographiviridae | Portal | WP_079638083.1 | 520 | E. coli | Sphingopyxis |
| | | | | | | | flava R11H |
| | | | | | | | (prophage) |
| 8 | D. archaeon* | Autographiviridae | Terminase | OYT57761.1 | 559 | E. coli | Desulfurococcales |
| | | | | | | | archaeon |
| | | | | | | | ex4484_217_2 |
| | | | | | | | (prophage) |
| 8 | D. archaeon* | Autographiviridae | Portal | OYT57739.1 | 533 | E. coli | Desulfurococcales |
| | | | | | | | archaeon |
| | | | | | | | ex4484_217_2 |
| | | | | | | | (prophage) |
| 9 | SP6 | Autographiviridae | Terminase | NP_853601.1 | 631 | E. coli | |
| 9 | SP6 | Autographiviridae | Portal | NP_853590.1 | 515 | E. coli | |
| 10 | ECBP5 | Autographiviridae | Terminase | YP_009146419.1 | 616 | E. coli | |
| 10 | ECBP5 | Autographiviridae | Portal | YP_009146408.1 | 530 | E. coli | |
| 11 | PGT2 | Autographiviridae | Terminase | YP_009795474.1 | 602 | E. coli | |
| 11 | PGT2 | Autographiviridae | Portal | YP_009795464.1 | 517 | E. coli | |
| 12 | Minorna | Autographiviridae | Terminase | YP_009820994.1 | 618 | E. coli | |
| 12 | Minorna | Autographiviridae | Portal | YP_009821007.1 | 531 | E. coli | |
| 13 | Lidtsur | Autographiviridae | Terminase | YP_009821647.1 | 580 | E. coli | |
| 13 | Lidtsur | Autographiviridae | Portal | YP_009821637.1 | 521 | E. coli | |
| 14 | E. coli-1* | Unknown | Terminase | WP_001619041.1 | 536 | E. coli | E. coli NCTC9020 |
| | | | | | | | (prophage) |
| 14 | E. coli-1* | Unknown | Portal | WP_059328803.1 | 541 | E. coli | E. coli NCTC9020 |
| | | | | | | | (prophage) |
| 15 | N4 | Schitoviridae | Terminase | YP_950546.1 | 530 | E. coli | |
| 15 | N4 | Schitoviridae | Portal | YP_950537.1 | 763 | E. coli | |
| 16 | ZL19 | Drexlerviridae | Terminase | YP_009789200.1 | 521 | Native | |
| 16 | ZL19 | Drexlerviridae | Portal | | 422 | Native | |
| 17 | ST32 | Chaseviridae | Terminase | YP_009790711.1 | 672 | E. coli | |
| 17 | ST32 | Chaseviridae | Portal | YP_009790713.1 | 438 | E. coli | |
| 18 | E. coli-2* | Podoviridae | Terminase | WP_000132532.1 | 489 | E. coli | E. coli M885 |
| | | | | | | | (prophage) |
| 18 | E. coli-2* | Podoviridae | Portal | WP_000852419.1 | 559 | E. coli | E. coli M885 |
| | | | | | | | (prophage) |
| 19 | P1 | Myoviridae | Terminase | WP_000124150.1 | 494 | Native | |
| 19 | P1 | Myoviridae | Portal | | 569 | Native | |
| 20 | Lambda | Siphoviridae | Terminase | NP_040581.1 | 641 | Native | |
| 20 | Lambda | Siphoviridae | Portal | | 512 | Native | Mature version |
| | | | | | | | following |
| | | | | | | | proteolytic |
| | | | | | | | cleavage |
| 21 | P22 | Podoviridae | Terminase | WP_015975189.1 | 499 | E. coli | |
| 21 | P22 | Podoviridae | Portal | WP_015975195.1 | 725 | E. coli | |
| 22 | T5 | Demerecviridae | Terminase | YP_006983.1 | 438 | Native | |
| 22 | T5 | Demerecviridae | Portal | | 394 | Native | Mature version |
| | | | | | | | following |
| | | | | | | | proteolytic |
| | | | | | | | cleavage |
| 23 | CBA120 | Ackermannviridae | Terminase | YP_004957856.1 | 736 | E. coli | |
| 23 | CBA120 | Ackermannviridae | Portal | YP_004957852.1 | 560 | E. coli | |
| 24 | T4 | Myoviridae | Terminase | NP_049776.1 | 610 | Native | |
| 24 | T4 | Myoviridae | Portal | WP_015969330.1 | 524 | Native | |

See also Data S3 of Gao et al., Science 377, eabm4096 (2022), which is incorporated herein by reference as if expressed in its entirety herein.

### STAND NTPases

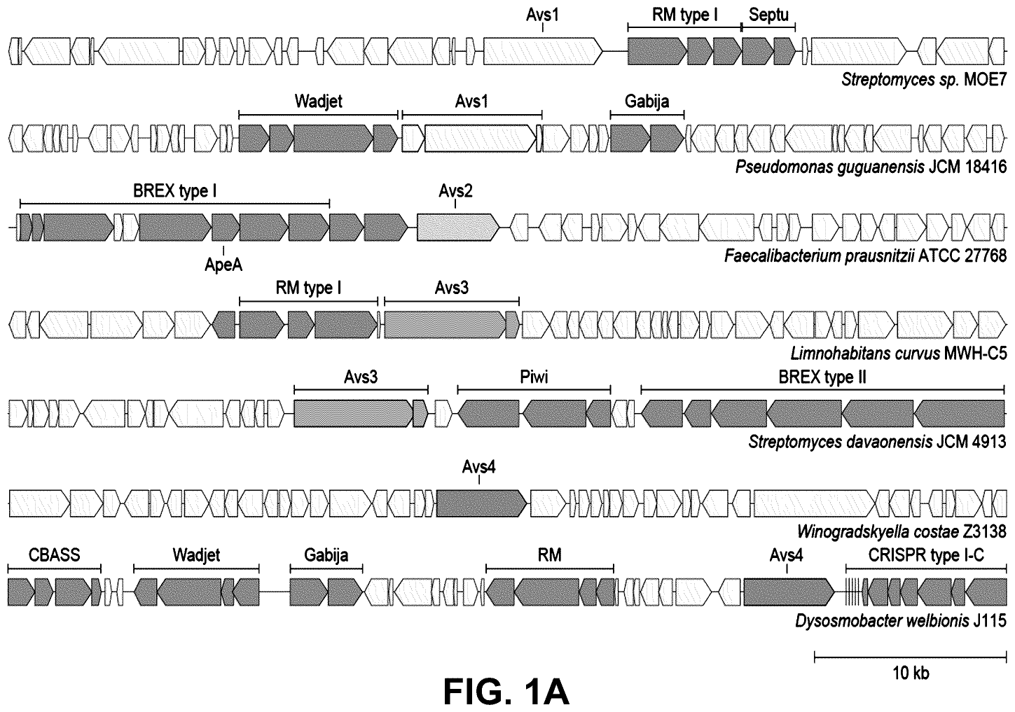

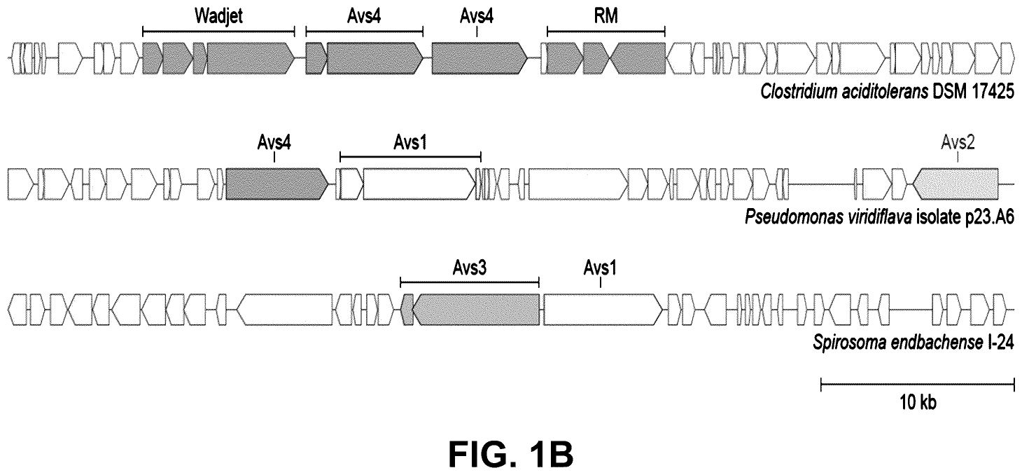

As discussed elsewhere herein, the engineered protein composition comprises a STAND protein or component thereof. In an embodiment, the STAND protein or component thereof is a STAND NTPase. In an embodiment, the engineered protein comprises components derived from an Avs (anti-viral STAND) or a homolog thereof. In an embodiment, the STAND NTPase is an Avs NTPase. In an embodiment, the Avs comprises Avs1-4 protein families as shown in FIG. 1A. In an embodiment, the Avs is an Avs1, Avs2, Avs3, or Avs4 protein or a protein from an Avs1, Avs2, Avs3, Avs4 protein family as shown in FIG. 1A. In an embodiment, the Avs protein or homologs thereof further comprise an N-terminal effector domain, and a PANT recognition region comprising a central core region and a C-terminal tetratricopeptide repeat (TPR) domain. In an embodiment the PAMP recognition region comprising the central core region is or comprises a STAND NTPase.

In an embodiment, the engineered protein comprises a protein, a STAND Protein, STAND NTPase, Avs or homologue thereof that is 80-100 percent identical to any a protein, a STAND Protein, STAND NTPase, Avs or homologue thereof of any one or more of Tables 2, 3, 4, 5, 6, and 7. In an embodiment, the engineered protein comprises a protein, a STAND Protein, STAND NTPase, Avs or homologue thereof that is 80% to/or 81%, 82%, 83%, 84%, 85%, 86%, 87%, 88%, 89%, 90%, 91%, 92%, 93%, 94%, 95%, 96%, 97%, 98%, 99%, 100% identical to a protein, a STAND Protein, STAND NTPase, Avs or homologue thereof of any one or more of Tables 2, 3, 4, 5, 6, and 7 or is a homolog thereof. In an embodiment, the protein, STAND Protein, STAND NTPase, Avs, or homologue thereof is from an organism having a genome as in Data S8 of Gao et al. “Prokaryotic innate immunity via pattern recognition of conserved viral proteins,” Science, 377, eabm4096 (2022), which is incorporated by reference as if expressed in its entirety herein.

In an embodiment, the Avs1-4 recognize PAMPs in phage proteins such as gp19, a large terminase subunit, and gp8, a portal protein. Other target PAMPs are described in greater detail elsewhere herein. In an embodiment, Avs1-3 recognize PAMPs in gp19, and Avs4, recognize PAMPs in gp8. Other target PAMPs are described in greater detail elsewhere herein.

Lengthy table referenced here
US20250243471A1-20250731-T00001
Please refer to the end of the specification for access instructions.

Lengthy table referenced here
US20250243471A1-20250731-T00002
Please refer to the end of the specification for access instructions.

Lengthy table referenced here
US20250243471A1-20250731-T00003
Please refer to the end of the specification for access instructions.

Lengthy table referenced here
US20250243471A1-20250731-T00004
Please refer to the end of the specification for access instructions.

Lengthy table referenced here
US20250243471A1-20250731-T00005
Please refer to the end of the specification for access instructions.

### Other Exemplary Domains

In an embodiment, the engineered protein composition comprises one or more other domains. In an embodiment the one or more additional domains are in the N-terminal region, C-terminal region, or both of the engineered protein of the present invention. In an embodiment, the one or more additional domains are contained between the N-terminal region and the C-terminal region of the engineered protein. In an embodiment, the engineered protein of the present invention contains one or more structural motifs or interaction domains. Exemplary structural motifs and/or interaction domains include, without limitation, a TPR domain, a dimerization domain, an oligomerization domain, a signaling domain, and/or the like. Exemplary dimerization domains include, without limitation, zinc finger domains and leucine zipper domains. In an embodiment, one or more of the domains of the engineered proteins of the present invention allow interaction with other proteins, including by not limited to engineered proteins of the present invention, but others as well, such as those present on target cells and engage in cell signaling.

### Engineered Protein Oligomers

In an embodiment, the engineered proteins of the present invention form oligomers. In an embodiment, effector activity and/or activation of one or more engineered proteins includes oligomer formation. Without being bound by theory in these embodiments, activation occurs upon oligomer formation. In an embodiment, oligomer formation involves binding of a pattern recognition domain to a target polypeptide. In an embodiment, the oligomer is a tetramer, a trimer, or a dimer. In an embodiment, the oligomer is heterogeneous (i.e., contains at least two different engineered protein monomers). In an embodiment, at least two engineered protein monomers are different. In an embodiment, each engineered protein monomer is different. In an embodiment, the at least two different engineered protein monomers have different effector domains. In an embodiment, the oligomer is homogenous (i.e., contains all the same engineered protein monomers).

### Polynucleotides and Vectors

Described herein are polynucleotides encoding one or more components (e.g., polypeptides and/or guide polynucleotides) of the programmable pattern recognition proteins, oligomers, or system (such as a detection composition or system) comprising the programmable pattern recognition composition. Also described herein are vectors and vector systems containing one or more programmable pattern recognition protein or system encoding polynucleotides. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

### Polynucleotides

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

### Codon Optimization

In an embodiment, the polynucleotide can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In an embodiment, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The polynucleotide can be codon optimized for expression in a specific cell-type, tissue type, organ type, and/or subject type. In an embodiment, a codon optimized sequence is a sequence optimized for expression in a eukaryote, e.g., humans (i.e. being optimized for expression in a human or human cell), or for another eukaryote, such as another animal (e.g. a mammal or avian) as is described elsewhere herein. Such codon optimized sequences are within the ambit of the ordinary skilled artisan in view of the description herein. In an embodiment, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In an embodiment, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In an embodiment, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In an embodiment, a polynucleotide coding sequence encoding one or more elements of programmable pattern recognition proteins or system described herein is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate.

### Vectors and Vector Systems

Also provided herein are vectors and vector system that can contain one or more of the programmable pattern recognition protein or system polynucleotides (such as an encoding polynucleotide) described herein. In an embodiment, the vector can contain one or more polynucleotides encoding one or more elements of a CRISPR-Cas system described herein. The vectors can be useful in producing bacterial, fungal, yeast, plant cells, animal cells, and transgenic animals that can express one or more components of the programmable pattern recognition protein or system described herein. Within the scope of this disclosure are vectors containing one or more of the polynucleotide sequences described herein. One or more of the polynucleotides that are part of the programmable pattern recognition protein or system described herein can be included in a vector or vector system. The vectors and/or vector systems can be used, for example, to express one or more of the polynucleotides in a cell, such as a producer cell, to produce programmable pattern recognition protein or system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In an embodiment, the vector can be a bicistronic vector. In an embodiment, a bicistronic vector can be used for one or more elements of the programmable pattern recognition protein or system described herein. In an embodiment, expression of elements of the programmable pattern recognition protein or system described herein can be driven by the CBh promoter or other ubiquitous promoter. Where the element of the programmable pattern recognition protein or system is an RNA, its expression can be driven by a Pol III promoter, such as a U6 promoter. In an embodiment, the two are combined.

In an embodiment, a vector capable of delivering an effector protein and optionally at least one guide RNA to a cell can be composed of or contain a minimal promoter operably linked to a polynucleotide sequence encoding the effector protein and a second minimal promoter operably linked to a polynucleotide sequence encoding at least one guide RNA, wherein the length of the vector sequence comprising the minimal promoters and polynucleotide sequences is less than 4.4 Kb. In an embodiment, the vector can be a viral vector. In an embodiment, the viral vector is an is an adeno-associated virus (AAV) or an adenovirus vector.

In an embodiment, the vector capable of delivering a lentiviral vector for an effector protein and at least one guide RNA to a cell can be composed of or contain a promoter operably linked to a polynucleotide sequence encoding a STAND NTPase, a target containing a pattern recognized by the STAND NTPase, an effector and a second promoter operably linked to a polynucleotide sequence encoding at least one guide RNA, wherein the polynucleotide sequences are in reverse orientation.

In one embodiment, the invention provides a vector system comprising one or more vectors. In an embodiment, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the programmable pattern recognition protein or system complex to the one or more target sequence(s) in a eukaryotic cell, wherein the programmable pattern recognition protein or system complex comprises a STAND NTPase polypeptide and/or effector polypeptide complexed with the one or more guide sequence(s) that is hybridized to the one or more target sequence(s); and (b) a second regulatory element operably linked to an enzyme-coding sequence encoding said STAND NTPase polypeptide and/or effector polypeptide, preferably comprising at least one nuclear localization sequence and/or at least one NES; wherein components (a) and (b) are located on the same or different vectors of the system. Where applicable, a tracr sequence may also be provided. In an embodiment, component (a) further comprises two or more guide sequences operably linked to the first regulatory element, wherein when expressed, each of the two or more guide sequences direct sequence specific binding of a programmable pattern recognition protein or system complex to a different target sequence in a eukaryotic cell. In an embodiment, the programmable pattern recognition protein or system complex comprises one or more nuclear localization sequences and/or one or more NES of sufficient strength to drive accumulation of said programmable pattern recognition protein or system complex in a detectable amount in or out of the nucleus of a eukaryotic cell. In an embodiment, the first regulatory element is a polymerase III promoter. In an embodiment, the second regulatory element is a polymerase II promoter. In an embodiment, each of the guide sequences is at least 16, 17, 18, 19, 20, 25 nucleotides, or between 16-30, or between 16-25, or between 16-20 nucleotides in length.

These and others are further detailed and described elsewhere herein.

### Cell-based Vector Amplification and Expression

Vectors may be introduced and propagated in a prokaryote or prokaryotic cell. In an embodiment, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In an embodiment, a prokaryote is used to amplify copies of a vector and express one or more nucleic acids, such as to provide a source of one or more proteins for delivery to a host cell or host organism.

Vectors can be designed for expression of one or more elements of the programmable pattern recognition protein or system described herein (e.g., nucleic acid transcripts, proteins, enzymes, and combinations thereof) in a suitable host cell. In an embodiment, the suitable host cell is a prokaryotic cell. Suitable host cells include, but are not limited to, bacterial cells, yeast cells, insect cells, and mammalian cells. In an embodiment, the suitable host cell is a eukaryotic cell.

In an embodiment, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In an embodiment, the host cell is a suitable insect cell. Suitable insect cells include those from Spodoptera frugiperda. Suitable strains of S. frugiperda cells include, but are not limited to Sf9 and Sf21. In an embodiment, the host cell is a suitable yeast cell. In an embodiment, the yeast cell can be from Saccharomyces cerevisiae. In an embodiment, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In an embodiment, the vector can be a yeast expression vector. Examples of vectors for expression in yeast Saccharomyces cerevisiae include pYepSecl (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2^{nd} edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2p plasmids, yeast integrative plasmids, yeast replicative plasmids, shuttle vectors, and episomal plasmids.

In an embodiment, the vector is a baculovirus vector or expression vector and can be suitable for expression of polynucleotides and/or proteins in insect cells. In an embodiment, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In an embodiment, the vector is a mammalian expression vector. In an embodiment, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2^{nd} ed., Cold Spring Harbor Laboratory, Cold Spring Harbor Laboratory Press, Cold Spring Harbor, N.Y., 1989.

In an embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In an embodiment, a regulatory element can be operably linked to one or more elements of a CRISPR-Cas system so as to drive expression of the one or more elements of the CRISPR-Cas system described herein.

In an embodiment, the vector can be a fusion vector or fusion expression vector. In an embodiment, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In an embodiment, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In an embodiment, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In an embodiment, one or more vectors driving expression of one or more elements of a programmable pattern recognition proteins or system described herein are introduced into a host cell such that expression of the elements of the engineered delivery system described herein direct formation a programmable pattern recognition protein or system complex at one or more target sites. For example, a programmable pattern recognition protein or system effector protein describe herein and a nucleic acid component (e.g., a guide polynucleotide) can each be operably linked to separate regulatory elements on separate vectors. RNA(s) of different elements of programmable pattern recognition protein or system described herein can be delivered to an animal, plant, microorganism or cell thereof to produce an animal (e.g., a mammal, reptile, avian, etc.), plant, microorganism or cell thereof that constitutively, inducibly, or conditionally expresses different elements of the programmable pattern recognition protein or system described herein that incorporates one or more elements of the programmable pattern recognition protein or system described herein or contains one or more cells that incorporates and/or expresses one or more elements of the programmable pattern recognition protein or system described herein.

In an embodiment, two or more of the elements expressed from the same or different regulatory element(s), can be combined in a single vector, with one or more additional vectors providing any components of the system not included in the first vector. In an embodiment, the specific regulator elements used are chosen to reduce or eliminate regulatory element competition, such as promoter competition. Programmable pattern recognition protein or system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In an embodiment, a single promoter drives expression of a transcript encoding one or more programmable pattern recognition protein or system proteins, embedded within one or more intron sequences (e.g., each in a different intron, two or more in at least one intron, or all in a single intron). In an embodiment, the programmable pattern recognition protein or system polynucleotides can be operably linked to and expressed from the same promoter.

### Cell-Free Vector and Polynucleotide Expression

In an embodiment, the polynucleotide encoding one or more features of the programmable pattern recognition protein or system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In an embodiment, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In an embodiment, the polynucleotides and/or vectors thereof described herein (such as the programmable pattern recognition protein or system polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In an embodiment, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EFlα promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit P-globin (Proc. Natl. Acad. Sci. USA., Vol. 78(3), p. 1527-31, 1981).

In an embodiment, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In an embodiment, the vector can contain a minimal promoter. In an embodiment, the minimal promoter is the Mecp2 promoter, tRNA promoter, or U6. In a further embodiment, the minimal promoter is tissue specific. In an embodiment, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In an embodiment a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In an embodiment, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In an embodiment, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Ferl14), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the CRISPR-Cas system described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In an embodiment, one or more of the programmable pattern recognition protein or system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the programmable pattern recognition protein or system are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include one or more elements of the programmable pattern recognition protein or system described herein, a light-responsive cytochrome heterodimer (e.g., from Arabidopsis thaliana), and a transcriptional activation/repression domain. In an embodiment, the vector can include one or more of the inducible DNA binding proteins provided in International Patent Publication No. WO 2014/018423 and US Patent Publication Nos., 2015/0291966, 2017/0166903, 2019/0203212, which describe e.g., embodiments of inducible DNA binding proteins and methods of use and can be adapted for use with the present invention.

In an embodiment, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

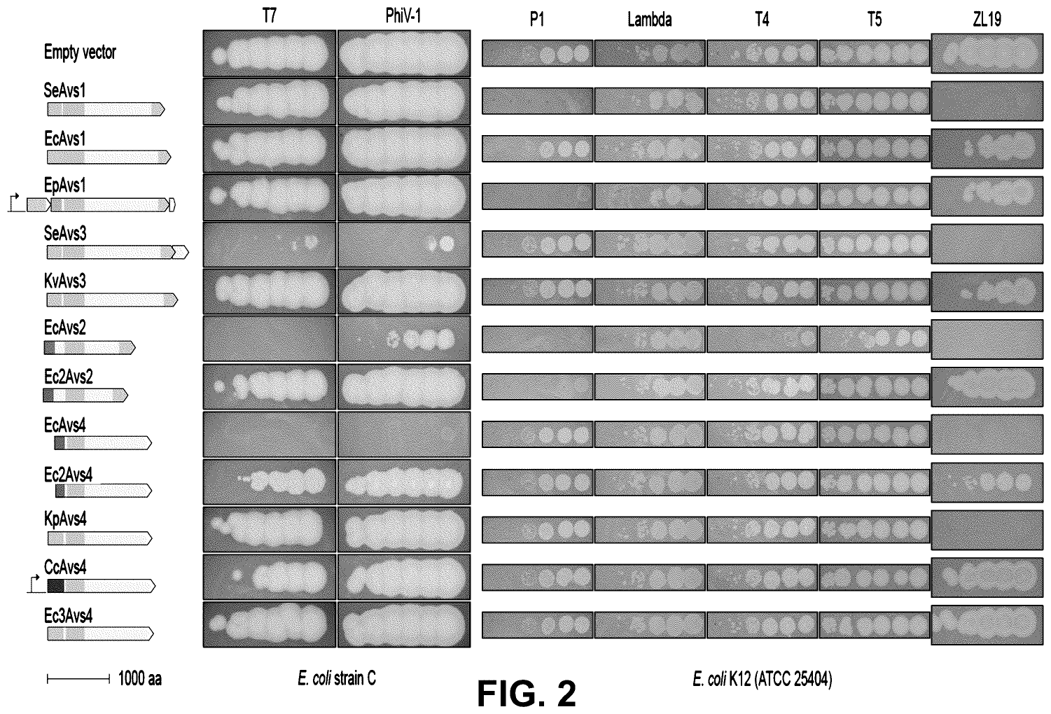

In an embodiment, the polynucleotide, vector or system thereof can include one or more elements capable of translocating and/or expressing a programmable pattern recognition protein or system polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL (SEQ ID NO: 10) and others described elsewhere herein), endoplasmic reticulum localization/retention signals (e.g., KDEL (SEQ ID NO: 61), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g., (S/A/C)-(K/R/H)-(L/A), SLK, (R/K)-(LN/I)-XXXXX-(H/Q)-(L/A/F). Suitable protein targeting motifs can also be designed or identified using any suitable database or prediction tool, including but not limited to Minimotif Miner (minimotifininer.org, mitominer.mrc-mbu.cam.ac.uk/release-4.0/embodiment.do?name=Protein %20MTS), LocDB (see above), PTSs predictor ( ), TargetP-2.0 (www.cbs.dtu.dk/services/TargetP/), ChloroP (www.cbs.dtu.dk/services/ChloroP/); NetNES (www.cbs.dtu.dk/services/NetNES/), Predotar (urgi.versailles.inra.fr/predotar/), and SignalP (www.cbs.dtu.dk/services/SignalP/).

### Selectable Markers and Tags

One or more of the programmable pattern recognition protein or system polynucleotides can be operably linked, fused to, or otherwise modified to include a polynucleotide that encodes or is a selectable marker or tag, which can be a polynucleotide or polypeptide. In an embodiment, the polypeptide encoding a polypeptide selectable marker can be incorporated in the programmable pattern recognition protein or system polynucleotide such that the selectable marker polypeptide, when translated, is inserted between two amino acids between the N- and C-terminus of the programmable pattern recognition protein or system polypeptide or at the N- and/or C-terminus of the programmable pattern recognition protein or system polypeptide. In an embodiment, the selectable marker or tag is a polynucleotide barcode or unique molecular identifier (UMI).

It will be appreciated that the polynucleotide encoding such selectable markers or tags can be incorporated into a polynucleotide encoding one or more components of the programmable pattern recognition protein or system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the CRISPR-Cas system described herein via suitable linker, such as a glycine or glycine serine linkers as short as GS or GG up to (GGGGG)₃ (SEQ ID NO: 11) or (GGGGS)₃(SEQ ID NO: 12). Other suitable linkers are described elsewhere herein.

The vector or vector system can include one or more polynucleotides encoding one or more targeting moieties. In an embodiment, the targeting moiety encoding polynucleotides can be included in the vector or vector system, such as a viral vector system, such that they are expressed within and/or on the virus particle(s) produced such that the virus particles can be targeted to specific cells, tissues, organs, etc. In an embodiment, the targeting moiety encoding polynucleotides can be included in the vector or vector system such that the programmable pattern recognition protein or system polynucleotide(s) and/or products expressed therefrom include the targeting moiety and can be targeted to specific cells, tissues, organs, etc. In an embodiment, such as non-viral carriers, the targeting moiety can be attached to the carrier (e.g., polymer, lipid, inorganic molecule etc.) and can be capable of targeting the carrier and any attached or associated programmable pattern recognition protein or system polynucleotide(s) to specific cells, tissues, organs, etc.

### Vector Construction

The vectors described herein can be constructed using any suitable process or technique. In an embodiment, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In an embodiment, a vector comprises one or more insertion sites, such as a restriction endonuclease recognition sequence (also referred to as a “cloning site”). In an embodiment, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In an embodiment, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more elements of a programmable pattern recognition composition or system described herein are as used in the foregoing documents, such as International Patent Publication No. WO 2014/093622 (PCT/US2013/074667) and are discussed in greater detail herein.

### Viral Vectors

In an embodiment, the vector is a viral vector. The term of art “viral vector” and as used herein in this context refers to polynucleotide based vectors that contain one or more elements from or based upon one or more elements of a virus that can be capable of expressing and packaging a polynucleotide, such as a programmable pattern recognition polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the programmable pattern recognition composition or system described herein. The viral vector can be part of a viral vector system involving multiple vectors. In an embodiment, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In an embodiment, the viral vectors are configured to produce replication incompetent viral particles for improved safety of these systems.

In an embodiment, the virus structural component, which can be encoded by one or more polynucleotides in a viral vector or vector system, comprises one or more capsid proteins including an entire capsid. In an embodiment, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In an embodiment, the viral vector is configured such that when the cargo is packaged the cargo(s) (e.g., one or more components of the programmable pattern recognition composition or system, including but not limited to a STAND NTPase and/or optional effector, is external to the capsid or virus particle. In the sense that it is not inside the capsid (enveloped or encompassed with the capsid) but is externally exposed so that it can contact the target genomic DNA. In an embodiment, the viral vector is configured such that all the carog(s) are contained within the capsid after packaging.

### Split Viral Vector Systems

When the programmable pattern recognition composition or system viral vector or vector system (be it a retroviral (e.g., AAV) or lentiviral vector) is designed so as to position the cargo(s) (e.g., one or more programmable pattern recognition composition or system components) at the internal surface of the capsid once formed, the cargo(s) will fill most or all of internal volume of the capsid. In other embodiments, the effector protein may be modified or divided so as to occupy a less of the capsid internal volume. Accordingly, in an embodiment, the programmable pattern recognition composition or system or component thereof (can be divided in two portions, one portion comprises in one viral particle or capsid and the second portion comprised in a second viral particle or capsid. In an embodiment, by splitting the programmable pattern recognition composition or system or component thereof in two portions, space is made available to link one or more heterologous domains to one or both programmable pattern recognition composition or system component portions. Such systems can be referred to as “split vector systems” or in the context of the present disclosure a “split programmable pattern recognition composition or system” a “split programmable pattern recognition composition or system polypeptide”, a “split STAND NTPase protein” and the like. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of the programmable pattern recognition composition or system that can be achieved with a split system or split protein design.

Split programmable pattern recognition composition or system polypeptides that can be incorporated into the AAV or other vectors described herein are set forth elsewhere herein and in documents incorporated herein by reference in further detail herein. In an embodiment, each part of a split programmable pattern recognition composition or system polypeptides are attached to a member of a specific binding pair, and when bound with each other, the members of the specific binding pair maintain the parts of the programmable pattern recognition composition or system polypeptide in proximity. In an embodiment, each part of a split programmable pattern recognition composition or system polypeptide is associated with an inducible binding pair. An inducible binding pair is one which is capable of being switched “on” or “off” by a protein or small molecule that binds to both members of the inducible binding pair. In general, according to the invention, programmable pattern recognition composition or system polypeptides may preferably split between domains, leaving domains intact. Preferred, non-limiting examples of such programmable pattern recognition composition or system polypeptides include, without limitation, STAND NTPase polypeptides, effector polypeptides, and orthologues.

In an embodiment, any AAV serotype is preferred. In an embodiment, the VP2 domain associated with the programmable pattern recognition composition or system polypeptide is an AAV serotype 2 VP2 domain. In an embodiment, the VP2 domain associated with the programmable pattern recognition composition or system polypeptide is an AAV serotype 8 VP2 domain. The serotype can be a mixed serotype as is known in the art.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the CRISPR-Cas systems can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/UJS94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In an embodiment, an HIV-based lentiviral vector system can be used. In an embodiment, a FIV-based lentiviral vector system can be used.

In an embodiment, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the programmable pattern recognition composition or system described herein.

In an embodiment, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In an embodiment, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In an embodiment, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In an embodiment, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In an embodiment, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included upstream of the LTRs), and they can include one or more deletions in the 3′LTR to create self-inactivating (SIN) vectors having disrupted promoter/enhancer activity of the LTR. In an embodiment, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In an embodiment, the third-generation lentiviral vector system can include at least two packaging vectors, with the gag-pol being present on a different vector than the rev gene.

In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the programmable pattern recognition composition or system of the present invention.

In an embodiment, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In an embodiment, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In an embodiment, the tropism of the resulting lentiviral particle can be tuned by incorporating cell targeting peptides into a lentiviral vector such that the cell targeting peptides are expressed on the surface of the resulting lentiviral particle. In an embodiment, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In an embodiment, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In an embodiment, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In an embodiment, a lentiviral vector can include an N-terminal PDZ domain of InaD protein (PDZ1) and its pentapeptide ligand (TEFCA (SEQ ID NO: 13)) from NorpA, which can conjugate the cell targeting peptide to the virus particle via a covalent bond (e.g., a disulfide bond). In an embodiment, the PDZ1 protein can be fused to an envelope protein, which can optionally be binding deficient and/or fusion competent virus envelope protein and included in a lentiviral vector. In an embodiment, the TEFCA (SEQ ID NO: 13) can be fused to a cell targeting peptide and the TEFCA-CPT (SEQ ID NO: 13)fusion construct can be incorporated into the same or a different lentiviral vector as the PDZ1-envenlope protein construct. During virus production, specific interaction between the PDZ1 and TEFCA (SEQ ID NO: 13) facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver a programmable pattern recognition composition or system polynucleotide described herein to a cell.

In an embodiment, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudo-typed retroviral or lentiviral vector particles are contemplated (see, e.g., US Patent Publication No. 20120164118 assigned to the Fred Hutchinson Cancer Research Center). Cocal virus is in the Vesiculovirus genus and is a causative agent of vesicular stomatitis in mammals. Cocal virus was originally isolated from mites in Trinidad (Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses. Jonkers et al., Am. J. Vet. Res. 25:236-242 (1964) and Travassos da Rosa et al., Am. J. Tropical Med. & Hygiene 33:999-1006 (1984). The Cocal vesiculovirus envelope pseudo-typed retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In an embodiment of these embodiments, the Gag, Pol, and accessory proteins are lentiviral and/or gammaretroviral. In an embodiment, a retroviral vector can contain encoding polypeptides for one or more Cocal vesiculovirus envelope proteins such that the resulting viral or pseudoviral particles are Cocal vesiculovirus envelope pseudo-typed.

Adenoviral Vectors. Helper-Dependent Adenoviral Vectors, and Hybrid Adenoviral Vectors

In an embodiment, the vector can be an adenoviral vector. In an embodiment, the adenoviral vector can include elements such that the virus particle produced using the vector or system thereof can be serotype 2 or serotype 5. In an embodiment, the polynucleotide to be delivered via the adenoviral particle can be up to about 8 kb. Thus, in an embodiment, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In an embodiment the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In an embodiment of the helper-dependent adenoviral vector system one vector (the helper) can contain all the viral genes required for replication but contains a conditional gene defect in the packaging domain. The second vector of the system can contain only the ends of the viral genome, one or more CRISPR-Cas polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the programmable pattern recognition composition or system polynucleotides described herein. In an embodiment, the polynucleotide to be delivered via the viral particle produced from a helper-dependent adenoviral vector or system thereof can be up to about 37 kb. Thus, in an embodiment, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In an embodiment, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In an embodiment, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the programmable pattern recognition composition or system of the present invention. In an embodiment, a hybrid-adenoviral vector can include one or more features of a retrovirus and/or an adeno-associated virus. In an embodiment the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the programmable pattern recognition composition or system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the programmable pattern recognition composition or system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In an embodiment the AAV can integrate into a specific site on chromosome 19 of a human cell with no observable side effects. In an embodiment, the capacity of the AAV vector, system thereof, and/or AAV particles can be up to about 4.7 kb.

The AAV vector or system thereof can include one or more regulatory molecules. In an embodiment the regulatory molecules can be promoters, enhancers, repressors and the like, which are described in greater detail elsewhere herein. In an embodiment, the AAV vector or system thereof can include one or more polynucleotides that can encode one or more regulatory proteins. In an embodiment, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In an embodiment, the AAV capsid can contain 60 capsid proteins. In an embodiment, the ratio of VP1:VP2:VP3 in a capsid can be about 1:1:10.

In an embodiment, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In an embodiment, a producing host cell line expresses one or more of the adenovirus helper factors.

The AAV vector or system thereof can be configured to produce AAV particles having a specific serotype. In an embodiment, the serotype can be AAV-1, AAV-2, AAV-3, AAV-4, AAV-5, AAV-6, AAV-8, AAV-9 or any combinations thereof. In an embodiment, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in an embodiment, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In an embodiment, an AAV vector or system thereof capable of producing AAV particles capable of targeting cardiac tissue can be configured to generate an AAV particle having an AAV-4 serotype. In an embodiment, an AAV vector or system thereof capable of producing AAV particles capable of targeting the liver can be configured to generate an AAV having an AAV-8 serotype. In an embodiment, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008).

In an embodiment, the AAV vector or system thereof is configured as a “gutless” vector, similar to that described in connection with a retroviral vector. In an embodiment, the “gutless” AAV vector or system thereof can have the cis-acting viral DNA elements involved in genome amplification and packaging in linkage with the heterologous sequences of interest (e.g., the programmable pattern recognition composition or system polynucleotide(s)).

In an embodiment, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In another embodiment, the invention provides a non-naturally occurring or engineered programmable pattern recognition composition or system protein associated with Adeno Associated Virus (AAV), e.g., an AAV comprising a programmable pattern recognition composition or system protein as a fusion, with or without a linker, to or with an AAV capsid protein such as VP1, VP2, and/or VP3; and, for shorthand purposes, such a non-naturally occurring or engineered programmable pattern recognition composition or system protein is herein termed a “AAV-programmable pattern recognition composition or system protein” More in particular, modifying the knowledge in the art, e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. [Epub ahead of print.] doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609, each incorporated herein by reference, one can obtain a modified AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as a programmable pattern recognition composition or system-protein. Likewise, these can be fusions, with the protein, e.g., large payload protein such as a programmable pattern recognition composition or system-protein fused in a manner analogous to prior art fusions. See, e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that large payload proteins can be fused to the AAV capsid. Applicants provide AAV capsid programmable pattern recognition composition or system R protein fusions and those AAV-capsid programmable pattern recognition composition or system protein fusions can be a recombinant AAV that contains nucleic acid molecule(s) encoding or providing programmable pattern recognition composition or system or complex RNA guide(s), whereby the programmable pattern recognition composition or system protein fusion delivers a programmable pattern recognition composition or system complex by the fusion, e.g., VP1, VP2, or VP3 fusion, and the guide RNA is provided by the coding of the recombinant virus, whereby in vivo, in a cell, the programmable pattern recognition composition or system is assembled from the nucleic acid molecule(s) of the recombinant providing the guide RNA and the outer surface of the virus providing the programmable pattern recognition composition or system polypeptide. Accordingly, the instant invention is also applicable to a virus in the genus Dependoparvovirus or in the family Parvoviridae, for instance, AAV, or a virus of Amdoparvovirus, e.g., Carnivore amdoparvovirus 1, a virus of Aveparvovirus, e.g., Galliform aveparvovirus 1, a virus of Bocaparvovirus, e.g., Ungulate bocaparvovirus 1, a virus of Copiparvovirus, e.g., Ungulate copiparvovirus 1, a virus of Dependoparvovirus, e.g., Adeno-associated dependoparvovirus A, a virus of Erythroparvovirus, e.g., Primate erythroparvovirus 1, a virus of Protoparvovirus, e.g., Rodent protoparvovirus 1, a virus of Tetraparvovirus, e.g., Primate tetraparvovirus 1. Thus, a virus of within the family Parvoviridae or the genus Dependoparvovirus or any of the other foregoing genera within Parvoviridae is contemplated as within the invention with discussion herein as to AAV applicable to such other viruses.

In an embodiment, the programmable pattern recognition composition or system polypeptide is external to the capsid or virus particle. In the sense that it is not inside the capsid (enveloped or encompassed with the capsid) but is externally exposed so that it can contact the target genomic DNA). In an embodiment, the programmable pattern recognition composition or system polypeptide is associated with the AAV VP2 domain by way of a fusion protein. In an embodiment, the association may be considered to be a modification of the VP2 domain. Where reference is made herein to a modified VP2 domain, then this will be understood to include any association discussed herein of the VP2 domain and the programmable pattern recognition composition or system polypeptide. In an embodiment, the AAV VP2 domain may be associated (or tethered) to the programmable pattern recognition composition or system polypeptide via a connector protein, for example using a system such as the streptavidin-biotin system. In an embodiment, the present invention provides a polynucleotide encoding the present programmable pattern recognition composition or system polypeptide and associated AAV VP2 domain. In one embodiment, the invention provides a non-naturally occurring modified AAV having a VP2-programmable pattern recognition composition or system polypeptide capsid protein, wherein the programmable pattern recognition composition or system polypeptide is part of or tethered to the VP2 domain. In some preferred embodiments, the programmable pattern recognition composition or system polypeptide is fused to the VP2 domain so that, in another embodiment, the invention provides a non-naturally occurring modified AAV having a VP2-programmable pattern recognition composition or system polypeptide fusion capsid protein. Thus, reference herein to a VP2-programmable pattern recognition composition or system polypeptide capsid protein may also include a VP2-programmable pattern recognition composition or system polypeptide fusion capsid protein. In an embodiment, the VP2-programmable pattern recognition composition or system polypeptide capsid protein further comprises a linker, whereby the VP2-programmable pattern recognition composition or system polypeptide is distanced from the remainder of the AAV. In an embodiment, the VP2-programmable pattern recognition composition or system polypeptide capsid protein further comprises at least one protein complex, e.g., programmable pattern recognition composition or system polypeptide complex, such as a programmable pattern recognition composition or system polypeptide complex guide RNA that targets a particular DNA, TALE, etc. A programmable pattern recognition composition or system polypeptide complex, such as programmable pattern recognition composition or system comprising the VP2-programmable pattern recognition composition or system polypeptide capsid protein and at least one programmable pattern recognition composition or system polypeptide complex, such as a programmable pattern recognition composition or system polypeptide complex guide RNA that targets a particular DNA, is also provided in one embodiment.

In one embodiment, the invention provides a non-naturally occurring or engineered composition comprising a programmable pattern recognition composition or system polypeptide which is part of or tethered to an AAV capsid domain, i.e., VP1, VP2, or VP3 domain of Adeno-Associated Virus (AAV) capsid. In an embodiment, part of or tethered to an AAV capsid domain includes associated with associated with a AAV capsid domain. In an embodiment, the programmable pattern recognition composition or system polypeptide may be fused to the AAV capsid domain. In an embodiment, the fusion may be to the N-terminal end of the AAV capsid domain. As such, in an embodiment, the C-terminal end of the programmable pattern recognition composition or system polypeptide is fused to the N-terminal end of the AAV capsid domain. In an embodiment, an NLS and/or a linker (such as a GlySer linker) may be positioned between the C-terminal end of the programmable pattern recognition composition or system polypeptide and the N-terminal end of the AAV capsid domain. In an embodiment, the fusion may be to the C-terminal end of the AAV capsid domain. In an embodiment, this is not preferred due to the fact that the VP1, VP2 and VP3 domains of AAV are alternative splices of the same RNA and so a C-terminal fusion may affect all three domains. In an embodiment, the AAV capsid domain is truncated. In an embodiment, some or all of the AAV capsid domain is removed. In an embodiment, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the CRISPR protein. A branched linker may be used, with the programmable pattern recognition composition or system polypeptide fused to the end of one of the branches. This allows for some degree of spatial separation between the capsid and the programmable pattern recognition composition or system polypeptide. In this way, the programmable pattern recognition composition or system polypeptide is part of (or fused to) the AAV capsid domain.

In other embodiments, the CRISPR enzyme may be fused in frame within, i.e. internal to, the AAV capsid domain. Thus, in an embodiment, the AAV capsid domain again preferably retains its N-terminal and C-terminal ends. In this case, a linker is preferred, in an embodiment, either at one or both ends of the programmable pattern recognition composition or system polypeptide. In this way, the programmable pattern recognition composition or system polypeptide is again part of (or fused to) the AAV capsid domain. In an embodiment, the positioning of the programmable pattern recognition composition or system polypeptide is such that the programmable pattern recognition composition or system polypeptide is at the external surface of the viral capsid once formed. In one embodiment, the invention provides a non-naturally occurring or engineered composition comprising a programmable pattern recognition composition or system polypeptide associated with a AAV capsid domain of Adeno-Associated Virus (AAV) capsid. Here, associated may mean in an embodiment fused, or in an embodiment bound to, or in an embodiment tethered to. The programmable pattern recognition composition or system polypeptide may, in an embodiment, be tethered to the VP1, VP2, or VP3 domain. This may be via a connector protein or tethering system such as the biotin-streptavidin system. In one example, a biotinylation sequence (15 amino acids) could therefore be fused to the programmable pattern recognition composition or system polypeptide. When a fusion of the AAV capsid domain, especially the N-terminus of the AAV AAV capsid domain, with streptavidin is also provided, the two will therefore associate with very high affinity. Thus, in an embodiment, provided is a composition or system comprising a programmable pattern recognition composition or system polypeptide-biotin fusion and a streptavidin-AAV capsid domain arrangement, such as a fusion. The programmable pattern recognition composition or system polypeptide-biotin and streptavidin-AAV capsid domain forms a single complex when the two parts are brought together. NLSs may also be incorporated between the programmable pattern recognition composition or system polypeptide and the biotin; and/or between the streptavidin and the AAV capsid domain.

As such, provided is a fusion of a programmable pattern recognition composition or system polypeptide with a connector protein specific for a high affinity ligand for that connector, whereas the AAV VP2 domain is bound to said high affinity ligand. For example, streptavidin may be the connector fused to the programmable pattern recognition composition or system polypeptide, while biotin may be bound to the AAV VP2 domain. Upon co-localization, the streptavidin will bind to the biotin, thus connecting the programmable pattern recognition composition or system polypeptide to the AAV VP2 domain. The reverse arrangement is also possible. In an embodiment, a biotinylation sequence (15 amino acids) could therefore be fused to the AAV VP2 domain, especially the N-terminus of the AAV VP2 domain. A fusion of the programmable pattern recognition composition or system polypeptide with streptavidin is also preferred, in an embodiment. In an embodiment, the biotinylated AAV capsids with streptavidin-programmable pattern recognition composition or system polypeptide are assembled in vitro. This way the AAV capsids should assemble in a straightforward manner and the programmable pattern recognition composition or system polypeptide-streptavidin fusion can be added after assembly of the capsid. In other embodiments a biotinylation sequence (15 amino acids) could therefore be fused to the programmable pattern recognition composition or system polypeptide, together with a fusion of the AAV VP2 domain, especially the N-terminus of the AAV VP2 domain, with streptavidin. For simplicity, a fusion of the programmable pattern recognition composition or system polypeptide and the AAV VP2 domain is preferred in an embodiment. In an embodiment, the fusion may be to the N-terminal end of the programmable pattern recognition composition or system polypeptide. In other words, in an embodiment, the AAV and programmable pattern recognition composition or system polypeptide are associated via fusion. In an embodiment, the AAV and programmable pattern recognition composition or system polypeptide are associated via fusion including a linker. Suitable linkers are discussed herein but include Gly Ser linkers. Fusion to the N-term of AAV VP2 domain is preferred, in an embodiment. In an embodiment, the programmable pattern recognition composition or system polypeptide comprises at least one Nuclear Localization Signal (NLS). In a further embodiment, the present invention provides compositions comprising the programmable pattern recognition composition or system polypeptide and associated AAV VP2 domain or the polynucleotides or vectors described herein. Such compositions and formulations are discussed elsewhere herein.

An alternative tether may be to fuse or otherwise associate the AAV capsid domain to an adaptor protein which binds to or recognizes to a corresponding RNA sequence or motif. In an embodiment, the adaptor is or comprises a binding protein which recognizes and binds (or is bound by) an RNA sequence specific for said binding protein. In an embodiment, a preferred example is the MS2 (see Konermann et al. December 2014, cited infra, incorporated herein by reference) binding protein which recognizes and binds (or is bound by) an RNA sequence specific for the MS2 protein.

With the AAV capsid domain associated with the adaptor protein, the CRISPR protein may, in an embodiment, be tethered to the adaptor protein of the AAV capsid domain. The programmable pattern recognition composition or system polypeptide may, in an embodiment, be tethered to the adaptor protein of the AAV capsid domain via the CRISPR enzyme being in a complex with a modified guide, see Konermann et al. The modified guide is, in an embodiment, a sgRNA. In an embodiment, the modified guide comprises a distinct RNA sequence; see, e.g., International Patent Application No. PCT/US14/70175, incorporated herein by reference.

In an embodiment, distinct RNA sequence is an aptamer. Thus, corresponding aptamer-adaptor protein systems are preferred. One or more functional domains may also be associated with the adaptor protein. An example of a preferred arrangement would be: [AAV AAV capsid domain—adaptor protein]—[modified guide—programmable pattern recognition composition or system polypeptide].

In an embodiment, the positioning of the programmable pattern recognition composition or system polypeptide is such that the programmable pattern recognition composition or system polypeptide is at the internal surface of the viral capsid once formed. In one embodiment, the invention provides a non-naturally occurring or engineered composition comprising a programmable pattern recognition composition or system polypeptide associated with an internal surface of an AAV capsid domain. Here again, associated may mean in an embodiment fused, or in an embodiment bound to, or in an embodiment tethered to. The programmable pattern recognition composition or system polypeptide may, in an embodiment, be tethered to the VP1, VP2, or VP3 domain such that it locates to the internal surface of the viral capsid once formed. This may be via a connector protein or tethering system such as the biotin-streptavidin system as described above and/or elsewhere herein.

### Herpes Simplex Viral Vectors

In an embodiment, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the CRISPR-Cas system of the present invention. In an embodiment where an HSV vector or system thereof is utilized, the host cell can be a complementing cell. In an embodiment, HSV vector or system thereof can be capable of producing virus particles capable of delivering a polynucleotide cargo of up to 150 kb. Thus, in some embodiment the programmable pattern recognition composition or system polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the CRISPR-Cas system of the present invention.

### Poxvirus Vectors

In an embodiment, the vector can be a poxvirus vector or system thereof. In an embodiment, the poxvirus vector can result in cytoplasmic expression of one or more programmable pattern recognition composition or system polynucleotides of the present invention. In an embodiment the capacity of a poxvirus vector or system thereof can be about 25 kb or more. In an embodiment, a poxvirus vector or system thereof can include one or more programmable pattern recognition composition or system polynucleotides described herein.

### Viral Vectors for Delivery to Plants

The systems and compositions may be delivered to plant cells using viral vehicles. In an embodiment, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Virus Particle Production from Viral Vectors

### Retroviral Production

In an embodiment, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In an embodiment, the suitable host cell for virus production from viral vectors and systems thereof described herein can stably express one or more genes involved in packaging (e.g., pol, gag, and/or VSV-G) and/or other supporting genes.

In an embodiment, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a programmable pattern recognition composition or system polynucleotide), and virus particle assembly, and secretion of mature virus particles into the culture media. Various other methods and techniques are generally known to those of ordinary skill in the art.

Mature virus particles can be collected from the culture media by a suitable method. In an embodiment, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In an embodiment, the resulting composition containing virus particles can contain 1×10¹−1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 μg of psPAX2 (gag/pol/rev/tat)). Transfection can be carried out in 4 mL OptiMEM with a cationic lipid delivery agent (50 μL Lipofectamine 2000 and 100 μl Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 μl of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In an embodiment, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the CRISPR-Cas system polynucleotide(s)). In an embodiment, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the CRISPR-Cas system polynucleotide(s)) between 2 ITRs; (2) a vector that carries the AAV Rep-Cap encoding polynucleotides; and (3) helper polynucleotides. One of skill in the art will appreciate various methods and variations thereof that are both helper and -helper free and as well as the different advantages of each system.

### Non-Viral Vectors

In an embodiment, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating programmable pattern recognition composition or system polynucleotide(s) and delivering said programmable pattern recognition composition or system polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In an embodiment one or more programmable pattern recognition composition or system polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the programmable pattern recognition composition or system polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In an embodiment, the naked polynucleotide contains only the programmable pattern recognition composition or system polynucleotide(s) of the present invention. In an embodiment, the naked polynucleotide can contain other nucleic acids and/or polynucleotides in addition to the programmable pattern recognition composition or system polynucleotide(s) of the present invention. The naked polynucleotides can include one or more elements of a transposon system. Transposons and system thereof are described in greater detail elsewhere herein.

### Non-Viral Polynucleotide Vectors

In an embodiment, one or more of the programmable pattern recognition composition or system polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In an embodiment, the non-viral polynucleotide vector can have a conditional origin of replication. In an embodiment, the non-viral polynucleotide vector can be an ORT plasmid. In an embodiment, the non-viral polynucleotide vector can have a minimalistic immunologically defined gene expression. In an embodiment, the non-viral polynucleotide vector can have one or more post-segregationally killing system genes. In an embodiment, the non-viral polynucleotide vector is AR-free. In an embodiment, the non-viral polynucleotide vector is a minivector. In an embodiment, the non-viral polynucleotide vector includes a nuclear localization signal. In an embodiment, the non-viral polynucleotide vector can include one or more CpG motifs. In an embodiment, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In an embodiment, the S/MAR sequence is located downstream of an actively transcribed polynucleotide (e.g., one or more CRISPR-Cas system polynucleotides of the present invention) included in the non-viral polynucleotide vector. In an embodiment, the S/MAR can be a S/MAR from the beta-interferon gene cluster. See e.g. Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In an embodiment, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In an embodiment, the non-viral polynucleotide vector can be a retrotransposon vector. In an embodiment, the retrotransposon vector includes long terminal repeats. In an embodiment, the retrotransposon vector does not include long terminal repeats. In an embodiment, the non-viral polynucleotide vector can be a DNA transposon vector. DNA transposon vectors can include a polynucleotide sequence encoding a transposase. In an embodiment, the transposon vector is configured as a non-autonomous transposon vector, meaning that the transposition does not occur spontaneously on its own. In some of these embodiments, the transposon vector lacks one or more polynucleotide sequences encoding proteins required for transposition. In an embodiment, the non-autonomous transposon vectors lack one or more Ac elements.

In an embodiment a non-viral polynucleotide transposon vector system can include a first polynucleotide vector that contains the programmable pattern recognition composition or system polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the programmable pattern recognition composition or system polynucleotide(s) of the present invention) and integrate it into one or more positions in the host cell's genome. In an embodiment the transposon vector or system thereof can be configured as a gene trap. In an embodiment, the TIRs can be configured to flank a strong splice acceptor site followed by a reporter and/or other gene (e.g., one or more of the programmable pattern recognition composition or system polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tc1/mariner superfamily) (see e.g. Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Delivery of the Polynucleotides, Vectors, and Vector Systems

The polynucleotides, vectors, and/or vector systems can be delivered, such as to a cell or cells, by any suitable method or technique. In an embodiment, delivery can include association or otherwise incorporating the polynucleotides, vectors and/or vector systems with one or more delivery vehicles. Exemplary delivery methods and vehicles are discussed in greater detail below.

### Physical Delivery

In an embodiment, the polynucleotides, vectors, and vector systems or any delivery vehicle containing the same may be introduced to cells by physical delivery methods. Examples of physical methods include microinjection, electroporation, and hydrodynamic delivery. Both nucleic acid and proteins may be delivered using such methods. For example, proteins of the present invention may be prepared in vitro, isolated, (refolded, purified if needed), and introduced to cells.

### Microinjection

Microinjection of the cargo directly to cells can achieve high efficiency, e.g., above 90% or about 100%. In an embodiment, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for proteins of the programmable pattern recognition composition or system and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and programmable pattern recognition composition or system polypeptide-encoding mRNA to the cytoplasm, e.g., facilitating translation and shuttling of said polypeptides or polynucleotides to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In an embodiment, the programmable pattern recognition composition or system polypeptide or polynucleotides and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the programmable pattern recognition composition or system polypeptides and/or polynucleotides, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The programmable pattern recognition composition or system polypeptides and/or polynucleotides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The programmable pattern recognition composition or system polypeptides and/or polynucleotides can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In an embodiment, the virus titer of a composition containing viral and/or pseudoviral particles can be obtained and a specific titer be used to transduce cells.

### Biolistics

The programmable pattern recognition composition or system polypeptides and/or polynucleotides can be introduced to cells using a biolistic method or technique. The term of art “biolistic”, as used herein refers to the delivery of nucleic acids to cells by high-speed particle bombardment. In an embodiment, the cargo(s) can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In an embodiment, the particles can be gold, tungsten, palladium, rhodium, platinum, or iridium particles.

### Implantable Devices

In an embodiment, the delivery system includes an implantable device that incorporates or is coated with a programmable pattern recognition composition or system polypeptides and/or polynucleotides described herein. Various implantable devices are described in the art, and include any device, graft, or other composition that can be implanted into a subject.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles in accordance with the present invention may a greatest dimension (e.g., diameter) of less than 100 microns (μm). In an embodiment, the delivery vehicles have a greatest dimension of less than 10 μm. In an embodiment, the delivery vehicles may have a greatest dimension of less than 2000 nanometers (nm). In an embodiment, the delivery vehicles may have a greatest dimension of less than 1000 nanometers (nm). In an embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In an embodiment, the delivery vehicles may have a greatest dimension ranging between 25 nm and 200 nm.

In an embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to plant cells, e.g., as described in WO 2008042156, US 20130185823, and WO2015089419. In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry(MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, describing particles, methods of making and using them and measurements thereof.

### Vector Based Delivery Vehicles

Vectors and Vector systems that can be used to deliver programmable pattern recognition composition or system polypeptides and/or polynucleotides are described in greater detail elsewhere herein.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin 0, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In an embodiment, an LNP delivery vehicle can be used to deliver a virus particle containing a CRISPR-Cas system and/or component(s) thereof. In an embodiment, the virus particle(s) can be adsorbed to the lipid particle, such as through electrostatic interactions, and/or can be attached to the liposomes via a linker.

In an embodiment, the LNP contains a nucleic acid, wherein the charge ratio of nucleic acid backbone phosphates to cationic lipid nitrogen atoms is about 1:1.5-7 or about 1:4.

In an embodiment, the LNP also includes a shielding compound, which is removable from the lipid composition under in vivo conditions. In an embodiment, the shielding compound is a biologically inert compound. In an embodiment, the shielding compound does not carry any charge on its surface or on the molecule as such. In an embodiment, the shielding compounds are polyethylenglycoles (PEGs), hydroxyethylglucose (HEG) based polymers, polyhydroxyethyl starch (polyHES) and polypropylene. In an embodiment, the PEG, HEG, polyHES, and a polypropylene weight between about 500 to 10,000 Da or between about 2000 to 5000 Da. In an embodiment, the shielding compound is PEG2000 or PEG5000.

In an embodiment, the LNP can include one or more helper lipids. In an embodiment, the helper lipid can be a phosphor lipid or a steroid. In an embodiment, the helper lipid is between about 20 mol % to 80 mol % of the total lipid content of the composition. In an embodiment, the helper lipid component is between about 35 mol % to 65 mol % of the total lipid content of the LNP. In an embodiment, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: 50168-3659(17)30038-X. doi: 10.1016/j.jconrel.2017.01.037. [Epub ahead of print]; Altinoǧlu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In an embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In an embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In an embodiment, a liposome delivery vehicle can be used to deliver a virus particle containing a CRISPR-Cas system and/or component(s) thereof. In an embodiment, the virus particle(s) can be adsorbed to the liposome, such as through electrostatic interactions, and/or can be attached to the liposomes via a linker.

In an embodiment, the liposome can be a Trojan Horse liposome (also known in the art as Molecular Trojan Horses), see e.g. cshprotocols.cshlp.org/content/2010/4/pdb.prot5407.long, the teachings of which can be applied and/or adapted to generated and/or deliver the CRISPR-Cas systems described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE.RTM. (e.g., LIPOFECTAMINE.RTM. 2000, LIPOFECTAMINE.RTM. 3000, LIPOFECTAMINE.RTM. RNAiMAX, LIPOFECTAMINE.RTM. LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In an embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,N,dimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the CRISPR-Cas systems described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In an embodiment, the delivery vehicle can be or include a lipidoid, such as any of those set forth in, for example, US 20110293703.

In an embodiment, the delivery vehicle can be or include an amino lipid, such as any of those set forth in, for example, Jayaraman, Angew. Chem. Int. Ed. 2012, 51, 8529-8533.

In an embodiment, the delivery vehicle can be or include a lipid envelope, such as any of those set forth in, for example, Korman et al., 2011. Nat. Biotech. 29:154-157.

### Lipoplexes/Polyplexes

In an embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 (e.g., forming DNA/Ca^{2α} microcomplexes), polyethenimine (PEI) (e.g., branched PEI), and poly(L-lysine) (PLL).

### Sugar-Based Particles

In an embodiment, the delivery vehicle can be a sugar-based particle. In an embodiment, the sugar-based particles can be or include GalNAc, such as any of those described in WO2014118272; US 20020150626; Nair, J K et al., 2014, Journal of the American Chemical Society 136 (49), 16958-16961; Østergaard et al., Bioconjugate Chem., 2015,26 (8), pp 1451-1455;

### Cell Penetrating Peptides

In an embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin 03 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In an embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

In an embodiment, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include, tungsten, palladium, rhodium, platinum, and iridium particles. Other non-limiting, exemplary metal nanoparticles are described in US 20100129793.

iTOP

In an embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In an embodiment, the delivery vehicles may comprise polymer-based particles (e.g., nanoparticles). In an embodiment, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In an embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles may be streptolysin 0 (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In an embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/dObm00427h.

Other non-limiting, exemplary exosomes include any of those set forth in Alvarez-Erviti et al. 2011, Nat Biotechnol 29: 341; [1401] El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In an embodiment, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In an embodiment, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In an embodiment, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007,vol. 104, no. 39; Davis et al., Nature, Vol 464, 15 Apr. 2010.

### Supercharged Proteins

In an embodiment, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In an embodiment, the delivery vehicle can allow for targeted delivery to a specific cell, tissue, organ, or system. In such embodiments, the delivery vehicle can include one or more targeting moieties that can direct targeted delivery of the cargo(s). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference and the teachings of which can be applied and/or adapted for targeted delivery of one or more CRISPR-Cas molecules described herein. Mention is also made of International Patent Publication No. WO 2016/027264, and the documents it cites, all of which are incorporated herein by reference, the teachings of which can be applied and/or adapted for targeted delivery of one or more CRISPR-Cas molecules described herein. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference, the teachings of which can be applied and/or adapted for targeted delivery of one or more CRISPR-Cas molecules described herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid by layers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody(or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG (SEQ ID NO: 14) such as APRPG-PEG-modified (SEQ ID NO: 14). VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA (SEQ ID NO: 15), cholesteryl-GALA (SEQ ID NO: 15) and PEG-GALA (SEQ ID NO: 15) may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein discussed, there is an embodiment of the invention wherein the delivery system comprises such a targeting or active targeting moiety. Likewise, Table 8 provides exemplary targeting moieties that can be used in the practice of the invention an as to each an embodiment of the invention provides a delivery system that comprises such a targeting moiety.

TABLE 8
Exemplary, non-limiting, Targeting Moieties

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine- | avβ3 | glioblastoma cells, human |
| aspartic acid-tyrosine- | | umbilical vein endothelial cells, |
| cysteine peptide | | tumor angiogenesis |
| (c(RGDyC)-LP (SEQ | | |
| ID NO: 62)) | | |
| ASSHN peptide (SEQ | | endothelial progenitor cells; anti- |
| ID NO: 63) | | cancer |
| PR_b peptide | α₅β₁ integrin | cancer cells |
| AG86 peptide | α₆β₄ integrin | cancer cells |
| KCCYSL (SEQ ID NO: | HER-2 receptor | cancer cells |
| 64) (P6.1 peptide) | | |
| affinity peptide LN | Aminopeptidase N | APN-positive tumor |
| (YEVGHRC (SEQ ID | (APN/CD13) | |
| NO: 65)) | | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a-receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more CRISPR-Cas molecules described herein.

Other exemplary targeting moieties are described elsewhere herein, such as epitope tags and the like.

### Responsive Delivery

In an embodiment, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In an embodiment, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extracellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g. MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 16)) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe304 or γ-Fe2O3, e.g., those that are less than 10 nm in size. Targeted delivery can be then by exposure to a magnetic field.

### Engineered Cells and Cell Populations

Described herein are various aspects of engineered cells or cell populations that can include one or more of the programmable pattern recognition composition or system polynucleotides, polypeptides, vectors, and/or vector systems, and/or programmable pattern recognition composition or system particles (e.g., those particles, such as virus particles, produced from a programmable pattern recognition composition or system polynucleotide and/or vector(s)) described elsewhere herein. In an embodiment, the engineered cells can express one or more of the programmable pattern recognition composition or system polynucleotides and/or can produce one or more particles, such as virus particles or exosomes, containing a programmable pattern recognition composition or system, which are described in greater detail herein. Such cells are also referred to herein as “producer cells”.

Described in an embodiment herein are engineered cells modified to express elements (i) and (iii) of the detection composition described herein. In an embodiment, where the engineered cells are further modified to express element (iv) of the detection composition described herein. In an embodiment, where the engineered cells are further modified to express element (ii) of the detection composition described herein.

In an embodiment, the invention provides a non-human eukaryotic organism; for example, a multicellular eukaryotic organism, including a eukaryotic host cell containing one or more components of an engineered delivery system described herein according to any of the described embodiments. In other aspects, the invention provides a eukaryotic organism; preferably a multicellular eukaryotic organism, comprising a eukaryotic host cell containing one or more components of a programmable pattern recognition composition or system described herein according to any of the described embodiments. In an embodiment, the organism is a host of AAV.

The engineered cell can be any eukaryotic cell, including but not limited to, human, non-human animal, plant, algae, and the like.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In an embodiment, the engineered cell can be a cell line. Examples of cell lines include, but are not limited to, C8161, CCRF-CEM, MOLT, mIMCD-3, NHDF, HeLa-S3, Huhl, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO—IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK IL, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see e.g., the American Type Culture Collection (ATCC) (Manassas, Va.)).

Further, the engineered cell may be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastomycota. fungal cells may include yeasts, molds, and filamentous fungi. In an embodiment, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In an embodiment, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientali, a.k.a. Pichia kudriav″evii and Candida acidothermophilum). In an embodiment, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus (e.g., Aspergillus niger), Trichoderma spp. (e.g., Trichoderma reesei), Rhizopus spp. (e.g., Rhizopus oryza”), and Mortierella spp. (e.g., Mortierella isabellina).

In an embodiment, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Example of “industrial” strains can include, without limitation, JAY270 and ATCC4124.

In an embodiment, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may “refer” to a cell that is polyploid in a particular genomic locus of interest.

In an embodiment, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In an embodiment, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In an embodiment, the engineered cell is a cell obtained from a subject. In an embodiment, the subject is a healthy or non-diseased subject. In an embodiment, the subject is a subject with a desired physiological and/or biological characteristic such that when an engineered delivery vesicle is produced it can package one or more molecules that are within the producer cell that can be related to the desired physiological and/or biological characteristic. In this context, the cargo molecules incorporated into the delivery vesicles can be capable of transferring the desired characteristic to a recipient cell.

In an embodiment, a cell can be obtained from a subject, modified such that it is an engineered delivery vesicle producer cell, and administered back to the subject from which it was obtained (autologous) or delivered to an allogenic subject. In other words, a producer cell described herein can be used in an autologous or allogenic context, such as in a cell therapy. In these embodiments, the cells can deliver a cargo, such as a therapeutic cargo or a cargo that can manipulate a cellular microenvironment within the subject.

Conventional viral and non-viral based gene transfer methods can be used to introduce nucleic acids (e.g., such as one or more of the polynucleotides of the engineered delivery system described herein) in cells or target tissues. Such methods can be used to administer nucleic acids encoding components of a nucleic acid-targeting system to cells in culture, or in a host organism. In an embodiment, a delivery is via a polynucleotide molecule (e.g., a DNA or RNA molecule) not contained in a vector. In an embodiment, delivery is via a vector. In an embodiment, delivery is via viral particles. In aspects, delivery is via a particle, (e.g., a nanoparticle) carrying one or more engineered delivery system polynucleotides, vectors, or viral particles. Particles, including nanoparticles, are discussed in greater detail elsewhere herein.

Vector delivery can be appropriate in an embodiment, where in vivo expression is envisaged. It will be appreciated that the engineered cells can be generated in vitro, ex vivo, in situ, or in vivo by delivery of one or more components of the engineered delivery systems as described elsewhere herein.

### Engineered Microbiomes

As described elsewhere herein the engineered protein compositions of the present invention can be configured to engineer a microbiome by targeting specific microbes within a microbiome, via target recognition specific to polypeptides, molecules, and/or molecular patterns, optionally a PAMP, on desired target microbes within the microbiome. The target cells can be acted upon by the effector functions of the engineered protein composition to kill the target cells or otherwise modify them so as to e.g., have an inhibited or stimulated growth or proliferation so as to influence their relative or absolute amount or abundance within the microbiome. By altering the microbe population within a microbiome, the structure of the microbiome can be engineered as desired. In an embodiment, the engineered microbiome has positive effects on the health or other functionality of the organ, environment, or organism in which the microbiome exists. Such engineered microbiomes are within the scope of the present invention.

Suitable conventional viral and non-viral based methods of engineering cells to contain and/or express the engineered delivery system polynucleotides and/or vectors described herein are generally known in the art and/or described elsewhere herein.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) of the present invention described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In an embodiment, the pharmaceutical formulation can include, such as an active ingredient, a programmable pattern recognition composition or system or component thereof described in greater detail elsewhere herein.

In an embodiment, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In an embodiment, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, imaging agents, radiation sensitizers, and combinations thereof.

### Effective Amounts

In an embodiment, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, pg, mg, or g or be any numerical value or subrange within any of these ranges.

In an embodiment, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In an embodiment, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In an embodiment where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In an embodiment, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In an embodiment, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In an embodiment, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In an embodiment, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In an embodiment, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In an embodiment, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In an embodiment, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In an embodiment, the given site is proximal to the administration site. In an embodiment, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In an embodiment, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In an embodiment, compounds, molecules, compositions, vectors, vector systems, cells, or a combination thereof described herein can be the ingredient whose release is delayed. In an embodiment the primary active agent is the ingredient whose release is delayed. In an embodiment, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In an embodiment for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In an embodiment, a primary active ingredient, optional secondary active ingredient, and/or pharmaceutically acceptable salt thereof where appropriate can be in a dosage form adapted for inhalation is in a particle-size-reduced form that is obtained or obtainable by micronization. In an embodiment, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In an embodiment, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In an embodiment, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in an embodiment, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In an embodiment, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In an embodiment, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, radiation sensitizer, and any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In an embodiment, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In an embodiment, the first one or a few initial amount(s) administered can be a higher dose than subsequent doses. This is typically referred to in the art as a loading dose or doses and a maintenance dose, respectively. In an embodiment, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Devices

Described in various embodiments herein are devices that are configured to carry out e.g., one or more of the assays, such as a detection, labeling, or screening, assay described herein. The devices can contain one or more of the programmable pattern recognition compositions, detection compositions, and/or systems or one or more components thereof. The assays or component thereof can be carried out on a device, such as tube, capillary, lateral flow strip, chip, cartridge or another device. The systems and/or assays described herein can be embodied on diagnostic devices. Devices can include very simple devices such as tubes for containing a single sample that contains all the reagents necessary to carry out a programmable pattern recognition and/or CRISPR-Cas collateral activity reaction described herein and provide a result (such as a colometric, turbidity shift, or fluorescent signal) all within the single tube. Other devices can be complex fully automated devices that are capable of handling tens to thousands of samples at time. As is described in greater detail elsewhere herein, one or more compositions (e.g., sample preparation, target amplification reaction, and/or programmable pattern recognition and/or CRISPR-Cas collateral activity detection reagents) can be included in the device. In an embodiment, they are included in one or more compartments and/or locations within the device in a free-dried, lyophilized or some other form. Devices can contain or be configured for optical-based readouts, lateral flow readouts, electrical readouts or others that are described herein and will be appreciated in view of the description provided herein.

In some examples, a device contains a detection composition that comprises an engineered protein of the present invention and a detection construct. Binding of a target polypeptide, target molecule, and/or target molecular pattern on said target polypeptide and/or target molecule to the recognition domain activates the effector domain and mediates effector domain modification of the detection construct resulting in generation of a detectable signal thereby allowing detection of a target polypeptide, target molecule, and/or target molecular pattern on said target polypeptide and/or target molecule.

### Discrete Volumes

In an embodiment, the devices can include individual discrete volumes. In an embodiment, an effector protein of the compositions or systems of the present invention is bound to each discrete volume in the device. In an embodiment, a detection composition or component thereof (e.g., an engineered protein of the present invention and/or a detection construct) of the present invention is contained or bound to one or more or each discrete volumes in the device. Each discrete volume may comprise a different guide RNA specific for a different target molecule. Each discrete volume may contain a different engineered protein of the present invention, each specific to a different target polypeptide, target molecule, and/or target molecular pattern.

In an embodiment, a sample is exposed to the one or more individual discrete volumes. In an embodiment, a sample is exposed to a solid substrate that comprises the individual discrete volumes. In an embodiment, a sample is exposed to a solid substrate comprising more than one discrete volume each comprising an engineered protein of the present invention that is specific for a target polypeptide, target molecule, and/or target molecular pattern. In an embodiment, a sample is exposed to a solid substrate comprising more than one discrete volume each comprising a guide RNA specific for a target molecule. Not being bound by a theory, each engineered protein of the present invention and/or each guide RNA will capture its target molecule from the sample and the sample does not need to be divided into separate assays. Thus, a valuable sample may be preserved.

An effector protein in the device (e.g., an effector domain of the engineered protein composition or detection construct) may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, an effector protein compositions or systems of the present invention is bound by an antibody specific for the effector protein compositions or systems of the present invention. Methods of binding a CRISPR enzyme has been described previously (see, e.g., US20140356867A1) and can be adapted for use with the present invention.

Several substrates and configurations of devices capable of defining multiple individual discrete volumes within the device may be used. As used herein “individual discrete volume” refers to a discrete space, such as a container, receptacle, or other arbitrary defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electromagnetic, or light illumination, or any combination thereof that can contain a target molecule and a indexable nucleic acid identifier (for example nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electromagnetic properties of the target molecules or their supports such as charge, or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In an embodiment, the compartment is an aqueous droplet in a water-in-oil emulsion. In specific embodiments, any of the applications, methods, or systems described herein requiring exact or uniform volumes may employ the use of an acoustic liquid dispenser.

### Samples

The device can be configured to hold, store, collect, receive, process and/or otherwise manipulate a sample and/or detect a component thereof. In an embodiment, the sample is a solid, semisolid, or liquid. In an embodiment, the sample is a biological sample. In an embodiment, the sample is obtained from a subject. In an embodiment, the sample is a bodily fluid. In an embodiment, the bodily fluid is saliva or nasal secretions. In an embodiment, the sample is not a bodily fluid but contains one or more cells from the subject, such as hair cells, skin cells, solid tissue or tumor cells. In an embodiment, the sample is obtained from a plant. In an embodiment, the sample is an environmental sample, such as air, soil, water, or a sample of molecules, organisms, viruses, and other particles present on an object surface. In an embodiment, the sample is a feedstuff or foodstuff or component thereof. Other exemplary samples that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

A sample for use with the invention may be a biological or environmental sample, such as a surface sample, a fluid sample, or a food sample (fresh fruits or vegetables, meats). Food samples may include a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, spinal fluid, cerebrospinal fluid, ascites, pleural effusion, seroma, pus, bile, aqueous or vitreous humor, transudate, exudate, or swab of skin or a mucosal membrane surface. In an embodiment, the biological sample is a bodily fluid. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In an embodiment, the methods and systems can be utilized for direct detection from patient samples. In an aspect, the methods and systems can further allow for direct detection from patient samples with a visual readout to further facilitate field-deployability. In an aspect, a field deployable version can include, for example the lateral flow devices and systems as described herein, and/or colorimetric detection. The methods and systems can be utilized to distinguish multiple viral species and strains and identify clinically relevant mutations, important with viral outbreaks such as the coronavirus outbreak in Wuhan (2019-nCoV). In an aspect, the sample is from a nasophyringeal swab or a saliva sample. See, e.g., Wyllie et al., “Saliva is more sensitive for SARS-CoV-2 detection in COVID-19 patients than nasopharyngeal swabs,” DOI: 10.1101/2020.04.16.20067835.

### Flexible Substrates

In an embodiment, the device comprises a flexible material substrate on which a number of spots or discrete volumes may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different engineered protein of the present invention, different guide RNA or set of guide RNAs, or where applicable, a different detection aptamer to screen for multiple targets at once. Thus, the systems and devices herein may be able to screen samples from multiple sources (e.g., multiple clinical samples from different individuals) for the presence of the same target, or a limited number of target, or aliquots of a single sample (or multiple samples from the same source) for the presence of multiple different targets in the sample. In an embodiment, the elements of the systems described herein are freeze dried onto the paper or cloth substrate. Example flexible material based substrates that may be used in certain example devices are disclosed in Pardee et al. Cell. 2016, 165(5):1255-66 and Pardee et al. Cell. 2014, 159(4):950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34(11):909-21 (2016). Further flexible based materials may include nitrocellulose, polycarbonate, methylethyl cellulose, polyvinylidene fluoride (PVDF), polystyrene, or glass (see e.g., US20120238008). In an embodiment, discrete volumes are separated by a hydrophobic surface, such as but not limited to wax, photoresist, or solid ink.

In an embodiment, the substrate, such as a flexible substrate, is a single use substrate, such as swab, strip, or cloth that is used to swab a surface or sample fluid or is placed in a prepared sample for detection by an assay described herein. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the compositions and systems of the present invention are designed to detect, for example identifying DNA SNPs, proteins, and other biomarkers, that may be used to identify a suspect, or certain tissue or cell markers to determine the type of biological matter present in a sample. Likewise, the single use substrate could be used to collect a sample from a patient—such as a saliva sample from the mouth—or a swab of the skin. In other embodiments, a sample or swab may be taken of a meat product on order to detect the presence of absence of contaminants on or within the meat product.

### Microfluidic Devices

In an embodiment, the device is configured as a microfluidic device. It will be appreciated that the microfluidic device can incorporate a chip, cartridge, flexible substrate, lateral flow strip, and/or other components described elsewhere herein. In an embodiment, the microfluidic device can be configured to drive a sample through the device such that it contacts one or more detection reaction reagents (such as those that may be present on a flexible substrate within the device) and thus carries out a polypeptide cleavage detection reaction. In an embodiment, the microfluidic device is configured to generate and/or merge different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In an embodiment, the system and/or device may be adapted for conversion to a flow-cytometry readout in or allow to sensitive and quantitative measurements of millions of cells in a single experiment and improve upon existing flow-based methods, such as the PrimeFlow assay. In an embodiment, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiators) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hou et al. “Direct Detection and drug-resistance profiling of bacteremias using inertial microfluidics” Lap Chip. 15(10):2297-2307 (2016). Further LOC embodiments are described elsewhere herein.

In one aspect, the embodiments disclosed herein are directed to a nucleic acid, polypeptide, cell, or other molecule detection system comprising a programmable pattern recognition composition or system of the present invention and/or one or more guide RNAs designed to bind to corresponding target molecules (e.g., a target nucleic acid), a reporter construct (also referred to herein as a detection construct in this context), and optional amplification reagents (discussed in greater detail elsewhere herein) to amplify target nucleic acid molecules and/or detectable signals in a sample. Detection compositions and detection constructs of the present invention are described in greater detail elsewhere herein.

### Lateral Flow Devices

In an embodiment, the device is a lateral flow device. In an embodiment, the detection assay can be provided on a lateral flow device, as described in International Publication WO 2019/071051, incorporated herein by reference. The lateral flow device can be adapted to detect one or more coronaviruses and/or other viruses in combination of the coronavirus. The lateral flow device may comprise a flexible substrate, such as a paper substrate or a flexible polymer-based substrate, which can include freeze-dried reagents for detection assays with a visual readout of the assay results. See, WO 2019/071051 at [0145]-[0151] and Example 2, specifically incorporated herein by reference. In an aspect, lyophilized reagents can include preferred excipients that aid in rate of reaction, specificity, or other variables. The excipients may comprise trehalose, histidine, and/or glycine. In an embodiment, the coronavirus assay can be utilized with isothermal amplification reagents, allowing amplification without complex instrumentation that may be unavailable in the field, as described in WO 2019/071051. Accordingly, the assay can be adapted for field diagnostics, including use of visual readout on a lateral flow device, rapid, sensitive detection and can be deployed for early and direct detection. Colorimetric detection can be utilized and may be particularly suited for field deployable applications, as described in International Application PCT/US2019/015726, published as WO2019/148206. In particular, colorimetric detection can be as described in WO2019/148206 at FIGS. 102, 105, 107-111 and [00306]-[00324], incorporated herein by reference.

In one embodiment, the invention provides a lateral flow device comprising a substrate comprising a first end and a second end. The first end may comprise a sample loading portion, a first region comprising a detectable ligand, two or more effector systems of the present invention (e.g., programmable pattern recognition compositions), two or more detection constructs, and one or more first capture regions, each comprising a first binding agent. The substrate may also comprise two or more second capture regions between the first region of the first end and the second end, each second capture region comprising a different binding agent. Each of the two or more effector systems of the present invention may comprise one or more effector proteins and one or more guide sequences, each guide sequence configured to bind one or more target molecules.

The device may comprise a lateral flow substrate for detecting a polynucleotide and/or polypeptide cleavage, such as a collateral polynucleotide and/or polynucleotide detection reaction. Substrates suitable for use in lateral flow assays are known in the art. These may include but are not necessarily limited to membranes or pads made of cellulose and/or glass fiber, polyesters, nitrocellulose, or absorbent pads (J Saudi Chem Soc 19(6):689-705; 2015), and other embodiments further described herein. The detection system, i.e., one or more programmable pattern recognition compositions or systems and corresponding detection constructs are added to the lateral flow substrate at a defined reagent portion of the lateral flow substrate, typically on one end of the lateral flow substrate. Detection constructs used within the context of the present invention are described in greater detail elsewhere herein. The lateral flow substrate further comprises a sample portion. The sample portion may be equivalent to, continuous with, or adjacent to the reagent portion. In an aspect, the lateral flow substrate can be utilized for visual readout of a detectable signal in one-pot reactions, e.g., wherein steps of extracting nucleic acids, amplifying nucleic acids, and detecting are performed in the same or single individual discrete volume.

### Lateral Flow Substrate

In an embodiment, the device is a lateral flow device. In an embodiment, the lateral flow device can be composed of a composition or system and detection construct of the present invention described elsewhere herein and a lateral flow substrate for carrying out the detection reaction and/or nucleic acid release from the sample.

In an embodiment, a lateral flow device comprises a lateral flow substrate on which detection can be performed. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6):689-705; 2015).

Lateral support substrates comprise a first and second end, and one or more capture regions that each comprise binding agents. The first end may comprise a sample loading portion, a first region comprising a detectable ligand, two or more effector compositions or systems of the present invention, two or more detection constructs, and one or more first capture regions, each comprising a first binding agent. The substrate may also comprise two or more second capture regions between the first region of the first end and the second end, each second capture region comprising a different binding agent. Each of the two or more of the effector compositions or systems of the present invention may comprise one or more effector proteins and one or more guide sequences, each guide sequence configured to bind one or more target molecules. The lateral flow substrates may be configured to detect a reaction mediated by an effector domain of the engineered protein of the present invention, such as a nuclease, protease and/or peptidase.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The embodiments disclosed herein can be prepared in freeze-dried format for convenient distribution and point-of-care (POC) applications. Such embodiments are useful in multiple scenarios in human health including, for example, viral detection, bacterial strain typing, sensitive genotyping, and detection of disease-associated cell free DNA. Accordingly, the lateral substrate comprising one or more of the elements of the system, including detectable ligands, effector systems, detection constructs and binding agents may be freeze-dried to the lateral flow substrate and packaged as a ready to use device. Alternatively, all or a portion of the elements of the system may be added to the reagent portion of the lateral flow substrate at the time of using the device.

### First End and Second End of the Substrate

The substrate of the lateral flow device comprises a first and second end. The effector composition or system of the present invention described herein (including any corresponding detection constructs) are added to the lateral flow substrate at a defined reagent portion of the lateral flow substrate, typically on a first end of the lateral flow substrate. Detection constructs used within the context of the present invention are described in greater detail elsewhere herein. The lateral flow substrate can further include a sample portion. The sample portion may be equivalent to, continuous with, or adjacent to the reagent portion.

In an embodiment, the first end comprises a first region. The first region comprises a detectable ligand, two or more effector systems of the present invention (e.g., one or more engineered proteins of the present invention), two or more detection constructs, and one or more first capture regions, each comprising a first binding agent.

### Capture Regions

The lateral flow substrate can comprise one or more capture regions. In embodiments the first end of the lateral flow substrate comprises one or more first capture regions, with two or more second capture regions between the first region of the first end of the substrate and the second end of the substrate. The capture regions may be provided as a capture line, typically a horizontal line running across the device, but other configurations are possible. The first capture region is proximate to and on the same end of the lateral flow substrate as the sample loading portion.

### Binding Agents

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

A first binding agent that specifically binds the first molecule of the reporter construct is fixed or otherwise immobilized to the first capture region. The second capture region is located towards the opposite end of the lateral flow substrate from the first capture region. A second binding agent is fixed or otherwise immobilized at the second capture region. The second binding agent specifically binds the second molecule of the reporter construct, or the second binding agent may bind a detectable ligand. For example, the detectable ligand may be a particle, such as a colloidal particle, that when it aggregates can be detected visually, and generates a detectable positive signal. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding region comprises a second binding agent capable of specifically or non-specifically binding the detectable ligand on the antibody of the detectable ligand. Binding agents can be, for example, antibodies, that recognize a particular affinity tag. Such binding agents can further contain, for example, detectable labels, such as isotope labels and/or nucleic acid barcodes. A barcode is a short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the effector compositions and systems of the present invention may be used to detect the barcode.

### Detectable Ligands

The first region is loaded with a detectable ligand, such as those disclosed herein, for example a gold nanoparticle. The detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G. In some examples, the detectable ligand is a gold nanoparticle, which may be modified with a first antibody, such as an anti-FITC antibody.

### Lateral Flow Detection Constructs

The first region also comprises a detection construct. In one example embodiment, and for purposes of further illustration, the detection construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the detection construct is present it its initial state, i.e., in the absence of target, the FAM molecule on the first end will bind the anti-FITC antibody on the gold nanoparticle, and the biotin on the second end of the construct will bind the biotin ligand allowing for the detectable ligand to accumulate at the first test, generating a detectable signal. Generation of a detectable signal at the first band indicates the absence of the target ligand. In the presence of target, an effector complex of the present invention forms and an effector protein is activated resulting in cleavage of the detection construct containing a target polypeptide. In the absence of an intact detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FITC antibody on the colloidal gold. Therefore, in the presence of one or more targets, the detectable ligand will accumulate at the second band, indicating the presence of the one or more targets in the sample. Other detection constructs besides the one utilizing colloidal gold may be used in connection with the lateral flow devices herein. Other detection construct are described elsewhere herein.

In an embodiment, the first end of the lateral flow device comprises two detection constructs and each of the two detection constructs comprises a target polypeptide, comprising a first molecule on a first end and a second molecule on a second end. The first molecule and the second molecule may be linked by a polypeptide linker, such as a target polypeptide.

In an embodiment, the first molecule on the first end of the first detection construct may be FAM (or a first detection molecule) and the second molecule on the second end of the first detection construct may be biotin (or second detection molecule), or vice versa. In an embodiment, the first molecule on the first end of the second detection construct may be FAM and the second molecule on the second end of the second detection construct may be Digoxigenin (DIG), or vice versa.

In an embodiment, the first end may comprise three detection constructs, wherein each of the three detection constructs comprises a target polypeptide, comprising a first molecule on a first end and a second molecule on a second end. In specific embodiments, the first and second molecules on the detection constructs comprise Tye 665 and Alexa 488; Tye 665 and FAM, and Tye 665 and Digoxigenin (DIG), respectively. Other detection molecules are described elsewhere herein and can be used in connection with the lateral flow device described herein in view of the guiding principles above.

In an embodiment, the first end of the lateral flow device comprises two or more effector compositions or systems of the present invention. In an embodiment, such an effector system may include a one or more effector proteins and one or more guide sequences configured to bind to one or more target sequences.

### Samples

When utilizing the detection systems with a lateral flow substrate, samples to be screened are loaded at the sample loading portion of the lateral flow substrate. The samples must be liquid samples or samples dissolved in an appropriate solvent, usually aqueous. The liquid sample reconstitutes the detection reagents such that a detection reaction can occur. The liquid sample begins to flow from the sample portion of the substrate towards the first and second capture regions. Exemplary samples are described in greater detail elsewhere herein. See also WO 2019/071051, which is incorporated by reference herein.

### Cartridges and Chips

The cartridge, also referred to herein as a chip, according to the present invention comprises a series of components of ampoules and chambers that are communicatively coupled with one or more other components on the cartridge. The coupling is typically a fluidic communication, for example, via channels. The cartridge may comprise a membrane that seals one or more of the chambers and/or ampoules. In an aspect, the membrane allows for storage of reagents, buffers and other solid or fluid components which cover and seal the cartridge. The membrane can be configured to be punctured, pierced or otherwise released from sealing or covering one or more components of the cartridge by a means for releasing reagents. In an embodiment, the cartridge contains one or more wells, substrates (e.g., a flexible substrate), or other discrete volumes.

In an embodiment, the device is configured as lab-on-chip (LOC) diagnostic system. In an embodiment, the LOC is configured as a wireless lab-on-chip (LOC) diagnostic sensor system (see e.g., U.S. Pat. No. 9,470,699). In an embodiment, the pattern recognition based detection assay is performed in a LOC controlled and/or read by a wireless device (e.g., a cell phone, a personal digital assistant (PDA), a tablet) and results and/or reaction are reported to and/or measured by said device. In an embodiment, the LOC may be a microfluidic device. The LOC may be a passive chip, wherein the chip is powered and controlled through a wireless device. In an embodiment, the LOC includes a microfluidic channel for holding reagents and a channel for introducing a sample. In an embodiment, a signal from the wireless device delivers power to the LOC and activates mixing of the sample and assay reagents. Specifically, in the case of the present invention, the system may include a masking agent, effector protein of the composition or system of the present invention (e.g., an engineered protein of the present invention and/or detection composition), and optionally guide RNAs specific for a target molecule. Upon activation of the LOC, the microfluidic device may mix the sample and assay reagents. Upon mixing, a sensor detects a signal and transmits the results to the wireless device. In an embodiment, the unmasking agent is a conductive RNA or polypeptide molecule. The conductive RNA or polypeptide molecule may be attached to the conductive material. Conductive molecules can be conductive nanoparticles, conductive proteins, metal particles that are attached to the protein or latex or other beads that are conductive. In an embodiment, if DNA or RNA is used then the conductive molecules can be attached directly to the matching DNA or RNA strands. The release of the conductive molecules may be detected across a sensor. The assay may be a one step process. Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

As noted above, certain embodiments enable the use of nucleic acid binding beads to concentrate target nucleic acid but that do not require elution of the isolated nucleic acid. Thus, in an embodiment, the cartridge may further comprise an activatable magnet, such as an electro-magnet. A means for activating the magnet may be located on the device, or the means for supplying the magnet or activating the magnet on the cartridge may be provided by a second device, such as those disclosed in further detail below.

The overall size of the device may be between 10, 15, 20, 25, 30, 35, 40, 45, 55, 60, 65, 70, 75, 80, 85, 90, 95, or 100 mm in width, and 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mm. The sizing of ampoules, chambers, and channels can be selected to be in line with the reaction volumes discussed herein and to fit within the general size parameters of the overall cartridge.

### Ampoules

The ampoules, also referred to as blisters, allow for storage and release of reagents throughout the cartridge. Ampoules can include liquid or solid reagents, for example, lysis reagents in one ampoule and reaction reagents in another ampoule. The reagents can be as described elsewhere herein and can be adapted for the use in the cartridge or microfluidic or other device. The ampoule may be sealed by a film that allows for the bursting, puncture or other release of the contents of the ampoules. See, e.g., Becker, H. & Girtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Considerations for ampoules can include as discussed in, for example, Smith, S., et al., Blister pouches for effective reagent storage on microfluidic chips for blood cell counting. Microfluid Nanofluid 20, 163 (2016). DOI:10.1007/s10404-016-1830-2. In an aspect, the seal is a frangible seal formed of a composite-layer film that is assembled to the cartridge main body or other part of the device. While referred to herein as an ampoule, the ampoule may comprise a cavity on a chip which comprises a sealed film that is opened by the release means.

### Chambers

The chip, microfluidic device, and/or other device described herein can have one or more chambers. The chambers on the chip may located and sized for fluidic communication via channels or other communication means with ampoules and/or other chambers on the chip. A chamber for receiving a sample can be provided. The sample can be injected, placed in a receptacle into the chamber for receiving a sample, or otherwise transferred to the chamber. A lysis chamber may comprise, for example, capture beads, that may be used for concentration and/or extraction of the desired target material from the sample. Alternatively, the beads may be comprised in an ampoule comprising lysis reagents that are in fluidic communication with the lysis chamber. An amplification chamber may also be provided with, for example, one or more lyophilized components of the system in the amplification chamber and/or communicatively connected to an ampoule comprising one or more components of the amplification reaction.

When the cartridge comprises a magnet, it may be configured near one or more of the chambers. In an aspect, the magnet is near the lysis well, and may be configured such that the device has a means for activating the magnet. Embodiments comprising a magnet in the cartridge may be utilized with methodologies using magnetic beads for extraction of particular target molecules.

### System for Detection Assays

A system configured for use with the cartridge and to perform an assay, also referred to as a sample analysis apparatus, detection system or detection device, is configured system to receive the cartridge and conduct an assay comprising isothermal amplification of nucleic acids and detection of target nucleic acids on the cartridge. The system may comprise: a body; a door housing which may be provided in an opened state or a closed state and configured to be coupled to the body of the sample analysis apparatus by a hinge or other closure means; a cartridge accommodating unit included in the detection system and configured to accommodate the cartridge. The system may further comprise one or more means for releasing reagents for extractions, amplification and/or detection; one or more heating means for extractions, amplification and/or detection, a means for mixing reagents for extraction, amplification, and/or detections, and/or a means for reading the results of the assay. The device may further comprise a user interface for programming the device and/or readout of the results of the assay.

### Means for Release of Reagents

The system may comprise means for releasing reagents for extraction, amplification and/or detection. Release of reagents can be performed by a crushing, puncturing, applying heat or pressure until burst, cutting, or other means for the opening of the ampoule and release of contents. e.g., Becker, H. & Girtner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Mechanical actuators

### Heating Means

The heating means or heating element can be provided, for example, by electrical or chemical elements. One or more heating means can be utilized, or circuits providing regulation of temperature to one or more locations within the detection device can be utilized. In an embodiment, the device is configured to comprise a heating means for heating the lysis (extraction) chamber and at the amplification chamber of the cartridge, sample vessel or other part of the device. In an aspect, the heating element is disposed under the extraction well. The system can be designed with one or more heating means for extraction, amplification and/or detection. In an embodiment, the device does not include a power source. In an embodiment, the heating element provides heat to about 65, 60, 55, 50, 45, 40, 39, 38, 37, 36, 35, 34, 33, 32, 31, 30, 29, 28, 27, 26, 25 degrees C. or less. In an embodiment, the device does not contain any heating element.

### Power Sources

In an embodiment, the device can include a power source. The power source can be coupled to one or more of the components of the device. In an embodiment, the power source is electrically coupled to one or more components of the device so as to provide electrical energy to the cone or more components. Suitable power sources that can be incorporated with the device are batteries (single use and rechargeable), solar powered power sources and batteries. In an embodiment, the power source can be coupled to an outside power source (e.g., an electric power grid) so as to recharge the on-board power source. In an embodiment, the device does not include a power source.

### Mixing Means

A means for mixing reagents for extraction, amplification and/or detection can be provided. A means for mixing reagents may comprise a means for mixing one or more fluids, or a fluid with a solid or lyophilized reaction mixture can also be provided. Means for mixing that disturb the laminar flow can be provided. In an aspect, the mixing means is a passive mixer, in another aspect, the mixing means is an active mixer. See, e.g., Nam-Trung Nguyen and Zhigang Wu 2005 J. Micromech. Microeng. 15 R1, doi: 10.1088/0960-1317/15/2/RO1 for discussion of mixing approaches. In an aspect, the active mixer can be based on external sources such as pressure, temperature, hydrodynamics (with electrical or magnetic forces), dielectrophoresis, electrokinetics, or acoustics. Examples of passive mixing means can be provided by use of geometric approaches, such as a curved path or channel, see, e.g., U.S. Pat. No. 7,160,025, or an expansion/contraction of a channel cross section or diameter. When the cartridge is utilized with beads, channels and wells are configured and sized for the flow of beads.

### Means for Reading the Results of the Assay

A means for reading the results of the assay can be provided in the system. The means for reading the results of the assay will depend in part on the type of detectable signal generated by the assay. In an embodiment, the assay generates a detectable fluorescent or color readout. In these instances, the means for reading the results of the assay will be an optic means, for example a single channel or multi-channel optical means such as a fluorimeter, colorimeter or other spectroscopic sensor.

A combination of means for reading the results of the assay can be utilized, and may include readings such as turbidity, temperature, magnetic, radio, or electrical properties and or optical properties, including scattering, polarization effects, etc.

The system may further comprise a user interface for programming the device and/or readout of the results of the assay. The user interface may comprise an LED screen. The system can be further configured for a USB port that can allow for docking of four or more devices.

In an aspect, the system comprises a means for activating a magnet that is disposed within or on the cartridge.

### Wearable Devices

The systems described herein, may further be incorporated into wearable medical devices that assess biological samples, such as biological fluids or an environmental sample, of a subject or in a subject's environment outside the clinic setting and report the outcome of the assay remotely to a central server accessible by a medical care professional. In an embodiment the device may include the ability to self-sample blood, saliva, sweat, such as the devices disclosed in U.S. Patent Application Publication No. 2015/0342509 entitled “Needle-free Blood Draw” to Peeters et al., U.S. Patent Application Publication No. 2015/0065821 entitled “Nanoparticle Phoresies” to Andrew Conrad.

In an embodiment, the device is configured as a dosimeter or badge that serves as a sensor or indicator such that the wearer is notified of exposure to certain microbes or other agents. For example, the systems described herein may be used to detect a particular pathogen. Likewise, aptamer-based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous microbes as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

### Other Device Features

In an embodiment, the device may comprise individual wells, such as microplate wells. The size of the microplate wells may be the size of standard 6, 24, 96, 384, 1536, 3456, or 9600 sized wells. In an embodiment, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In an embodiment, the devices are connected to controllers with programmable valves that work together to move fluids through the device. In an embodiment, the devices are connected to the controllers discussed in further detail below. The devices may be connected to flow actuators, controllers, and sample loading devices by tubing that terminates in metal pins for insertion into inlet ports on the device.

As shown herein, the elements of the system are stable when freeze dried or lyophilized, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In an embodiment, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In an embodiment, an optical sensor detects unmasking of a fluorescent masking agent.

In an embodiment, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in an embodiment utilizing quantum dot-based detection constructs, use of a handheld UV light, or other suitable device, may be successfully used to detect a signal owing to the near complete quantum yield provided by quantum dots.

### Kits

Any of the compounds, compositions, formulations, particles, cells, devices, and combinations thereof, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, dipsticks, substrates, bottles, and the like. The separate kit components can be contained in a single package or in separate packages within the kit.

In an embodiment, the combination kit also includes instructions printed on or otherwise contained in a tangible medium of expression. The instructions can provide information regarding the content of the compounds, compositions, formulations, particles, cells, devices, described herein or a combination thereof contained therein, safety information regarding the content of the compounds, compositions, formulations, particles, devices, and cells described herein or a combination thereof contained therein, information regarding the dosages, working amounts, indications for use, and/or recommended treatment regimen(s) for the compound(s) formulations, devices, and combinations thereof contained therein. In an embodiment, the instructions can provide directions for sample collection, sample preparation, and/or use of the compounds, compositions, formulations, particles, devices and cells described herein or a combination thereof. In an embodiment, the instructions can be specific to the target(s) being detected by an effector composition or system of the present invention (e.g., a programmable pattern recognition composition or system described herein).

### Methods of Use

### Target Molecule Modification

The compositions and systems of the present invention can be used to modify a target cell or molecule, such as a target polypeptide and/or target polynucleotide. In an embodiment, a method of modifying a target molecule and/or cell comprises delivering an engineered protein of the present invention, a polynucleotide of the present invention, a vector or vector system of the present invention, a formulation thereof, or any combination thereof to the target molecule and/or cell, or a sample containing the same, wherein the target molecule and/or cell is or comprises a target polypeptide and activating an effector domain of the engineered protein by allowing binding of the target polypeptide to the recognition domain thereby activating the effector domain via the effector activation domain, wherein effector domain activity modifies the target molecule and/or cell. In an embodiment, modification comprises nucleotide or nucleic acid modification, such including, but not limited to, cleavage, nicking, methylation, demethylation, sequence mutation or modification, base exchange, base editing, any combination thereof and/or the like. In an embodiment, modification comprises polypeptide or amino acid modification, including but not limited to, cleavage, hydrolyzing, acetylation, deacetylation, glycosylation, deglycosylation, phosphorylation, dephosphorylation, any combination thereof, and/or the like.

In an embodiment the target molecule (e.g., a target polypeptide and/or target polynucleotide) contains or is otherwise associated with a target molecular recognition pattern, such as a PAMP. In an embodiment, the target molecule (e.g., a target polypeptide or polynucleotide) is contained within or on the surface of a cell. In an embodiment, the target molecule (e.g., a target polypeptide or polynucleotide) is exogenous (i.e., not native) to a cell or organism. In an embodiment, the target molecule (e.g., a target polypeptide or polynucleotide) is endogenous or native to the cell or organism to which is introduced. In an embodiment, the exogenous target molecule (e.g., a target polypeptide or polynucleotide) is or is part of a detection composition (e.g., a detection construct) of the present invention. In an embodiment, such as in those methods where an endogenous or exogenous target molecule (e.g., a target polypeptide or polynucleotide) is to be modified, compositions and systems of the present invention are configured to detect an exogenous target molecule (e.g., a target polypeptide or polynucleotide) and thus activation of the engineered protein of the present invention and target molecule modification can be controlled, at least in part, by controlling delivery of the target polynucleotide. In an embodiment, such as in those methods where an endogenous or exogenous target molecule (e.g., a target polypeptide or polynucleotide) is to be modified, compositions and systems of the present invention are configured to detect an endogenous target molecule (e.g., a target polypeptide or polynucleotide), activation of the system and thus target polypeptide modification, occurs only in cells that contain the target molecule (e.g., a target polypeptide or polynucleotide), such as target proteins, DNA, and/or RNA. In an embodiment target molecule (e.g., a target polypeptide or polynucleotide) modification is cleavage of the target molecule (e.g., a target polypeptide or polynucleotide).

In an embodiment, the target molecule (e.g., a target polypeptide or polynucleotide) is not contained in or associated with a cell. In an embodiment, the target molecule (e.g., a target polypeptide or polynucleotide) is or is part of a detection construct, such as one that is configured for use in an in vitro detection assay. Such embodiments are described in greater detail elsewhere herein. In an embodiment, the target molecule (e.g., a target polypeptide or polynucleotide) is not contained in or associated with a cell.

In an embodiment, introducing into the sample comprises in vitro, ex vivo, or in vivo delivery of the programmable nuclease-peptidase composition into a cell or cell population.

In an embodiment, modification of the one or more target polypeptides and/or polynucleotides results in activation or deactivation of one or more cell-signaling proteins and/or pathways. In an embodiment the cell-signaling protein is a protein involved in any one or more of the following pathways: Akt signaling pathway, AMPK signaling pathway, apoptosis signaling pathway, estrogen signaling pathway, insulin signaling pathway, JAK-STAT signaling pathway, MAPK signaling pathway, mTOR signaling pathway, NF-kappaB signaling pathway, Notch signaling pathway, p53 signaling pathway, TGF-beta signaling pathway, Toll-like receptor signaling pathway, VEGF signaling pathway, Wnt signaling pathway, hedgehog signaling pathway, a cytokine signaling pathway, a growth factor signaling pathway, a PI3K signaling pathway, a PKC signaling pathway, a MEK signaling pathway, a GSK3 beta signaling pathway, and/or the like. In an embodiment the cell-signaling protein is a protein involved in a cytokine receptor mediated pathway, a survival factor receptor mediated signaling pathway, a G-protein coupled receptor mediated signaling pathway, a growth factor receptor, mediated signaling pathway, an integrin mediated signaling pathway, a Frizzled receptor mediated signaling pathway, a Fas receptor mediated signaling pathway, a Patched/SMO receptor mediated signaling pathway.

In an embodiment, the cell signaling protein is JAK, STAT3, STAT5, Bcl-xL, cytochrome C, caspase 9, caspase 8, FADD, Bad, Bim, Bcl-2, PI3K, Akt, Akkalpha, IkapppaB, PLC, PKC, NFkappaB, G-protein, adenylate cyclase, PKA, Grb2, SOS, Ras, Raf, MEK, MEKK, MAPK, MKK, Myc, Mad, Max, CREB, ARF, mdm2, Mt, Bax, p53, ERK, Fos, a JNK, Jun, beta cadherin, TCF, a disheveled protein, GSK3beta, APC, Gli, p16, p15, p21, CycIE, CDK2, CycID, CDK4, Rb, E2F, a heat shock protein, insulin, ghrelin, preproghrelin, obestatin, neuropeptideY, erythropoietin, growth hormone, glucagon, vasopressin, calcitonin, adrenocortical hormone, amylin, angiotensin, atrial natriuretic peptide, cholecystokinin, gastrin, secretin, C-peptide, relaxin, pancreatic polypeptide, follicle-stimulating hormone, leptin, luteinizing hormone, melanocyte stimulating hormone, melanotropin, oxytocin, parathyroid hormone, prolactin, renin, somatostatin, thyroid-stimulating hormone, thyrotropin-releasing hormone, substance P, vasoactive intestinal peptide, IFN-gamma, MHC, TCRs, BCRs, activin, inhibin, bone-morophogeneitc proteins, TGF-beta, Smad transcription factors, RXR, IL-1, TNF, and/or the like.

In an embodiment, the one or more target polynucleotides are a specific transcript or set of transcripts and wherein modification of the one or more target polypeptides triggers cell death upon activating the peptidase in response to binding of the nuclease-peptidase to the specific transcript or set of transcripts. In an embodiment, the guide molecule is configured to detect one or more mutations in the specific transcript or set of transcripts.

In an embodiment, the method of modifying a polypeptide can be used for, e.g., treating a disease or eliminating a pathogenic microorganism, by triggering apoptosis in the cell or otherwise disrupting signaling, or other function activity of the cell by modifying a polypeptide within said cell. Other applications of the methods of modifying a polypeptide will be appreciated in view of the description herein and, in particular, the polypeptides modified.

### Biologic Activity Modulation

The engineered proteins of the present invention can have application for biologic activity modulation. In an embodiment, the engineered proteins of the present invention are included in an effector system that generally includes a substrate for an effector domain of the programmable pattern recognition composition that is coupled to an effector of interest. Cleavage of the substrate for the effector of the programmable pattern recognition composition directly or indirectly results in activity of the effector of interest, which in turn initiates a biological activity, stops a biological activity, or modulates/modifies a biological activity.

In an embodiment, one or more components of an effector of interest is expressed in an organism or a cell or cell population thereof. Activity of the effector of interest is stimulated, stopped, increased, or decreased when the programmable pattern recognition composition of the present invention is activated by recognizing and/or binding a target molecule (e.g., a target polypeptide and/or target polynucleotide) that is contained in, coupled to, or otherwise associated with a target cell, polypeptide, and/or polynucleotide. In an embodiment, the target polynucleotide is endogenous to the cell in which the effector system is expressed. In an embodiment, the target polynucleotide is exogenous to the cell in which the effector system of interest is expressed.

In an embodiment, the effector of interest is separately expressed from the programmable pattern recognition composition, a target polynucleotide, a target polypeptide, or any combination thereof. Thus, in an embodiment, effector of interest activity is controlled by controlling the timing of co-expression of the effector of interest, the programmable pattern recognition composition, the target polypeptide, and/or the target polynucleotide.

The effector system of interest can be used to modify a biological activity in a cell or cells so as to impart a functionality to an organism or cell(s) thereof and/or treat and/or prevent a disease, condition, infection, disorder, or any combination thereof in an organism or cell(s) thereof.

### Perturbation Screening

The programmable pattern recognition compositions and systems of the present invention can be used for functional screening, such as a method of perturbation screening. Described in several exemplary embodiments herein are methods for screening cell perturbations comprising introducing a perturbation to a cell population comprising engineered cells as described in greater detail elsewhere herein, along with any elements of a detection composition not already expressed by the engineered cells, and wherein the programmable pattern recognition composition of the present invention is configured to introduce a perturbation in a polynucleotide and/or polypeptide via activation of an effector domain of the engineered programmable pattern recognition composition of the present invention by recognizing and/or binding, a target molecule (e.g., a target polypeptide or target polynucleotide) that is contained in, coupled to, or otherwise associated with (e.g., on a cell surface containing a target polynucleotide or polypeptide) the polynucleotide and/or polypeptide that is to be perturbed. In an embodiment, the programmable pattern recognition composition or component thereof (e.g., an effector domain contained therein), comprises a nuclease, nickase, protease, peptidase, methylase, acetylase, deacetylase, demethylase, transferase, phosphorylase, dephosphorylatse glycosylase, deglycosylase, and/or the like that is effective to introduce a perturbation in the target polynucleotide and/or polypeptide. In an embodiment the effector is a STAND protein, optionally a STAND NTPase or component thereof. In an embodiment the nuclease or nickase is a Cas. In an embodiment, the programmable pattern recognition composition includes or is delivered with a guide molecule for a CRISPR-Cas system. In an embodiment, when the Cas nuclease is activated by target recognition by the programmable pattern recognition composition of the present invention, a perturbation is introduced into a target polynucleotide. In an embodiment, the target molecule (e.g., target polypeptide or target polynucleotide) and/or target molecular pattern (e.g, a PAMP) recognized by the pattern recognition composition is associated with a specific cell type, cell stated, or cell species (or subspecies). In an embodiment, the guide molecules are configured to detect one or more target transcripts associated with a specific cell type or cell state. In an embodiment, activation of the programmable pattern recognition composition results in production of a detectable product and/or signal, optionally from a detection construct, thus allowing for detection of the perturbation to modify expression of a target polynucleotide and/or target polypeptide by measuring a change in the detectable product or signal relative to a control.

As is described in greater detail elsewhere herein, the engineered cells into which one or more perturbations are introduced contain a programmable pattern recognition composition or system, such as a detection composition system, of the present invention. Detection constructs and detection assays and devices are described in greater detail elsewhere herein.

In general perturbation screening is a method of introducing one or more modifications (e.g., perturbations) into the genome and evaluating any change in gene and/or protein expression, phenotype, characteristic, functionality, and/or the like. Methods and tools for genome-scale screening of perturbations in cells, including single cells, using CRISPR-Cas9 have been described, herein referred to as perturb-seq (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; and International publication serial number WO/2017/075294). A similar approach may be used with the compositions and systems of the present invention provided herein.

The compositions and systems present invention are compatible with a detection reaction utilizing a detection composition of the present invention, such that genes, such as signature and/or target, genes may be perturbed, and the perturbation may be identified and assigned to the proteomic and gene expression readouts of single cells or cell populations. In an embodiment, genes, such as signature or target genes, may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in an embodiment, perturbation is used to discover novel drug and other targets to allow treatment of specific diseases, conditions, etc. at the population, subpopulation, and/or individual patient level.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In an embodiment, the measuring of phenotypic differences and assigning a perturbation to a cell or single cell is determined by performing a detection reaction utilizing a detection composition described herein. In an embodiment, barcodes such as nucleic acid barcodes, can be included in the detection composition and/or detection construct such that single cells, or cell populations, detection compositions, detection constructs, target molecules, target polypeptides of the compositions of the present invention, can be distinguished and/or associated with a particular perturbation and/or result. In an embodiment, the barcode comprises a Unique Molecular Identifier (UMI).

Perturbations may be introduced into an engineered cell described herein using any suitable method or technique. In an embodiment, perturbations are introduced at least in part using a CRISPR-Cas system or component thereof. In an embodiment, a programmable pattern recognition protein composition of the present invention and/or CRISPR system or component thereof is used to create an INDEL at one or more target genes. In other embodiments, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/naturel4136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimising deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). Numerous genetic variants associated with disease phenotypes are found to be in non-coding region of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation. In one embodiment, a CRISPR system may be used to activate gene transcription. A nuclease-dead RNA-guided DNA binding domain, dCas9, tethered to transcriptional repressor domains that promote epigenetic silencing (e.g., KRAB) may be used for “CRISPRi” that represses transcription. To use dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required. In an embodiment, the CRISPR-Cas system used to introduce the perturbation(s) includes a Cpf1.

The engineered cells into which the perturbation(s) are introduced may comprise a cell in a model non-human organism, a model non-human mammal, such as a mouse, non-human primate, and/or the like, that expresses a composition or system of the present invention or component(s) thereof, a mouse that expresses a composition or system of the present invention or component(s) thereof, a cell in vivo, or a cell ex vivo, or a cell in vitro (see e.g., WO 2014/093622 (PCT/US13/074667); US Patent Publication Nos. 20120017290 and 20110265198 assigned to Sangamo BioSciences, Inc.; US Patent Publication No. 20130236946 assigned to Cellectis; Platt et al., “CRISPR-Cas9 Knockin Mice for Genome Editing and Cancer Modeling” Cell (2014), 159(2): 440-455; “Oncogenic models based on delivery and use of the crispr-cas systems, vectors and compositions” WO2014204723A1 “Delivery and use of the crispr-cas systems, vectors and compositions for hepatic targeting and therapy” WO2014204726A1; “Delivery, use and therapeutic applications of the crispr-cas systems and compositions for modeling mutations in leukocytes” WO2016049251; and Chen et al., “Genome-wide CRISPR Screen in a Mouse Model of Tumor Growth and Metastasis” 2015, Cell 160, 1246-1260), which can be adapted for use with the present invention described herein.

In an embodiment, the cell or cells into which perturbations are introduced are tumor cells, such as tumor cells obtained from a subject in need of treatment. In an embodiment, the subject has or is suspected of having a cancer.

In one embodiment, one or more perturbations are introduced into one or more protein-coding genes or non-protein-coding DNA. In an embodiment, a programmable pattern recognition protein composition of the present invention and/or a CRISPR system or component thereof may be used to knockout protein-coding genes by frameshifts, point mutations, inserts, or deletions. An extensive toolbox may be used for efficient and specific CRISPR system mediated knockout as described herein, including a double-nicking CRISPR to efficiently modify both alleles of a target gene or multiple target loci and a smaller Cas protein for delivery on smaller vectors (Ran, F. A., et al., In vivo genome editing using Staphylococcus aureus Cas9. Nature. 520, 186-191 (2015)), which can be used as a guide and adapted for use with the present invention. A genome-wide sgRNA mouse library (˜10 sgRNAs/gene) may also be used in a mouse that expresses a suitable Cas protein (see, e.g., WO2014204727A1).

In one embodiment, perturbation is by deletion of regulatory elements. Non-coding elements may be targeted by using pairs of guide RNAs to delete regions of a defined size, and by tiling deletions covering sets of regions in pools.

In an embodiment, whole genome screens can be used for understanding the phenotypic readout of perturbing potential target genes. In preferred embodiments, perturbations target expressed genes as defined by a gene signature using a focused sgRNA library. Libraries may be focused on expressed genes in specific networks or pathways. In other preferred embodiments, regulatory drivers are perturbed.

Not being bound by a theory, perturbation studies targeting the genes and gene signatures described herein could (1) generate new insights regarding regulation and interaction of molecules within the system that contribute to suppression of an immune response, such as in the case within the tumor microenvironment, and (2) establish potential therapeutic targets or pathways that could be translated into clinical application.

### Target Detection

The programmable pattern recognition compositions and detection compositions described herein can be used in a method of detecting target cells, polypeptides, polynucleotides and/or combinations thereof, such as those present in a sample that contain, are coupled to, or are otherwise associated with a target molecule (e.g., a target polypeptide, target polynucleotide) and/or target molecular pattern, such as a PAMP. Such methods employ one or more of the detection compositions described herein, systems, cells, described herein, and/or devices described herein. Exemplary aspects of the method, e.g., detection constructs and detectable signal generation, are also described in greater detail elsewhere herein. Generally, a method of detection includes binding, complexing, or associating a programmable pattern recognition composition (such as a detection composition) of the present invention with a target molecule, such as one containing, coupled to, or otherwise associated with a molecular pattern (e.g., a PAMP), whereby the programmable pattern recognition protein composition or component thereof (e.g., an effector domain) is activated so as to modify a polypeptide or polynucleotide of a detection composition or component thereof (e.g., a detection construct) to produce a detectable signal, thereby indicating detection of a target cell, polypeptide, polynucleotide or other molecule. Detection can occur, in vitro, in vivo, in situ, or ex vivo. The system can be configured to detect one or more, e.g., 1, 2, 3, 4, 5, 6, 7, 8, 9, 10 or more different target polynucleotides. In an embodiment, the STAND NTPase contains peptidase activity so as to cleave a target peptide in a detection construct. In an embodiment, the programmable pattern recognition composition includes an effector, such as a molecule with nuclease activity that cleaves a polynucleotide of a detection construct. In an embodiment such an effector can be a Cas with or without collateral cleavage activity.

Described in an embodiment are methods of detecting a target molecule and/or cell, the method comprising combining a detection composition of the present invention (i e., one comprising an engineered protein of the present invention) or a formulation thereof and a sample or component thereof and activating an effector domain of the engineered protein via binding of a target polypeptide in the sample to the recognition domain thereby mediating effector domain modification of the detection construct and generation of a detectable signal.

In an embodiment, the method further comprising amplifying and/or enriching the target polynucleotide. In an embodiment, activating the peptidase further results in activation or generation of one or more signal amplification molecules.

Methods employing Cas13 or Cas12 based detection can be used as a general guide for configuration and design of a method, including sample processing, for target molecule, such as nucleic acids, detection methods employing the programmable nuclease-peptidase compositions of the present invention as they related to target nucleic acid preparation and processing (see e.g., Jong et al. N Engl J Med. 2020. 383(15):1492-1494; Broughton, et al. CRISPR-Cas12-based detection of SARS-CoV-2. Nat Biotechnol (2020), doi:10.1038/s41587-020-0513-4 (DETECTR detection); Gootenberg et al., Science. 2018 Apr. 27; 360(6387):439-444. doi: 10.1126/science.aaq0179 (multiplexing lateral flow platform for point-of-care diagnostics); and Chen, et al., Science. 2018 Apr. 27; 360(6387):436-439. doi: 10.1126/science.aar6245 (Cas12 detection), Myrhvold et al., Science 27 Apr. 2018: 360:6387, pp. 444-448; doi:10.1126/science.aas8836 (field deployable viral diagnostics), Joung et al., Point-of-care testing for COVID-19 using SHERLOCK diagnostics” doi: 10.1101/2020.05.04.20091231; Schmid-Burgk, et al., “LAMP-Seq: Population-Scale COVID-19 Diagnostics Using Combinatorial Barcoding,” doi: 10.1101/2020.04.06.025635, Gootenberg, 2018; Gootenberg, et al, Science. 2017 Apr. 28; 356(6336):438-442 (2017); Myhrvold, et al., Science 360, 444-448 (2018)). Nucleic acid detection with SHERLOCK relies on the collateral activity of Type VI and Type V Cas proteins, such as Cas13 and Cas12, which unleashes promiscuous cleavage of reporters upon target detection (Gooteneberg et al., 2018(Abudayyeh, et al., Science. 353(6299)(2016); East-Seletsky et al. Nature 538:270-273 (2016); Smargon et al. Mol Cell 65(4):618-630 (2017)), Gootenberg, 2018; Myhrvold et al. Science 360(6387):444-448 (2018); Gootenberg, 2017; Chen et al. Science 360(6387):436-439 (2018); Li et al. Cell Rep 25(12):3262-3272 (2018); Li et al. Nat Protoc 13(5):899-914 (2018), WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6, Science. 2018 Apr. 27; 360(6387):439-444; Gootenberg J S, et al., Nucleic acid detection with CRISPR-Cas13a/C2c2, Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016 Nov. 2; 7:13330, Myhrvold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety. Differences in the mechanism of nucleic acid detection and signal generation by a detection construct from such guiding methods and systems will be readily apparent in view of the description herein.

The low cost and adaptability of the assay platform described herein lends itself to a number of applications including (i) general bacterial and viral protein RNA/DNA quantitation, (ii) rapid, multiplexed RNA/DNA/protein expression detection, and (iii) sensitive detection of target nucleic acids, proteins, and cells, in both clinical and environmental samples. Additionally, the systems disclosed herein may be adapted for detection of transcripts within biological settings, such as cells. Given the highly specific nature of the effectors described herein, it may be possible to track allelic specific expression of transcripts or disease-associated mutations and/or the presence of microorganisms in live cells.

In an embodiment, a library of programmable pattern recognition compositions of the present invention is generated with each programmable pattern recognition compositions being capable of recognizing and/or binding a different target molecule (e.g., a target polypeptide and/or polynucleotide) and/or target molecular pattern (e.g., a PAMP), thus being activated by a different target molecule (e.g., a target polypeptide and/or polynucleotide) and/or target molecular pattern and thus recognizing a different target or groups of targets having the same target molecule (e.g., a target polypeptide and/or polynucleotide) and/or target molecular pattern. In an embodiment, each of the programmable pattern recognition compositions are placed in separate volumes and/or compartments. Each volume and/or compartment may then receive a different sample or aliquot of the same sample. In an embodiment, two or more programmable pattern recognition compositions, each recognizing and/or binding a different target molecule (e.g., a target polypeptide and/or polynucleotide) and/or target molecular pattern, are placed in a single volume, such as droplet, cell, well, or other discrete individual volume. Each volume may then receive a different sample or aliquot of the same sample.

In an embodiment where a guide molecule is co-delivered or is a component of the programmable pattern recognition composition that is a guide nucleic acid for a CRISPR-Cas system, a single guide RNA specific to a single target is placed in separate volumes. Each volume may then receive a different sample or aliquot of the same sample. In an embodiment, multiple guide RNA each to separate target may be placed in a single well such that multiple targets may be screened in a different well. In order to detect multiple guide RNAs in a single volume, in an embodiment, multiple effector proteins with different specificities may be used. For example, different orthologs with different sequence specificities may be used. For example, one orthologue may preferentially cut A, while others preferentially cut C, U, or T. Accordingly, guide RNAs that are all, or comprise a substantial portion, of a single nucleotide may be generated, each with a different fluorophore. In this way up to four different targets may be screened in a single individual discrete volume.

In an embodiment, the programmable pattern recognition compositions and systems and methods herein are capable of detecting down to at least attomolar concentrations of target molecules, such as bacterial or viral polynucleotides or polypeptides. In an embodiment, the programmable pattern recognition compositions and systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of bacterial or viral DNA, RNA, or polypeptides per microliter (cp/μL). In an embodiment, the programmable pattern recognition compositions and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of bacterial or viral DNA, RNA, or polypeptides per microliter (cp/μL) using a fluorescent or colorimetric readout.

In an embodiment, the detection reaction can occur as a two-step reaction in which amplification of target(s) and target detection via the effector composition/system of the present invention occur in separate reactions. In an embodiment, the detection reaction (including any target and/or signal amplification) can occur as a single, one-pot reaction. In an embodiment where the detection reaction is a one-pot reaction, target amplification is achieved using LAMP or RPA (see also below).

In an embodiment, the total time to perform the detection method (from sample preparation to detection) can be greater than 0 hours but less than about 4, 3.5, 3, 2.5, 2, 1.5, 1, or 0.5 hours. In an embodiment, the total time to perform the detection method (from sample preparation to detection) can occur within about 20 to 120 minutes, such as within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, to/or 120 minutes. In an embodiment, the total time to perform the detection method (from sample preparation to detection) can occur within about 20 to about 60 minutes, e.g. within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or/to 60 minutes. In an embodiment, the total time to perform the detection method (from sample preparation to detection) can occur within about 20 to about 45 minutes, e.g., within about 20, 21, 22, 23, 24, 25, 26, 27, 28, 29, 30, 31, 32, 33, 34, 35, 36, 37, 38, 39, 40, 41, 42, 43, 44, and/or 45 minutes. In an embodiment, the total time to perform the detection method (from sample preparation to detection) can occur within about 20 to about 30 minutes, e.g., within about 20, 21, 22, 23, 24, 25, 26, 27, 28, 29, and/or 30 minutes.

In an embodiment, the detection reaction can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In an embodiment, the detection reaction can occur within about 1 to about 45 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In an embodiment, the reaction can occur within about 1 to about 30 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In an embodiment, the detection reaction can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In an embodiment, the detection reaction can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In an embodiment, the detection reaction can occur within about 1 to about 15 minutes, e.g., within about 1, 2, 3, 4, 5, 6, 7, 8, 9, 10, 11, 12, 13, to/or about 15 minutes. In an embodiment, the detection reaction can occur within about 1 to about 10 minutes, e.g., within about 1, 2, 3, 4, 5, 6, 7, 8, 9, to/or about 10 minutes. In an embodiment, the detection reaction can occur within about 1 to about 5 minutes, e.g., within about 1, 2, 3, 4, to/or about 5 minutes.

### Sample and Target Nucleic Acid Processing, Isolation, Amplification, and Enrichment

In an embodiment, a sample and/or target polynucleotides or polypeptides is/are isolated, amplified, and/or enriched, and/or otherwise processed prior to amplification, enrichment, and/or detection. Such processing can include lysis of one or more cells or particles (e.g., viruses, exosomes, virus like particles, and/or the like) present in the sample to release target nucleic acids. In an embodiment, nucleic acids are isolated or otherwise separated from the one or more cells or particles (e.g., viruses, exosomes, virus like particles, and/or the like) present in the sample or sample lysate. In an embodiment, the method does not require or include extraction of the nucleic acids from the sample prior to amplification and/or target detection. In an embodiment, the sample preparation (e.g., lysis) and amplification occur in the same reaction vessel or location.

In an embodiment, the sample preparation (e.g., lysis), target amplification, and detection occur in the same reaction vessel or location. In an embodiment, the reaction vessel or location contains the sample preparation, amplification, and/or detection compositions and/or systems. In these embodiments, the sample can be added to the vessel and processing, amplification and detection can occur in the same vessel with no requirement to remove or add reagents to the vessel prior to obtaining a result. In an embodiment, the reagents, compositions, and systems are included in a vessel in a dehydrated (e.g., freeze dried, lyophilized, etc.) form and can be reconstituted when ready to use.

In an embodiment, the method includes preparation of the reagents for one or more steps, such as sample preparation, amplification, and/or detection, for storage. Such storage preparation can include, but is not limited to lyophilizing, freeze drying, or otherwise dehydrating them. They can be prepared for storage inside of individual reaction vessels or locations within a device or other vessel. In some of these embodiments, the reagents, compositions, systems or combinations thereof are e.g., lyophilized or freeze dried inside of the reaction vessel or at the specific discreet locations on a substrate or otherwise in a device. They can be stored at a suitable temperature ranging from ambient temperature (e.g., about 25-32 degrees C.) to about −20 or −80 degrees Celsius. In an embodiment, they are stored for 1, 2, 3, 4, 5, 6, 7, 8, 9, 10, 11, or 12 days, weeks, months or years. In an embodiment, the reagents, compositions, systems or combinations thereof are prepared and stored at about 4 degrees C. for about 1, 2, 3, 4, 5, 6, 7, 8, 9, 10, 11, or 12 days, weeks, months or years or more.

Due to the sensitivity of said systems, a number of applications that require from the rapid and sensitive detection may benefit from the embodiments disclosed herein and are contemplated to be within the scope of the invention. Further, any of the sample and/or nucleic acid processing methods described in this section can be applied, as relevant, to other methods employing the programmable nuclease-peptidase and detection compositions of the present invention herein. It is not intended to limit these features to just methods specifically designed to detect target polynucleotides.

### Sample Preparation

In an embodiment, the sample preparation can include release of polynucleotides (e.g., DNA and/or RNA) and/or polypeptides from cells and/or microorganisms, such as viruses, bacteria, engineered or other cells, particles (e.g., exosomes) etc., present in the sample. In an embodiment, the sample preparation can include virus, bacteria, inactivation and/or nuclease inactivation. The step of sample preparation can occur prior to any target amplification and/or detection. In an embodiment, sample preparation can include nuclease inactivation and/or viral inactivation by 1, 2, 3, 4 or more thermal (heat or cold) inactivation steps, chemical inactivation steps, biologic inactivation, physiologic inactivation, physical inactivation steps, or any combination thereof. The phrase “physiological inactivation” refers to conditions that deviate from the normal working physiological conditions (e.g., pH, osmolarity, temperature, salinity, etc.) necessary for causing or maintaining the activation of a component (e.g., an enzyme) present in a sample that result in the inactivation or inhibition of the function or activity of the component. Inactivation can, in an embodiment, result in lysis of the cells, microorganisms, viruses, and/or particles. In an embodiment, the same methods and reagents can be applied to other microbes (e.g., bacteria and eukaryotic cells).

Amplification and Enrichment of Target and/or Signal

### Target Amplification

In an embodiment, target RNAs and/or DNAs may be amplified prior to activating the effector protein of the composition and/or system of the present invention. Any suitable RNA or DNA amplification technique may be used. In an embodiment, the RNA or DNA amplification is an isothermal amplification. In an embodiment, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In an embodiment, non-isothermal amplification methods may be used which include, but are not limited to, PCR, multiple displacement amplification (MDA), rolling circle amplification (RCA), ligase chain reaction (LCR), or ramification amplification method (RAM). In an embodiment, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), or a helicase-based amplification method (see e.g. WO 2020/006036, which is incorporated by reference herein as if expressed in its entirety). In an embodiment, amplification is via LAMP. In an embodiment, amplification is via RPA.

In an embodiment, the RNA or DNA amplification is nucleic acid sequence-based amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the effector protein effector protein of the composition and/or system of the present invention and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In an embodiment, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the effector system effector protein of the composition and/or system of the present invention. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in an embodiment the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in an embodiment, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In an embodiment, amplification reagents as described herein may be appropriate for use in hot-start amplification. Hot start amplification may be beneficial in an embodiment to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In an embodiment, reagents or components appropriate for use with hot-start amplification may be used in place of one or more of the composition components as appropriate. For example, a polymerase or other reagent may be used that exhibits a desired activity at a particular temperature or other reaction condition. In an embodiment, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification reagents can include one or more primers and/or probes optimized for amplification of a target sequence by one or more of the amplification methods previously described. Primer and probe design for the methods described herein will be within the purview of one of ordinary skill in the art in view of the context and disclosure only provided herein.

Amplification of nucleic acids may be performed using specific thermal cycle machinery or equipment and may be performed in single reactions or in bulk, such that any desired number of reactions may be performed simultaneously. In an embodiment, amplification may be performed using microfluidic or robotic devices, or may be performed using manual alteration in temperatures to achieve the desired amplification. In an embodiment, optimization may be performed to obtain the optimum reactions conditions for the particular application or materials. One of skill in the art will understand and be able to optimize reaction conditions to obtain sufficient amplification.

In an embodiment, detection of DNA with the methods or systems of the invention requires transcription of the (amplified) DNA into RNA prior to detection.

In an embodiment, the amplification reagent or component thereof is shelf-stable. In an embodiment, the amplification reagent or component thereof is shelf-stable at ambient temperature.

### Target Polynucleotide Enrichment

In an embodiment, target polypeptides, RNA, and/or DNA may first be enriched prior to detection or amplification of the target polypeptides, RNA, and/or DNA. In an embodiment, this enrichment may be achieved by binding of the target nucleic acids by a CRISPR effector system or other suitable affinity based capture strategy capable of specifically capturing target nucleic acids so as to allow separation from non-target nucleic acids. In an embodiment, polypeptides are enriched by using a suitable immunoseparation technique or other pull-down type assay. Such techniques for enriching polypeptides are generally known in the art.

Current target-specific enrichment protocols require single-stranded nucleic acid prior to hybridization with probes. Among various advantages, the present embodiments can skip this step and enable direct targeting to double-stranded DNA (either partly or completely double-stranded). In addition, the embodiments disclosed herein are enzyme-driven targeting methods that offer faster kinetics and easier workflow allowing for isothermal enrichment. In an embodiment, a set of guide RNAs to different target nucleic acids are used in a single assay, allowing for detection of multiple targets an/or multiple variants of a single target.

In an embodiment, a dead CRISPR effector protein may bind the target nucleic acid in solution and then subsequently be isolated from said solution. For example, the dead CRISPR effector protein bound to the target nucleic acid, may be isolated from the solution using an antibody or other molecule, such as an aptamer, that specifically binds the dead CRISPR effector protein.

In other example embodiments, the dead CRISPR effector protein may bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In an embodiment, the solid support comprises a patterned surface suitable for immobilization of molecules in an ordered pattern. In an embodiment a patterned surface refers to an arrangement of different regions in or on an exposed layer of a solid support. In an embodiment, the solid support comprises an array of wells or depressions in a surface. The composition and geometry of the solid support can vary with its use. In an embodiment, the solids support is a planar structure such as a slide, chip, microchip and/or array. As such, the surface of the substrate can be in the form of a planar layer. In an embodiment, the solid support comprises one or more surfaces of a flowcell. The term “flowcell” as used herein refers to a chamber comprising a solid surface across which one or more fluid reagent can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In an embodiment, the solid support or its surface is non-planar, such as the inner or outer surface of a tube or vessel. In an embodiment, the solid support comprise microspheres or beads. “Microspheres,” “bead,” “particles,” are intended to mean within the context of a solid substrate to mean small discrete particles made of various material including, but not limited to, plastics, ceramics, glass, and polystyrene. In an embodiment, the microspheres are magnetic microspheres or beads. Alternatively or additionally, the beads may be porous. The bead sizes range from nanometers, e.g., 100 nm, to millimeters, e.g., 1 mm.

A sample containing, or suspected of containing, the target nucleic acids may then be exposed to the substrate to allow binding of the target nucleic acids to the bound dead CRISPR effector protein. Non-target molecules may then be washed away. In an embodiment, the target nucleic acids may then be released from the CRISPR effector protein/guide RNA complex for further detection using the methods disclosed herein. In an embodiment, the target nucleic acids may first be amplified as described herein. In an embodiment, the CRISPR effector may be labeled with a binding tag. In an embodiment the CRISPR effector may be chemically tagged. For example, the CRISPR effector may be chemically biotinylated. In another example embodiment, a fusion may be created by adding additional sequence encoding a fusion to the CRISPR effector. One example of such a fusion is an AviTag™, which employs a highly targeted enzymatic conjugation of a single biotin on a unique 15 amino acide peptide tag. In an embodiment, the CRISPR effector may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the CRISPR effector system once it has bound a target nucleic acid or to fix the CRISPR effector system on the solid substrate.

In an embodiment, a guide RNA may be labeled with a binding tag. In an embodiment, the entire guide RNA may be labeled using in vitro transcription (IVT) incorporating one or more biotinylated nucleotides, such as, biotinylated uracil. In an embodiment, biotin can be chemically or enzymatically added to the guide RNA, such as, the addition of one or more biotin groups to the 3′ end of the guide RNA. The binding tag may be used to pull down the guide RNA/target nucleic acid complex after binding has occurred, for example, by exposing the guide RNA/target nucleic acid to a streptavidin coated solid substrate.

Accordingly, in an embodiment, an engineered or non-naturally-occurring CRISPR effector may be used for enrichment purposes. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of the RNA strand at the target locus of interest. In an embodiment, the one or more mutations may comprise two mutations. In an embodiment the one or more amino acid residues are modified in a C2c2 effector protein, e.g., an engineered or non-naturally-occurring effector protein or C2c2. In an embodiment, the one or more modified of mutated amino acid residues are one or more of those in C2c2 corresponding to R597, H602, R1278 and H1283 (referenced to Lsh C2c2 amino acids), such as mutations R597A, H602A, R1278A and H1283A, or the corresponding amino acid residues in Lsh C2c2 orthologues.

In an embodiment, the one or more modified of mutated amino acid residues are one or more of those in C2c2 corresponding to K2, K39, V40, E479, L514, V518, N524, G534, K535, E580, L597, V602, D630, F676, L709, 1713, R717 (HEPN), N718, H722 (HEPN), E773, P823, V828, I879, Y880, F884, Y997, L1001, F1009, L1013, Y1093, L1099, L1111, Y1114, L1203, D1222, Y1244, L1250, L1253, K1261, 11334, L1355, L1359, R1362, Y1366, E1371, R1372, D1373, R1509 (HEPN), H1514 (HEPN), Y1543, D1544, K1546, K1548, V1551, 11558, according to C2c2 consensus numbering. In an embodiment, the one or more modified of mutated amino acid residues are one or more of those in C2c2 corresponding to R717 and R1509. In an embodiment, the one or more modified of mutated amino acid residues are one or more of those in C2c2 corresponding to K2, K39, K535, K1261, R1362, R1372, K1546 and K1548. In an embodiment, said mutations result in a protein having an altered or modified activity. In an embodiment, said mutations result in a protein having a reduced activity, such as reduced specificity. In an embodiment, said mutations result in a protein having no catalytic activity (i.e., “dead” C2c2). In an embodiment, said amino acid residues correspond to Lsh C2c2 amino acid residues, or the corresponding amino acid residues of a C2c2 protein from a different species.

The above enrichment systems may also be used to deplete a sample of certain nucleic acids. For example, guide RNAs may be designed to bind non-target RNAs to remove the non-target RNAs from the sample. In one example embodiment, the guide RNAs may be designed to bind nucleic acids that do carry a particular nucleic acid variation. For example, in a given sample a higher copy number of non-variant nucleic acids may be expected. Accordingly, the embodiments disclosed herein may be used to remove the non-variant nucleic acids from a sample, to increase the efficiency with which the detection effector system effector protein of the composition and/or system of the present invention can detect the target variant sequences in a given sample.

Amplification and/or Enhancement of Detectable Signal

In an embodiment, further modification or reagents may be introduced that further amplify the detectable positive signal. For example, an activated effector domain of an engineered protein the present invention may be used to generate a secondary target or additional guide sequence, or both. In one example embodiment, the reaction solution would contain a secondary target polypeptide that is spiked in at high concentration. The secondary target polypeptide may be distinct from the primary target polypeptide (i.e., the first target polypeptide e for which the assay is designed to detect) and in certain instances may be common across all reaction volumes. A secondary polypeptide may include a protecting group such that is not active until acted upon by the effector protein. Cleavage of the protecting group by an activated effector protein (i.e., after activation by formation of complex with the primary target(s) in solution) and formation of a complex with free effector protein in solution and activation from the spiked in secondary target polypeptide.

In an embodiment a CRISPR system can be used to enrich or amplify the detectable signal. In an embodiment, the programmable pattern recognition compositions and systems of the present invention that is/are activated upon target recognition and/or binding can produce, such as via collateral (e.g., peptidase, nuclease, etc.) activity of one or more components of the programmable pattern recognition composition, species that can activate (or be targets of) a CRISPR system (such as a Cas-12 or Cas-13 detection system) thus amplifying the signal for detection. In an embodiment a CRISPR type-III effector can be used as the signal amplifying system. In an embodiment, the type III effector is Csm6, which is which is activated by cyclic adenylate molecules or linear adenine homopolymers terminated with a 2′,3′-cyclic phosphate. In an embodiment, the first CRISPR system includes a Cas13 (e.g., Cas 13a, 13b, 13c, or 13d) and/or a Cas 12a effector(s) and the amplification system or molecule is or includes Csm6. See also Gootenberg et al. 2018. Science. 360:439-44 and WO 2019/051318, which are incorporated by reference herein as if expressed in their entireties.

### Exemplary Applications of the Target Polynucleotide Detection Methods

### Microbe and Virus Detection and Applications

In an embodiment, the systems, devices, and methods, disclosed herein are directed to detecting the presence of one or more microbial agents in a sample, such as a biological sample obtained from a subject. In an embodiment, the microbe may be a bacterium, a fungus, a yeast, a protozoa, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g., bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a point of care (POC) device, the embodiments disclosed herein may be used guide therapeutic regimens, such as selection of the appropriate antibiotic or antiviral. The embodiments disclosed herein may also be used to screen environmental samples (air, water, surfaces, food etc.) for the presence of microbial contamination.

Disclosed is a method to identify microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multi-level analysis can be performed for a particular subject in which any number of microbes can be detected at once. In an embodiment, simultaneous detection of multiple microbes may be performed using a set of probes that can identify one or more microbial species.

Multiplex analysis of samples enables large-scale detection of samples, reducing the time and cost of analyses. However, multiplex analyses are often limited by the availability of a biological sample. In accordance with the invention, however, alternatives to multiplex analysis may be performed such that multiple effector proteins can be added to a single sample and each detection construct may be combined with a separate quencher dye. In this case, positive signals may be obtained from each quencher dye separately for multiple detection in a single sample.

Disclosed herein are methods for distinguishing between two or more species of one or more organisms in a sample. The methods are also amenable to detecting one or more species of one or more organisms in a sample.

### Microbe Detection

In an embodiment, a method for detecting microbes in samples is provided comprising distributing a sample or set of samples into one or more individual discrete volumes, the individual discrete volumes comprising a programmable pattern recognition composition of the present invention; incubating the sample or set of samples under conditions sufficient to allow recognition and/or binding of the programmable pattern recognition composition to a target molecule (e.g., a target polypeptide and/or polynucleotide) and/or target molecular pattern on, in or secreted by one or more microbe targets; activating one or more effector domains of the programmable pattern recognition composition and/or component thereof via recognition and/or binding of the programmable pattern recognition composition to a target molecule (e.g., a target polypeptide and/or polynucleotide) and/or target molecular pattern of the one or more target microbes or molecules, wherein activating the programmable pattern recognition composition results in modification of a detection construct, such as a polypeptide or polynucleotide (e.g., RNA)-based detection construct, such that a detectable positive signal is generated; and detecting the detectable positive signal, wherein detection of the detectable positive signal indicates a presence of one or more target molecules or cells in the sample containing a target molecule (e.g., a target polypeptide and/or polynucleotide) and/or target molecular pattern or other recognized molecular pattern. The one or more target molecules may be mRNA, gDNA (coding or non-coding), trRNA, RNA, or peptides or polypeptides. The guide RNAs may be designed to detect target sequences. Where the systems include or involve guide RNAs, certain embodiments disclosed herein may also utilize certain steps to improve hybridization between guide RNA and target RNA sequences. Methods for enhancing ribonucleic acid hybridization are disclosed in WO 2015/085194, entitled “Enhanced Methods of Ribonucleic Acid Hybridization” which is incorporated herein by reference. The microbe-specific target may be RNA or DNA or a protein. If DNA, the method may further comprise the use of DNA primers that introduce an RNA polymerase promoter as described herein. If the target is a protein then aptamers can be utilized and the method includes one or more specific to protein detection described herein.

### Detection of Single Nucleotide Variants

In an embodiment, one or more identified target sequences may be detected using engineered proteins of the present invention and/or guide RNAs that are specific for and bind to the target sequence as described herein. The systems and methods of the present invention can distinguish even between single nucleotide polymorphisms present among different microbial species and therefore, use of multiple guide RNAs in accordance with the invention may further expand on or improve the number of target sequences that may be used to distinguish between species. For example, in an embodiment, the one or more guide RNAs may distinguish between microbes at the species, genus, family, order, class, phylum, kingdom, or phenotype, or a combination thereof. This application can also apply to non-microbial cells, such as human cells in detection of disease or genotyping.

Detection Based on rRNA Sequences

In an embodiment, the devices, systems, and methods disclosed herein may be used to distinguish multiple microbial species in a sample. In an embodiment, identification may be based on ribosomal RNA sequences, including the 16S, 23S, and 5S subunits. Methods for identifying relevant rRNA sequences are disclosed in U.S. Patent Application Publication No. 2017/0029872. In an embodiment, a set of guide RNA may be designed to distinguish each species by a variable region that is unique to each species or strain. Guide RNAs may also be designed to target RNA genes that distinguish microbes at the genus, family, order, class, phylum, kingdom levels, or a combination thereof. In an embodiment where amplification is used, a set of amplification primers may be designed to flanking constant regions of the ribosomal RNA sequence and a guide RNA designed to distinguish each species by a variable internal region. In an embodiment, the primers and guide RNAs may be designed to conserved and variable regions in the 16S subunit respectfully. Other genes or genomic regions that uniquely variable across species or a subset of species such as the RecA gene family, RNA polymerase p subunit, may be used as well. Other suitable phylogenetic markers, and methods for identifying the same, are discussed for example in Wu et al. arXiv:1307.8690 [q-bio.GN].

In an embodiment, a method or diagnostic is designed to screen microbes across multiple phylogenetic and/or phenotypic levels at the same time. For example, the method or diagnostic may comprise the use of multiple detection compositions or systems of the present invention with different guide RNAs. A first set of guide RNAs may distinguish, for example, between mycobacteria, gram positive, and gram-negative bacteria. These general classes can be even further subdivided. For example, guide RNAs could be designed and used in the method or diagnostic that distinguish enteric and non-enteric within gram negative bacteria. A second set of guide RNA can be designed to distinguish microbes at the genus or species level. Thus, a matrix may be produced identifying all mycobacteria, gram positive, gram negative (further divided into enteric and non-enteric) with each genus of species of bacteria identified in a given sample that fall within one of those classes. In an embodiment, identification of microbes is based on other target molecules, such as polypeptides, or other microbe specific structural features. In an embodiment, identification of microbes is based on target molecular patterns, such as PAMPS. The foregoing is for example purposes only. Other means for classifying other microbe types are also contemplated and would follow the general structure described above.

### Screening for Drug Resistance

In an embodiment, the devices, systems and methods disclosed herein may be used to screen for microbial genes and/or proteins of interest, for example antibiotic and/or antiviral resistance genes/proteins. Guide RNAs may be designed to distinguish between known genes of interest. Samples, including clinical samples, may then be screened using the embodiments disclosed herein for detection of such genes. The ability to screen for drug resistance at POC would have tremendous benefit in selecting an appropriate treatment regime. In an embodiment, the antibiotic resistance genes are carbapenemases including KPC, NDM1, CTX-M15, OXA-48. Other antibiotic resistance genes are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

Ribavirin is an effective antiviral that hits a number of RNA viruses. Several clinically important viruses have evolved ribavirin resistance including Foot and Mouth Disease Virus doi:10.1128/JVI.03594-13; polio virus (Pfeifer and Kirkegaard. PNAS, 100(12):7289-7294, 2003); and hepatitis C virus (Pfeiffer and Kirkegaard, J. Virol. 79(4):2346-2355, 2005). A number of other persistent RNA viruses, such as hepatitis and HIV, have evolved resistance to existing antiviral drugs: hepatitis B virus (lamivudine, tenofovir, entecavir) doi:10/1002/hep22900; hepatitis C virus (telaprevir, BILN2061, ITMN-191, SCh6, boceprevir, AG-021541, ACH-806) doi:10.1002/hep.22549; and HIV (many drug resistance mutations) hivb.standford.edu. The embodiments disclosed herein may be used to detect such variants among others.

Aside from drug resistance, there are a number of clinically relevant mutations that could be detected with the embodiments disclosed herein, such as persistent versus acute infection in LCMV (doi:10.1073/pnas.1019304108), and increased infectivity of Ebola (Diehl et al. Cell. 2016, 167(4):1088-1098.

As described herein elsewhere, closely related microbial species (e.g., having only a single nucleotide difference in a given target sequence) may be distinguished by introduction of a synthetic mismatch in the gRNA.

### Set Cover Approaches

In an embodiment, a set of guide RNAs is employed and designed that can identify, for example, all microbial species within a defined set of microbes. In an embodiment, the methods for generating guide RNAs as described herein may be compared to methods disclosed in WO 2017/040316, incorporated herein by reference. As described in WO 2017040316, a set cover solution may identify the minimal number of target sequences probes or guide RNAs needed to cover an entire target sequence or set of target sequences, e.g. a set of genomic sequences. Set cover approaches have been used previously to identify primers and/or microarray probes, typically in the 20 to 50 base pair range. See, e.g., Pearson et al, cs.virginia.edu/-robins/papers/primers_dam11_final.pdf., Jabado et al. Nucleic Acids Res. 2006 34(22):6605-11, Jabado et al. Nucleic Acids Res. 2008, 36(1):e3 doi10.1093/nar/gkm1106, Duitama et al. Nucleic Acids Res. 2009,37(8):2483-2492, Phillippy et al. BMC Bioinformatics. 2009, 10:293 doi:10. 1186/1471-2105-10-293. However, such approaches generally involved treating each primer/probe as k-mers and searching for exact matches or allowing for inexact matches using suffix arrays. In addition, the methods generally take a binary approach to detecting hybridization by selecting primers or probes such that each input sequence only needs to be bound by one primer or probe and the position of this binding along the sequence is irrelevant. Alternative methods may divide a target genome into pre-defined windows and effectively treat each window as a separate input sequence under the binary approach—i.e., they determine whether a given probe or guide RNA binds within each window and require that all of the windows be bound by the same of some probe or guide RNA. Effectively, these approaches treat each element of the “universe” in the set cover problem as being either an entire input sequence or a pre-defined window of an input sequence, and each element is considered “covered” if the start of a probe or guide RNA binds within the element. These approaches limit the fluidity to which different probe or guide RNA designs are allowed to cover a given target sequence.

In contrast, the embodiments disclosed herein are directed to detecting longer probe or guide RNA lengths, for example, in the range of 70 bp to 200 bp that are suitable for hybrid selection sequencing. In addition, the methods disclosed WO 2017/040316 herein may be applied to take a pan-target sequence approach capable of defining a probe or guide RNA sets that can identify and facilitate the detection sequencing of all species and/or strains sequences in a large and/or variable target sequence set. For example, the methods disclosed herein may be used to identify all variants of a given virus, or multiple different viruses in a single assay. Further, the method disclosed herein treat each element of the “universe” in the set cover problem as being a nucleotide of a target sequence, and each element is considered “covered” as long as a probe or guide RNA binds to some segment of a target genome that includes the element. These type of set cover methods may be used instead of the binary approach of previous methods, the methods disclosed in herein better model how a probe or guide RNA may hybridize to a target sequence. Rather than only asking if a given guide RNA sequence does or does not bind to a given window, such approaches may be used to detect a hybridization pattern—i.e., where a given probe or guide RNA binds to a target sequence or target sequences—and then determines from those hybridization patterns the minimum number of probes or guide RNAs needed to cover the set of target sequences to a degree sufficient to enable both enrichment from a sample and sequencing of any and all target sequences. These hybridization patterns may be determined by defining certain parameters that minimize a loss function, thereby enabling identification of minimal probe or guide RNA sets in a way that allows parameters to vary for each species, e.g., to reflect the diversity of each species, as well as in a computationally efficient manner that cannot be achieved using a straightforward application of a set cover solution, such as those previously applied in the probe or guide RNA design context.

The ability to detect multiple transcript abundances may allow for the generation of unique microbial signatures indicative of a particular phenotype. Various machine learning techniques may be used to derive the gene signatures. Accordingly, the guide RNAs of the detection compositions/systems of the present invention may be used to identify and/or quantitate relative levels of biomarkers defined by the gene signature in order to detect certain phenotypes. In an embodiment, the gene signature indicates susceptibility to an antibiotic, resistance to an antibiotic, or a combination thereof.

In one aspect of the invention, a method comprises detecting one or more pathogens. In this manner, differentiation between infection of a subject by individual microbes may be obtained. In an embodiment, such differentiation may enable detection or diagnosis by a clinician of specific diseases, for example, different variants of a disease. Preferably the pathogen sequence is a genome of the pathogen or a fragment thereof. The method further may comprise determining the evolution of the pathogen. Determining the evolution of the pathogen may comprise identification of pathogen mutations, e.g., nucleotide deletion, nucleotide insertion, nucleotide substitution. Amongst the latter, there are non-synonymous, synonymous, and noncoding substitutions. Mutations are more frequently non-synonymous during an outbreak. The method may further comprise determining the substitution rate between two pathogen sequences analyzed as described above. Whether the mutations are deleterious or even adaptive would require functional analysis, however, the rate of non-synonymous mutations suggests that continued progression of this epidemic could afford an opportunity for pathogen adaptation, underscoring the need for rapid containment. Thus, the method may further comprise assessing the risk of viral adaptation, wherein the number non-synonymous mutations is determined. (Gire, et al., Science 345, 1369, 2014).

### Monitoring Microbe Outbreaks

In an embodiment, a detection composition of the present invention or methods of use thereof as described herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, or a mechanism involved in a disease outbreak caused by a pathogen.

The pattern of pathogen transmission may comprise continued new transmissions from the natural reservoir of the pathogen or subject-to-subject transmissions (e.g., human-to-human transmission) following a single transmission from the natural reservoir or a mixture of both. In one embodiment, the pathogen transmission may be bacterial or viral transmission, in such case, the target sequence is preferably a microbial genome or fragments thereof. In one embodiment, the pattern of the pathogen transmission is the early pattern of the pathogen transmission, i.e., at the beginning of the pathogen outbreak. Determining the pattern of the pathogen transmission at the beginning of the outbreak increases likelihood of stopping the outbreak at the earliest possible time thereby reducing the possibility of local and international dissemination.

Determining the pattern of the pathogen transmission may comprise detecting a pathogen sequence according to the methods described herein. Determining the pattern of the pathogen transmission may further comprise detecting shared intra-host variations of the pathogen sequence between the subjects and determining whether the shared intra-host variations show temporal patterns. Patterns in observed intrahost and interhost variation provide important insight about transmission and epidemiology (Gire, et al., 2014).

Detection of shared intra-host variations between the subjects that show temporal patterns is an indication of transmission links between subject (in particular between humans) because it can be explained by subject infection from multiple sources (superinfection), sample contamination recurring mutations (with or without balancing selection to reinforce mutations), or co-transmission of slightly divergent viruses that arose by mutation earlier in the transmission chain (Park, et al., Cell 161(7):1516-1526, 2015). Detection of shared intra-host variations between subjects may comprise detection of intra-host variants located at common single nucleotide polymorphism (SNP) positions. Positive detection of intra-host variants located at common (SNP) positions is indicative of superinfection and contamination as primary explanations for the intra-host variants. Superinfection and contamination can be parted on the basis of SNP frequency appearing as inter-host variants (Park, et al., 2015). Otherwise, superinfection and contamination can be ruled out. In this latter case, detection of shared intra-host variations between subjects may further comprise assessing the frequencies of synonymous and nonsynonymous variants and comparing the frequency of synonymous and nonsynonymous variants to one another. A nonsynonymous mutation is a mutation that alters the amino acid of the protein, likely resulting in a biological change in the microbe that is subject to natural selection. Synonymous substitution does not alter an amino acid sequence. Equal frequency of synonymous and nonsynonymous variants is indicative of the intra-host variants evolving neutrally. If frequencies of synonymous and nonsynonymous variants are divergent, the intra-host variants are likely to be maintained by balancing selection. If frequencies of synonymous and nonsynonymous variants are low, this is indicative of recurrent mutation. If frequencies of synonymous and nonsynonymous variants are high, this is indicative of co-transmission (Park, et al., 2015).

Like Ebola virus, Lassa virus (LASV) can cause hemorrhagic fever with high case fatality rates. Andersen et al. generated a genomic catalog of almost 200 LASV sequences from clinical and rodent reservoir samples (Andersen, et al., Cell Volume 162, Issue 4, p 738-750, 13 Aug. 2015). Andersen et al. show that whereas the 2013-2015 EVD epidemic is fueled by human-to-human transmissions, LASV infections mainly result from reservoir-to-human infections. Andersen et al. elucidated the spread of LASV across West Africa and show that this migration was accompanied by changes in LASV genome abundance, fatality rates, codon adaptation, and translational efficiency. The method may further comprise phylogenetically comparing a first pathogen sequence to a second pathogen sequence, and determining whether there is a phylogenetic link between the first and second pathogen sequences. The second pathogen sequence may be an earlier reference sequence. If there is a phylogenetic link, the method may further comprise rooting the phylogeny of the first pathogen sequence to the second pathogen sequence. Thus, it is possible to construct the lineage of the first pathogen sequence. (Park, et al., 2015).

The method may further comprise determining whether the mutations are deleterious or adaptive. Deleterious mutations are indicative of transmission-impaired viruses and dead-end infections, thus normally only present in an individual subject. Mutations unique to one individual subject are those that occur on the external branches of the phylogenetic tree, whereas internal branch mutations are those present in multiple samples (i.e., in multiple subjects). Higher rate of nonsynonymous substitution is a characteristic of external branches of the phylogenetic tree (Park, et al., 2015).

In internal branches of the phylogenetic tree, selection has had more opportunity to filter out deleterious mutants. Internal branches, by definition, have produced multiple descendent lineages and are thus less likely to include mutations with fitness costs. Thus, lower rate of nonsynonymous substitution is indicative of internal branches (Park, et al., 2015).

Synonymous mutations, which likely have less impact on fitness, occurred at more comparable frequencies on internal and external branches (Park, et al., 2015).

By analyzing the sequenced target sequence, such as viral genomes, it is possible to discover the mechanisms responsible for the severity of the epidemic episode such as during the 2014 Ebola outbreak. For example, Gire et al. made a phylogenetic comparison of the genomes of the 2014 outbreak to all 20 genomes from earlier outbreaks suggests that the 2014 West African virus likely spread from central Africa within the past decade. Rooting the phylogeny using divergence from other ebolavirus genomes was problematic (6, 13). However, rooting the tree on the oldest outbreak revealed a strong correlation between sample date and root-to-tip distance, with a substitution rate of 8×10⁻⁴ per site per year (13). This suggests that the lineages of the three most recent outbreaks all diverged from a common ancestor at roughly the same time, around 2004, which supports the hypothesis that each outbreak represents an independent zoonotic event from the same genetically diverse viral population in its natural reservoir. They also found out that the 2014 EBOV outbreak might be caused by a single transmission from the natural reservoir, followed by human-to-human transmission during the outbreak. Their results also suggested that the epidemic episode in Sierra Leon might stem from the introduction of two genetically distinct viruses from Guinea around the same time (Gire, et al., 2014).

It has been also possible to determine how the Lassa virus spread out from its origin point, in particular thanks to human-to-human transmission and even retrace the history of this spread 400 years back (Andersen, et al., Cell 162(4):738-50, 2015).

In relation to the work needed during the 2013-2015 EBOV outbreak and the difficulties encountered by the medical staff at the site of the outbreak, and more generally, the method of the invention makes it possible to carry out sequencing using fewer selected probes such that sequencing can be accelerated, thus shortening the time needed from sample taking to results procurement. Further, kits and systems can be designed to be usable on the field so that diagnostics of a patient can be readily performed without need to send or ship samples to another part of the country or the world.

In any method described above, sequencing the target sequence or fragment thereof may used any of the sequencing processes described above. Further, sequencing the target sequence or fragment thereof may be a near-real-time sequencing. Sequencing the target sequence or fragment thereof may be carried out according to previously described methods (Experimental Procedures: Matranga et al., 2014; and Gire, et al., 2014). Sequencing the target sequence or fragment thereof may comprise parallel sequencing of a plurality of target sequences. Sequencing the target sequence or fragment thereof may comprise Illumina sequencing.

Analyzing the target sequence or fragment thereof that hybridizes to one or more of the selected probes may be an identifying analysis, wherein hybridization of a selected probe to the target sequence or a fragment thereof indicates the presence of the target sequence within the sample.

Currently, primary diagnostics are based on the symptoms a patient has. However, various diseases may share identical symptoms so that diagnostics rely much on statistics. For example, malaria triggers flu-like symptoms: headache, fever, shivering, joint pain, vomiting, hemolytic anemia, jaundice, hemoglobin in the urine, retinal damage, and convulsions. These symptoms are also common for septicemia, gastroenteritis, and viral diseases. Amongst the latter, Ebola hemorrhagic fever has the following symptoms fever, sore throat, muscular pain, headaches, vomiting, diarrhea, rash, decreased function of the liver and kidneys, internal and external hemorrhage.

When a patient is presented to a medical unit, for example in tropical Africa, basic diagnostics will conclude to malaria because statistically, malaria is the most probable disease within that region of Africa. The patient is consequently treated for malaria although the patient might not actually have contracted the disease and the patient ends up not being correctly treated. This lack of correct treatment can be life-threatening especially when the disease the patient contracted presents a rapid evolution. It might be too late before the medical staff realizes that the treatment given to the patient is ineffective and comes to the correct diagnostics and administers the adequate treatment to the patient.

The method of the invention provides a solution to this situation. Indeed, because the number of guide RNAs can be dramatically reduced, this makes it possible to provide on a single chip selected probes divided into groups, each group being specific to one disease, such that a plurality of diseases, e.g., viral infection, can be diagnosed at the same time. Thanks to the invention, more than 3 diseases can be diagnosed on a single chip, preferably more than 4, 5, 6, 7, 8, 9, 10, 11, 12, 13, 14, 15, 16, 17, 18, 19, 20 diseases at the same time, preferably the diseases that most commonly occur within the population of a given geographical area. Since each group of selected probes is specific to one of the diagnosed diseases, a more accurate diagnostics can be performed, thus diminishing the risk of administering the wrong treatment to the patient.

In other cases, a disease such as a viral infection may occur without any symptoms, or had caused symptoms but they faded out before the patient is presented to the medical staff. In such cases, either the patient does not seek any medical assistance or the diagnostics is complicated due to the absence of symptoms on the day of the presentation.

The present invention may also be used in concert with other methods of diagnosing disease, identifying pathogens and optimizing treatment based upon detection of nucleic acids, such as mRNA in crude, non-purified samples.

The method of the invention also provides a powerful tool to address this situation. Indeed, since a plurality of groups of selected guide RNAs, each group being specific to one of the most common diseases that occur within the population of the given area, are comprised within a single diagnostic, the medical staff only need to contact a biological sample taken from the patient with the chip. Reading the chip reveals the diseases the patient has contracted.

In some cases, the patient is presented to the medical staff for diagnostics of particular symptoms. The method of the invention makes it possible not only to identify which disease causes these symptoms but at the same time determine whether the patient suffers from another disease he was not aware of.

This information might be of utmost importance when searching for the mechanisms of an outbreak. Indeed, groups of patients with identical viruses also show temporal patterns suggesting a subject-to-subject transmission links.

In an embodiment, a programmable pattern recognition protein composition or method of use thereof as described herein may be used to predict disease outcome in patients suffering from viral diseases. In specific embodiments, such viral diseases may include, but are not necessarily limited to, Lassa fever. Specific factors related to Lassa fever disease outcome may include but are not necessarily limited to, age, extent of kidney injury, and/or CNS injury.

### Screening Microbial Genetic Perturbations

In an embodiment, the programmable pattern recognition compositions and systems of the present invention disclosed herein may be used to screen microbial genetic perturbations. Such methods may be useful, for example to map out microbial pathways and functional networks. Microbial cells may be genetically modified and then screened under different experimental conditions. As described above, the embodiments disclosed herein can screen for multiple target molecules in a single sample, or a single target in a single individual discrete volume in a multiplex fashion. Genetically modified microbes may be modified to include a nucleic acid barcode sequence that identifies the particular genetic modification carried by a particular microbial cell or population of microbial cells. A barcode is s short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the effector compositions and systems of the present invention described herein may be used to detect the barcode. Detection of the positive detectable signal indicates the presence of a particular genetic modification in the sample. The methods disclosed herein may be combined with other methods for detecting complimentary genotype or phenotypic readouts indicating the effect of the genetic modification under the experimental conditions tested. Genetic modifications to be screened may include, but are not limited to, a gene knock-in, a gene knock-out, inversions, translocations, transpositions, or one or more nucleotide insertions, deletions, substitutions, mutations, or addition of nucleic acids encoding an epitope with a functional consequence such as altering protein stability or detection. In a similar fashion, the methods described herein may be used in synthetic biology application to screen the functionality of specific arrangements of gene regulatory elements and gene expression modules.

In an embodiment, the methods may be used to screen hypomorphs. Generation of hypomorphs and their use in identifying key bacterial functional genes and identification of new antibiotic therapeutics as disclosed in PCT/JS2016/060730 entitled “Multiplex High-Resolution Detection of Micro-organism Strains, Related Kits, Diagnostic Methods and Screening Assays” filed Nov. 4, 2016, which is incorporated herein by reference.

The different experimental conditions may comprise exposure of the microbial cells to different chemical agents, combinations of chemical agents, different concentrations of chemical agents or combinations of chemical agents, different durations of exposure to chemical agents or combinations of chemical agents, different physical parameters, or both. In an embodiment the chemical agent is an antibiotic or antiviral. Different physical parameters to be screened may include different temperatures, atmospheric pressures, different atmospheric and non-atmospheric gas concentrations, different pH levels, different culture media compositions, or a combination thereof.

### Screening Environmental Samples

The methods disclosed herein may also be used to screen environmental samples for contaminants by detecting the presence of target nucleic acids. For example, in an embodiment, the invention provides a method of detecting microbes, comprising: exposing a detection composition (e.g., a programmable pattern recognition composition configured to detect one or more target cells or molecules) of the present invention as described herein to a sample; activating the programmable pattern recognition composition and/or system and/or an effector component thereof, by binding a PAMP or other recognized pattern associated with a target cell or molecule so as to modify a detection construct to produce a detectable signal. In an embodiment the programmable pattern recognition composition and/or system and/or an effector component thereof includes an RNA effector protein that is activated via binding of one or more guide RNAs to one or more microbe-specific target RNAs or one or more trigger RNAs such that a detectable positive signal is produced. The positive signal can be detected and is indicative of the presence of one or more microbes in the sample. In an embodiment, the detection composition or system of the present invention or component thereof may be on a substrate as described herein, and the substrate may be exposed to the sample. In other embodiments, the same detection composition or system of the present invention, and/or a different detection composition or system of the present invention may be applied to multiple discrete locations on the substrate. In further embodiments, the different detection composition or system of the present invention may detect a different microbe at each location. As described in further detail above, a substrate may be a flexible materials substrate, for example, including, but not limited to, a paper substrate, a fabric substrate, or a flexible polymer-based substrate.

In accordance with the invention, the substrate may be exposed to the sample passively, by temporarily immersing the substrate in a fluid to be sampled, by applying a fluid to be tested to the substrate, or by contacting a surface to be tested with the substrate. Any means of introducing the sample to the substrate may be used as appropriate.

As described herein, a sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In an embodiment, checking for food contamination by bacteria, such as E. coli, in restaurants or other food providers; food surfaces; Testing water for pathogens like Salmonella, Campylobacter, or E. coli; also checking food quality for manufacturers and regulators to determine the purity of meat sources; identifying air contamination with pathogens such as legionella; Checking whether beer is contaminated or spoiled by pathogens like Pediococcus and Lactobacillus; contamination of pasteurized or un-pasteurized cheese by bacteria or fungi during manufacture.

A microbe in accordance with the invention may be a pathogenic microbe or a microbe that results in food or consumable product spoilage. A pathogenic microbe may be pathogenic or otherwise undesirable to humans, animals, or plants. For human or animal purposes, a microbe may cause a disease or result in illness. Animal or veterinary applications of the present invention may identify animals infected with a microbe. For example, the methods and systems of the invention may identify companion animals with pathogens including, but not limited to, kennel cough, rabies virus, and heartworms. In other embodiments, the methods and systems of the invention may be used for parentage testing for breeding purposes. A plant microbe may result in harm or disease to a plant, reduction in yield, or alter traits such as color, taste, consistency, odor, for food or consumable contamination purposes, a microbe may adversely affect the taste, odor, color, consistency or other commercial properties of the food or consumable product. In an embodiment, the microbe is a bacterial species. The bacteria may be a psychrotroph, a coliform, a lactic acid bacteria, or a spore-forming bacteria. In an embodiment, the bacteria may be any bacterial species that causes disease or illness, or otherwise results in an unwanted product or trait. Bacteria in accordance with the invention may be pathogenic to humans, animals, or plants.

### Example Microbes

The embodiment disclosed herein may be used to detect a number of different microbes. The term microbe as used herein includes bacteria, fungus, protozoa, parasites and viruses.

### Bacteria

The following provides an example list of the types of microbes that might be detected using the embodiments disclosed herein. In an embodiment, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium dficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcahfaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus monihiformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibriofluvialis, Vibrio metchnikovii, Vibrio damsela and Vibriofurnisii), Yersinia sp. (such as Yersinia enterocolitica, Yersinia pestis, and Yersinia pseudotuberculosis) and Xanthomonas maltophilia among others.

Near-real-time microbial diagnostics are needed for food, clinical, industrial, and other environmental settings (see e.g., Lu T K, Bowers J, and Koeris M S., Trends Biotechnol. 2013 June; 31(6):325-7). In an embodiment, the assay described herein is configured for detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnmficus, Yersinia enterocolitica and Yersinia pseudotuberculosis, Brucella spp., Corynebacterium ulcerans, Coxiella burnetii, or Plesiomonas shigelloides).

### Fungi

In an embodiment, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In an embodiment, the fungus is a yeast. Examples of yeast that can be detected in accordance with disclosed methods include without limitation one or more of (or any combination of), Aspergillus species (such as Aspergillusfumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In an embodiment, the fungi is a mold. Example molds include, but are not limited to, a Penicillium species, a Cladosporium species, a Byssochlamys species, or a combination thereof.

### Protozoa

In an embodiment, the microbe is a protozoan. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.Babesia microti, Cryptosporidium parvum, Cyclospora cayetanensis, Plasmodium falciparum, P. vivax, P. ovale, P. malariae, and Toxoplasma gondii.

### Parasites

In an embodiment, the microbe is a parasite. Examples of parasites that can be detected in accordance with disclosed methods include without limitation one or more of (or any combination of), an Onchocerca species and a Plasmodium species.

### Viruses

In an embodiment, the systems, devices, and methods, disclosed herein are directed to detecting viruses in a sample. The embodiments disclosed herein may be used to detect viral infection (e.g., of a subject or plant), or determination of a viral strain, including viral strains that differ by a single nucleotide polymorphism. The virus may be a DNA virus, an RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In an embodiment, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In an embodiment, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In an embodiment, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In an embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e., after infection has occurred or prophylactically, i.e., before infection has occurred.

In an embodiment, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In an embodiment, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In an embodiment, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

### SARS-CoV-2

The present disclosure relates to and/or involves detection of SARS-CoV-2.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In an embodiment, a variant has developed a “specific group of mutations” that causes the variant to behave differently than that of the strain it originated from. While there are many thousands of variants of SARS-CoV-2, (Koyama, Takahiko Koyama; Platt, Daniela; Parida, Laxmi (June 2020). “Variant analysis of SARS-CoV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-CoV-2 as hCoV-19 identified seven clades (0, S, L, V, G, GH, and GR) (Alm E, Broberg E K, Connor T, et al. Geographical and temporal distribution of SARS-CoV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, 1378 and D392) (Guan Q, Sadykov M, Mfarrej S, et al. A genetic barcode of SARS-CoV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, A.; et al. “A dynamic nomenclature proposal for SARS-CoV-2 lineages to assist genomic epidemiology”. 5: 1403-1407).

Genetic variants of SARS-CoV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-CoV-2, particularly those having substitutions of therapeutic concern. Table 9 below shows exemplary, non-limiting genetic substitutions in SARS-CoV-2 variants.

TABLE 9

| | Common Pango Lineages with Spike |
|---|---|
| Spike Protein Substitution | Protein Substitutions |
| L452R | A.2.5, B.1, B.1.429, B.1.427, B.1.617.1, |
| | B.1.526.1, B.1.617.2, C.36.3 |
| E484K | B.1.1.318, B.1.1.7, B.1.351, B.1.525, |
| | B.1.526, B.1.621, B.1.623, P.1, P.1.1, |
| | P.1.2, R.1 |
| K417N, E484K, N501Y | B.1.351, B.1.351.3 |
| K417T, E484K, N501Y | P.1, P.1.1, P.1.2 |
| A67V, del69-70, T95I, del142-144, Y145D, del211, | B.1.1.529 and BA lineages |
| L212I, ins214EPE, G339D, S371L, S373P, S375F, | |
| K417N, N440K, G446S, S477N, T478K, E484A, | |
| Q493R, G496S, Q498R, N501Y, Y505H, T547K, | |
| D614G, H655Y, N679K, P681H, N764K, D796Y, | |
| N856K, Q954H, N969K, L981F | |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature. It is available at cov-lineages.org.

In an embodiment, the SARS-CoV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T716I, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13L, W152C, L452R, and D614G; B.1.617.2, also known as Delta (WHO) or India variant, having the following spike protein substitutions: T19R, (G142D), 156del, 157del, R158G, L452R, T478K, D614G, P681R, and D950N; P.1, also known as Gamma (WHO) or Japan/Brazil variant, having the following spike protein substitutions: L18F, T20N, P26S, D138Y, R190S, K417T, E484K, N501Y, D614G, H655Y, and T1027I; and B.1.1.529 also known as Omicron (WHO), having the following spike protein substitutions: A67V, del69-70, T95I, del142-144, Y145D, del211, L212L, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, or any combination thereof.

In an embodiment, the SARS-CoV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In an embodiment, the SARS-Cov-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In an embodiment, the SARS-Cov-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In an embodiment, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In an embodiment the SARS-Cov-2 variant is a VOC. In an embodiment, the SARS-CoV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), a Omicon variant (B.1.1.529) or any combination thereof.

In an embodiment the SARS-Cov-2 variant is a VOI. In an embodiment, the SARS-CoV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95L, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T95I), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); or any combination thereof.

In an embodiment SARS-Cov-2 variant is a VON. In an embodiment, the SARS-Cov-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: https://doi.org/10.1101/2021.02.08.21251393) (spike protein substitutions: F157L, V367F, Q613H, P681R); or any combination thereof.

### Drug Resistant Viruses

In an embodiment, the virus is a drug resistant virus. By means of example, and without limitation, the virus may be a ribavirin resistant virus. Ribavirin is a very effective antiviral that hits a number of RNA viruses. Below are a few important viruses that have evolved ribavirin resistance. Foot and Mouth Disease Virus: doi:10.1128/JVI.03594-13. Polio virus: www.pnas.org/content/100/12/7289.full.pdf. Hepatitis C Virus: jvi.asm.org/content/79/4/2346.full. A number of other persistent RNA viruses, such as hepatitis and HIV, have evolved resistance to existing antiviral drugs. Hepatitis B Virus (lamivudine, tenofovir, entecavir): doi:10.1002/hep.22900. Hepatitis C Virus (Telaprevir, BILN2061, ITMN-191, SCH6, Boceprevir, AG-021541, ACH-806): doi:10.1002/hep.22549. HIV has many drug resistant mutations, see hivdb.stanford.edu/for more information. Aside from drug resistance, there are a number of clinically relevant mutations that could be targeted with the CRISPR systems according to the invention as described herein. For instance, persistent versus acute infection in LCMV: doi:10.1073/pnas.1019304108; or increased infectivity of Ebola: doi.org/10.1016/j.cell.2016.10.014 and doi.org/10.1016/j.cell.2016.10.013.

### Malaria Detection and Monitoring

Malaria is a mosquito-borne pathology caused by Plasmodium parasites. The parasites are spread to people through the bites of infected female Anopheles mosquitoes. Five Plasmodium species cause malaria in humans: Plasmodium falciparum, Plasmodium vivax, Plasmodium ovale, Plasmodium malariae, and Plasmodium knowlesi. Among them, according to the World Health Organization (WHO), Plasmodium falciparum; and Plasmodium vivax are responsible for the greatest threat. P. falciparum is the most prevalent malaria parasite on the African continent and is responsible for most malaria-related deaths globally. P. vivax is the dominant malaria parasite in most countries outside of sub-Saharan Africa.

Treatment against Plasmodium sp. include aryl-amino alcohols such as quinine or quinine derivatives such as chloroquine, amodiaquine, mefloquine, piperaquine, lumefantrine, primaquine; lipophilic hydroxynaphthoquinone analog, such as atovaquone; antifolate drugs, such as the sulfa drugs sulfadoxine, dapsone and pyrimethamine; proguanil; the combination of atovaquone/proguanil; atemisins drugs; and combinations thereof. In an embodiment, the method includes screening for resistance against one or more of these compounds.

Target sequences for the assays described herein include those that are diagnostic for the presence of a mosquito-borne pathogen include a sequence that diagnostic for the presence of Plasmodium, notably Plasmodia species affecting humans such as Plasmodium falciparum, Plasmodium vivax, Plasmodium ovale, Plasmodium malariae, and Plasmodium knowlesi, including sequences from the genomes thereof.

Target sequences for the assays described herein include those that are diagnostic for monitoring drug resistance to treatment against Plasmodium, including but not limited to, Plasmodia species affecting humans such as Plasmodium falciparum, Plasmodium vivax, Plasmodium ovale, Plasmodium malariae, and Plasmodium knowlesi.

Further target sequences include sequences include target molecules/nucleic acid molecules coding for proteins involved in essential biological process for the Plasmodium parasite and notably transporter proteins, such as protein from drug/metabolite transporter family, the ATP-binding cassette (ABC) protein involved in substrate translocation, such as the ABC transporter C subfamily or the Na+/H+ exchanger, membrane glutathione S-transferase; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional target may also include the gene(s) coding for the heme polymerase.

Further target sequences include target molecules/nucleic acid molecules coding for proteins involved in essential biological process may be selected from the P. falciparum chloroquine resistance transporter gene (pfcrt), the P. falciparum multidrug resistance transporter 1 (pfmdrl), the P. falciparum multidrug resistance-associated protein gene (Pfmrp), the P. falciparum Na+/H+ exchanger gene (pfnhe), the gene coding for the P. falciparum exported protein 1, the P. falciparum Ca2+ transporting ATPase 6 (pfatp6); the P. falciparum dihydropteroate synthase (pfdhps), dihydrofolate reductase activity (pfdhpr) and dihydrofolate reductase-thymidylate synthase (pfdhfr) genes, the cytochrome b gene, gtp cyclohydrolase and the Kelch13 (K13) gene as well as their functional heterologous genes in other Plasmodium species.

A number of mutations, notably single point mutations, have been identified in the proteins which are the targets of the current malaria treatments and associated with specific resistance phenotypes. Accordingly, the invention allows for the detection of various resistance phenotypes of mosquito-borne parasites, such as plasmodium by detection of those targets that are associated with the specific resistance phenotypes.

In an embodiment, the method detects one or more mutation(s) and/or one or more single nucleotide polymorphisms in target nucleic acids/molecules. In an embodiment, any one of the mutations below, or their combination thereof, can be used as drug resistance marker and can be detected using the methods, assays, devices, compositions, and/or devices described herein.

Single point mutations in P. falciparum K13 that can be detected by an assay described herein include the following single point mutations in positions 252, 441, 446, 449, 458, 493, 539, 543, 553, 561, 568, 574, 578, 580, 675, 476, 469, 481, 522, 537, 538, 579, 584 and 719 and notably mutations E252Q, P441L, F446L, G449A, N458Y, Y493H, R539T, I543T, P553L, R561H, V568G, P574L, A578S, C580Y, A675V, M4761; C469Y; A481V; S522C; N537I; N537D; G538V; M579I; D584V; and H719N. These mutations are generally associated with artemisins drugs resistance phenotypes (Artemisinin and artemisinin-based combination therapy resistance, April 2016 WHO/HTM/GMP/2016.5).

Mutations in the P. falciparum dihydrofolate reductase (DHFR) (PfDHFR-TS, PFD0830w) that can be detected by the assays described herein include mutations in positions 108, 51, 59 and 164, notably 108 D, 164L, 511 and 59R which modulate resistance to pyrimethamine. Other polymorphisms that can be detected by the methods described herein include 437G, 581G, 540E, 436A and 613S, which are associated with resistance to sulfadoxine. Additional mutations that can be detected by the assays described herein include Ser108Asn, Asn51Ile, Cys59Arg, Ile164Leu, Cys50Arg, Ile164Leu, Asn188Lys, Ser189Arg and Val213Ala, Ser108Thr and Ala16Val. Mutations Ser108Asn, Asn51Ile, Cys59Arg, Ile164Leu, Cys50Arg, Ile164Leu are notably associated with pyrimethamine based therapy and/or chloroguanine-dapsone combination therapy resistances and can be detected by the assays described herein. Cycloguanil resistance appears to be associated with the double mutations Ser108Thr and Ala16Val, which can be detected by the assays described herein. Amplification of dhfr may also be of high relevance for therapy resistance notably pyrimethamine resistance and can be detected ny the assays described herein.

Mutations in the P. falciparum dihydropteroate synthase (DHPS) (PfDHPS, PF08_0095) can be detected by the assays described herein, and include, without limitation, mutations in positions 436, 437, 581 and 613 Ser436Ala/Phe, Ala437Gly, Lys540Glu, Ala581Gly and Ala613Thr/Ser. Polymorphism in position 581 and/or 613 have also been associated with resistance to sulfadoxine-pyrimethamine base therapies and can be detected by an assay described herein.

Mutations in the P. falciparum chloroquine-resistance transporter (PfCRT) can be detected by the assays described herein. In an embodiment, the polymorphism in position 76, notably the mutation Lys76Thr, is associated with resistance to chloroquine and can be detected by an assay described herein. Further polymorphisms include Cys72Ser, Met74Ile, Asn75Glu, Ala220Ser, Gln271Glu, Asn326Ser, Ile356Thr and Arg371Ile which may be associated with chloroquine resistance can be detected by an assay described herein. PfCRT is also phosphorylated at the residues S33, S411 and T416, which may regulate the transport activity or specificity of the protein, which can be detected by an assay described herein.

Mutations in the P. falciparum multidrug-resistance transporter 1 (PfMDR1) (PFEI150w) can be detected by an assay described herein. For example, polymorphisms in positions 86, 184, 1034, 1042, notably Asn86Tyr, Tyr184-Phe, Ser1034Cys, Asn1042Asp and Asp1246Tyr have been identified and reported to influence have been reported to influence susceptibilities to lumefantrine, artemisinin, quinine, mefloquine, halofantrine and chloroquine and can be detected by an assay described herein. Additionally, amplification of PfMDR1 is associated with reduced susceptibility to lumefantrine, artemisinin, quinine, mefloquine, and halofantrine and can be detected by an assay described herein. Deamplification of PfMDR1 leads to an increase in chloroquine resistance and can be detected by an assay described herein. Amplification of pfindr1 may also be detected. The phosphorylation status of PfMDR1 is also of high relevance and can be detected by an assay described herein.

Mutations in the P. falciparum multidrug-resistance associated protein (PfMRP) (gene reference PFA0590w) can be detected by an assay described herein. For example polymorphisms in positions 191 and/or 437, such as Y191H and A437S have been identified and associated with chloroquine resistance phenotypes and can be detected by an assay described herein.

Mutations in the P. falciparum NA+/H+ enchanger (PfNHE) (ref PF13_0019) can be detected by an assay described herein. For example, increased repetition of the DNNND in microsatellite ms4670 may be a marker for quinine resistance and can be detected by an assay described herein.

Mutations altering the ubiquinol binding site of the cytochrome b protein encoded by the cytochrome be gene (cytb, mal_mito_3) are associated with atovaquone resistance and can be detected by an assay described herein. Mutations in positions 26, 268, 276, 133 and 280 and notably Tyr26Asn, Tyr268Ser, M1331 and G280D may be associated with atovaquone resistance and can be detected by an assay described herein.

In P Vivax, mutations in PvMDR1, the homolog of Pf MDR1 have been associated with chloroquine resistance, notably polymorphism in position 976 such as the mutation Y976F and can be detected by an assay described herein.

The above mutations are defined in terms of protein sequences. However, the skilled person is able to determine the corresponding mutations, including SNPs, to be identified as a nucleic acid target sequence.

Other identified drug-resistance markers are known in the art, for example as described in “Susceptibility of Plasmodium falciparum to antimalarial drugs (1996-2004)”; WHO; Artemisinin and artemisinin-based combination therapy resistance (April 2016 WHO/HTM/GMP/2016.5); “Drug-resistant malaria: molecular mechanisms and implications for public health” FEBS Lett. 2011 Jun. 6; 585(11):1551-62. doi:10.1016/j.febslet.2011.04.042. Epub 2011 Apr. 23. Review. PubMed PMID: 21530510; the contents of which are herewith incorporated by reference and can be detected by an assay described herein.

As to polypeptides that may be detected in accordance with the present invention, gene products of all genes mentioned herein may be used as targets. Correspondingly, it is contemplated that such polypeptides could be used for species identification, typing and/or detection of drug resistance.

In an embodiment, the systems, devices, and methods, disclosed herein are directed to detecting the presence of one or more mosquito-borne parasite in a sample, such as a biological sample obtained from a subject. In an embodiment, the parasite may be selected from the species Plasmodium falciparum, Plasmodium vivax, Plasmodium ovale, Plasmodium malariae or Plasmodium knowlesi. Accordingly, the methods disclosed herein can be adapted for use in other methods (or in combination) with other methods that require quick identification of parasite species, monitoring the presence of parasites and parasite forms (for example corresponding to various stages of infection and parasite life-cycle, such as exo-erythrocytic cycle, erythrocytic cyle, sporpogonic cycle; parasite forms include merozoites, sporozoites, schizonts, gametocytes); detection of certain phenotypes (e.g., pathogen drug resistance), monitoring of disease progression and/or outbreak, and treatment (drug) screening. Further, in the case of malaria, a long time may elapse following the infective bite, namely a long incubation period, during which the patient does not show symptoms. Similarly, prophylactic treatments can delay the appearance of symptoms, and long asymptomatic periods can also be observed before a relapse. Such delays can easily cause misdiagnosis or delayed diagnosis, and thus impair the effectiveness of treatment.

Because of the rapid and sensitive diagnostic capabilities of the embodiments disclosed here, detection of parasite type, down to a single nucleotide difference, and the ability to be deployed as a POC device, the embodiments disclosed herein may be used guide therapeutic regimens, such as selection of the appropriate course of treatment. The embodiments disclosed herein may also be used to screen environmental samples (mosquito population, etc.) for the presence and the typing of the parasite. The embodiments may also be modified to detect mosquito-borne parasites and other mosquito-borne pathogens simultaneously. In some instances, malaria and other mosquito-borne pathogens may present initially with similar symptoms. Thus, the ability to quickly distinguish the type of infection can guide important treatment decisions. Other mosquito-born pathogens that may be detected in conjunction with malaria include dengue, West Nile virus, chikungunya, yellow fever, filariasis, Japanese encephalitis, Saint Louis encephalitis, western equine encephalitis, eastern equine encephalitis, Venezuelan equine encephalitits, La Crosse encephalitis, and zika.

In an embodiment, the devices, systems, and methods disclosed herein may be used to distinguish multiple mosquito-borne parasite species in a sample. In an embodiment, identification may be based on ribosomal RNA sequences, including the 18S, 16S, 23S, and 5S subunits. In an embodiment, identification may be based on sequences of genes that are present in multiple copies in the genome, such as mitochondrial genes like CYTB. In an embodiment, identification may be based on sequences of genes that are highly expressed and/or highly conserved such as GAPDH, Histone H2B, enolase, or LDH. Methods for identifying relevant rRNA sequences are disclosed in U.S. Patent Application Publication No. 2017/0029872. In an embodiment, a set of guide RNA may be designed to distinguish each species by a variable region that is unique to each species or strain. Guide RNAs may also be designed to target RNA genes that distinguish microbes at the genus, family, order, class, phylum, kingdom levels, or a combination thereof. In an embodiment where amplification is used, a set of amplification primers may be designed to flanking constant regions of the ribosomal RNA sequence and a guide RNA designed to distinguish each species by a variable internal region. In an embodiment, the primers and guide RNAs may be designed to conserved and variable regions in the 16S subunit respectfully. Other genes or genomic regions that uniquely variable across species or a subset of species such as the RecA gene family, RNA polymerase p subunit, may be used as well. Other suitable phylogenetic markers, and methods for identifying the same, are discussed for example in Wu et al. arXiv:1307.8690 [q-bio.GN].

In an embodiment, species identification can be performed based on genes that are present in multiple copies in the genome, such as mitochondrial genes like CYTB. In an embodiment, species identification can be performed based on highly expressed and/or highly conserved genes such as GAPDH, Histone H2B, enolase, or LDH.

In an embodiment, a method or diagnostic is designed to screen mosquito-borne parasites across multiple phylogenetic and/or phenotypic levels at the same time. For example, the method or diagnostic may comprise the use of multiple programmable pattern recognition compositions, CRISPR systems, with different guide RNAs when used. A first set of guide RNAs may distinguish, for example, between Plasmodium falciparum or Plasmodium vivax. These general classes can be even further subdivided. For example, guide RNAs could be designed and used in the method or diagnostic that distinguish drug-resistant strains, in general or with respect to a specific drug or combination of drugs. A second set of guide RNA can be designed to distinguish microbes at the species level. Thus, a matrix may be produced identifying all mosquito-borne parasites species or subspecies, further divided according to drug resistance. The foregoing is for example purposes only. Other means for classifying other types of mosquito-borne parasites are also contemplated and would follow the general structure described above.

In an embodiment, the devices, systems and methods disclosed herein may be used to screen for mosquito-borne parasite genes of interest, for example drug resistance genes. Guide RNAs may be designed to distinguish between known genes of interest. Samples, including clinical samples, may then be screened using the embodiments disclosed herein for detection of one or more such genes. The ability to screen for drug resistance at POC would have tremendous benefit in selecting an appropriate treatment regime. In an embodiment, the drug resistance genes are genes encoding proteins such as transporter proteins, such as protein from drug/metabolite transporter family, the ATP-binding cassette (ABC) protein involved in substrate translocation, such as the ABC transporter C subfamily or the Na+/H+ exchanger; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional targets may also include the gene(s) coding for the heme polymerase. In an embodiment, the drug resistance genes are selected from the P. falciparum chloroquine resistance transporter gene (pfcrt), the P. falciparum multidrug resistance transporter 1 (pfmdrl), the P. falciparum multidrug resistance-associated protein gene (Pfmrp), the P. falciparum Na+/H+ exchanger gene (pfnhe), the P. falciparum Ca2+ transporting ATPase 6 (pfatp6), the P. falciparum dihydropteroate synthase (pfdhps), dihydrofolate reductase activity (pfdhpr) and dihydrofolate reductase-thymidylate synthase (pfdhfr) genes, the cytochrome b gene, gtp cyclohydrolase and the Kelch13 (K13) gene as well as their functional heterologous genes in other Plasmodium species. Other identified drug-resistance markers are known in the art, for example as described in “Susceptibility of Plasmodium falciparum to antimalarial drugs (1996-2004)”; WHO; Artemisinin and artemisinin-based combination therapy resistance (April 2016 WHO/HTM/GMP/2016.5); “Drug-resistant malaria: molecular mechanisms and implications for public health” FEBS Lett. 2011 Jun. 6; 585(11):1551-62. doi:10.1016/j.febslet.2011.04.042. Epub 2011 Apr. 23. Review. PubMed PMID: 21530510; the contents of which are herewith incorporated by reference.

In an embodiment, programmable pattern recognition compositions, detection system or methods of use thereof as described herein may be used to determine the evolution of a mosquito-borne parasite outbreak. The method may comprise detecting one or more target molecules or cells from a plurality of samples from one or more subjects, wherein the target molecule or cells are from a mosquito-borne parasite spreading or causing the outbreaks. Such a method may further comprise determining a pattern of mosquito-borne parasite transmission, or a mechanism involved in a disease outbreak caused by a mosquito-borne parasite. The samples may be derived from one or more humans, and/or be derived from one or more mosquitoes.

The pattern of pathogen transmission may comprise continued new transmissions from the natural reservoir of the mosquito-borne parasite or other transmissions (e.g., across mosquitoes) following a single transmission from the natural reservoir or a mixture of both. In one embodiment, the target molecule is preferably a nucleic acid sequence within the mosquito-borne parasite genome or fragments thereof. In one embodiment, the pattern of the mosquito-borne parasite transmission is the early pattern of the mosquito-borne parasite transmission, i.e., at the beginning of the mosquito-borne parasite outbreak. Determining the pattern of the mosquito-borne parasite transmission at the beginning of the outbreak increases likelihood of stopping the outbreak at the earliest possible time thereby reducing the possibility of local and international dissemination.

Determining the pattern of the mosquito-borne parasite transmission may comprise detecting a mosquito-borne parasite target molecule or cell according to the methods described herein. Determining the pattern of the pathogen transmission may further comprise detecting shared intra-host variations of the mosquito-borne parasite sequence between the subjects and determining whether the shared intra-host variations show temporal patterns. Patterns in observed intrahost and interhost variation provide important insight about transmission and epidemiology (Gire, et al., 2014).

In addition to other sample types disclosed herein, the sample may be derived from one or more mosquitoes, for example the sample may comprise mosquito saliva.

### Biomarker Detection and Applications

In an embodiment, the programmable pattern recognition compositions systems, devices, and methods disclosed herein may be used for biomarker detection. For example, the systems, devices and method disclosed herein may be used for SNP detection and/or genotyping, transcript detection, and/or protein detection. The programmable pattern recognition compositions systems, devices and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular, immune disorders, and cancer among other diseases may be detected. In an embodiment, the embodiments disclosed herein may be used for cell free DNA detection of diseases that involve lysis, such as liver fibrosis and restrictive/obstructive lung disease. In an embodiment, the pattern recognition compositions are utilized for faster and more portable detection for pre-natal testing of cell-free DNA. The embodiments disclosed herein may be used for screening panels of different SNPs or other biomarkers associated with, among others, cardiovascular health, lipid/metabolic signatures, ethnicity identification, paternity matching, human ID (e.g., matching suspect to a criminal database of SNP signatures). The embodiments disclosed herein may also be used for cell free DNA detection of mutations related to and released from cancer tumors. The embodiments disclosed herein may also be used for detection of meat quality, for example, by providing rapid detection of different animal sources in a given meat product. Embodiments disclosed herein may also be used for the detection of GMOs or gene editing related to DNA. As described herein elsewhere, closely related genotypes/alleles or biomarkers (e.g., having only a single nucleotide difference in a given target sequence) may be distinguished by introduction of a synthetic mismatch in a gRNA employed or included with the pattern recognition compositions and/or systems described herein.

In an aspect, the invention relates to a method for detecting target nucleic acids and/or polypeptides, and/or cells in samples, comprising distributing a sample or set of samples into one or more individual discrete volumes, the individual discrete volumes comprising a detection composition according to the invention as described herein; incubating the sample or set of samples under conditions sufficient to allow recognition and/or binding of the pattern recognition compositions to one or more PAMPs so as to activate the pattern recognition compositions; activating an effector protein or domain of the detection composition via binding of the one or more guide RNAs to the one or more target molecules, wherein activating the detection composition effector protein results in modification of the detection construct such that a detectable signal is generated; and detecting the detectable signal, wherein detection of the detectable e signal indicates a presence of one or more target molecules in the sample.

### Detecting Circulating Tumor Cells

In one embodiment, circulating cells (e.g., circulating tumor cells (CTC)) can be assayed with the present invention. Isolation of circulating tumor cells (CTC) for use in any of the methods described herein may be performed. Exemplary technologies that achieve specific and sensitive detection and capture of circulating cells that may be used in the present invention have been described (Mostert B, et al., Circulating tumor cells (CTCs): detection methods and their clinical relevance in breast cancer. Cancer Treat Rev. 2009; 35:463-474; and Talasaz A H, et al., Isolating highly enriched populations of circulating epithelial cells and other rare cells from blood using a magnetic sweeper device. Proc Natl Acad Sci USA. 2009; 106:3970-3975). As few as one CTC may be found in the background of 105-106 peripheral blood mononuclear cells (Ross A A, et al., Detection and viability of tumor cells in peripheral blood stem cell collections from breast cancer patients using immunocytochemical and clonogenic assay techniques. Blood. 1993,82:2605-2610). The CellSearch® platform uses immunomagnetic beads coated with antibodies to Epithelial Cell Adhesion Molecule (EpCAM) to enrich for EPCAM-expressing epithelial cells, followed by immunostaining to confirm the presence of cytokeratin staining and absence of the leukocyte marker CD45 to confirm that captured cells are epithelial tumor cells (Momburg F, et al., Immunohistochemical study of the expression of a Mr 34,000 human epithelium-specific surface glycoprotein in normal and malignant tissues. Cancer Res. 1987; 47:2883-2891; and Allard W J, et al., Tumor cells circulate in the peripheral blood of all major carcinomas but not in healthy subjects or patients with nonmalignant diseases. Clin Cancer Res. 2004; 10:6897-6904). The number of cells captured have been prospectively demonstrated to have prognostic significance for breast, colorectal and prostate cancer patients with advanced disease (Cohen S J, et al., J Clin Oncol. 2008; 26:3213-3221; Cristofanilli M, et al. N Engl J Med. 2004; 351:781-791; Cristofanilli M, et al., J Clin Oncol. 2005; 23: 1420-1430; and de Bono J S, et al. Clin Cancer Res. 2008; 14:6302-6309).

The present invention also provides for isolating CTCs with CTC-Chip Technology. CTC-Chip is a microfluidic based CTC capture device where blood flows through a chamber containing thousands of microposts coated with anti-EpCAM antibodies to which the CTCs bind (Nagrath S, et al. Isolation of rare circulating tumour cells in cancer patients by microchip technology. Nature. 2007; 450: 1235-1239). CTC-Chip provides a significant increase in CTC counts and purity in comparison to the CellSearch® system (Maheswaran S, et al. Detection of mutations in EGFR in circulating lung-cancer cells, N Engl J Med. 2008; 359:366-377), both platforms may be used for downstream molecular analysis.

### Cell-Free Chromatin

In an embodiment, cell free chromatin fragments are isolated and analyzed according to the present invention. Nucleosomes can be detected in the serum of healthy individuals (Stroun et al., Annals of the New York Academy of Sciences 906: 161-168 (2000)) as well as individuals afflicted with a disease state. Moreover, the serum concentration of nucleosomes is considerably higher in patients suffering from benign and malignant diseases, such as cancer and autoimmune disease (Holdenrieder et al (2001) Int J Cancer 95, 1 14-120, Trejo-Becerril et al (2003) Int J Cancer 104, 663-668; Kuroi et al 1999 Breast Cancer 6, 361-364; Kuroi et al (2001) Int j Oncology 19, 143-148; Amoura et al (1997) Arth Rheum 40,2217-2225; Williams et al (2001) J Rheumatol 28, 81-94). Not being bound by a theory, the high concentration of nucleosomes in tumor bearing patients derives from apoptosis, which occurs spontaneously in proliferating tumors. Nucleosomes circulating in the blood contain uniquely modified histones. For example, U.S. Patent Publication No. 2005/0069931 (Mar. 31, 2005) relates to the use of antibodies directed against specific histone N-terminus modifications as diagnostic indicators of disease, employing such histone-specific antibodies to isolate nucleosomes from a blood or serum sample of a patient to facilitate purification and analysis of the accompanying DNA for diagnostic/screening purposes. Accordingly, the present invention may use chromatin bound DNA to detect and monitor, for example, tumor mutations. The identification of the DNA associated with modified histones can serve as diagnostic markers of disease and congenital defects.

Thus, in another embodiment, isolated chromatin fragments are derived from circulating chromatin, preferably circulating mono and oligonucleosomes. Isolated chromatin fragments may be derived from a biological sample. The biological sample may be from a subject or a patient in need thereof. The biological sample may be sera, plasma, lymph, blood, blood fractions, urine, synovial fluid, spinal fluid, saliva, circulating tumor cells or mucous.

Cell-Free DNA (cDNA)

In an embodiment, the present invention may be used to detect cell free DNA (cfDNA). Cell free DNA in plasma or serum may be used as a non-invasive diagnostic tool. For example, cell free fetal DNA has been studied and optimized for testing on-compatible RhD factors, sex determination for X-linked genetic disorders, testing for single gene disorders, identification of preeclampsia. For example, sequencing the fetal cell fraction of cfDNA in maternal plasma is a reliable approach for detecting copy number changes associated with fetal chromosome aneuploidy. For another example, cfDNA isolated from cancer patients has been used to detect mutations in key genes relevant for treatment decisions.

In an embodiment, the present disclosure provides detecting cfDNA directly from a patient sample. In certain other example embodiment, the present disclosure provides enriching cfDNA using the enrichment embodiments disclosed above and prior to detecting the target cfDNA.

### Exosomes

In one embodiment, exosomes can be assayed with the present invention. Exosomes are small extracellular vesicles that have been shown to contain RNA. Isolation of exosomes by ultracentrifugation, filtration, chemical precipitation, size exclusion chromatography, and microfluidics are known in the art. In one embodiment exosomes are purified using an exosome biomarker. Isolation and purification of exosomes from biological samples may be performed by any known methods (see e.g., WO2016172598A1).

### SNP Detection and Genotyping

In an embodiment, the present invention may be used to detect the presence of single nucleotide polymorphisms (SNP) in a biological sample. The SNPs may be related to maternity testing (e.g., sex determination, fetal defects). They may be related to a criminal investigation. In one embodiment, a suspect in a criminal investigation may be identified by the present invention. Not being bound by a theory nucleic acid based forensic evidence may require the most sensitive assay available to detect a suspect or victim's genetic material because the samples tested may be limiting.

In other embodiments, SNPs associated with a disease are encompassed by the present invention. SNPs associated with diseases are well known in the art and one skilled in the art can apply the methods of the present invention to design suitable guide RNAs (see e.g., www.ncbi.nlm.nih.gov/clinvar?term=human %5Borgn %5D).

In an aspect, the invention relates to a method for genotyping, such as SNP genotyping, comprising: distributing a sample or set of samples into one or more individual discrete volumes, the individual discrete volumes comprising a detection composition or system according to the invention as described herein; incubating the sample or set of samples under conditions sufficient to allow binding of the one or more guide RNAs to one or more target molecules; activating the detection composition effector protein via binding of the one or more guide RNAs to the one or more target molecules, wherein activating the detection composition effector protein results in modification of the detection construct such that a detectable signal is generated; and detecting the detectable signal, wherein detection of the detectable signal indicates a presence of one or more target molecules characteristic for a particular genotype in the sample.

In an embodiment, the detectable signal is compared to (e.g., by comparison of signal intensity) one or more standard signal, preferably a synthetic standard signal). In an embodiment, the standard is or corresponds to a particular genotype. In an embodiment, the standard comprises a particular SNP or other (single) nucleotide variation. In an embodiment, the standard is a (PCR-amplified) genotype standard. In an embodiment, the standard is or comprises DNA. In an embodiment, the standard is or comprises RNA. In an embodiment, the standard is or comprised RNA which is transcribed from DNA. In an embodiment, the standard is or comprises DNA which is reverse transcribed from RNA. In an embodiment, the detectable signal is compared to one or more standard, each of which corresponds to a known genotype, such as a SNP or other (single) nucleotide variation. In an embodiment, the detectable signal is compared to one or more standard signal and the comparison comprises statistical analysis, such as by parametric or non-parametric statistical analysis, such as by one- or two-way ANOVA, etc. In an embodiment, the detectable signal is compared to one or more standard signal and when the detectable signal does not (statistically) significantly deviate from the standard, the genotype is determined as the genotype corresponding to said standard.

In other embodiments, the present invention allows rapid genotyping for emergency pharmacogenomics. In one embodiment, a single point of care assay may be used to genotype a patient brought into the emergency room. The patient may be suspected of having a blood clot and an emergency physician needs to decide a dosage of blood thinner to administer. In exemplary embodiments, the present invention may provide guidance for administration of blood thinners during myocardial infarction or stroke treatment based on genotyping of markers such as VKORC1, CYP2C9, and CYP2C19. In one embodiment, the blood thinner is the anticoagulant warfarin (Holford, NH (December 1986). “Clinical Pharmacokinetics and Pharmacodynamics of Warfarin Understanding the Dose-Effect Relationship”. Clinical Pharmacokinetics. Springer International Publishing. 11 (6): 483-504). Genes associated with blood clotting are known in the art (see e.g., US20060166239A1; Litin S C, Gastineau D A (1995) “Current concepts in anticoagulant therapy”. Mayo Clin. Proc. 70 (3): 266-72; and Rusdiana et al., Responsiveness to low-dose warfarin associated with genetic variants of VKORC1, CYP2C9, CYP2C19, and CYP4F2 in an Indonesian population. Eur J Clin Pharmacol. 2013 March; 69(3):395-405). Specifically, in the VKORCI 1639 (or 3673) single-nucleotide polymorphism, the common (“wild-type”) G allele is replaced by the A allele. People with an A allele (or the “A haplotype”) produce less VKORC1 than do those with the G allele (or the “non-A haplotype”). The prevalence of these variants also varies by race, with 37% of Caucasians and 14% of Africans carrying the A allele. The end result is a decreased number of clotting factors and therefore, a decreased ability to clot.

In an embodiment, the availability of genetic material for detecting a SNP in a patient allows for detecting SNPs without amplification of a DNA or RNA sample. In the case of genotyping, the biological sample tested is easily obtained. In an embodiment, the incubation time of the present invention may be shortened. The assay may be performed in a period of time required for an enzymatic reaction to occur. One skilled in the art can perform biochemical reactions in 5 minutes (e.g., 5 minute ligation). The present invention may use an automated DNA extraction device to obtain DNA from blood. The DNA can then be added to a reaction that generates a target molecule for the effector protein. Immediately upon generating the target molecule the masking agent can be cut and a signal detected. In exemplary embodiments, the present invention allows a POC rapid diagnostic for determining a genotype before administering a drug (e.g., blood thinner). In the case where an amplification step is used, all of the reactions occur in the same reaction in a one step process. In preferred embodiments, the POC assay may be performed in less than an hour, preferably 10 minutes, 20 minutes, 30 minutes, 40 minutes, or 50 minutes.

In an embodiment, the systems, devices, and methods disclosed herein may be used for detecting the presence or expression level of long non-coding RNAs (lncRNAs). Expression of certain lncRNAs are associated with disease state and/or drug resistance. In particular, certain lncRNAs (e.g., TCONS_00011252, NR_034078, TCONS_00010506, TCONS 00026344, TCONS 00015940, TCONS 00028298, TCONS 00026380, TCONS_0009861, TCONS_00026521, TCONS_00016127, NR_125939, NR_033834, TCONS_00021026, TCONS_00006579, NR_109890, and NR_026873) are associated with resistance to cancer treatment, such as resistance to one or more BRAF inhibitors (e.g., Vemurafenib, Dabrafenib, Sorafenib, GDC-0879, PLX-4720, and LGX818) for treating melanoma (e.g., nodular melanoma, lentigo maligna, lentigo maligna melanoma, acral lentiginous melanoma, superficial spreading melanoma, mucosal melanoma, polypoid melanoma, desmoplastic melanoma, amelanotic melanoma, and soft-tissue melanoma). The detection of lncRNAs using the various embodiments described herein can facilitate disease diagnosis and/or selection of treatment options.

In one embodiment, the present invention can guide DNA- or RNA-targeted therapies (e.g., CRISPR, TALE, Zinc finger proteins, RNAi), particularly in settings where rapid administration of therapy is important to treatment outcomes.

### LOH Detection

Cancer cells undergo a loss of genetic material (DNA) when compared to normal cells. This deletion of genetic material which almost all, if not all, cancers undergo is referred to as “loss of heterozygosity” (LOH). Loss of heterozygosity (LOH) is a gross chromosomal event that results in loss of the entire gene and the surrounding chromosomal region. The loss of heterozygosity is a common occurrence in cancer, where it can indicate the absence of a functional tumor suppressor gene in the lost region. However, a loss may be silent because there still is one functional gene left on the other chromosome of the chromosome pair. The remaining copy of the tumor suppressor gene can be inactivated by a point mutation, leading to loss of a tumor suppressor gene. The loss of genetic material from cancer cells can result in the selective loss of one of two or more alleles of a gene vital for cell viability or cell growth at a particular locus on the chromosome.

An “LOH marker” is DNA from a microsatellite locus, a deletion, alteration, or amplification in which, when compared to normal cells, is associated with cancer or other diseases. An LOH marker often is associated with loss of a tumor suppressor gene or another, usually tumor related, gene.

The term “microsatellites” refers to short repetitive sequences of DNA that are widely distributed in the human genome. A microsatellite is a tract of tandemly repeated (i.e., adjacent) DNA motifs that range in length from two to five nucleotides, and are typically repeated 5-50 times. For example, the sequence TATATATATA (SEQ ID NO: 17) is a dinucleotide microsatellite, and GTCGTCGTCGTCGTC (SEQ ID NO: 18) is a trinucleotide microsatellite (with A being Adenine, G Guanine, C Cytosine, and T Thymine). Somatic alterations in the repeat length of such microsatellites have been shown to represent a characteristic feature of tumors. Guide RNAs may be designed to detect such microsatellites. Furthermore, the present invention may be used to detect alterations in repeat length, as well as amplifications and deletions based upon quantitation of the detectable signal. Certain microsatellites are located in regulatory flanking or intronic regions of genes, or directly in codons of genes. Microsatellite mutations in such cases can lead to phenotypic changes and diseases, notably in triplet expansion diseases such as fragile X syndrome and Huntington's disease.

Frequent loss of heterozygosity (LOH) on specific chromosomal regions has been reported in many kinds of malignancies. Allelic losses on specific chromosomal regions are the most common genetic alterations observed in a variety of malignancies, thus microsatellite analysis has been applied to detect DNA of cancer cells in specimens from body fluids, such as sputum for lung cancer and urine for bladder cancer. (Rouleau, et al. Nature 363, 515-521 (1993); and Latif, et al. Science 260, 1317-1320 (1993)). Moreover, it has been established that markedly increased concentrations of soluble DNA are present in plasma of individuals with cancer and some other diseases, indicating that cell free serum or plasma can be used for detecting cancer DNA with microsatellite abnormalities. (Kamp, et al. Science 264, 436-440 (1994); and Steck, et al. Nat Genet. 15(4), 356-362 (1997)). Two groups have reported microsatellite alterations in plasma or serum of a limited number of patients with small cell lung cancer or head and neck cancer. (Hahn, et al. Science 271, 350-353 (1996); and Miozzo, et al. Cancer Res. 56, 2285-2288 (1996)). Detection of loss of heterozygosity in tumors and serum of melanoma patients has also been previously shown (see, e.g., United States patent number U.S. Pat. No. 6,465,177B1).

Thus, it is advantageous to detect of LOH markers in a subject suffering from or at risk of cancer. The present invention may be used to detect LOH in tumor cells. In one embodiment, circulating tumor cells may be used as a biological sample. In preferred embodiments, cell free DNA obtained from serum or plasma is used to noninvasively detect and/or monitor LOH. In other embodiments, the biological sample may be any sample described herein (e.g., a urine sample for bladder cancer). Not being bound by a theory, the present invention may be used to detect LOH markers with improved sensitivity as compared to any prior method, thus providing early detection of mutational events. In one embodiment, LOH is detected in biological fluids, wherein the presence of LOH is associated with the occurrence of cancer. The method and systems described herein represents a significant advance over prior techniques, such as PCR or tissue biopsy by providing a non-invasive, rapid, and accurate method for detecting LOH of specific alleles associated with cancer. Thus, the present invention provides a methods and systems which can be used to screen high-risk populations and to monitor high risk patients undergoing chemoprevention, chemotherapy, immunotherapy or other treatments.

Because the method of the present invention requires only DNA extraction from bodily fluid such as blood, it can be performed at any time and repeatedly on a single patient. Blood can be taken and monitored for LOH before or after surgery; before, during, and after treatment, such as chemotherapy, radiation therapy, gene therapy or immunotherapy; or during follow-up examination after treatment for disease progression, stability, or recurrence. Not being bound by a theory, the method of the present invention also may be used to detect subclinical disease presence or recurrence with an LOH marker specific for that patient since LOH markers are specific to an individual patient's tumor. The method also can detect if multiple metastases may be present using tumor specific LOH markers.

### Detection of Epigenetic Modifications

Histone variants, DNA modifications, and histone modifications indicative of cancer or cancer progression may be used in the present invention. For example, U.S. patent publication 20140206014 describes that cancer samples had elevated nucleosome H2AZ, macroH2A1.1, 5-methylcytosine, P-H2AX(Ser139) levels as compared to healthy subjects. The presence of cancer cells in an individual may generate a higher level of cell free nucleosomes in the blood as a result of the increased apoptosis of the cancer cells. In one embodiment, an antibody directed against marks associated with apoptosis, such as H2B Ser 14(P), may be used to identify single nucleosomes that have been released from apoptotic neoplastic cells. Thus, DNA arising from tumor cells may be advantageously analyzed according to the present invention with high sensitivity and accuracy.

### Pre-Natal Screening

In an embodiment, the method and systems of the present invention may be used in prenatal screening. In an embodiment, cell-free DNA is used in a method of prenatal screening. In an embodiment, DNA associated with single nucleosomes or oligonucleosomes may be detected with the present invention. In preferred embodiments, detection of DNA associated with single nucleosomes or oligonucleosomes is used for prenatal screening. In an embodiment, cell-free chromatin fragments are used in a method of prenatal screening.

Prenatal diagnosis or prenatal screening refers to testing for diseases or conditions in a fetus or embryo before it is born. The aim is to detect birth defects such as neural tube defects, Down syndrome, chromosome abnormalities, genetic disorders and other conditions, such as spina bifida, cleft palate, Tay Sachs disease, sickle cell anemia, thalassemia, cystic fibrosis, Muscular dystrophy, and fragile X syndrome. Screening can also be used for prenatal sex discernment. Common testing procedures include amniocentesis, ultrasonography including nuchal translucency ultrasound, serum marker testing, or genetic screening. In some cases, the tests are administered to determine if the fetus will be aborted, though physicians and patients also find it useful to diagnose high-risk pregnancies early so that delivery can be scheduled in a tertian,′ care hospital where the baby can receive appropriate care.

It has been realized that there are fetal cells which are present in the mother's blood, and that these cells present a potential source of fetal chromosomes for prenatal DNA-based diagnostics. Additionally, fetal DNA ranges from about 2-10% of the total DNA in maternal blood. Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses. Cell-free fetal DNA has been shown to exist in plasma and serum of pregnant women as early as the sixth week of gestation, with concentrations rising during pregnancy and peaking prior to parturition. Because these cells appear very early in the pregnancy, they could form the basis of an accurate, noninvasive, first trimester test. Not being bound by a theory, the present invention provides unprecedented sensitivity in detecting low amounts of fetal DNA. Not being bound by a theory, abundant amounts of maternal DNA is generally concomitantly recovered along with the fetal DNA of interest, thus decreasing sensitivity in fetal DNA quantification and mutation detection. The present invention overcomes such problems by the unexpectedly high sensitivity of the assay.

The H3 class of histones consists of four different protein types: the main types, H3.1 and H3.2; the replacement type, H3.3; and the testis specific variant, H3t. Although H3.1 and H3.2 are closely related, only differing at Ser96, H3.1 differs from H3.3 in at least 5 amino acid positions. Further, H3.1 is highly enriched in fetal liver, in comparison to its presence in adult tissues including liver, kidney and heart. In adult human tissue, the H3.3 variant is more abundant than the H3.1 variant, whereas the converse is true for fetal liver. The present invention may use these differences to detect fetal nucleosomes and fetal nucleic acid in a maternal biological sample that comprises both fetal and maternal cells and/or fetal nucleic acid.

In one embodiment, fetal nucleosomes may be obtained from blood. In other embodiments, fetal nucleosomes are obtained from a cervical mucus sample. In an embodiment, a cervical mucus sample is obtained by swabbing or lavage from a pregnant woman early in the second trimester or late in the first trimester of pregnancy. The sample may be placed in an incubator to release DNA trapped in mucus. The incubator may be set at 37° C. The sample may be rocked for approximately 15 to 30 minutes. Mucus may be further dissolved with a mucinase for the purpose of releasing DNA. The sample may also be subjected to conditions, such as chemical treatment and the like, as well known in the art, to induce apoptosis to release fetal nucleosomes. Thus, a cervical mucus sample may be treated with an agent that induces apoptosis, whereby fetal nucleosomes are released. Regarding enrichment of circulating fetal DNA, reference is made to U.S. patent publication Nos. 20070243549 and 20100240054. The present invention is especially advantageous when applying the methods and systems to prenatal screening where only a small fraction of nucleosomes or DNA may be fetal in origin.

Prenatal screening according to the present invention may be for a disease including, but not limited to Trisomy 13, Trisomy 16, Trisomy 18, Klinefelter syndrome (47, XXY), (47, XYY) and (47, XXX), Turner syndrome, Down syndrome (Trisomy 21), Cystic Fibrosis, Huntington's Disease, Beta Thalassaemia, Myotonic Dystrophy, Sickle Cell Anemia, Porphyria, Fragile-X-Syndrome, Robertsonian translocation, Angelman syndrome, DiGeorge syndrome and Wolf-Hirschhorn Syndrome.

Several further aspects of the invention relate to diagnosing, prognosing and/or treating defects associated with a wide range of genetic diseases which are further described on the website of the National Institutes of Health under the topic subsection Genetic Disorders (website at health.nih.gov/topic/Genetic Disorders).

### Cancer and Cancer Drug Resistance Detection

In an embodiment, the present invention may be used to detect genes and mutations associated with cancer. In an embodiment, mutations associated with resistance are detected. The amplification of resistant tumor cells or appearance of resistant mutations in clonal populations of tumor cells may arise during treatment (see, e.g., Burger J A, et al., Clonal evolution in patients with chronic lymphocytic leukaemia developing resistance to BTK inhibition. Nat Commun. 2016 May 20; 7:11589; Landau D A, et al., Mutations driving CLL and their evolution in progression and relapse. Nature. 2015 Oct. 22; 526(7574):525-30; Landau D A, et al., Clonal evolution in hematological malignancies and therapeutic implications. Leukemia. 2014 January; 28(1):34-43; and Landau D A, et al., Evolution and impact of subclonal mutations in chronic lymphocytic leukemia. Cell. 2013 Feb. 14; 152(4):714-26). Accordingly, detecting such mutations requires highly sensitive assays and monitoring requires repeated biopsy. Repeated biopsies are inconvenient, invasive and costly. Resistant mutations can be difficult to detect in a blood sample or other noninvasively collected biological sample (e.g., blood, saliva, urine) using the prior methods known in the art. Resistant mutations may refer to mutations associated with resistance to a chemotherapy, targeted therapy, or immunotherapy.

In an embodiment, mutations occur in individual cancers that may be used to detect cancer progression. In one embodiment, mutations related to T cell cytolytic activity against tumors have been characterized and may be detected by the present invention (see e.g., Rooney et al., Molecular and genetic properties of tumors associated with local immune cytolytic activity, Cell. 2015 January 15; 160(1-2): 48-61). Personalized therapies may be developed for a patient based on detection of these mutations (see e.g., WO2016100975A1). In an embodiment, cancer specific mutations associated with cytolytic activity may be a mutation in a gene selected from the group consisting of CASP8, B2M, PIK3CA, SMC1A, ARID5B, TET2, ALPK2, COL5A1, TP53, DNER, NCOR1, MORC4, CIC, IRF6, MYOCD, ANKLE1, CNKSR1, NF1, SOS1, ARID2, CUL4B, DDX3X, FUBP1, TCP11L2, HLA-A, B or C, CSNK2A1, MET, ASXL1, PD-L1, PD-L2, IDO1, ID02, ALOX12B and ALOX15B, or copy number gain, excluding whole-chromosome events, impacting any of the following chromosomal bands: 6q16.1-q21, 6q22.31-q24.1, 6q25.1-q26, 7p11.2-q11.1, 8p23.1, 8p11.23-p11.21 (containing IDO1, ID02), 9p24.2-p23 (containing PDL1, PDL2), 10p15.3, 10p15.1-p13, 11p14.1, 12p13.32-p13.2, 17p13.1 (containing ALOX12B, ALOX15B), and 22q11.1-q11.21.

In an embodiment, the present invention is used to detect a cancer mutation (e.g., resistance mutation) during the course of a treatment and after treatment is completed. The sensitivity of the present invention may allow for noninvasive detection of clonal mutations arising during treatment and can be used to detect a recurrence in the disease.

In an embodiment, detection of microRNAs (miRNA) and/or miRNA signatures of differentially expressed miRNA, may used to detect or monitor progression of a cancer and/or detect drug resistance to a cancer therapy. As an example, Nadal et al. (Nature Scientific Reports, (2015) doi:10.1038/srep12464) describe mRNA signatures that may be used to detect non-small cell lung cancer (NSCLC).

In an embodiment, the presence of resistance mutations in clonal subpopulations of cells may be used in determining a treatment regimen. In other embodiments, personalized therapies for treating a patient may be administered based on common tumor mutations. In an embodiment, common mutations arise in response to treatment and lead to drug resistance. In an embodiment, the present invention may be used in monitoring patients for cells acquiring a mutation or amplification of cells harboring such drug resistant mutations.

Treatment with various chemotherapeutic agents, particularly with targeted therapies such as tyrosine kinase inhibitors, frequently leads to new mutations in the target molecules that resist the activity of the therapeutic. Multiple strategies to overcome this resistance are being evaluated, including development of second generation therapies that are not affected by these mutations and treatment with multiple agents including those that act downstream of the resistance mutation. In an exemplary embodiment, a common mutation to ibrutinib, a molecule targeting Bruton's Tyrosine Kinase (BTK) and used for CLL and certain lymphomas, is a Cysteine to Serine change at position 481 (BTK/C481S). Erlotinib, which targets the tyrosine kinase domain of the Epidermal Growth Factor Receptor (EGFR), is commonly used in the treatment of lung cancer and resistant tumors invariably develop following therapy. A common mutation found in resistant clones is a threonine to methionine mutation at position 790.

Non-silent mutations shared between populations of cancer patients and common resistant mutations that may be detected with the present invention are known in the art (see e.g., WO/2016/187508). In an embodiment, drug resistance mutations may be induced by treatment with ibrutinib, erlotinib, imatinib, gefitinib, crizotinib, trastuzumab, vemurafenib, RAF/MEK, check point blockade therapy, or antiestrogen therapy. In an embodiment, the cancer specific mutations are present in one or more genes encoding a protein selected from the group consisting of Programmed Death-Ligand 1 (PD-L1), androgen receptor (AR), Bruton's Tyrosine Kinase (BTK), Epidermal Growth Factor Receptor (EGFR), BCR-Abl, c-kit, PIK3CA, HER2, EML4-ALK, KRAS, ALK, ROS1, AKT1, BRAF, MEK1, MEK2, NRAS, RAC1, and ESR1.

Immune checkpoints are inhibitory pathways that slow down or stop immune reactions and prevent excessive tissue damage from uncontrolled activity of immune cells. In an embodiment, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Recently, gene expression in tumors and their microenvironments have been characterized at the single cell level (see e.g., Tirosh, et al. Dissecting the multicellular ecosystem of metastatic melanoma by single cell RNA-seq. Science 352, 189-196, doi:10.1126/science.aad0501 (2016)); Tirosh et al., Single-cell RNA-seq supports a developmental hierarchy in human oligodendroglioma. Nature. 2016 Nov. 10; 539(7628):309-313. doi: 10.1038/nature20123. Epub 2016 Nov. 2; and International patent publication serial number WO 2017004153 A1). In an embodiment, gene signatures may be detected using the present invention. In one embodiment complement genes are monitored or detected in a tumor microenvironment. In one embodiment MITF and AXL programs are monitored or detected. In one embodiment, a tumor specific stem cell or progenitor cell signature is detected. Such signatures indicate the state of an immune response and state of a tumor. In an embodiment, the state of a tumor in terms of proliferation, resistance to treatment and abundance of immune cells may be detected.

Thus, in an embodiment, the invention provides low-cost, rapid, multiplexed cancer detection panels for circulating DNA, such as tumor DNA, particularly for monitoring disease recurrence or the development of common resistance mutations.

### Immunotherapy Applications

The embodiments disclosed herein can also be useful in further immunotherapy contexts. For instance, In an embodiment methods of diagnosing, prognosing and/or staging an immune response in a subject comprise detecting a first level of expression, activity and/or function of one or more biomarker and comparing the detected level to a control level wherein a difference in the detected level and the control level indicates that the presence of an immune response in the subject.

In an embodiment, the present invention may be used to determine dysfunction or activation of tumor infiltrating lymphocytes (TIL). TILs may be isolated from a tumor using known methods. The TILs may be analyzed to determine whether they should be used in adoptive cell transfer therapies. Additionally, chimeric antigen receptor T cells (CAR T cells) may be analyzed for a signature of dysfunction or activation before administering them to a subject. Exemplary signatures for dysfunctional and activated T cell have been described (see e.g., Singer M, et al., A Distinct Gene Module for Dysfunction Uncoupled from Activation in Tumor-Infiltrating T Cells. Cell. 2016 Sep. 8; 166(6):1500-1511.e9. doi: 10.1016/j.cell.2016.08.052).

In an embodiment, C2c2 is used to evaluate that state of immune cells, such as T cells (e.g., CD8+ and/or CD4+ T cells). In particular, T cell activation and/or dysfunction can be determined, e.g., based on genes or gene signatures associated with one or more of the T cell states. In this way, c2c2 can be used to determine the presence of one or more subpopulations of T cells.

In an embodiment, C2c2 can be used in a diagnostic assay or may be used as a method of determining whether a patient is suitable for administering an immunotherapy or another type of therapy. For example, detection of gene or biomarker signatures may be performed via c2c2 to determine whether a patient is responding to a given treatment or, if the patient is not responding, if this may be due to T cell dysfunction. Such detection is informative regarding the types of therapy the patient is best suited to receive. For example, whether the patient should receive immunotherapy.

In an embodiment, the systems and assays disclosed herein may allow clinicians to identify whether a patient's response to a therapy (e.g., an adoptive cell transfer (ACT) therapy) is due to cell dysfunction, and if it is, levels of up-regulation and down-regulation across the biomarker signature will allow problems to be addressed. For example, if a patient receiving ACT is non-responsive, the cells administered as part of the ACT may be assayed by an assay disclosed herein to determine the relative level of expression of a biomarker signature known to be associated with cell activation and/or dysfunction states. If a particular inhibitory receptor or molecule is up-regulated in the ACT cells, the patient may be treated with an inhibitor of that receptor or molecule. If a particular stimulatory receptor or molecule is down-regulated in the ACT cells, the patient may be treated with an agonist of that receptor or molecule.

In an embodiment, the systems, methods, and devices described herein may be used to screen gene signatures that identify a particular cell type, cell phenotype, or cell state. Likewise, through the use of such methods as compressed sensing, the embodiments disclosed herein may be used to detect transcriptomes. Gene expression data are highly structured, such that the expression level of some genes is predictive of the expression level of others. Knowledge that gene expression data are highly structured allows for the assumption that the number of degrees of freedom in the system are small, which allows for assuming that the basis for computation of the relative gene abundances is sparse. It is possible to make several biologically motivated assumptions that allow Applicants to recover the nonlinear interaction terms while under-sampling without having any specific knowledge of which genes are likely to interact. In particular, if Applicants assume that genetic interactions are low rank, sparse, or a combination of these, then the true number of degrees of freedom is small relative to the complete combinatorial expansion, which enables Applicants to infer the full nonlinear landscape with a relatively small number of perturbations. Working around these assumptions, analytical theories of matrix completion and compressed sensing may be used to design under-sampled combinatorial perturbation experiments. In addition, a kernel-learning framework may be used to employ under-sampling by building predictive functions of combinatorial perturbations without directly learning any individual interaction coefficient Compresses sensing provides a way to identify the minimal number of target transcripts to be detected in order obtain a comprehensive gene-expression profile. Methods for compressed sensing are disclosed in PCT/US2016/059230 “Systems and Methods for Determining Relative Abundances of Biomolecules” filed Oct. 27, 2016, which is incorporated herein by reference. Having used methods like compressed sensing to identify a minimal transcript target set, a set of corresponding guide RNAs may then be designed to detect said transcripts. Accordingly, in an embodiment, a method for obtaining a gene-expression profile of cell comprises detecting, using the embodiments disclosed, herein a minimal transcript set that provides a gene-expression profile of a cell or population of cells.

### Detecting Nucleic Acid Tagged Molecules

In an embodiment, the detection compositions of the present invention described herein may be used to detect nucleic acid identifiers. Nucleic acid identifiers are non-coding nucleic acids that may be used to identify a particular article. Example nucleic acid identifiers, such as DNA watermarks, are described in Heider and Barnekow. “DNA watermarks: A proof of concept” BMC Molecular Biology 9:40 (2008). The nucleic acid identifiers may also be a nucleic acid barcode. A nucleic-acid based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify target molecules and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

### Cell Labeling

The programmable pattern recognition compositions and/or detection compositions of the present invention can be used, for example, to label a cell. As previously described in relation to e.g., methods of detecting target polynucleotides, when a detection composition of the present invention is activated by binding a target polynucleotide a detectable signal or product is produced. In an embodiment, the detectable signal or product is such that it allows a cell to which the system is delivered to and activated in to be “labeled” via the detectable signal or product. For example, if the detectable signal is an optical signal (e.g., fluorescence) produced from a protein, then the cell is effectively labeled with fluorescence that can be tracked, imaged, and used for e.g., fluorescence-based sorting or separation techniques. Other signals and products that can be used as labels are described in greater detail elsewhere herein and will be appreciated in view of the description provided herein. In this way cells containing a target polynucleotide can be effectively labeled. Labeling via a method described herein can occur in vivo, ex vivo, in vitro, or in situ. Such methods can be applied to various cell detection, imaging, diagnostic, prognostic, screening, functionality, cell isolation and separation, and other assays and techniques where cell labeling is traditionally employed. Such labeling approaches can be helpful for cell type and cell state evaluation, particularly at the single cell level.

Described in an embodiment herein are methods of labeling cells comprising introducing a pattern recognition composition of the present invention or a detection composition comprising an engineered protein of the present invention as described in greater detail elsewhere herein into a population of cells, wherein the pattern recognition compositions and/or a guide molecule employed is configured to detect one or more target nucleic acids (e.g., DNA or RNA) or polypeptides associated with a particular cell type or cell state; and activating the pattern recognition composition via recognition and/or binding of the protein composition to the one or more target transcripts such that a detection construct is modified by the activated pattern recognition composition such that a detectable product and/or signal is generated, thereby labeling cells within the cell population expressing the one or more target transcripts.

In an embodiment, a substrate the pattern recognition composition or component thereof (e.g., an effector domain) of the present invention is tethered or anchored to a structure within the cell. Exemplary cell structures to which the substrate can be anchored is the cell or nuclear membrane, mitochondria membrane, endoplasmic reticulum, lysosome, Golgi apparatus, microtubules or other cytoskeleton components, and/or the like. In an embodiment the substrate is coupled to a signal producing molecule or product producing molecule that is inactive until released from the peptidase substrate or is otherwise modified by activity of the pattern recognition composition or effector thereof on the substrate upon recognition of a target molecule (e.g., a target polypeptide and/or polynucleotide) and/or target molecular pattern, which can optionally be associated or contained within or on the surface of a target cell or other molecule (e.g., exosomes or the like).

Therapeutic Delivery and/or Effector Function

Similar to embodiments of cell labeling, the programmable pattern recognition compositions of the present invention can be configured for in vivo effector function and/or delivery of a cargo, such as a therapeutic molecule. A substrate for the pattern recognition composition or component thereof (e.g., an effector domain) can be tethered or otherwise anchored to a cellular structure. Exemplary cell structures to which the substrate can be anchored is the cell or nuclear membrane, mitochondria membrane, endoplasmic reticulum, lysosome, Golgi apparatus, microtubules or other cytoskeleton components, and/or the like. The substrate can also be coupled to (either directly or via a linker) an effector molecule (e.g., a Cre recombinase, CRISPR-Cas system, or other effector molecule) and/or to a cargo (e.g., a therapeutic molecule). In an embodiment, the effector molecule or other molecule (e.g., a therapeutic molecule), is inactive while coupled to the substrate. When a target molecule (e.g., a target polynucleotide or target polypeptide) and/or target molecular pattern is present in cell that also contains the programmable pattern recognition composition, one or more effector domains of the programmable pattern recognition composition are activated upon recognition and/or binding of the target molecule (e.g., a target polynucleotide or target polypeptide) and/or target molecular pattern that can be associated with a target cell, polypeptide and/or RNA and acts to cleave the tethered substrate. Cleaving of the substrate releases and/or otherwise activates the effector or cargo (e.g., therapeutic molecule) that was linked to the peptidase substrate. Target molecules (e.g., a target polynucleotide or target polypeptide) and/or molecules having a target molecular pattern can be endogenous to the cell that expresses the programmable pattern recognition composition and/or tethered effector and/or cargo (or therapeutic) complex. In other embodiments, target molecules (e.g., a target polynucleotide or target polypeptide) and/or molecules having a target molecular pattern are exogenous to the cell. Exogenous Target molecules (e.g., a target polynucleotide or target polypeptide) and/or molecules having a target molecular pattern can provide an additional measure of temporal and/or spatial control of effector function and/or therapeutic delivery. Exemplary effectors that can be included in these embodiments are described in greater detail elsewhere herein and will be appreciated by those of ordinary skill in the art in view of the description herein.

In an embodiment, a method of in vivo effector activation or delivery includes introducing a programmable pattern recognition composition or system of the present invention into a cell comprising a substrate of the programmable pattern recognition composition or component thereof (e.g., a STAND NTPase or other effector domain), wherein the substrate of the programmable pattern recognition composition is optionally tethered to a cellular structure and wherein the substrate the programmable pattern recognition composition is coupled to an effector. In an embodiment, the effector is capable of producing a detectable signal when activated. In an embodiment, the cargo is a therapeutic molecule or prodrug, genetic modifying molecule, or any combination thereof. In an embodiment, the effector is inactive when coupled to an uncleaved substrate. In an embodiment, the effector is inactive when coupled to a cleaved substrate portion (and thus is active when coupled to an uncleaved substrate). In an embodiment, the method further comprises cleaving the substrate in response to target molecule recognition and/or binding and activation of an effector domain of the programmable pattern recognition composition of the present invention. In an embodiment, the target molecule is endogenous to the cell or is exogenous to the cell. In an embodiment, the substrate is tethered to a cell membrane or a nuclear membrane.

In an embodiment, the method of cargo delivery comprises delivering to a cell (a) an engineered protein of the present invention: (b) a cargo, (c) a detection composition), or any combination thereof, wherein the engineered protein comprises the cargo or wherein the cargo comprises the target polypeptide, wherein the cell optionally comprises the target polypeptide, and wherein activation of the effector domain by binding of the target polypeptide to the recognition domain results in delivery of the cargo and optionally activation of the detection construct thereby monitoring cargo delivery.

In an embodiment, the cargo is a prodrug or proenzyme. In an embodiment, the target molecule (e.g., a target polypeptide or polynucleotide) modification is cleavage of the target molecule (e.g., a target polypeptide or polynucleotide). In an embodiment, the one or more target molecule (e.g., a target polypeptide or polynucleotide) are proenzymes, proproteins, and/or prodrugs, and the modification results in conversion of the proenzyme into an active enzyme, active protein, or active prodrug, respectively. Thus, In an embodiment, the engineered proteins of the present invention can be used to deliver and/or control delivery of a drug or enzyme from its prodrug form.

### Targeted Cell Death

In an embodiment, the programmable pattern recognition compositions are used for targeted cell death. In such embodiments, the programmable pattern recognition compositions are configured to recognize and/or bind a target molecule (e.g., a target polypeptide or target polynucleotide) and/or target molecular pattern of target cells to be killed. The programmable pattern recognition composition can contain an effector domain (e.g., a protease or nuclease) that, when activated upon target molecule (e.g., target polynucleotide or target polypeptide) and/or target molecular pattern recognition and/or binding, induces cell death. In an embodiment, the method includes delivering an engineered protein of the present invention, a polynucleotide of encoding an engineered protein of the present invention, a vector or vector system of the present invention, a formulation thereof, or any combination thereof to the target molecule and/or cell, wherein the target molecule and/or cell is or comprises a target molecule (e.g., a target polypeptide, target polynucleotide) and/or target molecular pattern (e.g., a PAMP); and activating an effector domain of the engineered protein by allowing binding of the target molecule (e.g., target polypeptide or target polynucleotide) and/or target molecular pattern to the recognition domain thereby activating the effector domain via the effector activation domain, wherein effector domain activity induces cell death (e.g., via apoptosis or other cell death mechanism) of the target cell. Without being bound by theory, an activated nuclease or protease effector domain can induce apoptosis in the target cells, leading to cell death. In an embodiment, the target cells are prokaryotic cells. In an embodiment, the target cells are bacterial cells. Thus, In an embodiment, the engineered proteins of the present invention are antibiotic compositions. In an embodiment the target cells are eukaryotic cells. In an embodiment, the target cells are tumor or cancer cells. Thus, In an embodiment, the engineered proteins of the present invention are anti-cancer or anti-tumor compositions. In an embodiment, the target cells are diseased, infected or aberrantly functioning cells.

### Generating Phage-Resistant Cells

In an embodiment, the programmable pattern recognition compositions are used to generate or engineer phage-resistant cells. In such embodiments, the programmable pattern recognition compositions are configured to recognize a target molecule (e.g., a target polypeptide or target polynucleotide) and/or a target molecular pattern (e.g., a PAMP) of one or more phages. Further, the programmable pattern recognition compositions are configured to inactivate and/or destroy or eliminate phages comprising the target molecule (e.g., a target polypeptide or target polynucleotide) and/or a target molecular pattern (e.g., a PAMP) that is recognized by the programmable pattern recognition composition. Such a configuration can include a peptidase effector and/or a nuclease effector domain. In an embodiment, the programmable pattern recognition compositions are introduced into and/or expressed by cells, such as bacterial cells. When invaded by a phage, the programmable pattern recognition compositions recognizes a target molecule (e.g., a target polypeptide or target polynucleotide) and/or a target molecular pattern (e.g., a PAMP) that on the invading phage and is activated by such recognition. Activation results in effector domain function of the programmable pattern recognition composition, and inhibition and/or destruction of the invading phage. Such phage resistant cells can have various industrial applications, such as in industrial bioreactors.

### Microbiome Engineering

In an embodiment, the programmable pattern recognition compositions are used to engineer a microbiome, and more particularly the structure of a microbiome. As used herein, the term “microbiome structure” refers to the profile (e.g., relative or absolute numbers) of different microbes present in a microbiome. Engineering a microbiome can include modulating the microbiome structure to a desired structure. The programmable pattern recognition compositions can be configured to recognize target molecule (e.g., a target polypeptide or target polynucleotide) and/or a target molecular pattern (e.g., a PAMP) on a specific microbe type (e.g., species, subspecies, clade and/or the like) to be increased in number, decreased in number, modified, and/or eliminated, so as to modify and engineer the microbiome structure. In an embodiment, the modification results in a phage-resistant bacteria, which can result in an increase in the number of the phage-resistant bacteria within the microbiome as it has an evolutionary advantage of being phage-resistant. In an embodiment, the modification results in apoptosis of the target cell(s) thus removing it from the microbiome. In an embodiment, the modification in the target cells provides a competitive advantage so as to make the target cell type the dominate cell type within the microbiome population. In an embodiment, the targeted microbe(s) are increased in relative or absolute amount within the microbiome after modification by the programmable pattern recognition composition of the present invention. In an embodiment, the targeted microbe(s) are decreased in relative or absolute amount within the microbiome after modification by the programmable pattern recognition composition of the present invention.

In an embodiment a method of microbiome engineering includes delivering a programmable pattern recognition protein or composition to one or more microorganisms of microbiome to which the target molecule (e.g., a target polypeptide or target polynucleotide) and/or a target molecular pattern (e.g., a PAMP) is specific to and wherein an effector of the engineered protein modifies, stimulates, or kills the one or more microorganisms to which the protein is delivered, thus resulting in a change (e.g., increase or decrease) in the absolute or relative number of the microorganism within the microbiome, thereby modifying (or engineering) the microbiome structure.

Further embodiments are illustrated in the following Examples which are given for illustrative purposes only and are not intended to limit the scope of the invention.

### EXAMPLES

### Example 1—STAND NTPases

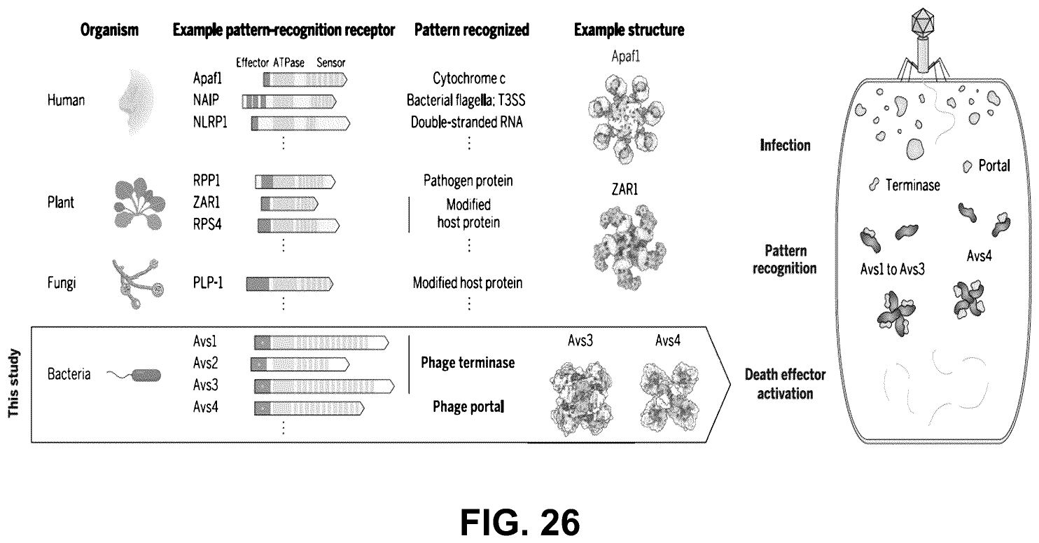

All organisms have evolved specialized immune proteins, including pattern recognition receptors consisting of nucleotide-binding oligomerization domain-like receptors (NLRs) of the STAND superfamily ubiquitous in eukaryotes. NLRs recognize conserved pathogen-associated molecular patterns, leading to activation of an effector domain and an inflammatory or apoptotic response. The roles of NLRs in eukaryotic immunity are well established, but it is unknown whether prokaryotes use similar defense mechanisms. Here Applicant shows that antiviral STAND (Avs) homologs in bacteria and archaea are pattern recognition receptors that detect conserved viral proteins and activate diverse N-terminal effectors, including DNA endonucleases. See e.g., FIG. 26. This work reveals remarkable similarity between the defense strategies of prokaryotes and eukaryotes and extends the paradigm of pattern recognition of pathogen-specific proteins across all domains of life.

Applicants recently identified a group of STAND NTPases, dubbed Avs (antiviral STAND) (4), that are often encoded next to restriction modification and other defense systems (FIG. 1A-1B) and protect bacteria from dsDNA phages (FIG. 2). Here, Applicants investigate the mechanism of Avs proteins, which consist of diverse N-terminal effector domains, a conserved central STAND NTPase core, and C-terminal tetratricopeptide repeat (TPR) domains. Applicants demonstrate that Avs systems specifically bind to and are activated by the conserved large terminase subunit and portal proteins of diverse tailed phages; that some Avs systems function as non-specific DNA endonucleases; and that some phages encode Avs inhibitors. Avs systems are activated by two conserved phage proteins.

### Avs Systems are Activated by Two Conserved Phage Proteins

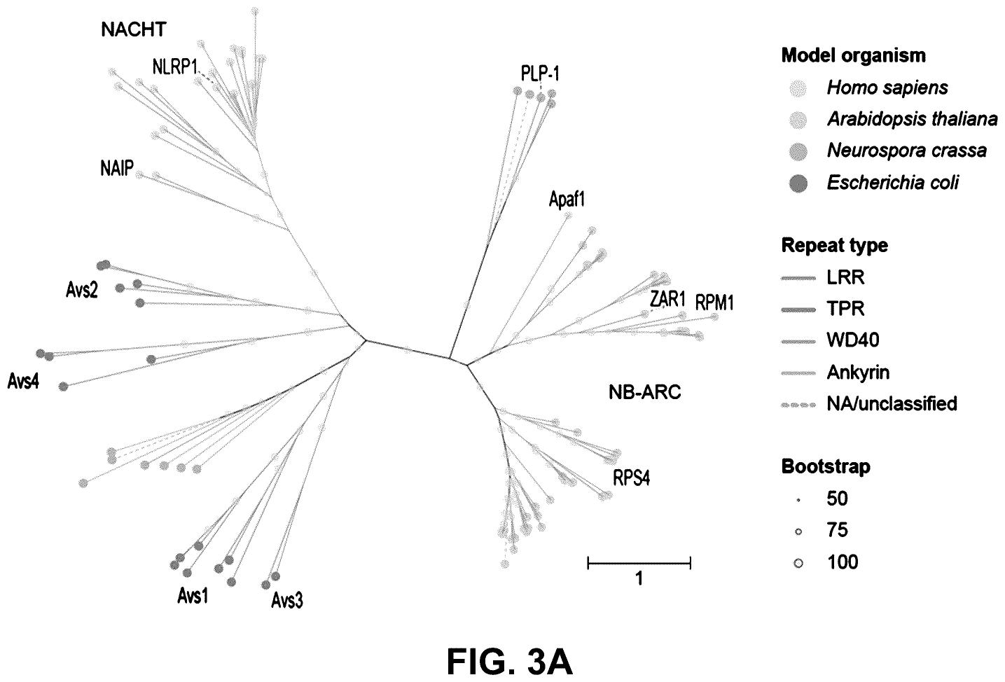

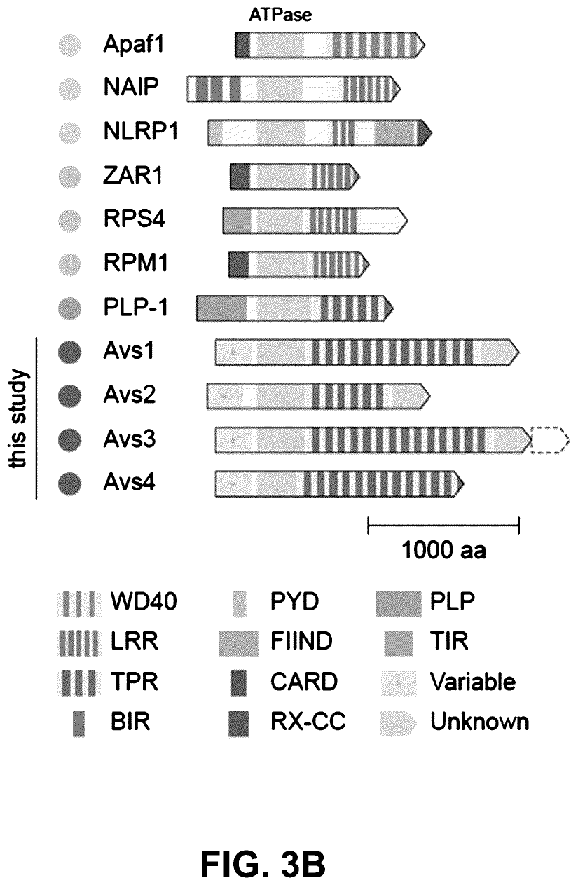

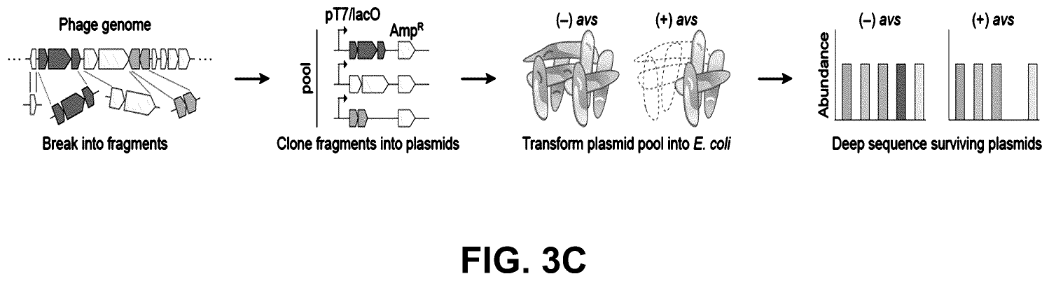

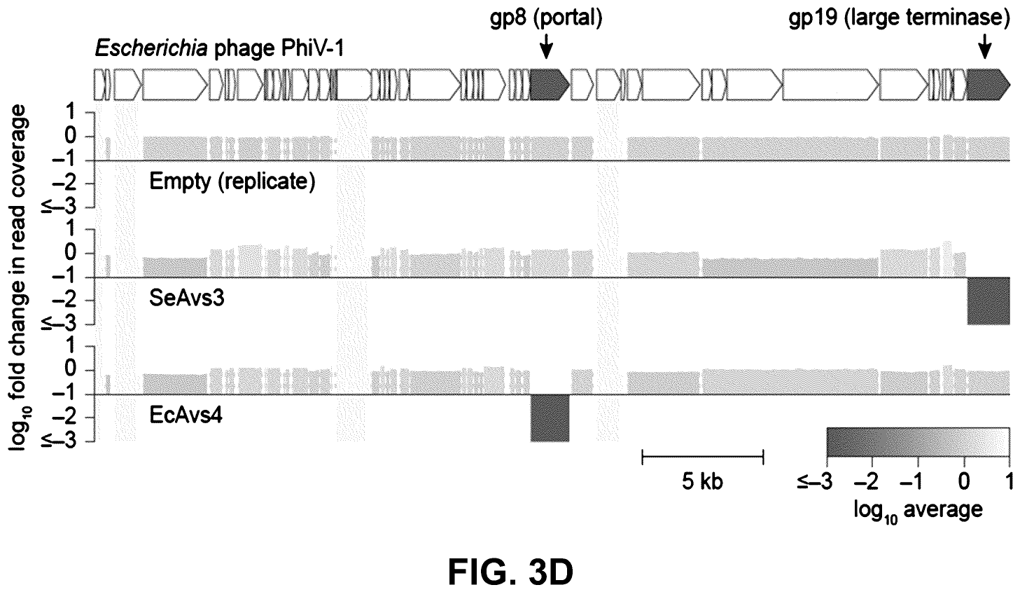

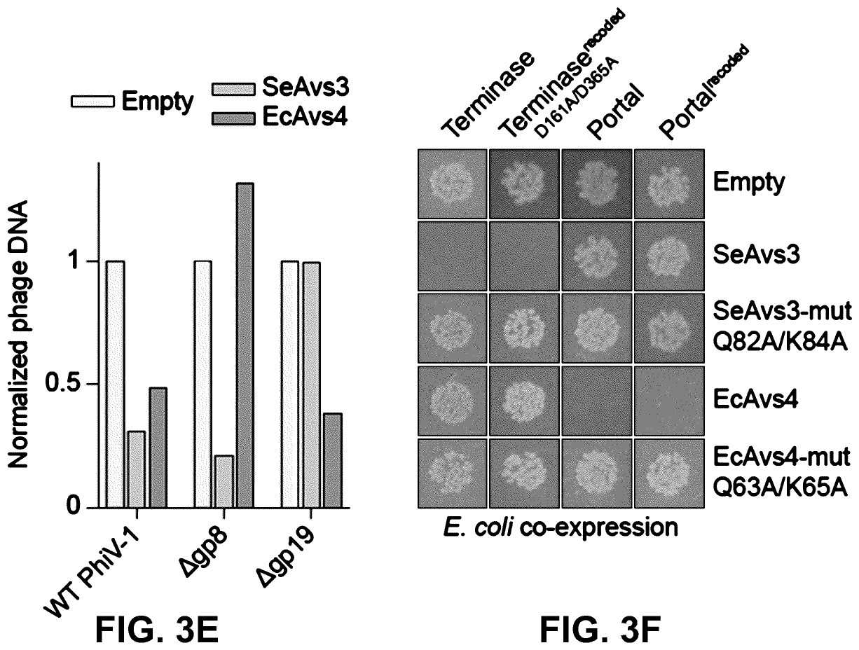

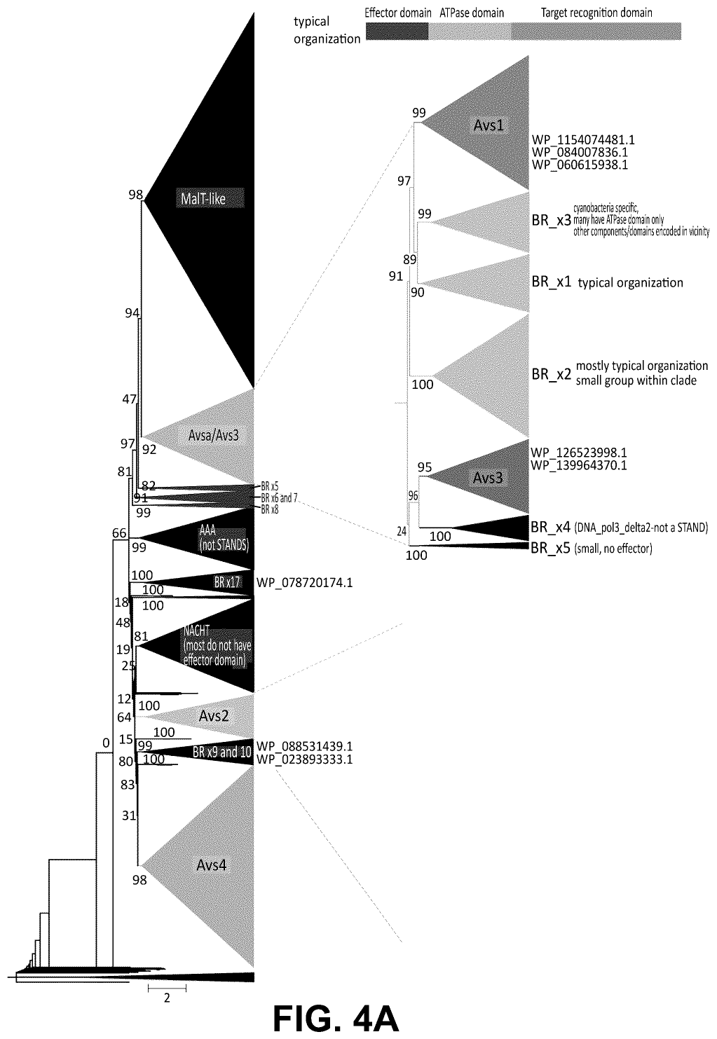

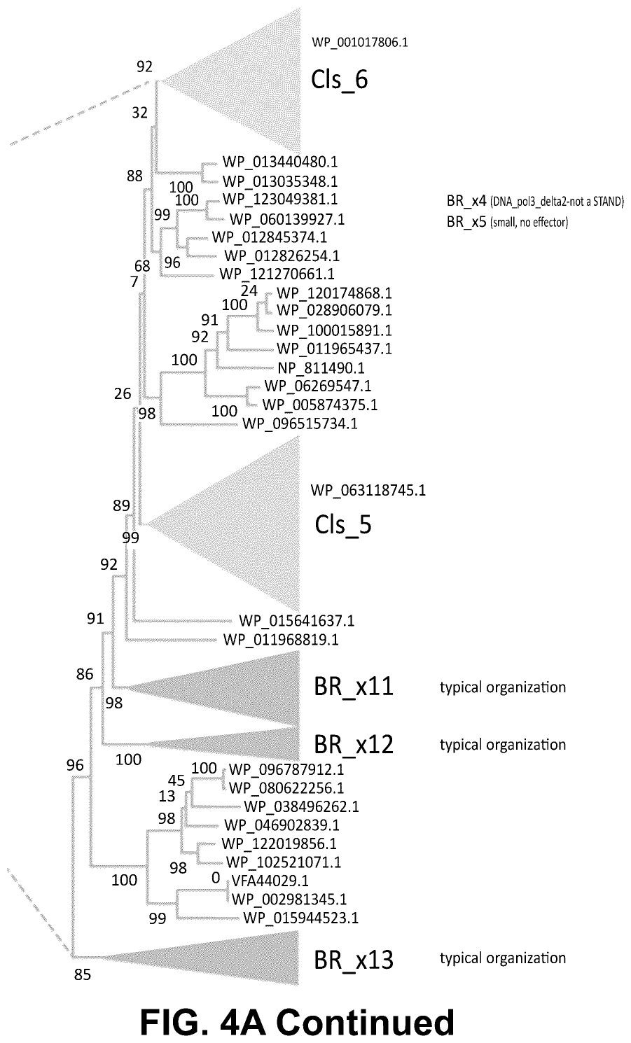

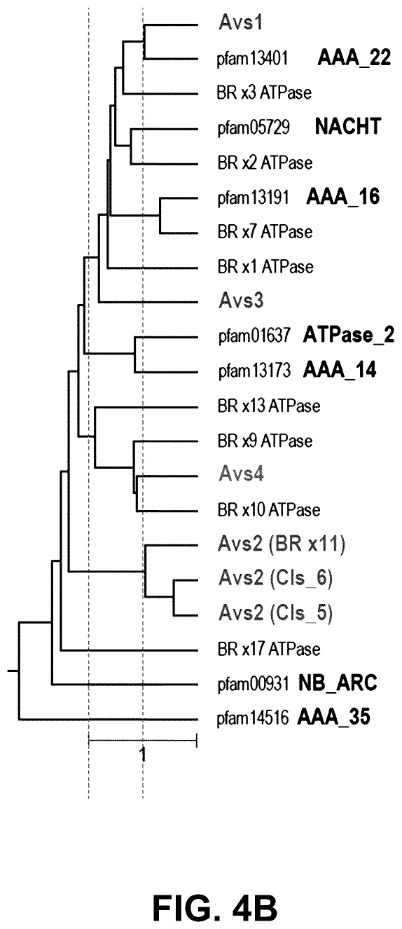

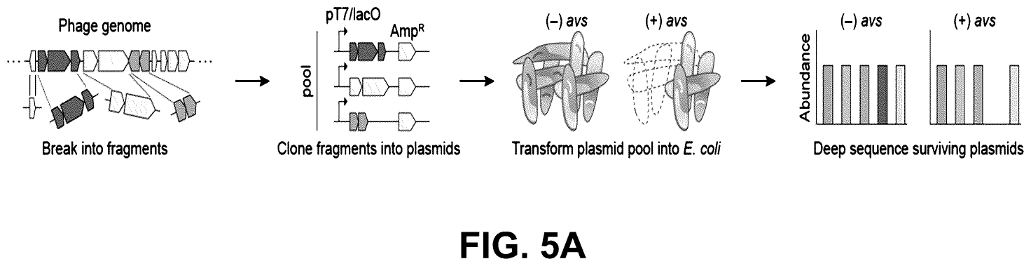

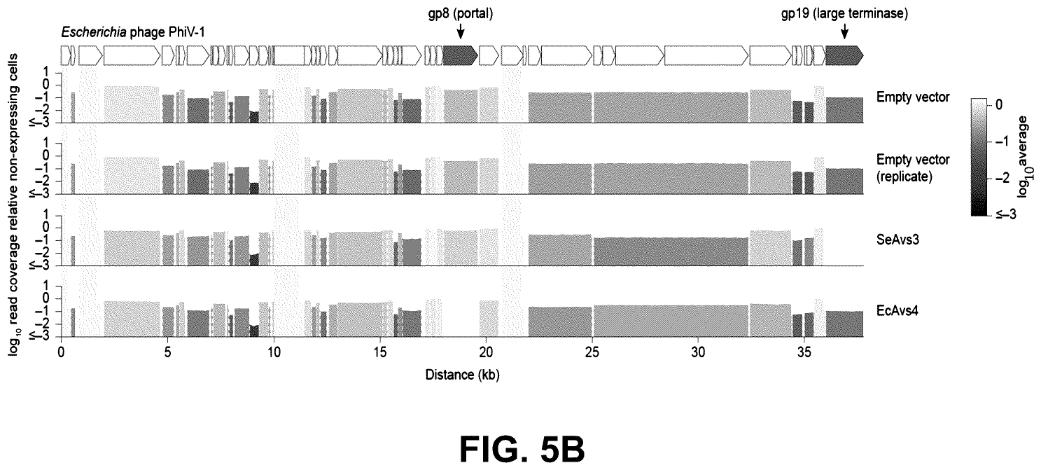

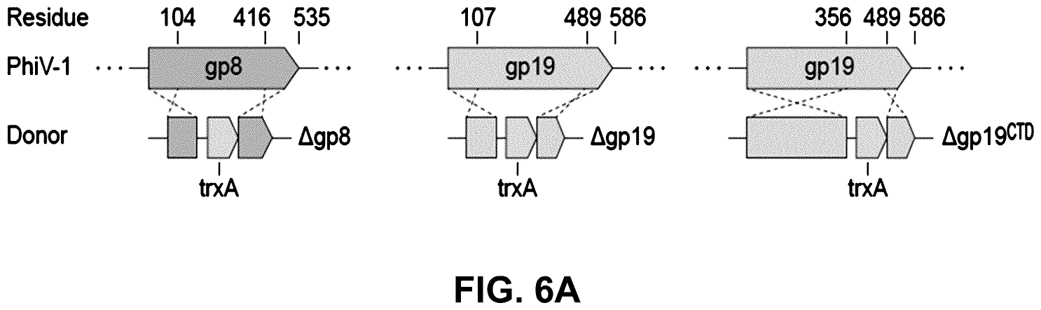

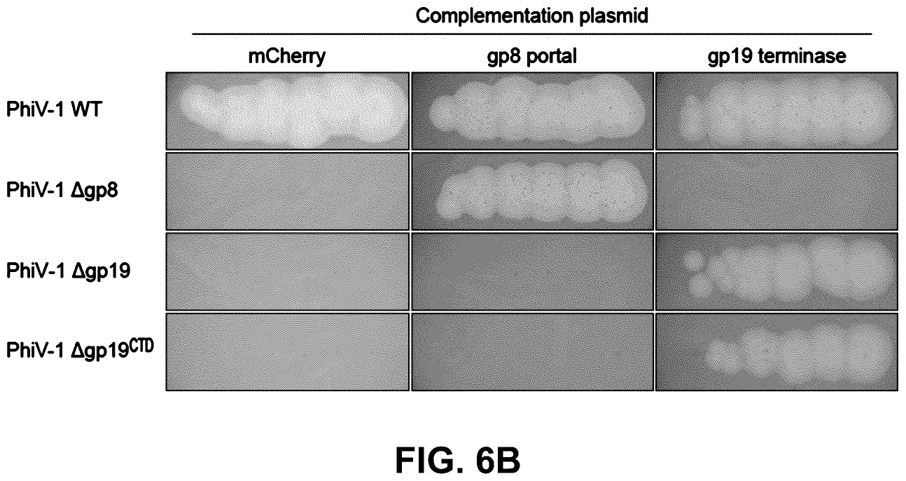

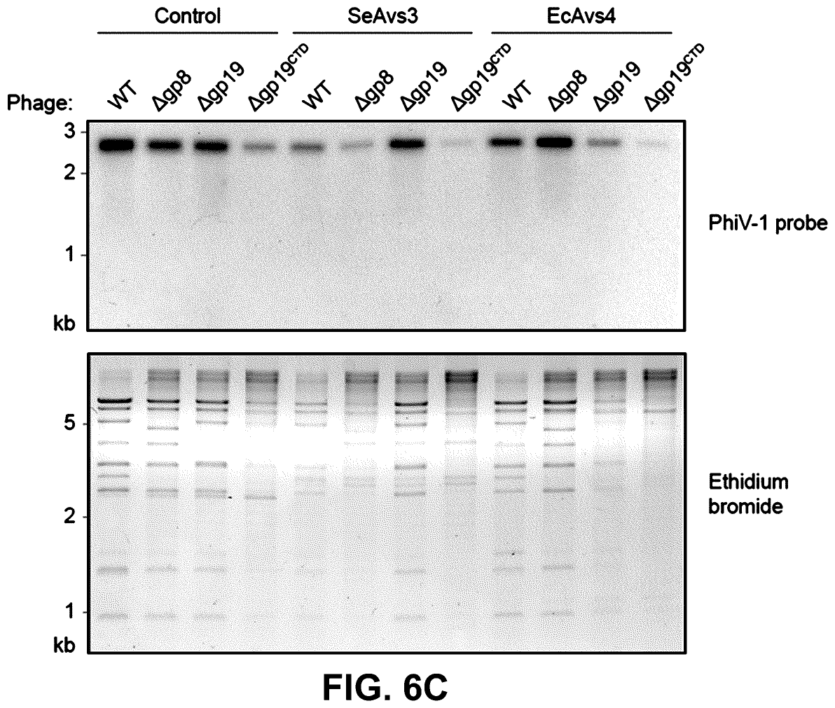

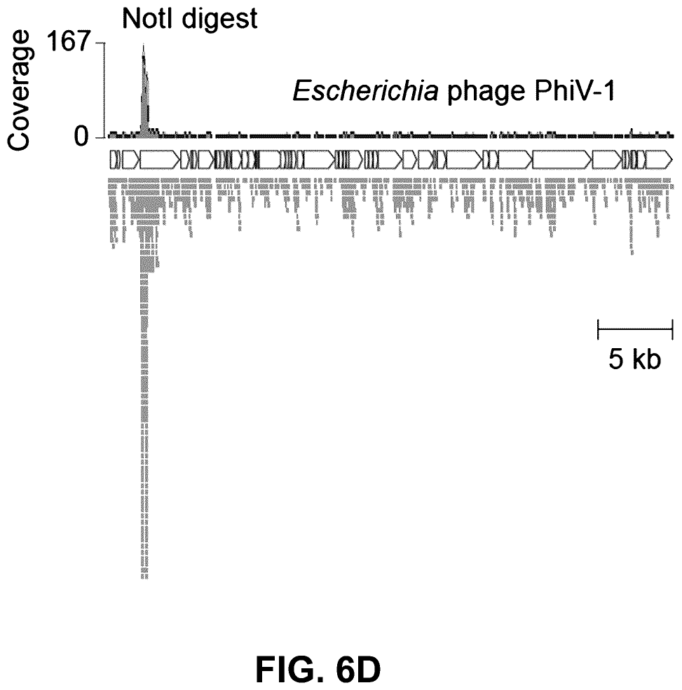

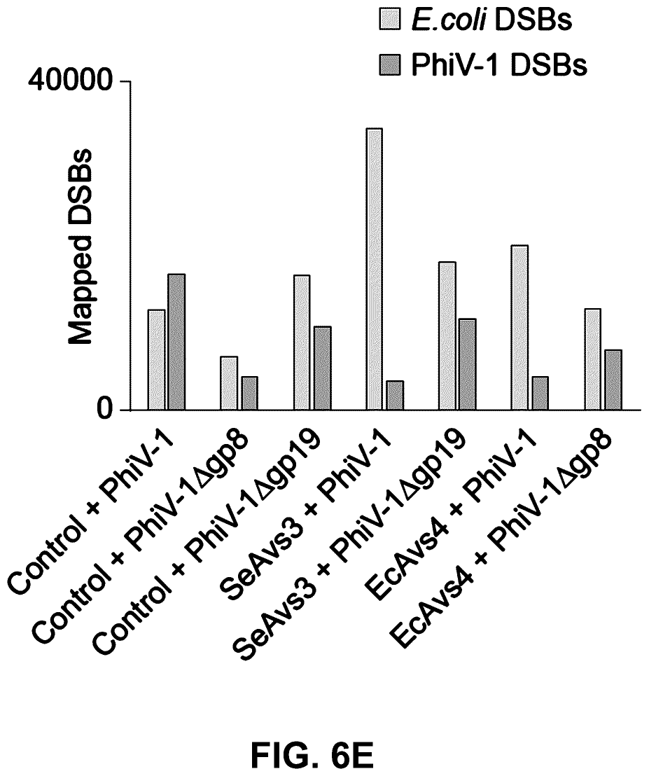

Although the domain architectures of Avs proteins resemble those of eukaryotic NLRs (FIG. 3A-3B), it is unclear whether they function via similar molecular mechanisms. Applicant identified four distinct families of Avs proteins (Avs1-4), each of which contains highly divergent tetratrico-peptide repeat (TPR)-like sensor domains (FIG. 4A-4B) and selected two representatives for further characterization: SeAvs3 from Salmonella enterica NCTC13175 and EcAvs4 from Escherichia coli NCTC11132, both of which provide robust protection against the T7-like coliphage PhiV-1 (FIG. 2). Applicants first asked how phage infection leads to Avs activation and whether a specific phage-encoded trigger exists for these defense systems. Applicants cloned fragments comprising the whole PhiV-1 phage genome into expression plasmids and transformed the resulting phage library into E. coli containing either Avs proteins or empty controls (FIG. 3C and Table 10). Applicant hypothesized that co-expression of putative Avs triggers might lead to cell death and depletion of the respective phage genes from the pool, and performed deep sequencing to detect enrichment or depletion of phage genes. Four phage genes were generally toxic to all cells; however, two genes were depleted only in the presence of Avs systems, namely the large terminase subunit (gp19) when co-expressed with SeAvs3, and the portal protein (gp8) when expressed with EcAvs4 (FIG. 3D and FIG. 5A-5B). By Southern blot, Applicant observed that Avs3 and Avs4-mediated depletion of phage DNA during infection was abolished in gp8 and gp19 knockout phage strains, respectively (FIG. 3E and FIG. 6A-6E), indicating that gp8 and gp19 are both necessary and sufficient for Avs activation.

To validate these findings, Applicant transformed plasmids expressing gp8 or gp19 into E. coli harboring SeAvs3 or EcAvs4 and measured cell growth. Consistent with Applicant's previous results, Applicant observed toxicity following co-expression of SeAvs3 and gp19, as well as co-expression of EcAvs4 and gp8, but not with the reciprocal pairs (FIG. 3E). This toxicity depended on the predicted nuclease activity of both Avs systems, and importantly, was not due to any intrinsic features of the natural phage gene sequence, as recoded gene sequences also led to cell death (FIG. 3F). Further, the enzymatic activity of the phage terminase, which contains triphosphate (ATPase) and nuclease domains unrelated to those of Avs proteins, was not required for SeAvs3-mediated toxicity (FIG. 3F).

TABLE 10
PhiV-1 phage fragments

| | | | | | | Abundance relative to E. coli | | | |
|---|---|---|---|---|---|---|---|---|---|
| | | | | | | NovaBlue control | | | |
| | | | | | | Empty | Empty | | |
| # | Gene(s) | DNA | bp | Toxic | Proteins(s) | vector | vector | SeAvs3 | EcAvs4 |
| 1 | gp0.3 | ATGATTCACACTAAAGAGCAAGCT | 456 | + | Anti- | — | — | — | — |
| | | AACGTTTTCTATGTACTGGTAAGC | | | restriction | | | | |
| | | GCATTCCGTTCTAACCTGCACCAC | | | protein Ocr | | | | |
| | | TTGGATAACATTAGCCGTCATCGT | | | | | | | |
| | | CGCTTAGTTAACAGCTTACGCAAT | | | | | | | |
| | | GCACCTCTGGAATACGGTAGCGTT | | | | | | | |
| | | GAGTCTACTGATTTAACTGGCTGT | | | | | | | |
| | | TATCATGAGGCTGGCATGGAGTCA | | | | | | | |
| | | CCCTCTGAAGAGCAAACCATCCGA | | | | | | | |
| | | GTACGCTGCACGACCAAGGCGCAA | | | | | | | |
| | | GCTATAAGTGTCGCACGGTTGGCT | | | | | | | |
| | | TGTAACGACTATGAGCAAGACTGT | | | | | | | |
| | | GTACTGGTCTATAAGTCACAGACT | | | | | | | |
| | | CACAGCGCTGCACTAATCTTTGCT | | | | | | | |
| | | GGTGGCTTGGACGGTTGGAAGACT | | | | | | | |
| | | GAGCGTATCGGGTCGTTCCAGATG | | | | | | | |
| | | GTCGCACCTGAAGTTGAACTGACA | | | | | | | |
| | | GGCAACTACACGGTCGATGAGTTC | | | | | | | |
| | | GGACGTGGCTGGAAGGTAATCTAA | | | | | | | |
| | | (SEQ ID NO: 19) | | | | | | | |
| 2 | 0.6A | ATGGTCATGGCACTCTTTGTAATC | 201 | − | Hypothetical | 0.28 | 0.26 | 0.24 | 0.18 |
| | | ACGATGTACGCTCTGATTGGGGCG | | | | | | | |
| | | TACTTCCTAAGAGACTTCCGCAAG | | | | | | | |
| | | GGTCTCAAGGTACACAAGGCTAAC | | | | | | | |
| | | TTCTCGCACTTGAAGTATGGCTTT | | | | | | | |
| | | CTACCACGATTCACAGTCCGTGAA | | | | | | | |
| | | CGTAACGGGCGCTTTAAGGCTAAC | | | | | | | |
| | | AAGGTTGGTATCTTCTATGTTGCA | | | | | | | |
| | | ACGCACTGA (SEQ ID NO: | | | | | | | |
| | | 20) | | | | | | | |
| 3 | gp0.7 | ATGAACTACACCGACATTCAGGCA | 1104 | + | Protein | — | — | — | — |
| | | CGTCTGGCAATCATCAAGTCACTC | | | kinase and | | | | |
| | | CCGATTAGCGAACTGGACAAGCGC | | | inhibitor of | | | | |
| | | CAGCCTCTTCTGGTTGCACTGGCT | | | host RNA | | | | |
| | | GCTGACATCGTGAATGGTGAGACA | | | polymerase | | | | |
| | | AGCGATGGCAACGACACAGACGGG | | | | | | | |
| | | TCGCAGGGTCTTGAGTATCAGGAC | | | | | | | |
| | | TGGTGGCACACCTTGGGTGCACTC | | | | | | | |
| | | ATGCGTGATGCAGGTTTCCGCATG | | | | | | | |
| | | CTGGGTAATGGTCACTTCAGTGCG | | | | | | | |
| | | GCCTACTCGCACGAAATGTTACCG | | | | | | | |
| | | GGTCGAGTCATCAAGGTTGGCTTT | | | | | | | |
| | | AAGAAAGAGGATTCAGGCGCTGCA | | | | | | | |
| | | TACACTGCATTCTGTAGGATGCAC | | | | | | | |
| | | CAAGGCCGTGCTGGTATCCCTAAC | | | | | | | |
| | | GTCTATCACGTAGCACGTCATGCA | | | | | | | |
| | | GGTTGCTATACGGTCGTACTGGAC | | | | | | | |
| | | CGCTTAAAAAGCTGTGACCGCTAC | | | | | | | |
| | | GCTAACGATGTTCACGCAAAGTAT | | | | | | | |
| | | GCAAGCGCTGCGCAGGAGTTCATT | | | | | | | |
| | | GAGTGCTCTGGAGGTGATGCCGAT | | | | | | | |
| | | TGGTACGCACAGTATGTTAGCAAG | | | | | | | |
| | | GAGTTCATTGAGACGTGCAAGATG | | | | | | | |
| | | ATTCGTGAGTTCTTCCACGGTATT | | | | | | | |
| | | GCATCTTTCGACATGCACAGTGGG | | | | | | | |
| | | AACATTATGTTCGATGACAACGAT | | | | | | | |
| | | GTCCCGTACATCACTGACCCGGTT | | | | | | | |
| | | AGTTTCTCACATGACCGTGAACGT | | | | | | | |
| | | GAGGATGGTTTCCCTCTGGACCCT | | | | | | | |
| | | GATGCGTTGATTGCCGAGGTCGAG | | | | | | | |
| | | GCGATAGCTAACGAGCGCATCATT | | | | | | | |
| | | GAACGGTGCCGCAACCGCAAGGCC | | | | | | | |
| | | AAATGTGACCCTAACGGAACCTTT | | | | | | | |
| | | CAGGTTAACCGCAAGGCCGCAATG | | | | | | | |
| | | AAACGCCGTAAGCGCAACCGCAAG | | | | | | | |
| | | TTACGTGCTAAACATGCAGCGGAA | | | | | | | |
| | | GGAATGCACCAGTTTGTCAAAGAG | | | | | | | |
| | | CGCCTTAAGCGTAAAGCTATTGAC | | | | | | | |
| | | CGAAATGAACGCCGTGCTGAGATG | | | | | | | |
| | | CTCATGGGGTCTGCATGGCATTAC | | | | | | | |
| | | GCATGGTTGCATAATGGTAACTCA | | | | | | | |
| | | ACGTTCCGCAAGGTTGAACAAGTG | | | | | | | |
| | | AACGGCCTCAAGTGGCAACTAGGG | | | | | | | |
| | | GACCGCCTAGCAATCCAAGCGGGT | | | | | | | |
| | | CTACCTCTGAACATTGACAAGTTA | | | | | | | |
| | | CTTGACGCACAACTGCAAGGCTGA | | | | | | | |
| | | (SEQ ID NO: 21) | | | | | | | |
| 4 | gp1 | ATGACTAACGTAATCAACGCACCA | 2652 | − | RNA | 0.98 | 0.97 | 0.63 | 0.67 |
| | | AAGAACGACTTCTCTGACATCGCT | | | polymerase | | | | |
| | | AACGCAATCCAGCCGTACAACATT | | | | | | | |
| | | CTGGCTGACCATTACGGCGCACAG | | | | | | | |
| | | CTTGCAGCTACGCAGTTACAGCTT | | | | | | | |
| | | GAGCACGAAGCACACACCGAGGGC | | | | | | | |
| | | GAGAAGCGTTTTCTTAAGGCAATG | | | | | | | |
| | | GAACGTCAAATCAAAGCGGGTGAG | | | | | | | |
| | | TTCGGTGATAACGCAGTGGCTAAA | | | | | | | |
| | | CCGCTCCTGTCATCTCTCGCACCG | | | | | | | |
| | | AAGTTCATCGAGGCGTGGAACACT | | | | | | | |
| | | TGGTTCACCGAGGTGGAAGCGAAG | | | | | | | |
| | | CGTGGCAAGCGTCCAGTAGCCTAC | | | | | | | |
| | | AATCTGGTACAGAAGGTTGCACCT | | | | | | | |
| | | GAAGCGGCCGCTTTCATCACACTG | | | | | | | |
| | | AAGGTCACACTCGCTTGCTTGACG | | | | | | | |
| | | AAAGAAGAGTTCACCAACCTGCAA | | | | | | | |
| | | TCGGTCGCCACTAAGATTGGTCGC | | | | | | | |
| | | AGTATTGAGGATGAACTGCGTTTT | | | | | | | |
| | | GGTCGCATCCGTGACGAAGAAGCG | | | | | | | |
| | | AAGCACTTTAAGAACCACGTACAG | | | | | | | |
| | | GAGGCACTGAACAAGCGCGTTGGT | | | | | | | |
| | | ATCGTCTACAAGAAAGCATTCATG | | | | | | | |
| | | CAAGCCGTAGAAGGCAAGATGTTA | | | | | | | |
| | | GACGCTGGTCAACTGCAAACCAAG | | | | | | | |
| | | TGGACTACATGGACACCTGAAGAA | | | | | | | |
| | | TCAATTCACGTTGGTGTGCGCATG | | | | | | | |
| | | TTGGAACTGCTCATCGGGTCTACT | | | | | | | |
| | | GGTCTCGTTGAGTTACACCGACCG | | | | | | | |
| | | TTTGCTGGTAACGTTGAGAAGGAT | | | | | | | |
| | | GGCGAGTACATCCAGTTAACAGAA | | | | | | | |
| | | CAATATGTAGACCTGCTATCCAAA | | | | | | | |
| | | CGTGCTGGCGCACTGGCTGCAATC | | | | | | | |
| | | GCTCCGATGTATCAGCCTTGCGTG | | | | | | | |
| | | GTCCCTCCGAAGCCTTGGACTTCT | | | | | | | |
| | | CCGGTTGGTGGTGGCTATTGGGCT | | | | | | | |
| | | GCTGGTCGCAAGCCTCTCTCTCTG | | | | | | | |
| | | GTTCGCACTGGTAGCAAGAAGGGA | | | | | | | |
| | | CTTGAGCGTTACAACGATGTGTAT | | | | | | | |
| | | ATGCCTGAAGTCTACAAGGCGGTG | | | | | | | |
| | | AACATCGCACAGAATACTCCTTGG | | | | | | | |
| | | AAGATTAACAAGAAGGTTCTGGCA | | | | | | | |
| | | GTGGTCAACGAGATTGTGAACTGG | | | | | | | |
| | | AAGCATTGCCCGGTTGAAGATGTT | | | | | | | |
| | | CCTGCTCTGGAACGTGGTGAACTG | | | | | | | |
| | | CCTGTCAAACCTGATGACATTGAC | | | | | | | |
| | | ACCAACGAGGCATCGCTCAAAGCG | | | | | | | |
| | | TGGAAGAAAGCGGCCTCCGCAATC | | | | | | | |
| | | TACCGCAAAGAGAAGGCACGAGTG | | | | | | | |
| | | TCTCGTCGTATGAGCATGGAGTTC | | | | | | | |
| | | ATGTTAGGTCAGGCCAATAAGTTC | | | | | | | |
| | | GCACAGTTTAAAGCTATCTGGTTC | | | | | | | |
| | | CCGATGAACATGGACTGGCGCGGC | | | | | | | |
| | | CGAGTGTATGCGGTCCCAATGTTT | | | | | | | |
| | | AACCCGCAAGGCAACGACATGACC | | | | | | | |
| | | AAAGGCTTGCTGACGCTCGCTAAA | | | | | | | |
| | | GGTAAACCAATCGGTGTCGATGGG | | | | | | | |
| | | TACTACTGGCTGAAGATTCACGGT | | | | | | | |
| | | GCTAACACTGCTGGTGTGGACAAG | | | | | | | |
| | | GTGGACTTCGCTGAACGCATCAAG | | | | | | | |
| | | TTCATTGATGACAACCACGAGAAC | | | | | | | |
| | | ATCATGAGCGTTGCGGCTGACCCG | | | | | | | |
| | | ATTGCTAACACTTGGTGGGCTGAA | | | | | | | |
| | | CAAGACTCTCCGTTCTGCTTCCTC | | | | | | | |
| | | GCGTTCTGCTTTGAGTATGCGGGA | | | | | | | |
| | | GTCCAGCATCACGGGATGAACTAC | | | | | | | |
| | | AACTGCTCTTTACCGCTGGCGTTC | | | | | | | |
| | | GATGGCTCTTGCTCTGGTATTCAG | | | | | | | |
| | | CACTTCTCCGCAATGCTCCGCGAT | | | | | | | |
| | | GAGGTCGGTGGTCGTGCGGTTAAC | | | | | | | |
| | | CTGCTTCCAAGCAAAGAGGTACAA | | | | | | | |
| | | GACATCTACCGCATCGTTGCTGAA | | | | | | | |
| | | CGTGTGAACGAGATTATGCAACAG | | | | | | | |
| | | GACGTAATCAACGGAACTGATAAC | | | | | | | |
| | | GAAGTGGAAACTGTTATCAACAAG | | | | | | | |
| | | GACACTGGAGAAATCACTGAGAAG | | | | | | | |
| | | CTCAAGCTGGGTACGAAGGAACTG | | | | | | | |
| | | GCGGGTCAATGGTTGGCCTACGGT | | | | | | | |
| | | GTGACTCGCAAGGTCACTAAGCGT | | | | | | | |
| | | TCGGTCATGACGTTGGCTTACGGC | | | | | | | |
| | | TCCAAAGAGTATGGCTTCCGTGAC | | | | | | | |
| | | CAAGTTCTTGAGGACACAATTCAG | | | | | | | |
| | | CCAGCTATCGACGATGGCAAGGGC | | | | | | | |
| | | TTGATGTTCACTCAACCGAACCAA | | | | | | | |
| | | GCGGCTGGCTACATGGCTAAACTG | | | | | | | |
| | | ATTTGGAATGCTGTAACGGTCACA | | | | | | | |
| | | GTTGTCGCAGCGGTTGAAGCGATG | | | | | | | |
| | | AACTGGTTGAAGTCTGCTGCTAAA | | | | | | | |
| | | CTTCTGGCTGCTGAAGTCAAGGAC | | | | | | | |
| | | AAGAAGACCAAAGAGGTTCTCCGC | | | | | | | |
| | | AAGCGTTGCGCAGTACATTGGGTA | | | | | | | |
| | | ACTCCTGATGGCTTCCCTGTGTGG | | | | | | | |
| | | CAGGAATACAAGAAGCCAGTACAG | | | | | | | |
| | | ACCCGCTTGAATCTGATGTTCTTA | | | | | | | |
| | | GGCCAGATTCGTCTACAGCCTACC | | | | | | | |
| | | GTGAACACCAACAAGGATAGCGGG | | | | | | | |
| | | ATTGATGCTCGTAAACAGGAGTCA | | | | | | | |
| | | GGCATCGCACCTAACTTTGTCCAC | | | | | | | |
| | | TCAATGGATGGAAGCCACCTGCGC | | | | | | | |
| | | ATGACCGTGGTTCGCTCCAATGAG | | | | | | | |
| | | GTCTACGGTGTGGAATCGTTCGCA | | | | | | | |
| | | CTGATTCACGACTCCTTTGGTACT | | | | | | | |
| | | ATCCCTGCTGATGCTGGCAACCTG | | | | | | | |
| | | TTTAAAGCAGTGCGTGAAACGATG | | | | | | | |
| | | GTCAACACTTACGAAGAGAACGAT | | | | | | | |
| | | GTACTTGCAGACTTCTATGAGCAA | | | | | | | |
| | | TTTGCTGACCAGTTGCACGAATCA | | | | | | | |
| | | CAGCTTGACAAGATGCCTGAGATG | | | | | | | |
| | | CCAGCTAAAGGTTCTCTGGACCTG | | | | | | | |
| | | CAAGAAATCCTTAAGTCAGACTTT | | | | | | | |
| | | GCGTTTGCCTAA (SEQ ID NO: | | | | | | | |
| | | 22) | | | | | | | |
| 5 | gp1.05 | ATGTTTACTTTCATCACTTCAGTA | 558 | − | Hypothetical | 0.18 | 0.18 | 0.27 | 0.22 |
| | | GGCGCTCTCGTATCAACAATTCTT | | | | | | | |
| | | GCAGCAACATACTATAATCGTTCA | | | | | | | |
| | | AAGGTATTTCTCACTAAGCTAATC | | | | | | | |
| | | AAAGAAGAGAAGAACTCTCATGCA | | | | | | | |
| | | CTCAAAGAGAGACTTGAAGATGGA | | | | | | | |
| | | TGGCGAGAGAAGCAGGAACTAGCT | | | | | | | |
| | | GGTCGAATCCGTACCTTAGAGTTC | | | | | | | |
| | | GAACAACAATACGACATGTCGAAC | | | | | | | |
| | | CAACGAGAACTCATTGAGCGTCTA | | | | | | | |
| | | CGCGCTGAGGCAGTGGAGTACCGC | | | | | | | |
| | | AAGGAACTTAAAGAGGCAAAGAGT | | | | | | | |
| | | ACCATCCGTGAGTACACTCACAGT | | | | | | | |
| | | GACATCACGTCTATCAAGTTTACC | | | | | | | |
| | | GCTCACATCCCGGATGCTGGGTGG | | | | | | | |
| | | AAGCGCACTGAGTTCAAGCTGGGT | | | | | | | |
| | | ATAGGGTCTTGTGGCAAAGAGGTT | | | | | | | |
| | | ACATGTTTGCACTGGAAAGAACAG | | | | | | | |
| | | TCGGACCGCTATCTGCTGACACAG | | | | | | | |
| | | ACGTGCACTGACGGTGAGCGCAAA | | | | | | | |
| | | GAGTTCACCTATTATAAGAAAGAT | | | | | | | |
| | | GTAGCTGGTCGCATTGAGATTGCA | | | | | | | |
| | | CGTAAGGTTGCAGACAATACGACT | | | | | | | |
| | | CACTAA (SEQ ID NO: 23) | | | | | | | |
| 6 | gp1.1 | ATGCGTACCAACTTTGAACCAACC | 150 | − | Hypothetical | 0.29 | 0.29 | 0.40 | 0.30 |
| | | ACTAAGCGCAACAACGTAATCAAC | | | | | | | |
| | | GAACACGGTGCTGAATGGCAGGAA | | | | | | | |
| | | CGTAAAGACCGCATGAAGAAACGC | | | | | | | |
| | | CACAAGACACAACGTGGCAACGCT | | | | | | | |
| | | GAGAAACGTAACTGGAAGGAGGCA | | | | | | | |
| | | CTCTAA (SEQ ID NO: 24) | | | | | | | |
| 7 | gp1.2 | ATGGGTCGCTTATATAGTGGTAAC | 273 | − | Inhibitor of | 0.49 | 0.46 | 0.76 | 0.6 |
| | | TTAATGGCGTTAAAGGATGTCATC | | | host dGTP | | | | |
| | | GAGAGACTTAAAGAAGACCACGAT | | | triphospho | | | | |
| | | GTCAGTGTTGTAACTGAGACTTTC | | | hydrolase | | | | |
| | | CGCTATGAGAACTACGCACAGCGC | | | | | | | |
| | | CGATGGGTCTCCGGTGAGACTCTC | | | | | | | |
| | | CGCATTGTTCTACGCAATGGCCTC | | | | | | | |
| | | GTACTGACATCCAAGTGTTTCGAA | | | | | | | |
| | | CAGTCAGACGAAGATGTCCGGTGC | | | | | | | |
| | | AATGCACAGACTGAGTGGCTCCGC | | | | | | | |
| | | AAGGTACACGCAGACCTGAAGCAC | | | | | | | |
| | | TGGAAGTAA (SEQ ID NO: | | | | | | | |
| | | 25) | | | | | | | |
| 8 | gp1.3 | ATGGAAATCACATTCAAGACTAAC | 1032 | − | DNA | 0.096 | 0.087 | 0.22 | 0.12 |
| | | CCTTTTAAAGCTGTATCCTACGTT | | | ligase | | | | |
| | | GAGTCTGCCATTAAGAAAGCACTC | | | | | | | |
| | | GACAAAGCTGGCTACCTGATTGCT | | | | | | | |
| | | GACACTAAGCATGATGGTGTACGT | | | | | | | |
| | | GGTAACATCGTGGTTGACAATACG | | | | | | | |
| | | GCTGACTGCCTGTGGCTATCCCGT | | | | | | | |
| | | GTCTCCAAGACCATCCCCGCGCTT | | | | | | | |
| | | GAGCACCTCAATGGGTTCGACCAA | | | | | | | |
| | | CGCTGGGCTAATGTTCTCAAAGAT | | | | | | | |
| | | GACCGCTGGATTTTCCCTGATGGC | | | | | | | |
| | | TTCATGCTGGATGGTGAACTGATG | | | | | | | |
| | | GTCAAAGGCGTAGACTTCAACACA | | | | | | | |
| | | GGGTCTGGCCTACTGCGTACCAAG | | | | | | | |
| | | TGGCTCAAGAAGACAAACTGTGGA | | | | | | | |
| | | TTTGATATTGGTGGAGTAGAGCCT | | | | | | | |
| | | CTCAAGGGGAGTAAAGTAGACTTC | | | | | | | |
| | | TGCCTAGACCCTAAGAAACTTAAG | | | | | | | |
| | | GTTGTCCTGTATGCCGCTATGCCG | | | | | | | |
| | | TATCACGATATGGTAGAAGGCAAA | | | | | | | |
| | | GAGTACGGCGTTATGTCCCTCCTG | | | | | | | |
| | | ATGCAAGAGCATGTGCGTTCCCTC | | | | | | | |
| | | ATTCCGGTTCTTAGTGAACACTTC | | | | | | | |
| | | CCGGAAATCACATGGGAACTCTCT | | | | | | | |
| | | GAGGCCAAAGAAGTATACGACATG | | | | | | | |
| | | GTAGAACTTCAGGAACTCTACGAG | | | | | | | |
| | | AAAGCCCGTGCCGATGGTCATGAA | | | | | | | |
| | | GGTCTGGTCGTCAAAGACCCGCTG | | | | | | | |
| | | GAAGTCTACAAGCGTGGCAAGAAG | | | | | | | |
| | | TCTGGCTGGTGGAAGATGAAGCCT | | | | | | | |
| | | GAGAATGAAGCTGATGGTATTATC | | | | | | | |
| | | GTTGGTATCAATTGGGGGACTAAA | | | | | | | |
| | | GGTCTCGCTAATGAAGGCAAAGTT | | | | | | | |
| | | ATTGGCTTCGAGGTTCTCTTGGAG | | | | | | | |
| | | TCCGGTCGTGTAGTCTCTGCCAAC | | | | | | | |
| | | AACATTAGTCAAGCATTGATGGAT | | | | | | | |
| | | GAGTTTACCGCTGCCGTCAAACAT | | | | | | | |
| | | GCTGAGTCTGTGGGTATGGGTATG | | | | | | | |
| | | CCGTACACTGGCTTCGCCTGTCAG | | | | | | | |
| | | GTAGCCTACATGGAGGAAACTCCT | | | | | | | |
| | | GATGGTTCTCTTCGTCACCCATCG | | | | | | | |
| | | TTCGTTATGTTCCGTGGTGTCGAG | | | | | | | |
| | | TCAGACCCAACGGTCAAGATGTAA | | | | | | | |
| | | (SEQ ID NO: 26) | | | | | | | |
| 9 | gp1.5 | ATGCTATTTCTTATTGCTGCTGTT | 81 | − | Hypothetical | 0.15 | 0.15 | 0.29 | 0.20 |
| | | ATCTTTGCCACGTTTGCCTTCGTT | | | | | | | |
| | | ATCATTGACGACAACTGCTGGCCT | | | | | | | |
| | | GATTGCTAA (SEQ ID NO: | | | | | | | |
| | | 27) | | | | | | | |
| 10 | gp1.6- | ATGTTACGACTACACTTCAACAAA | 575 | − | Hypothetical; | 0.34 | 0.33 | 0.51 | 0.42 |
| | gp1.7 | TCAAATGGCATCTTCTCTGCCCGT | | | hypothetical | | | | |
| | | GGCGCTGACCGTTCAACCGTTCTG | | | | | | | |
| | | GCCTCTGAGAAACACGCAGTCATC | | | | | | | |
| | | CCTAAAGTGCTTGGCTCGACCATT | | | | | | | |
| | | GAGTTAGCACCTCGTGTTCACGCT | | | | | | | |
| | | GTAATCACCACTGGCTTATATAAA | | | | | | | |
| | | GAAGCGACCACTGGCTCCCGGCCT | | | | | | | |
| | | TTCATTCCGGTTCTCGTCACCCGT | | | | | | | |
| | | TTCCCTAAAGTCCGTCTGGTAATT | | | | | | | |
| | | GCGCGTATCAAGGAGGTGTTCTAA | | | | | | | |
| | | TGGTCTGCATGGCATGTTTAGAGA | | | | | | | |
| | | ATGATGACCGCTACCCACATACAT | | | | | | | |
| | | GTGAACCTGTAGCTAAACCACAAG | | | | | | | |
| | | ATATAGAGGGTGTTCGAACTCCGA | | | | | | | |
| | | GCCACTATATGTTGTTTGATGACA | | | | | | | |
| | | TTGAGGCTATCGAAGTGATTGCCC | | | | | | | |
| | | GGTCGATGACCCGTGAACAGTTCA | | | | | | | |
| | | AAGGGTACTGCTTAGGGAACATCC | | | | | | | |
| | | TTAAGTATCGCCTACGTGCTGGTA | | | | | | | |
| | | AGAAATCCGAACTGGCATTCTTAG | | | | | | | |
| | | AGAAGGACATGGCGAAAGCTTGGT | | | | | | | |
| | | TCTACGGTGAACTGTACGAGAAAC | | | | | | | |
| | | ATAAGGGTAAGTGCTATGCTTAA | | | | | | | |
| | | (SEQ ID NO: 28) | | | | | | | |
| 11 | gp1.8 | ATGGAAAAGCCTAGCGAATGGTGT | 98 | - | Hypothetical | 0.45 | 0.41 | 0.74 | 0.34 |
| | | GAGCGTATGTTTGAAAAGACAGGC | | | | | | | |
| | | AACACCGACTACCTTGAATTATAT | | | | | | | |
| | | AATCACTGGAAGGAGAGAGGACAA | | | | | | | |
| | | TG (SEQ ID NO: 29) | | | | | | | |
| 12 | gp2 | ATGAGCACTCTGAATAAAGGGTCA | 192 | − | Inhibitor of | 0.048 | 0.043 | 0.10 | 0.052 |
| | | CTCGTAAACGGTGACAAGAAGTTC | | | host RNA | | | | |
| | | TTCGCAACGGTCGAAGGCCAAGGC | | | polymerase | | | | |
| | | CAATCGTTCGAAGTCCCTGTGTTC | | | | | | | |
| | | GCTGCGTCTCTTGAAGAGGCAAAC | | | | | | | |
| | | GAGTTAGCTGAATGGCAGTATGTT | | | | | | | |
| | | CCGGCTGGCTTCGCAGTGACACGC | | | | | | | |
| | | ATCCGTCCAGACCGAAAGGTTTAA | | | | | | | |
| | | (SEQ ID NO: 30) | | | | | | | |
| 13 | gp2.5 | ATGGCTTTCAACAAACGTAAAGTA | 699 | − | Single- | 0.14 | 0.14 | 0.23 | 0.18 |
| | | TTCACTTCCGCTCGTGGCGTTGCT | | | stranded | | | | |
| | | GAACCGTATTGCTATCTGGCTAAA | | | DNA | | | | |
| | | CCGGACTTCGGTAGCGGTGACTTC | | | binding | | | | |
| | | AAGAACGAACGTGGTGTCTACAAG | | | protein | | | | |
| | | GTGTCACTTACTGTATCCAACGAC | | | | | | | |
| | | GACCCCCGCTGTCAGAAAATGATT | | | | | | | |
| | | TACGAGATTGTCGAAGCCCATGAA | | | | | | | |
| | | GCGGACTATGCTGCTCGTCTCGAA | | | | | | | |
| | | GAGTACGAAGCGAACCCGCCGAAA | | | | | | | |
| | | GTTGTCAAGGGTAAGAAACCGCTG | | | | | | | |
| | | AAGCCGTATGTTGGCGACATGCCT | | | | | | | |
| | | TTCGTGGATAACGAAGACGGAACC | | | | | | | |
| | | ACTACGTTCAACTTCAAGTGCTAC | | | | | | | |
| | | GGCTCGTTCACTGACAAGAAGACT | | | | | | | |
| | | GGTGAGAACAAACCAATCGACTTG | | | | | | | |
| | | GCTATCGTGGACTCCAAGGGCAAG | | | | | | | |
| | | CGCATTCGTGGTGAACGTCCAGCT | | | | | | | |
| | | ATCTCTGGTGGCTCTGAACTGAAA | | | | | | | |
| | | ATCAAATACACCCTGTTCCCTTAT | | | | | | | |
| | | GGCTGGTCTGCTGTGGCTGGTGCA | | | | | | | |
| | | TCCGTTAAACTACAACTGGATTCT | | | | | | | |
| | | GTGATGCTGATTAAGCTGGTCGAG | | | | | | | |
| | | TTCGGTGGTGGTGATGACGATTGG | | | | | | | |
| | | GGTGATGAAGTCGAAGAAGACGGT | | | | | | | |
| | | TATGTAGCTGATGAAGCACAGACC | | | | | | | |
| | | CGCAAGCCGCAGAACGACTCTGGC | | | | | | | |
| | | TGGGACGAAGAACCAGAAGAGGAC | | | | | | | |
| | | GATGAAGATGAAGACGGTGACTTC | | | | | | | |
| | | TAA (SEQ ID NO: 31) | | | | | | | |
| 14 | gp3 | ATGGCTAAGGGTTATGCAGGTCGA | 447 | − | Endonuclease | 0.0081 | 0.0076 | 0.0081 | 0.0076 |
| | | GGCACTCGTGTGGGTGCCTTTCGC | | | I | | | | |
| | | TCTGGCTTGGAGGATAAGGTAAGC | | | | | | | |
| | | AAGCAACTCGAAGGGAAGAACATT | | | | | | | |
| | | AAGTTCGACTACGAGCGCTGGCGT | | | | | | | |
| | | GTACCATATGTTGTTCCTGCAAGC | | | | | | | |
| | | AACCATACCTATTGCCCGGACTTC | | | | | | | |
| | | CTATTACCTAATGGCATCTTTGTG | | | | | | | |
| | | GAAACAAAGGGTCTGTGGGATAGT | | | | | | | |
| | | GACGACCGCAAGAAACACTTACTG | | | | | | | |
| | | ATTCGTGAACAGTATCCTGAACTT | | | | | | | |
| | | GATGTGCGCATCGTGTTCTCTTCA | | | | | | | |
| | | AGCCGCTCTAAGCTATACAAAGGG | | | | | | | |
| | | TCTCCGACCAGTTATGGTGAGTGG | | | | | | | |
| | | TGCGAGAAGCATGGCATTCTGTTT | | | | | | | |
| | | GCTGACAAATTAATTCCAGTTGAT | | | | | | | |
| | | TGGCTGAAAGAGCCTTCTAAGGAT | | | | | | | |
| | | GTTCCGTTCGATAAATTTAAAGAG | | | | | | | |
| | | GTGAAGAAGAAATGA (SEQ ID | | | | | | | |
| | | NO: 32) | | | | | | | |
| 15 | gp3.5 | ATGAGTAAGGTACAATTCAAACCA | 456 | − | Lysozyme | 0.52 | 0.55 | 0.46 | 0.57 |
| | | CGCGCTGTGACAGAAGCAATCTTT | | | | | | | |
| | | GTCCACTGTAGCGCAACCAAGGCG | | | | | | | |
| | | TCCATGAATGTTGGACTGCGTGAA | | | | | | | |
| | | ATCCGTCAGTGGCATAAAGAACAG | | | | | | | |
| | | GGCTGGCTTGATGTAGGCTACCAC | | | | | | | |
| | | TTCATTATTCGCCGTGATGGTACA | | | | | | | |
| | | ATCGAAGAAGGCCGTCCGGTCGAT | | | | | | | |
| | | GTAGTTGGGTCTCACGTTAAGGAC | | | | | | | |
| | | TGGAACAGTAAGTCAGTCGGTGTG | | | | | | | |
| | | TGCCTCGTTGGTGGTATCGACGAT | | | | | | | |
| | | AAGGGCAGACACGAAGCTAACTTT | | | | | | | |
| | | ACGCCAGCACAGATGCACTCTCTT | | | | | | | |
| | | AAAGAGAAACTGGCAGACCTGAAA | | | | | | | |
| | | GACCTGTACCCGGATGCAGAAATC | | | | | | | |
| | | AAAGCACACCACGATGTGGCTCCT | | | | | | | |
| | | AAAGCGTGTCCAAGTTTTAACTTG | | | | | | | |
| | | AGTCGCTGGCTGAAGACTGGTGAA | | | | | | | |
| | | TTAGTAACAAGTGATTGGGGTTAA | | | | | | | |
| | | (SEQ ID NO: 33) | | | | | | | |
| 16 | hypo- | ATGATGATTATTCATGAGATGGGA | 90 | − | Hypothetical | 0.16 | 0.16 | 0.34 | 0.23 |
| | thetical | ATGTACGATATGATTTACCGCTGT | | | | | | | |
| | protein | GTATTCGTTCTGGCATTGACCTAT | | | | | | | |
| | | ATCTTAACTCGTAAGTAA (SEQ | | | | | | | |
| | | ID NO: 34) | | | | | | | |
| 17 | hypo- | ATGCGTAAGCTATTTGGTGAAGCA | 87 | − | Transmembrane | 0.44 | 0.41 | 0.67 | 0.61 |
| | thetical | CTTCCGGTGACACTTTTGGTCATC | | | | | | | |
| | protein | ATCATTATCGTTTGTGGCGCACTG | | | | | | | |
| | | CGTCCGTTCCTGTAG (SEQ ID | | | | | | | |
| | | NO: 35) | | | | | | | |
| 18 | gp4 | ATGGATATGGAAGAACAACAAGAG | 1707 | + | DNA | — | — | — | — |
| | | AGTATATTCCTCTATCATTTACCT | | | primase/ | | | | |
| | | TGTGAGAACTGTGGGTCGTCAGAT | | | helicase | | | | |
| | | GGTAATGGCATGTATTCTGATGGA | | | | | | | |
| | | CACCAGTATTGTTTTGTGTGTCAG | | | | | | | |
| | | AACCATGTGCGTGGAACCGAGGAA | | | | | | | |
| | | ACTCGTGAGACCGTGGCGAAGACT | | | | | | | |
| | | CGCAGAAGCTCATACAACACTGGA | | | | | | | |
| | | GGTGACAAAATGTCTAACCTGTTA | | | | | | | |
| | | AACTTTGGGGATTCAGACGGTCGC | | | | | | | |
| | | TACACGAACCTCAAAGCTCGTGGC | | | | | | | |
| | | CTTATGGAAGCTATCTGCCGTAAA | | | | | | | |
| | | TATGGCTATTGGGTAGCGAAGGTC | | | | | | | |
| | | AACGGTGAGATGCTGCAAGTTGCA | | | | | | | |
| | | AACTACTATGACGTTGAGGGAAAT | | | | | | | |
| | | CTCGTAGGTCAGAAGGTACGCGAC | | | | | | | |
| | | AAGCACAAAGAGTTCTCCGCAAAG | | | | | | | |
| | | GGGAAACTTAAAGCTGACCTATTG | | | | | | | |
| | | TTTGGTAAGCAGTTATGGAATGGT | | | | | | | |
| | | GGCAAGAAGATTGTGGTGACTGAG | | | | | | | |
| | | GGTGAGATTGATTGTCTGACCGTA | | | | | | | |
| | | GCGCAACTTCAAGAAGGGAAGTAT | | | | | | | |
| | | CCCGTTGTGAGTCTCCCTATGGGT | | | | | | | |
| | | GCTCAAGCCGCAAAGAAAACATGT | | | | | | | |
| | | GCTGCAAACTATGAATACTTCGAC | | | | | | | |
| | | CAGTTCGATGAGATTATCCTGATG | | | | | | | |
| | | TTCGACATGGATGAGCCGGGCCGC | | | | | | | |
| | | AAGGCCATCGAAGAGTGTGCTCCT | | | | | | | |
| | | GTCCTGCCTAGCGGGAAAGTCCGA | | | | | | | |
| | | GTCGCTGTACTGCCTCTTAAGGAT | | | | | | | |
| | | GCCAATGAGTGTTTGCTCAATGGA | | | | | | | |
| | | CAAGCCAAAGCAGTAACCGACCAG | | | | | | | |
| | | ATTTGGAATGCGCAACCTTGGGTG | | | | | | | |
| | | CCTGATGGTGTTGTCTCTGCTGTG | | | | | | | |
| | | TCACTTAAAGACCGTGTGCGTGAA | | | | | | | |
| | | GCGATGGTCAAAGAGGAAACAACA | | | | | | | |
| | | GGTCTACTCTTCACGGGTCAACCG | | | | | | | |
| | | AAGCTCAACGACATGACACTTGGT | | | | | | | |
| | | GCTCGTGGTGGTGAAGTCATCATG | | | | | | | |
| | | GTAACTTCCGGTTCCGGTATGGGT | | | | | | | |
| | | AAGTCAACATTCGTTCGTCAACAG | | | | | | | |
| | | ATGCTCATGTGGGGCAAGGGTGGT | | | | | | | |
| | | GCGAAGGTAGGCTTGGCGATGCTT | | | | | | | |
| | | GAGGAAGCCGTAGAGGAAACTGTT | | | | | | | |
| | | CAGGACTTGATGGGCTTGAACAAC | | | | | | | |
| | | CATGTCCGTCTACGTCAAGACAAA | | | | | | | |
| | | GAACTCAAGATGCAAATCTTAGAG | | | | | | | |
| | | GATGGACGCTTCGATGAGTGGTAC | | | | | | | |
| | | GATGTACTGTTCAACACAGATATG | | | | | | | |
| | | TTCCACCTGTATGATTCCTTTGCG | | | | | | | |
| | | GAGTCACAGGAAGACCGTCTGTTC | | | | | | | |
| | | GCAAAGCTGGCCTACATGGTCGAT | | | | | | | |
| | | GGCTTAGACTGTAACGTTATCCTG | | | | | | | |
| | | CTTGACCATATCTCTATTGTGGTC | | | | | | | |
| | | TCCGGGATGGAAGATAACTCCGAT | | | | | | | |
| | | GAGCGTAAGACGATTGACCGACTG | | | | | | | |
| | | ATGACCAAGCTGAAAGCCTTTGCG | | | | | | | |
| | | AAGACTAAAGGTGTGGTCGTGGTG | | | | | | | |
| | | GTAATCTGTCACCTCAAGAATCCT | | | | | | | |
| | | GAGAAGGGGAAAGCACATGAAGAA | | | | | | | |
| | | GGTCGTCCTGTTAGTATTACTGAC | | | | | | | |
| | | CTGCGTGGTTCTGGCGCATTACGC | | | | | | | |
| | | CAACTTAGCGATACTATCATTGCC | | | | | | | |
| | | TTGGAACGTAACCAGCAAGGTGAC | | | | | | | |
| | | TATCCTAATCTTGTCCAGTTGCGT | | | | | | | |
| | | GTTCTCAAGTGTCGTTTTACTGGC | | | | | | | |
| | | GATACTGGCGTGGCTGGACACATG | | | | | | | |
| | | GCCTACAACAAAGAAACGGGCTGG | | | | | | | |
| | | CTTGAACCGACTGTCTCACCTGAA | | | | | | | |
| | | GAAGATGGAGGTGGAGATTGGGAA | | | | | | | |
| | | CCGGAAGAAGACGGTCAAGACTTC | | | | | | | |
| | | TGA (SEQ ID NO: 36) | | | | | | | |
| 19 | gp4.2 | ATGAAGAAGGTCGTCCTGTTAGTA | 357 | − | Transmembrane | 0.9 | 0.92 | 0.68 | 0.82 |
| | | TTACTGACCTGCGTGGTTCTGGCG | | | | | | | |
| | | CATTACGCCAACTTAGCGATACTA | | | | | | | |
| | | TCATTGCCTTGGAACGTAACCAGC | | | | | | | |
| | | AAGGTGACTATCCTAATCTTGTCC | | | | | | | |
| | | AGTTGCGTGTTCTCAAGTGTCGTT | | | | | | | |
| | | TTACTGGCGATACTGGCGTGGCTG | | | | | | | |
| | | GACACATGGCCTACAACAAAGAAA | | | | | | | |
| | | CGGGCTGGCTTGAACCGACTGTCT | | | | | | | |
| | | CACCTGAAGAAGATGGAGGTGGAG | | | | | | | |
| | | ATTGGGAACCGGAAGAAGACGGTC | | | | | | | |
| | | AAGACTTCTGATAGTAAATCAATA | | | | | | | |
| | | GAACTCACTAATGGGACAGACACT | | | | | | | |
| | | CTGTTCCCACTTATCAATCCACTT | | | | | | | |
| | | AAGGAGAATCACAATGTTTAA | | | | | | | |
| | | (SEQ ID NO: 37) | | | | | | | |
| 20 | gp4.3 | ATGTTTAAATTCATCAACACTCTT | 213 | − | Hypothetical | 0.15 | 0.15 | 0.27 | 0.23 |
| | | GGTAAGCTGGTAGTTAAAATGTAC | | | | | | | |
| | | TTCCGTGAAGCTAAACGTCTGAAC | | | | | | | |
| | | GACAAGGCACGTAAGGATGCAGAA | | | | | | | |
| | | CAGGCTCAACGCTTGGCGAAACAG | | | | | | | |
| | | GCGCGTCTACTATCTGAAGATGCA | | | | | | | |
| | | AGTGCTGGCGTTACGTCTGCTGCT | | | | | | | |
| | | AAGATTGCAGCGAAAGCAACCGAC | | | | | | | |
| | | CTGAACAAGTTCTTCCTGTGA | | | | | | | |
| | | (SEQ ID NO: 38) | | | | | | | |
| 21 | hypo- | ATGGTTACGATAACCCTTGTGCTC | 180 | − | Hypothetical | 0.54 | 0.49 | 0.82 | 0.49 |
| | thetical | ACTCTGATAACTGTGAGCGGACAA | | | | | | | |
| | protein | ACAATAAGTCAAGTCCCTATCACG | | | | | | | |
| | | GCACCGAAGGCTGAGGCATACGAG | | | | | | | |
| | | ATATGCGAACAGCAAGGGCGCTCA | | | | | | | |
| | | TGGTCTAAGGACTTGATGAAGAAC | | | | | | | |
| | | GGCAGGGTTTTCACTGCCAACTAC | | | | | | | |
| | | AACTGTATCTAA (SEQ ID NO: | | | | | | | |
| | | 39) | | | | | | | |
| 22 | gp4.5 | ATGGCTATTAAATTCCCCGGCAAC | 303 | − | Hypothetical | 0.089 | 0.083 | 0.16 | 0.099 |
| | | ACAATCCGTCTGTCCGACACCATC | | | | | | | |
| | | GACCAGTATGCCCGTCGAGTGCAC | | | | | | | |
| | | ATCAACGTCCGCAACGGAAAGGTA | | | | | | | |
| | | ACACTGGTCTACCGTTGGAAGGAC | | | | | | | |
| | | AGTAAGTCCACCAAGGCGCACACA | | | | | | | |
| | | CAGCGTGTGACTCTGGATGACACA | | | | | | | |
| | | CAGGCAGGTCGTCTGCTGGCCTCC | | | | | | | |
| | | GTTGCTGTAGCCGCTACTGTGGCT | | | | | | | |
| | | GTAGGTGAAGACAATGTTCGTCAG | | | | | | | |
| | | GTTCTGCTGGGTAAAGCCGTGGAG | | | | | | | |
| | | TCTGAGGGCAACATGCTTGCTGAC | | | | | | | |
| | | AAGTGTGAAGAGTAA (SEQ ID | | | | | | | |
| | | NO: 40) | | | | | | | |
| 23 | gp4.7 | ATGCGTAATCGTAATGTAATCGAA | 417 | − | Hypothetical | 0.31 | 0.29 | 0.45 | 0.36 |
| | | GCTGAAATCGCTAAACTGGAAGCT | | | | | | | |
| | | GAACTGGCAGATGTTAAAGAGTAT | | | | | | | |
| | | GACTGTGCTCGTGACTCCGCTGTC | | | | | | | |
| | | CACATCCTGAAGAACTTAGGGTGG | | | | | | | |
| | | ACCCGCAAGTATGGCAAGTGGGTC | | | | | | | |
| | | AAACCAGTAGCCAACTGTGGTAGC | | | | | | | |
| | | GTTGGTATGCAGACTTTCGACGCA | | | | | | | |
| | | GACAAGATGACCCACATTAAGGCT | | | | | | | |
| | | GGGGACTTCTGCACCTCCACCAGC | | | | | | | |
| | | AACGCAGACATTTGGTATGTCCGT | | | | | | | |
| | | AAGGTCGAAGGGACAGACATTTGG | | | | | | | |
| | | TGCTCCCGTGTGACTCGCATTGAT | | | | | | | |
| | | GCAATGGGTACAGTCGCTGGGATT | | | | | | | |
| | | CAGTACTACAAGATGAACCACAAG | | | | | | | |
| | | AACCTGCGTGTGGTTGACCCTAAG | | | | | | | |
| | | AACTTCATGGGATACCGCAAATCT | | | | | | | |
| | | ACATTTTAA (SEQ ID NO: | | | | | | | |
| | | 41) | | | | | | | |
| 24 | gp5 | ATGTTACTATCCGACATCGAAACA | 2115 | − | DNA | 0.55 | 0.56 | 0.52 | 0.51 |
| | | AACGGGTTGCTGCACAACGTTAGT | | | polymerase | | | | |
| | | CAATTTCACTGTGCGGTGACTTAC | | | | | | | |
| | | GACTACACAACTGACCAGTACATC | | | | | | | |
| | | CGTTACCGACCTGATGACTTTGGG | | | | | | | |
| | | GCGTACATTGATGCACTCGAAGCC | | | | | | | |
| | | GAGGTTGCTCGTGGTGGTCTCATT | | | | | | | |
| | | GTGTTCCATAACGGTCACAAGTAT | | | | | | | |
| | | GACATCCCGGTAATCGAGTTGCTC | | | | | | | |
| | | GCCAAGAAACTGTTAGGCCGCGAC | | | | | | | |
| | | GTTAAGTTCCCGAAAGAGAACGTA | | | | | | | |
| | | CTGGATACCCTCGTGATGTCCCGC | | | | | | | |
| | | TTGATTTACTCTAACATCAAAGAC | | | | | | | |
| | | ACCGATGCTGGTCTGCTGCGTTCT | | | | | | | |
| | | GGCAAGCTGCCGGGTAAACGTTTT | | | | | | | |
| | | GGTTCCCACGCTCTGGAGGCTTGG | | | | | | | |
| | | GGTTATCGCTTAGGCGAAATGAAG | | | | | | | |
| | | GGTGAGTACAAGGACGACTTCAAG | | | | | | | |
| | | GCTTCACTGGCTGAGTCTGGTGAA | | | | | | | |
| | | GAATATGTTGATGGTATGGAATGG | | | | | | | |
| | | CTCCTGTTCAACGAAGATATGATG | | | | | | | |
| | | GAATATAACGTTCAAGACGTTGTA | | | | | | | |
| | | GTCACCAAGGCTCTCTTCGAGAAA | | | | | | | |
| | | CTTTGTAGCAACACATTCTATTTC | | | | | | | |
| | | CCTAACGAGACACCCGCTGGAAGC | | | | | | | |
| | | ACAGAAGCAGACCGTTTCTGGAAT | | | | | | | |
| | | GGTAGCCTTGAGGCTGTTAAACTG | | | | | | | |
| | | GAACATGATGCTGCATGGTTGTTA | | | | | | | |
| | | GCCAAGATGGAACGTAACGGTTTC | | | | | | | |
| | | CCGATTGACATTAAGTCTCTTGAG | | | | | | | |
| | | AACCTGTACGCAGAACTCGCTGGT | | | | | | | |
| | | AGACGTGGTGAGCTACTCGTAGAG | | | | | | | |
| | | TTAACCAATACGTTTGGCTCATGG | | | | | | | |
| | | TACTCCGCTAAGGGCGGTACTGAA | | | | | | | |
| | | GCATTCAGACACCCTCGTACCGGA | | | | | | | |
| | | AAGCCGCTCACCAAGTATCCTCGT | | | | | | | |
| | | GTTAAGTACCCGAAACAGGGTGGC | | | | | | | |
| | | ATCTTTAAGAAGCCTAAGAACAAG | | | | | | | |
| | | AAGCAACGTGAGGGATTAGAGCCG | | | | | | | |
| | | TGCGAGTTGGATACCCGCGATTAC | | | | | | | |
| | | GTTGAGGGTGCTCCGTATACTCCG | | | | | | | |
| | | GTTGAGCACGTTGTGTTTAACCCT | | | | | | | |
| | | GCGAGCCGAGACCACATCACCCGT | | | | | | | |
| | | GTTCTCACCAAGGCTGGTTGGGTT | | | | | | | |
| | | CCCGTTGAGTTCACCCCAAGTGGT | | | | | | | |
| | | GCACCAAAGGTAGACGACGAGGTC | | | | | | | |
| | | TTAGAGCACGTTAAGGTAGACGAC | | | | | | | |
| | | CCTGATGCACAGCGTTGCATTGAG | | | | | | | |
| | | TTGATTAAAGAATACCTTATGATT | | | | | | | |
| | | CAGAAACGTATCGGTCAGGTTGCT | | | | | | | |
| | | GAGGGTGATAACGGCTGGCTCCGA | | | | | | | |
| | | ATGATTGGAGAAGATGGTCGTATT | | | | | | | |
| | | CACGGAAGCGTTAACCCGAACGGT | | | | | | | |
| | | GCGGTAACTGGTCGTGCAACTCAT | | | | | | | |
| | | AGTTTCCCTAACGTGGCACAGGTG | | | | | | | |
| | | CCTTCAATCCGTGCTGCATATGGC | | | | | | | |
| | | GAACCTTGCCGTGCTGCTTTCGGT | | | | | | | |
| | | GCCGAGCACAACATGAAAGACGGT | | | | | | | |
| | | AAGCCAGACCCTTGGATTCAAGTT | | | | | | | |
| | | GGTGTGGATGCGTCCGGCCTTGAG | | | | | | | |
| | | CTACGATGCTTAGGTCACTTCATG | | | | | | | |
| | | TATCGCTATGATGAGGGAGAGTAC | | | | | | | |
| | | GTTGAGACTATCCTCACTGGTGAC | | | | | | | |
| | | ATCCATACGAAGAACCAATTGGCT | | | | | | | |
| | | GCTGGTCTGCCAACTCGTGACAAC | | | | | | | |
| | | GCTAAGACGTTTATCTATGGGTTC | | | | | | | |
| | | CTCTATGGTGCTGGCGCTGCGAAG | | | | | | | |
| | | ATAGGACAGATTGTTGGTGGTACT | | | | | | | |
| | | GCCGAAGACGGTAAGCGACTCATC | | | | | | | |
| | | AAGAACTTCCTTGAGCAAACTCCG | | | | | | | |
| | | GCTATCGCTGCATTACGTGAGGCC | | | | | | | |
| | | ATTACAGGTACTCTCGTCAAGGAC | | | | | | | |
| | | TCTAAGTGGGTTGGCGGTGAACAG | | | | | | | |
| | | AAAGTAACTTGGAAACGCCGTTGG | | | | | | | |
| | | GTTCGTGGATTGGATGGTCGTAAA | | | | | | | |
| | | GTACACGTCCGTAGTCCTCACGCT | | | | | | | |
| | | GCGTTGAACACCTTGTTGCAGTCT | | | | | | | |
| | | GCTGGTGCTCTGATTTGTAAACTG | | | | | | | |
| | | TGGATTGTCGAGACTGAACGACTG | | | | | | | |
| | | TTACTGGAAGCTGGCCTGAAGCAT | | | | | | | |
| | | GGCTGGGATGGTGACTTTGCGTAC | | | | | | | |
| | | ATGGCATGGGTTCACGACGAAATA | | | | | | | |
| | | CAGGTCGCTGCAAGGACTCAAGAG | | | | | | | |
| | | ATTGCCGATAAGATTGTCGAGTTG | | | | | | | |
| | | GCACAGCAAGCAATGCGTAATGTG | | | | | | | |
| | | GGAGACCACTTTAACTTTAGATGT | | | | | | | |
| | | CAGTTAGATACCGAAGGTAAGACT | | | | | | | |
| | | GGCGCAAACTGGAAGGATTGTCAC | | | | | | | |
| | | TAA (SEQ ID NO: 42) | | | | | | | |
| 25 | hypo- | ATGGCTTGGTTATATAGAATACGT | 228 | − | Hypothetical | 0.44 | 0.43 | 0.71 | 0.55 |
| | thetical | GAAGTTAGACTTACTACAGGAGAG | | | | | | | |
| | protein | ACACTCTACTACCCTCAATTCCGC | | | | | | | |
| | | CACAAGTGGTTACCGTTTTGGTGC | | | | | | | |
| | | CACTTCCGGGATTATTCAAATGAG | | | | | | | |
| | | AACAAAGTGTCATTCACTTATTAC | | | | | | | |
| | | TCTGAAGCTGTCAACTTTGTTAAC | | | | | | | |
| | | GAGTCGAAAGCAAATGACGAACCT | | | | | | | |
| | | GAAGTTAAACAGGTTGTCCGTAAT | | | | | | | |
| | | CACTATCTTTAA (SEQ ID NO: | | | | | | | |
| | | 43) | | | | | | | |
| 26 | gp5.5 | ATGGCTATTACTAAACGTTTTAAA | 285 | − | Binds to | 0.64 | 0.62 | 0.82 | 0.77 |
| | | GTATCATTTGAAGTTACCGCTGTA | | | host | | | | |
| | | ATCAACTCTGAGATGGAGAAAGGC | | | histone- | | | | |
| | | TTCACCGAGGAACTGGTTCATTTG | | | like protein | | | | |
| | | GCTAAGAAGGCTGCGGCAGGTGAG | | | H-NS and | | | | |
| | | AAGATTAGTCCTCGTGACCGTGAA | | | transfer | | | | |
| | | CTTCTGGTACAAGCGCTGACTCAT | | | RNAs | | | | |
| | | GGTGTCGGTGGTGCTATCTCCTTT | | | | | | | |
| | | GCCGCCAAACAGGGTATGCGAGAG | | | | | | | |
| | | TTCATCCGTACCGAGTTACCGGAA | | | | | | | |
| | | GATGGTCTGAAGGTTTCTCCAGCA | | | | | | | |
| | | ACTATCCGTGAGGTGAAGTAA | | | | | | | |
| | | (SEQ ID NO: 44) | | | | | | | |
| 27 | gp5.7 | ATGGGTGAATATCTACGTGTACTG | 210 | − | Inhibitor of | 0.068 | 0.06 | 0.077 | 0.061 |
| | | GCGGCTATCAAAAGCTGCCCTAAG | | | host RNA | | | | |
| | | ACCTTTCAGTCGAACTATGTGCGC | | | polymerase | | | | |
| | | AACAATGCGTCACTGGTGGCTGAG | | | | | | | |
| | | GCTGCGAGTCGAGGTCATATCTCT | | | | | | | |
| | | TGCCTATCAGTCGATGGGCGTAAT | | | | | | | |
| | | GCTGGCGCTTGGGAAGTAACAGGC | | | | | | | |
| | | GCTGGTGTCCGATTCCTGAAGAAG | | | | | | | |
| | | ATGGGAGGTTGCGTATGA (SEQ | | | | | | | |
| | | ID NO: 45) | | | | | | | |
| 28 | gp5.9 | ATGAGTCTGCACACAGACAACGTT | 186 | − | Inhibitor of | 0.25 | 0.23 | 0.42 | 0.20 |
| | | ACAGTGACCCGTGAGGCGTGGAAC | | | host | | | | |
| | | GACATGCAGTCGTACATCCAGTCA | | | RecBCD | | | | |
| | | CTGGAGAAGAAAGCTGAGAAGCTG | | | | | | | |
| | | GACGCTCTGGAGGCTGGAGGTGTG | | | | | | | |
| | | GACAATAGGTCCGGCTACTGGTTC | | | | | | | |
| | | TCTCTGGAAGAGGCTGGTCTCACA | | | | | | | |
| | | GGGGAGGACGACTTATGA (SEQ | | | | | | | |
| | | ID NO: 46) | | | | | | | |
| 29 | gp6 | ATGAGTGCAATTACACTGAAGGAG | 915 | − | dsDNA- | 0.082 | 0.081 | 0.14 | 0.12 |
| | | TTCTACGAGTTACGCCAAGGGAAA | | | specific | | | | |
| | | TCCGATAAGGGTGTTCTCGTAATG | | | exonuclease | | | | |
| | | GATGGTGACTGGTTGGTATTCCAA | | | | | | | |
| | | GCGATGGCAGCAAGTGAAGTCGAG | | | | | | | |
| | | ACAGATTGGGGTGACGATGTGTGG | | | | | | | |
| | | ACCTTAGAGTGTGACCACGGGAAA | | | | | | | |
| | | GCGTGGAACATTCTGGTTGACTCT | | | | | | | |
| | | ATCAAGTCCTATGCGAGCCGTAAG | | | | | | | |
| | | AAGGCTTGGCGTGATGCACCTATC | | | | | | | |
| | | GTATTGGCCTTCACTGACGCTGTG | | | | | | | |
| | | AACTGGCGTAAGGAACTGGTGGAC | | | | | | | |
| | | CCAACCTATAAGGAGAACCGTAAG | | | | | | | |
| | | GCATCCCGTAAGCCTGTAGGCTAC | | | | | | | |
| | | CGTGCGTTCATCGAGCGTGTCCAA | | | | | | | |
| | | GCCTGTGAGGACTGGACGTCTATC | | | | | | | |
| | | CTTGAGGAACGACTTGAGGGTGAT | | | | | | | |
| | | GACGTAATGGGAATCATCGGGTCT | | | | | | | |
| | | GGTGCTAAACATTTTGGTTTCAAG | | | | | | | |
| | | AAGGCTGTTCTTGTCTCCTGTGAT | | | | | | | |
| | | AAGGATTTTAAGACCATCCCTGAT | | | | | | | |
| | | TGTGACTTCTTGTGGTGCACGACT | | | | | | | |
| | | GGTAACATCTTGACTCAAGACCAA | | | | | | | |
| | | GCGACTGCTGACTACTGGCATATC | | | | | | | |
| | | TATCAGACCATTAAAGGTGACTTG | | | | | | | |
| | | ACAGATGGTTATGGTGGCATTCCG | | | | | | | |
| | | GGCTGGGGCGAGACTACCGCTGAG | | | | | | | |
| | | TGGCTGGAGAACCCATATGCCTTT | | | | | | | |
| | | GAACAGGTTGAGAAAACTTTCAAG | | | | | | | |
| | | TCTGGTGCCCGTAAGGGTCAGACA | | | | | | | |
| | | GTTCTCGAATGGAAGAAGGTTGAC | | | | | | | |
| | | ATGGGTGAGCGAACCTTATGGGAA | | | | | | | |
| | | GCCATTGTGACACTTGGTGCGAAA | | | | | | | |
| | | GCTGGGATGACCGAAGAGGAAATC | | | | | | | |
| | | ATTAAGCAAGGCCGGATGGCTCGC | | | | | | | |
| | | ATCCTTCGATTCGAAGAGTACAAC | | | | | | | |
| | | TTTATCGACAAGGAGATTTACCTA | | | | | | | |
| | | TGGTCGCCGGAACAGTTGCGTTCG | | | | | | | |
| | | TAA (SEQ ID NO: 47) | | | | | | | |
| 30 | gp6.5 | ATGTTAACCCCTATCAAGAAATAC | 246 | − | DUF2717 | 0.81 | 0.78 | 1.2 | 1.0 |
| | | ATGGAGAACCCACAAGATATTCCT | | | | | | | |
| | | AGCGTTCCACGTAGTGTTATGGAG | | | | | | | |
| | | TATTTACAGGTTCAATATAATGCT | | | | | | | |
| | | GGATATGCCTTGCAGTCTGGTTTA | | | | | | | |
| | | GTTAACCGATTGAAACAGGCTGGT | | | | | | | |
| | | TGGTCTGAAAGCTACATTGCTGGG | | | | | | | |
| | | TTCCTCGCGGGTCTCAACTATGCG | | | | | | | |
| | | TCTCAAACCTTAGACGACATGGAG | | | | | | | |
| | | GCAATCCGTAAGGAACAGGCTGAG | | | | | | | |
| | | TCCTAA (SEQ ID NO: 48) | | | | | | | |
| 31 | gp6.7 | ATGTGCTTTTCACCAAAGATTAAG | 273 | − | DUF5476 | 1.0 | 1.0 | 1.3 | 1.0 |
| | | ACACCTAAAGTAAAGACCGATACT | | | head | | | | |
| | | GTCCGAGCTATTGACCCAGCGCCA | | | protein | | | | |
| | | CTGACTGAGGAACCGAAAGGTATT | | | | | | | |
| | | CTCTTTGGAGGCGATGAGGACACA | | | | | | | |
| | | GATAGCGGAACCTCTTCTGAGGTG | | | | | | | |
| | | CCAACGGGTGGCAAGAAGTCCCTT | | | | | | | |
| | | AAGGTGAAACTGGATGACTCCGTT | | | | | | | |
| | | GAGAAGTCCAAGAAGGCTGATGAT | | | | | | | |
| | | GCAGCCAAGAAGAAGTCTAAATCT | | | | | | | |
| | | GGTATCCGTACCAGTATCTTTAAG | | | | | | | |
| | | AAGAAATAA (SEQ ID NO: | | | | | | | |
| | | 49) | | | | | | | |
| 32 | gp7.3 | ATGGGTTGGGGTAAAAAGATTCGA | 315 | − | Tail | 1.2 | 1.2 | 0.98 | 1.2 |
| | | AAATCAGTTAAGAAGGCTGTCAAG | | | assembly | | | | |
| | | AAAGTAACCAAGCCTATCGAGAAG | | | protein | | | | |
| | | GTAGTCAAGGAAACTGGCAAGGCA | | | | | | | |
| | | GTTGAGAAGGTTGGTAAGGAAGCT | | | | | | | |
| | | GGGAAAGTAGTTGGTGGTGTTGCT | | | | | | | |
| | | GGTACTGTTCAAGCTGCACCTCCT | | | | | | | |
| | | GCTGCACAGATTATTGAGCCACCG | | | | | | | |
| | | AGTAAAGACGATGTAGACTCCGAT | | | | | | | |
| | | GATGATGCACAGACTGAATCCGGT | | | | | | | |
| | | AAGAAGAAAGCCCGTGCTGGCGGT | | | | | | | |
| | | AAGAAATCTCTGAGTGTCGCTCGC | | | | | | | |
| | | AGTTCTGGCGGTGGTCTGAATATC | | | | | | | |
| | | TAA (SEQ ID NO: 50) | | | | | | | |
| 33 | gp8 | ATGGCAAGCTCACAGAAACGTGAA | 1608 | − | Portal | 0.45 | 0.43 | 0.65 | <0.0001 |
| | | GGCTTCGCGGAGAATGGTGCCAAG | | | | | | | |
| | | GCGGTGTATGACGCATTGAAGAAC | | | | | | | |
| | | GACCGGAACTCCTACGAGACCCGT | | | | | | | |
| | | GCGGAAAACTGTGCGAAGTACACC | | | | | | | |
| | | ATCCCTTCGTTGTTCCCTAAAGAC | | | | | | | |
| | | TCCGACAACGCCTCTACTGACTAC | | | | | | | |
| | | ACCACTCCGTGGCAAGCAGTTGGC | | | | | | | |
| | | GCTCGTGGCTTGAACAACTTGGCC | | | | | | | |
| | | TCTAAGCTCATGCTCGCTCTGTTC | | | | | | | |
| | | CCTATGCAGACTTGGATGAAGCTG | | | | | | | |
| | | ACCATCTCTGAGTTCGAAGCGAAA | | | | | | | |
| | | CAATTGGTTGCACAGCCAGCCGAA | | | | | | | |
| | | CTAGCGAAGGTCGAAGAAGGTCTC | | | | | | | |
| | | TCAATGGTCGAGCGAATCTTGATG | | | | | | | |
| | | AACTACATCGAGTCCAACTCGTAC | | | | | | | |
| | | CGTGTGACACTCTTTGAGACCCTC | | | | | | | |
| | | AAGCAGTTGGTCGTTGCAGGTAAC | | | | | | | |
| | | GCTCTGCTCTACATTCCTGAACCT | | | | | | | |
| | | GAAGGTGCTTACAATCCCATGAAG | | | | | | | |
| | | CTATACCGCCTGTCTTCTTATGTT | | | | | | | |
| | | GTCCAACGAGACGCATTCGGCACG | | | | | | | |
| | | GTGCTACAGATTGTAACCTTAGAT | | | | | | | |
| | | AAGACTGCCTATGCAGCACTTCCA | | | | | | | |
| | | GAAGACGTGCGAAACGCTATGGAC | | | | | | | |
| | | TCCGGTCAGGAACATAAAGGCGAC | | | | | | | |
| | | GAAATGATTGATGTGTACACTCAC | | | | | | | |
| | | ATTTATCTTGACGAAGAATCAGGA | | | | | | | |
| | | GAATACCTCAAGTACGAAGAGATT | | | | | | | |
| | | GATGGTGTTGAAGTCGATGGTACA | | | | | | | |
| | | GATGCTTCTTATCCGGTGGACGCT | | | | | | | |
| | | TGTCCTTATATCCCGGTTCGCATG | | | | | | | |
| | | GTACGCATTGATGGAGAATCATAT | | | | | | | |
| | | GGTCGTTCTTACTGTGAGGAATAC | | | | | | | |
| | | TTAGGTGACTTACGGTCCCTTGAG | | | | | | | |
| | | AACCTTCAGGAAGCTATCGTTAAG | | | | | | | |
| | | ATGTCAATGATTAGTGCAAAAGTT | | | | | | | |
| | | ATCGGCTTGGTTAACCCGGCTGGT | | | | | | | |
| | | ATCACACAGGTTCGTCGTTTGACC | | | | | | | |
| | | AAAGCACAGACAGGTGACTTCGTG | | | | | | | |
| | | TCCGGTCGTCCAGAAGACATCTCG | | | | | | | |
| | | TTCCTTCAGCTTGAAAAGGCCGCT | | | | | | | |
| | | GACTTCTCTGTAGCGAAAGCTGTG | | | | | | | |
| | | AGTGAACAGATTGAGGGCCGACTG | | | | | | | |
| | | TCCTATGCCTTTATGCTGAACTCC | | | | | | | |
| | | GCTGTGCAACGTACTGGTGAGCGT | | | | | | | |
| | | GTGACTGCTGAAGAGATTCGATAT | | | | | | | |
| | | GTCGCATCTGAACTCGAAGATACC | | | | | | | |
| | | CTTGGTGGTGTCTATTCGATTCTC | | | | | | | |
| | | TCACAGGAACTTCAGTTGCCAATG | | | | | | | |
| | | GTTCGTGTCCTGCTGAAGCAACTT | | | | | | | |
| | | CAAGCAACCAATCAGATTCCTGAG | | | | | | | |
| | | TTACCGAAAGAGGCCGTTGAGCCT | | | | | | | |
| | | ACTATCAGTACCGGGATGGAAGCA | | | | | | | |
| | | TTGGGTCGTGGTCAAGACCTCGAT | | | | | | | |
| | | AAGCTGGAACGTTGTATTGCTGCG | | | | | | | |
| | | TGGTCTGCGCTGGCTCCTATGCAG | | | | | | | |
| | | AATGATCCTGACATCAACATTGCG | | | | | | | |
| | | ACCATTAAGTTGCGCATTGCGAAC | | | | | | | |
| | | GCTATCGGCATCGACACTTCTGGT | | | | | | | |
| | | ATCCTCAAGACACCTGAAGAGAAA | | | | | | | |
| | | CAACAGGAGATGGCTGAGGCTGCT | | | | | | | |
| | | CAAGGCACTGCTCTGGAGAACGCT | | | | | | | |
| | | GCTGCGTCTGCTGGTGCTGGTGCT | | | | | | | |
| | | GGTGCACTGGCTACTGCAAGTCCT | | | | | | | |
| | | GAGAACATGGAAGCTGCGGCTGCA | | | | | | | |
| | | CAGGCTGGTATGGTTCCTAATTAA | | | | | | | |
| | | (SEQ ID NO: 51) | | | | | | | |
| 34 | gp9 | ATGGCTGGTGAATCTAACGCTGAC | 912 | − | Capsid | 0.71 | 0.71 | 0.95 | 0.76 |
| | | GTATACGCATCTTTCGGTGTGAAC | | | assembly | | | | |
| | | TCTGCGGTTGTTGGTGGGTCTACT | | | scaffolding | | | | |
| | | CATACTGAACACGAACAGGCTATG | | | protein | | | | |
| | | CTTGAACTCGATGTTTCTGCCCGT | | | | | | | |
| | | GATGGCGATGATGCAATCGAGCTA | | | | | | | |
| | | GTTAGCAACGATGACCCTTATGGC | | | | | | | |
| | | AATCCAGACCCATTCGGTGAAGAG | | | | | | | |
| | | GATGAGAGTCGTACTGAGATTCGC | | | | | | | |
| | | ATCTCCACTGATGGTGAACCTGAA | | | | | | | |
| | | GTAACCGAAGAAGGTGAAATCGAC | | | | | | | |
| | | TATGAAGGAACCGAAGATGAAGGC | | | | | | | |
| | | TCCGAAGAGTTTGAACCAGTTGGT | | | | | | | |
| | | GACGTTCCAGATGAACTGAACGAA | | | | | | | |
| | | GCCTCAAGTCAACTCGAAGAGCAC | | | | | | | |
| | | GAAGCTGGTTTCGAAGAGATGGTC | | | | | | | |
| | | AATCAGGCGGCTGAACGTGGTCTG | | | | | | | |
| | | TCAGAAGAAACCATCGTGCGTATC | | | | | | | |
| | | CAACAGGAATACGGAGAGGACGGT | | | | | | | |
| | | CTGTCTGAAGAGTCTTACGAAGAG | | | | | | | |
| | | TTAGCCAAAGCTGGTTACTCCAAG | | | | | | | |
| | | TCGTTTGTTGACTCCTACATCCGT | | | | | | | |
| | | GGTCAGGAAGCACTGGTAGAACAG | | | | | | | |
| | | TATGTTCAGTCCGTTATGGAATAC | | | | | | | |
| | | GCTGGCGGTGAAGCACAGTTCCAA | | | | | | | |
| | | GCAATCTACAATCATCTCGAAGTG | | | | | | | |
| | | TCCAACCCGGATGCTGCTGAGTCT | | | | | | | |
| | | CTGATTAGTGCCTTGGAGAACCGT | | | | | | | |
| | | GACCTCGCAACAGTTAAGGCTATC | | | | | | | |
| | | GTCAATCTCGCTGGTGCAAGCCGT | | | | | | | |
| | | GCTAAGACTTTCGGTAAGCCTGCT | | | | | | | |
| | | GCCCGTAGTATCTCTAAACGTGCC | | | | | | | |
| | | GTACCTGCTGCACCTGTACGCACC | | | | | | | |
| | | AAACAGTCTGGCTTTGAGTCACAG | | | | | | | |
| | | GCTGAGATGATTGCTGCAATGTCC | | | | | | | |
| | | GACCCTCGCTATCGCCACGACAGT | | | | | | | |
| | | AAGTTCCGTGCGGAAGTTGAGCGC | | | | | | | |
| | | AAGATGATGTACAGTCAGTTCTAA | | | | | | | |
| | | (SEQ ID NO: 52) | | | | | | | |
| 35 | gp10ab | ATGGCTAACATGAATGGTGGACAG | 1178 | + | Major and | — | — | — | — |
| | | CAGATGGGTAAAGACCAAGGTAAA | | | minor | | | | |
| | | GGCATGAGCGCTGGTGACAAACTG | | | capsid | | | | |
| | | GCTCTGTTCCTGAAGGTCTTCGGT | | | proteins | | | | |
| | | GGTGAAGTTCTGACTGCATTTACC | | | | | | | |
| | | CGTACCTCCGTAACTATGAACAAA | | | | | | | |
| | | CACCTTGTGCGTTCTATTCAGTCC | | | | | | | |
| | | GGTAAGTCCGCGCAGTTCCCTGTG | | | | | | | |
| | | CTGGGTCGTACCAAAGCTGCTTAC | | | | | | | |
| | | CTGCAACCGGGTGAGAACCTCGAT | | | | | | | |
| | | GACAAACGTAAAGACATGAAGCAC | | | | | | | |
| | | ACTGAGAAGGTCATCAACATTGAT | | | | | | | |
| | | GGTCTGCTGACTGCTGACGTTCTG | | | | | | | |
| | | ATTTACGACATCGAAGACGCAATG | | | | | | | |
| | | AACCACTATGACGTGCGCTCCGAG | | | | | | | |
| | | TATACCGCACAGTTGGGTGAATCT | | | | | | | |
| | | CTCGCGATGGCTGCTGATGGCGCG | | | | | | | |
| | | GTTCTGGCTGAAATGGCTAAACTG | | | | | | | |
| | | TGTAACCTGCCGTCTGCGTCTGAT | | | | | | | |
| | | GAGAACATCGCTGGTCTCGGTAAG | | | | | | | |
| | | GCTCATGTACTGGAAGTTGGCAAG | | | | | | | |
| | | CAGTCTGATCTTCAGGGAGACCAA | | | | | | | |
| | | GTTAAGCTGGGTCAGGCTATTATC | | | | | | | |
| | | GCACAGTTAACTCTGGCTCGTGCC | | | | | | | |
| | | AAGCTGACTAGCAACTATGTTCCG | | | | | | | |
| | | TCTGCTGACCGTGTGTTCTACACC | | | | | | | |
| | | ACTCCAGACAACTACTCTGCTATT | | | | | | | |
| | | CTGGCTGCTCTGATGCCTAACGCT | | | | | | | |
| | | GCAAACTATCAGGCTCTGATTGAC | | | | | | | |
| | | CCGTCTACTGGCTCTATCCGCAAC | | | | | | | |
| | | GTTATGGGCTTCGAAGTTATTGAG | | | | | | | |
| | | GTTCCGCATCTGACTGCTGGTGGT | | | | | | | |
| | | GCTGGCGAAGACCGTCCAGAAGAG | | | | | | | |
| | | GGTGTAAATCCGACTGGTCAGAAA | | | | | | | |
| | | CATGCGTTCCCTGAGACTTCCTCT | | | | | | | |
| | | GGTGACACCCGTGTTGCTCTGGAC | | | | | | | |
| | | AACGTTGTCGGTCTGTTCAATCAC | | | | | | | |
| | | CGCTCTGCCGTTGGTACTGTTAAG | | | | | | | |
| | | CTGAAAGATATGGCACTGGAACGT | | | | | | | |
| | | GCTCGTCGAGCTAACTTCCAAGCA | | | | | | | |
| | | GACCAGATTATCGCTAAGTACGCT | | | | | | | |
| | | ATGGGTCACGGTGGTCTGCGCCCT | | | | | | | |
| | | GAAGCATGTGGCGCGCTGGTTTTC | | | | | | | |
| | | AACAAGGCTTAACTTCTCGCAAAC | | | | | | | |
| | | TCGTGGGGGACATTGAGTCCCTTA | | | | | | | |
| | | CTGAAGTTGAGCAAGAGCTAACGC | | | | | | | |
| | | CACAACAGAAAGCTGCCCGTACCC | | | | | | | |
| | | GTGCGGCAAACAAGGCTCGAAAGG | | | | | | | |
| | | AAGCTGAGTTAGCTGCTACTGAAT | | | | | | | |
| | | GA (SEQ ID NO: 53) | | | | | | | |
| 36 | gp11- | ATGCGTTCCTATGAGACTACCCTA | 2995 | − | Tail | 0.27 | 0.26 | 0.3 | 0.23 |
| | gp12 | GAGACTGGCGAAGAGTTGGCTGCG | | | tubular | | | | |
| | | GTAAATGACATCTTGGCCTCTATC | | | proteins | | | | |
| | | GGTGAACCACCTGTGTCCACTTTG | | | | | | | |
| | | GAAGGTGATGCTAACGCCGATGTT | | | | | | | |
| | | GCCAATGCTCGTCGAGTTCTCAAT | | | | | | | |
| | | AAGATTAACCGACAGATTCAGTCC | | | | | | | |
| | | AAGGGTTGGACGTTCAACATTGAG | | | | | | | |
| | | GAAGACCAACAGTTGCTACCTGAT | | | | | | | |
| | | GTGTTCAACGGATTGATTCCTTAT | | | | | | | |
| | | ATGTCCGACTACCTGTCTGTGATG | | | | | | | |
| | | ACACCGGGAGGCGCTACTGCCTAT | | | | | | | |
| | | GTCAACCGGGGTGGTTACGTGTTT | | | | | | | |
| | | GACCGGACTACTGGAACTGACGTG | | | | | | | |
| | | TTTAAAGAACCAATCACTGTGACC | | | | | | | |
| | | ATTATCAAGCTGAGAGAGTTCTAC | | | | | | | |
| | | GAGATGCCTGAATGTTTCCGCTTC | | | | | | | |
| | | TGGATTGTCACCAAAGCTGCCCGT | | | | | | | |
| | | CAGTTTAACAACAGGTTCTTTGGT | | | | | | | |
| | | GCTCCCGAGATTGACGCAGTGTTA | | | | | | | |
| | | GCTGAAGAAGAACAAGAAGCAAAG | | | | | | | |
| | | ATGCAGTGTCACGAGTATGAACTG | | | | | | | |
| | | GACTTTGGTAACTTCAACATGCTT | | | | | | | |
| | | GATGGTGATGCCTTCACTGGCGGT | | | | | | | |
| | | CTGCTATCTCGCTAATGATAACAA | | | | | | | |
| | | CTTAAGGAGGTCTCTAATGGCTCT | | | | | | | |
| | | TATTTCACAATCCGTCAAGAACCT | | | | | | | |
| | | GAAGGGCGGTATCAGTCAACAGCC | | | | | | | |
| | | GGACATCTTAAGGTTCCCCGAACA | | | | | | | |
| | | GGGTTCCGAACAGATTAACGGCTG | | | | | | | |
| | | GTCTTCGGAGACTGAGGGTCTTCA | | | | | | | |
| | | GAAGCGTCCACCTTTTGTCTTCAC | | | | | | | |
| | | TAAGACCATTGGAGACCAGAATGC | | | | | | | |
| | | CCTTGGTGCAAAACCGCTCATCCA | | | | | | | |
| | | TTTAATCAACCGTGATAGCGTAGA | | | | | | | |
| | | ACAGTATTACGTTGTGTTCACAGG | | | | | | | |
| | | ACAGTCAGTCCGAGTGTTCGACCT | | | | | | | |
| | | CAATGGCAAAGAGTACACAGTGAA | | | | | | | |
| | | AGGTGACTTGAGCTACGTGAAGGT | | | | | | | |
| | | AGGGAACCCGCGTGATGACCTGCG | | | | | | | |
| | | AATGGTAACGGTTGCTGACTATAC | | | | | | | |
| | | GTTTATCGTCAACCGCAACATGGT | | | | | | | |
| | | GGTTCGTGCCGATAAGACACCACT | | | | | | | |
| | | CTATACTCTGAAAGAGAACGGAGA | | | | | | | |
| | | CTGTTTGATTAACGTTCGTGGTGG | | | | | | | |
| | | ACAGTATGGGAGAACGCTTGCATT | | | | | | | |
| | | TACAATAAACAACGTTCGAATTGA | | | | | | | |
| | | GTACAAGATTGCACAAGGAGATAA | | | | | | | |
| | | GCCTGAACATGTACAAGAGACCGA | | | | | | | |
| | | TGCACAGTGGCTCGTTAAGAAACT | | | | | | | |
| | | GGCAGGTCTTGCTCGTGCTCACGG | | | | | | | |
| | | TTCCTTCAAGGACTGGAGGTTCGA | | | | | | | |
| | | CGAAGGGTCTGGCTTCATCCACGT | | | | | | | |
| | | AATCGCTCCGGGTAACAGTCAGAT | | | | | | | |
| | | TAACCAGCTGTCCACTGAGGATGG | | | | | | | |
| | | CTATGCTAACCAGTTGATGAACGC | | | | | | | |
| | | GGTAATGCACACAAGCCAGTCATT | | | | | | | |
| | | CAGTAAGTTGCCAATCGAGGCTCC | | | | | | | |
| | | TAATGGGTACACTGTGAAGATTGT | | | | | | | |
| | | TGGTGACACCTCTAAGACTTCCGA | | | | | | | |
| | | CATGTTCTATGTCCAGTATGACAA | | | | | | | |
| | | CGTGAAGAAAGTCTGGAAGGAAGT | | | | | | | |
| | | CGCCGGATGGGGAGTTGAGAAGGG | | | | | | | |
| | | TCTCAATGGAGGCACTATGCCACA | | | | | | | |
| | | CGCATTGGTTCGTCAGTCTGATGG | | | | | | | |
| | | TTCCTTCCAGATGCAGGTGCTACC | | | | | | | |
| | | GTGGGCGCAACGAACTTGTGGTGA | | | | | | | |
| | | CATGGATACTAACCCGACTCCATC | | | | | | | |
| | | CATCGTGGACCAGAAGATTAACGA | | | | | | | |
| | | TGTGTTCTTCTTCCGTAACCGCTT | | | | | | | |
| | | AGGGTTCCTCGCTGGTGAGAACAT | | | | | | | |
| | | TGTGATGTCTCGTACCTCTAAGTA | | | | | | | |
| | | TTTCTCACTGTTCCCTGCGTCAGT | | | | | | | |
| | | AGCTAACCTGTCCGACGATGACCC | | | | | | | |
| | | GATTGATGTGGCTGTATCTCACAA | | | | | | | |
| | | CCGTATCTCAATCCTGAAGTATGC | | | | | | | |
| | | CGTACCGTTCTCTGAAGAGTTGCT | | | | | | | |
| | | GCTATGGTCTGACCAAGCACAGTT | | | | | | | |
| | | CGTCCTGAGCGCTTCCGGTATCCT | | | | | | | |
| | | ATCACCGAAGACAGTTGAGTTGAA | | | | | | | |
| | | CCTTACGACCGAGTTCGATGTGTC | | | | | | | |
| | | CGACCGAGCGAGACCTTTTGGCGT | | | | | | | |
| | | TGGGCGTGGTGTGTACTTTGCGTC | | | | | | | |
| | | ACCTCGTGCTTCCTACACGTCACT | | | | | | | |
| | | GAACCGTTACTATGCGGTACAGGA | | | | | | | |
| | | TGTTAGCTCCGTGAAGTCTGCTGA | | | | | | | |
| | | GGATATGAGTGCTCACGTTCCAAG | | | | | | | |
| | | TTACATTCCGAACGGTGTGTTCTC | | | | | | | |
| | | CATTCGCGGCTCCGGTACTGAGAA | | | | | | | |
| | | CTTTATCTCCGTGCTCTCTGCGAA | | | | | | | |
| | | CGCCCCAAGTAAAATCTTCCTATA | | | | | | | |
| | | TAAGTTCCTCTACTTGAATGAAGA | | | | | | | |
| | | GATTGCCCAACAGTCGTGGTCTCA | | | | | | | |
| | | TTGGGAACTTGGTAGTAACGTAAC | | | | | | | |
| | | GGTTCTGGCTTGTGACTCTATCGG | | | | | | | |
| | | CTCAACGATGTACCTTGTGTTGCG | | | | | | | |
| | | CAATCAGTCACACACTTGGATGTG | | | | | | | |
| | | CCGAGCACACTTTACGAAGAACTC | | | | | | | |
| | | CATTGACTTCAACGATGAACCATA | | | | | | | |
| | | TCGGCTGTACATCGACAACAAGGT | | | | | | | |
| | | CAAGCATGTGATTCCTAAAGGTGC | | | | | | | |
| | | GTACAACGATGACACCTACACGAC | | | | | | | |
| | | CACTATCAAGCCTGTGGACATCTA | | | | | | | |
| | | CGGTATGAAATATTGGACTGGTAG | | | | | | | |
| | | GTTCTACATCGTGGCCTCTGATGG | | | | | | | |
| | | TATGGTCTCGTGGTTCGACCCTCC | | | | | | | |
| | | GCGTGGTGGTTGGCCTAATGGTGT | | | | | | | |
| | | CCCTGTGTTGTCAATGAGTGGGAA | | | | | | | |
| | | CCGTGAGGATGAGACAATCTACGT | | | | | | | |
| | | CGGCTTGGCTATCAACTTCCGTTA | | | | | | | |
| | | TGTGTTCTCTAAGTTCCTCATCAA | | | | | | | |
| | | GAAGACTGCTGATGATGGCTCGAC | | | | | | | |
| | | AGCTACCGAGGACATTGGGCGCTT | | | | | | | |
| | | GCAATTACGCCGAGCGTGGGTGAA | | | | | | | |
| | | CTATGAGGACTCTGGTGCATTCAC | | | | | | | |
| | | TGTAGAAGTGGAGAACACCTCTCG | | | | | | | |
| | | ACTGTTCAGCTATGACATGGCGGG | | | | | | | |
| | | TGCTCGCTTGGGTTCCAATGCGTT | | | | | | | |
| | | ACGCACTGGTGGACTTAACGTTGG | | | | | | | |
| | | TACTGGTCAGTTCCGATTCCCGGT | | | | | | | |
| | | TGCTGGCAACGCACAGTTGAACGA | | | | | | | |
| | | AGTAAGGATTATCTCTGACCACAC | | | | | | | |
| | | TACACCACTTAACGTGATTGGCTG | | | | | | | |
| | | TGGATGGGAAGGTAACTACCTACG | | | | | | | |
| | | ACGCTCTTCTAGTATCTAA (SEQ | | | | | | | |
| | | ID NO: 54) | | | | | | | |
| 37 | gp13- | ATGCTAATAATCAGACCTACTAAA | 7290 | − | Probable | 0.29 | 0.27 | 0.18 | 0.32 |
| | gp14- | GAATCAGACTTTGCTAGGTTCACT | | | scaffold | | | | |
| | gp15- | CCGTCACCTGAAGACATCGCTGAG | | | protein | | | | |
| | gp16 | GCAAAAGCCTATGGCATTGAACCT | | | (gp13); | | | | |
| | | TCATTTCCACCAGCTAACGAGTGC | | | internal | | | | |
| | | GTCACTATGTCACTTCATGGTATG | | | core | | | | |
| | | CCCGTGGCTATCGGTGGTAATTGT | | | proteins | | | | |
| | | GGCGACCAAGTGTGGTTCGTAACG | | | (gp14, | | | | |
| | | TCCGCTCGTGTTTGGAAACTAAGT | | | gp15, | | | | |
| | | ATGAAGACCCGAAGAGAGTTCCGA | | | gp16) | | | | |
| | | AAGCTCATTCTGGAGTATCGTGAT | | | | | | | |
| | | ACCATGCTCAAACAGTATCCGATT | | | | | | | |
| | | ATCTGGAACTACGTTTGGGTCGGC | | | | | | | |
| | | AACAAGTCCCACATTAGGTTCTTG | | | | | | | |
| | | AAGTCTATTGGTGCGGTGTTCCAT | | | | | | | |
| | | AATGAGTTTACTGGTGACAGTAAT | | | | | | | |
| | | CAATTCCAACTATTCACTATAGGA | | | | | | | |
| | | GGTTAACTATGTGCTGGATGGCTG | | | | | | | |
| | | CAATCCCAATCGCCATGCAAGGTG | | | | | | | |
| | | CGTCTATGGTAATGGGCGGTATGC | | | | | | | |
| | | AGGGTGAACAGGCAAAGGCCGCAC | | | | | | | |
| | | AGATTGACCAAGGTCGTCGACAAA | | | | | | | |
| | | GCTGGCAGATGATTAAGGAGATGA | | | | | | | |
| | | ACTACAACGATGCTAACCTTAAGT | | | | | | | |
| | | TAGAATCTCGTGACCTCATCGACT | | | | | | | |
| | | CAACTGTCCAAGAGATGACCCAAG | | | | | | | |
| | | CGAATATGAACCGTGTGCGAAACA | | | | | | | |
| | | TGGGGACTATCCGTGCGGCAATTG | | | | | | | |
| | | GTGAAGGGATGCTTGAAGGTAACT | | | | | | | |
| | | CAATGGAACGTGTAGCTCGCGTTA | | | | | | | |
| | | CTGAGGGTGACTTCCTTCGAGAGT | | | | | | | |
| | | CTCAAGGTCTCACCGAGAACTACC | | | | | | | |
| | | AACGTGACTACAGTGTCATCTTAG | | | | | | | |
| | | GTAAGCGTTTAGCTAACCGTGAGA | | | | | | | |
| | | ATACCGTAAGTCAAATCAATGAAA | | | | | | | |
| | | TCAACAAGTCCGAACCACGTCTGA | | | | | | | |
| | | AAGGCAAGCTGGAACAAATCATTG | | | | | | | |
| | | ACCCATTAGGCTTAGGCTTAACGA | | | | | | | |
| | | AGCTGGTCGATGTGGCAACTGCTG | | | | | | | |
| | | GTAGCCCTCTGACTAACAAGCTGG | | | | | | | |
| | | GTAAGAAGGCAGCTGCTAAAGTTA | | | | | | | |
| | | AGGCTTCGGATGCTAAGTCTACCG | | | | | | | |
| | | GACAAGGCAAGTAAACACAGGAGG | | | | | | | |
| | | TAACTGTATGAGTAAGTTAGCACA | | | | | | | |
| | | AGCATTGGGTAACATGGGTGCTCC | | | | | | | |
| | | TGCTGCAAGCCGACTCCGTGGTAC | | | | | | | |
| | | TGGTCGTATGGACGTTAAGGCTGC | | | | | | | |
| | | GACTGTCTATGAAGACCCTAGCTA | | | | | | | |
| | | TGCCAATAAGTCCGCACTGATTGG | | | | | | | |
| | | GACTGTAGGTAAACTTGCTGAGAT | | | | | | | |
| | | GGGTGCTGATGCGTACATGAAGTA | | | | | | | |
| | | TGACCAGAAGCAGAAGGACAAGGC | | | | | | | |
| | | AGATGAGCGCTCCAACGAGATTAT | | | | | | | |
| | | CCGCAAGTTGACTCCTGAGCAACG | | | | | | | |
| | | ACGTGAGGCCATTAAGAATGGAAC | | | | | | | |
| | | ACTTCTGTATCAGGACGACCCTTA | | | | | | | |
| | | CGCGATGGAAGCATTGAAGATTAA | | | | | | | |
| | | GACTGGTCGCAACGCGGCCTACTT | | | | | | | |
| | | GGTTGACGATGAGGTTGCACAGAA | | | | | | | |
| | | GGTTAAGAACGGTGAGTTCCGTAC | | | | | | | |
| | | TCGTCAAGAGTTGGAAGAGTTCCG | | | | | | | |
| | | TCACAGCCGACTGCAAGAAGCCGC | | | | | | | |
| | | TAAGAACTATGCGGAACAGTTCGG | | | | | | | |
| | | TATCAATGAGTCAGATGAGTTTTA | | | | | | | |
| | | TCAGAAGGGTTTCAACTCCGACAT | | | | | | | |
| | | CACTGAACGTAACATCGCCATCTA | | | | | | | |
| | | TGGTGCTCATGATAACTTCCTGAG | | | | | | | |
| | | TGACCAAGCTAAGAAGGGTGCCGT | | | | | | | |
| | | TATCAACAGCCGTGTCGAACTGAA | | | | | | | |
| | | CTCTGTACTCAACGACCCTGAGAC | | | | | | | |
| | | CCTTCGTTCCCCATATGCTGGTGA | | | | | | | |
| | | GTTCTTTGAGAACTACTTCAGTGC | | | | | | | |
| | | TGGATTGACGACTGGTAGTATCCC | | | | | | | |
| | | TAGTGATGACCAAGCGTTCACAAT | | | | | | | |
| | | GATTAGCCAAGGTCTGTCTGACGT | | | | | | | |
| | | GGTTAACCGTGAAGGTGGCGGTCA | | | | | | | |
| | | GTTCCTACAACAGATTGAGAACCG | | | | | | | |
| | | TAAGGTGAAACTCCACGGAAAAGA | | | | | | | |
| | | GACAACCTTCAAGGAACTGATGGG | | | | | | | |
| | | TGCCGAACAGTGGAACAATCTGAT | | | | | | | |
| | | GGTTAAGGCTCAACATAATGAGTT | | | | | | | |
| | | CCAGTTGAATGCTAAGAAGACCGA | | | | | | | |
| | | GGCGTTCCAACTTAATGTGAACTC | | | | | | | |
| | | TGCGTTGAACCAAGAGAACGTGAA | | | | | | | |
| | | CACTGGCTGGGAACAGTTGCAATC | | | | | | | |
| | | CATCAAGGCTGAACTGGACACACT | | | | | | | |
| | | GCAACCGGGCGAAGAGATGACACC | | | | | | | |
| | | TGAACGTCAGGCGTTAATCAATGC | | | | | | | |
| | | ACAGACGCAGATGCAGGACAGGAT | | | | | | | |
| | | GAAGCGGGAGACTGCTGAGTTAGC | | | | | | | |
| | | AAAGCAGATGGACAAGCAACAGAA | | | | | | | |
| | | GTCGATGAACAAGATGAACGTCAT | | | | | | | |
| | | TGACGCACAGTTCCAGAAGCGACT | | | | | | | |
| | | CAATGGTCAGTATGTGTCTACTGC | | | | | | | |
| | | GTATGGCGATATGCCTACCAACGA | | | | | | | |
| | | GAACACTGGTGAGTTCACCCATAG | | | | | | | |
| | | TGACATGGTTAACTATGCGAACAA | | | | | | | |
| | | GAAGTTAGCCGACATTGACGCTAT | | | | | | | |
| | | GGACATCCCTGCTGAACAGAAGGA | | | | | | | |
| | | CCGAATGAAGCTGGACTACCTCAA | | | | | | | |
| | | GGCTGACTCCGAGAAGGGTGCTTT | | | | | | | |
| | | CCGTACTGCGGTGGGTGAACTGAT | | | | | | | |
| | | TGGTGACGCTGAGAAGGAATGGAC | | | | | | | |
| | | GTCCGCTGTGATTAATGGGAAGAT | | | | | | | |
| | | GCCTGAAGATGGCGGTGTTGCCCT | | | | | | | |
| | | TAACGCTCTTCGTCGTGTCCGTAA | | | | | | | |
| | | TGCTGACCCTGAGTTATTTGCTGC | | | | | | | |
| | | GCTGTATCCTGATAAAGCCGAGAT | | | | | | | |
| | | GTTCCTCACTATGGACATGATGGA | | | | | | | |
| | | CAACCAAGGGATTGACCCGCAGAT | | | | | | | |
| | | TCTCTTAGATGCAGACAAGGCTCG | | | | | | | |
| | | ACAATCTCTGACCAAGGATATGCA | | | | | | | |
| | | GTATGAGGACGACAAGGCATGGGC | | | | | | | |
| | | TGCTCTCAAGAACAACTCACAGTC | | | | | | | |
| | | TCCTGAACTGTCCCGTATGCCAGC | | | | | | | |
| | | CACCTTGGATGCTGGTGCTCGTAA | | | | | | | |
| | | GATTTATGACTCTGTGAAGTACCG | | | | | | | |
| | | TACTGGTAACGCTGACATGGCAAT | | | | | | | |
| | | GCAACAGGTCGATAAGTATCTCAA | | | | | | | |
| | | AGAGAACACAACGACTCTCACTGG | | | | | | | |
| | | TGATGATGTTGATGGCGATACCAT | | | | | | | |
| | | TGGTGTCTTGACGAGAAACTCTCT | | | | | | | |
| | | GCGTGTGACCGATGACCCGGACTC | | | | | | | |
| | | TTGGAAACAAGGTAAGGACATCAT | | | | | | | |
| | | CGACCAAGCCGCTAAGAAACTCGC | | | | | | | |
| | | TGAGACGAACCCTTGGGTGACTAA | | | | | | | |
| | | TAAGCAACTCACTGTGTTCGAGCG | | | | | | | |
| | | TGGTGACTCTATCTACCTGATGGA | | | | | | | |
| | | CACTACCGGACAGGTTAACGTAAG | | | | | | | |
| | | GTATGACAAGCAGTTGCTCTCGAA | | | | | | | |
| | | GATGTACCAAGAGAATCAGGCCAA | | | | | | | |
| | | GTTGGATGCGGAAGCACGCGACAA | | | | | | | |
| | | GGCACTCAAAGATGCCAACAAGCG | | | | | | | |
| | | AACCCTGCATACACGAGCTATGAA | | | | | | | |
| | | CCGTAAGCGTGAACGTGAAGCCAA | | | | | | | |
| | | GAAACCTAAGCGTTCTGGTAGCAT | | | | | | | |
| | | GTATGATTCGGTGAGTGGTAAAGG | | | | | | | |
| | | TATTCTGGACACGCTGACTGGTAA | | | | | | | |
| | | AGACTAACAGACCACGATAGGAGG | | | | | | | |
| | | TTCCAAGTGGATAAGTATAACCAG | | | | | | | |
| | | AACGAACCGCATGAATATGATGCG | | | | | | | |
| | | TTATTTCAACAAGCTGCTGACACA | | | | | | | |
| | | CATGGAGTCTCCTATGGCCTTCTT | | | | | | | |
| | | CGGAAGGTCGGTTGGGTAGAGTCC | | | | | | | |
| | | CGCTTTAAGCCTACGGCTCAATCA | | | | | | | |
| | | CCAACAGGGCCGAGAGGTGTCATG | | | | | | | |
| | | CAGTTCACTAAGGCAACTGGTCAG | | | | | | | |
| | | GCTTATGGTCTCCAGAACGATGAG | | | | | | | |
| | | GACTTCGCTGACCCGGCTAAGTCC | | | | | | | |
| | | ATTGATGCAGGTGCTCGTTACCTT | | | | | | | |
| | | GCAGACCTCGTTAAGAAATACAAT | | | | | | | |
| | | GGGGATGAACTTAAAGCTGCCCTA | | | | | | | |
| | | GCGTACAACCAAGGTGAGGGACGT | | | | | | | |
| | | AATGGTAAACCCCAATTGGAAGCC | | | | | | | |
| | | TACGACTCTGGTAACTTTGCTGGC | | | | | | | |
| | | ATCGGTGATGAGGGGCGCAACTAT | | | | | | | |
| | | CTCCGCTCACTTCTGGATGTCGCT | | | | | | | |
| | | AAGTCTCCTAAGAGTGGTGACATT | | | | | | | |
| | | GAGTCATTCGGGGGTATCACCCCA | | | | | | | |
| | | AAGGGTAAAGGGATTCCGTTCGAT | | | | | | | |
| | | ACTGCTATGAGTGGTATCGGGAAG | | | | | | | |
| | | AAAGGAAAGGTAACTACAGAACTC | | | | | | | |
| | | CCTGAGTCTCACTCAATGTCCTTT | | | | | | | |
| | | CAAGGTAAAGAACAGTCCGCTCCC | | | | | | | |
| | | AATCAGCCGTTCGGAAAAGACTAT | | | | | | | |
| | | TGGGAAGCAAAAGGAACTACACTC | | | | | | | |
| | | GACGAAGCTAATGAGCGTTCAACC | | | | | | | |
| | | TTCTTCGGATTCGGTAATGCTACT | | | | | | | |
| | | GAAGCAGAACTCTCCAACTCAACC | | | | | | | |
| | | TTGGGTGTGGCCTTCCGTGCTGGT | | | | | | | |
| | | AAGCGTGATAATGGTTTCGATGTT | | | | | | | |
| | | GTTACTGATGTACTTCAACCGACA | | | | | | | |
| | | CGGTTTAACAGCCACATTTGGTCG | | | | | | | |
| | | CCTGAAGAACTCGAAAGGATTCGC | | | | | | | |
| | | AACGAAGTGAAGAACCCTGCGTAC | | | | | | | |
| | | ATGAACGTAGTGCTTGGAGGTTCC | | | | | | | |
| | | TCTGAGAATCTGGATGAACTCATC | | | | | | | |
| | | AAGTTAGCCAATGAGAACTTCGAG | | | | | | | |
| | | TTGGACTCTAAGGCCGCTGATGCT | | | | | | | |
| | | GGTATTGGTGCCAAACTGTCTGCT | | | | | | | |
| | | GGCCTTATTGGTGCTGGTGTTGAC | | | | | | | |
| | | CCATTGACCTACGTTCCTATCGCT | | | | | | | |
| | | GGGACTGCGGCTAAAGGGTTCAAG | | | | | | | |
| | | CTGGTTAACAAGGCATTGATTGTA | | | | | | | |
| | | GGTGCTCAAGCTGGCGCTCTTAAC | | | | | | | |
| | | GTGGCCTCTGAAGGACTCCGCACA | | | | | | | |
| | | TCGGTTGCTGGCGGTGATGCTCAC | | | | | | | |
| | | TATGCTGAAGCTGCCCTTGCTGGT | | | | | | | |
| | | ATGCTGTTCGCTGGTGGCCTTACT | | | | | | | |
| | | GCTATCGCTGATGGTGTGGCTGCT | | | | | | | |
| | | GGTCTCCGTAAGTCCGGTGCTGAA | | | | | | | |
| | | CAGATTGAGAACCCATTCGCTGCT | | | | | | | |
| | | GCACAGATGCGCTTTGAGGCTCGT | | | | | | | |
| | | GAGACTGCACGTAACACTGGTGGT | | | | | | | |
| | | TATGACGCAAGCCGTATGCCTCCT | | | | | | | |
| | | AGTGAAGACCGAGTGTTCTCGAAT | | | | | | | |
| | | CACAATGGCGTTGAGTATTCACCT | | | | | | | |
| | | CTTGAAACTGAACCGGGTGCCGTA | | | | | | | |
| | | GTGTTGCGTGATGGTTCCATTATC | | | | | | | |
| | | AGTGATACAAACTTGGCTAACCCG | | | | | | | |
| | | ATGACAGCCAAAGAGTTCGCAGAA | | | | | | | |
| | | GTAGACCCTGAACGTGCTGCGTGG | | | | | | | |
| | | GGCTTGCCTATGCGTGGACTGAGT | | | | | | | |
| | | GAAATCGGCTTGAGGACTTTGCGT | | | | | | | |
| | | TCTGAACACGCTGAAATCCGTGGT | | | | | | | |
| | | CTCGCTAAGGACTTAGTGCGTTCA | | | | | | | |
| | | CCTACAGGGATGGAGTCTGGCTCA | | | | | | | |
| | | CATGGTAAGTTCGGTGCTACGGCC | | | | | | | |
| | | TCTGACATCAAAGAGCGTCTGCAC | | | | | | | |
| | | AGTACCAACCAGCGAACCTACAAT | | | | | | | |
| | | GACCTCTATGATGCAATGAAGGAA | | | | | | | |
| | | GCAATGGCTGACCCTGAGTGGTCA | | | | | | | |
| | | GTGGGTATGTTCAAGCGTGGTGCT | | | | | | | |
| | | CAAGGTGCCCGTCAGGAAATCTAT | | | | | | | |
| | | CGTCGTGCTGCTATCGCTATTGAG | | | | | | | |
| | | CGTCCAGAACTTCAAGCCAACTTA | | | | | | | |
| | | ACGAAGGCTGAACGTAAGGTAATG | | | | | | | |
| | | GACATCATGAAGGAACATTTCGAC | | | | | | | |
| | | CTCAAGCGTGAGATGATGGAGAAC | | | | | | | |
| | | CCGGCTATGTTCGGTAACAAGGCA | | | | | | | |
| | | ACTTCCATCTTCCCTAACAGTCGT | | | | | | | |
| | | CACAAAGGGACTTACGTTCCTCAT | | | | | | | |
| | | GTGTACTCACGGGAAGCCAAGCAG | | | | | | | |
| | | TTGTACGCACAGGCTCTTGGTGGC | | | | | | | |
| | | TCTGACGGTTTACAGGAAGCTATC | | | | | | | |
| | | GCTGCAAGCTGGATGACCTCTTAC | | | | | | | |
| | | CGTTCACGCCCTGAAGTTAAGGCT | | | | | | | |
| | | CGTGTGGATGAGCATTTAGCGGAG | | | | | | | |
| | | ACATTGGGTATCGACCCGAAGGCT | | | | | | | |
| | | GTAACCGAAGAGATGGTGATGAAA | | | | | | | |
| | | CATGCCAGCGACAAGGCTTATGGT | | | | | | | |
| | | ATTGCGAAGACTGATGAGTTCAAC | | | | | | | |
| | | TCTTCGAGTGTCATCGACGATAAC | | | | | | | |
| | | ATTGAGGGTCTCGTTGGGATTGAG | | | | | | | |
| | | AACAACTCGTTCCTTGAAGCCCGT | | | | | | | |
| | | AACCTGTTTGACTCTGACATGCCA | | | | | | | |
| | | GTCACTCTACCTAATGGACAGCAA | | | | | | | |
| | | TTCTCTGTGAATGACCTCCGTGAC | | | | | | | |
| | | TTCGATATGAAGCATGTGATGCCA | | | | | | | |
| | | GCCTATGACCGCCGTGTTGATGGT | | | | | | | |
| | | GACATTGCTATCATGGGTGGTACA | | | | | | | |
| | | GGAAAGACTACTGCTGAACTCAAG | | | | | | | |
| | | GATTCCATTATGGCTCTCGATAAG | | | | | | | |
| | | AAGTCAGAAGGGAAGGGTACGATG | | | | | | | |
| | | AAAGGTGAAGTTGAAGCACTCAAG | | | | | | | |
| | | GATACCGTTAAGATTCTCACAGGT | | | | | | | |
| | | CGCGCTCGACGCAATCAGGATACT | | | | | | | |
| | | ATGGGTGACACTATGGTTCGCGCT | | | | | | | |
| | | CTGTCCGATATGTCCTTCTTTACG | | | | | | | |
| | | AAGAACGCATACATGGGCTTGCAG | | | | | | | |
| | | AACCTAACTGAAATCTCTGGTCTC | | | | | | | |
| | | TTGGCGAAGGGTAACACCCGTGCG | | | | | | | |
| | | ATGCTGCATGGCATTCCTGCTCTG | | | | | | | |
| | | CGTGACCTAGCATTCCGTAACAAG | | | | | | | |
| | | CCAGTGTCAGGTAAGGAACTTAAG | | | | | | | |
| | | GAACTACACTCTATGGTGTTCGGT | | | | | | | |
| | | AAAGAGTTCGATGACTTAATCAGA | | | | | | | |
| | | CCGACACGTCAGGACATCATTCAG | | | | | | | |
| | | CGACTCCGTGAATCTACGGATACA | | | | | | | |
| | | CCTGATGTTGCCGCTAAGGTTGTT | | | | | | | |
| | | GGTACGGTTAAGCACACCACTCAA | | | | | | | |
| | | GAGTTTGCTGCTCGCTCTCCGTTC | | | | | | | |
| | | ACTAAGTTCCTGAATGGTACATCA | | | | | | | |
| | | AACTACATTCTCGACATGGCTCGA | | | | | | | |
| | | CAGGGTGTGATGGGTGATGTGGTC | | | | | | | |
| | | ACTCATGCTATCACTGGCAAGGGT | | | | | | | |
| | | GCCAACAAGTGGATTAAAGGCGAC | | | | | | | |
| | | ATGCTGAAGTCTGCGAGTATCTCA | | | | | | | |
| | | AAGGAACAGTGGGAAGGCATTCAG | | | | | | | |
| | | AACCTCATCCGTGAGAACGTCACT | | | | | | | |
| | | CAAGGTGCTGACGGTAAGTACACC | | | | | | | |
| | | TTCAAGGACAAACGCAAGCTGGCT | | | | | | | |
| | | AATGACCCTCGTGCTATGGACTTG | | | | | | | |
| | | TGGCGCTTAGCTGATAAGGTTGCC | | | | | | | |
| | | GATGAGACAATGCTGAGACCTCAC | | | | | | | |
| | | AAGGTTTCACTTCAGGATTCCCAT | | | | | | | |
| | | GCGTTTGGTGCTGCCGCTAAGTTG | | | | | | | |
| | | GTCCTTCAGTTCAAGTCTTTCGTA | | | | | | | |
| | | ATCAAGTCGATGAACTCTAAGTTT | | | | | | | |
| | | ATCCGCTCTGGTCACGAGGCTATG | | | | | | | |
| | | AAGAATCACCGAGCTATGGACATG | | | | | | | |
| | | GCATTAACCTATGCAATCTCTGGA | | | | | | | |
| | | GGCATCGCTGGTGCTTACTATGTT | | | | | | | |
| | | GCACAGGCACACATGAAGGCTGCT | | | | | | | |
| | | GGTCTACCTAAAGAGCAACAGAAG | | | | | | | |
| | | GATTACTTGAAGAAAGCTCTGGAC | | | | | | | |
| | | CCTAAGATGATTGCGTATGCTGCT | | | | | | | |
| | | GCATCCCGTAGTTCACATTTAGGT | | | | | | | |
| | | TCCCCGTTGAGTATCGCCAACTTC | | | | | | | |
| | | GCTATGGGTGCTGCTGGTTACGAC | | | | | | | |
| | | CAAGGTCTCATGGTGCGCTCCACG | | | | | | | |
| | | ATTCTACCGAAAGGAGAAGACAAG | | | | | | | |
| | | CGTGACCGGAATAAAGCAGTGACC | | | | | | | |
| | | TCTCGTGATATGGGAGAATCCATC | | | | | | | |
| | | ATGGGCGCTATTGGTGAACAGATT | | | | | | | |
| | | CCTGCTCTTGGTTTCGCTGGGGCA | | | | | | | |
| | | ACTCTTGCTGCTGGTCGAAATGCT | | | | | | | |
| | | TATGGTGTCCTAACGGCTCCTAAC | | | | | | | |
| | | AAAGTGACCGAGCGTGAGATGATG | | | | | | | |
| | | ACTGGCTTGATGAATGCTCATCGT | | | | | | | |
| | | GAGATGGTTCCTAACGACCCTATC | | | | | | | |
| | | ACTCAACAGATGCTCATTAAGTTC | | | | | | | |
| | | TATGAGGCTAACGGTGTTCACCTT | | | | | | | |
| | | AAGTCTGACAAGAAGTAA (SEQ | | | | | | | |
| | | ID NO: 55) | | | | | | | |
| 38 | gp17 | ATGGCTAACAAGATTCGTACTGTG | 1968 | − | Tail fiber | 0.45 | 0.44 | 0.66 | 0.40 |
| | | ATGACTTACCCACTTAATGGTGCC | | | protein | | | | |
| | | GTGAACTTCACGATTACCTTTGAG | | | | | | | |
| | | TATCTGGCACGTAAGTTCATCCAA | | | | | | | |
| | | GTGACACTCATTGGTAAAGACCGT | | | | | | | |
| | | AAAGAACTGGTTCTCAATCAGGAC | | | | | | | |
| | | TACCGTTTTACCAGTAAGACTCAA | | | | | | | |
| | | ATTACTACTACCCGTGCGTGGACT | | | | | | | |
| | | GCTGCTGATGGCTACCAGATGATT | | | | | | | |
| | | GAGATTCGTCGATTCACTTCGGCT | | | | | | | |
| | | ACTGACCGTCTGGTTGACTTTGCC | | | | | | | |
| | | GATGGCTCAATCCTCCGTGCGTAT | | | | | | | |
| | | GACCTGAACATCTCGCAGATTCAA | | | | | | | |
| | | ACACTGCATGTTGCCGAAGAAGCA | | | | | | | |
| | | CGAGACCTCACTGCGGATACCATT | | | | | | | |
| | | GGTGTCAACAATGATGGTCACTTA | | | | | | | |
| | | GATGCTCGTGGTCGTAAGATTGTT | | | | | | | |
| | | AACTTGGCGTATGCCACAAGTGAC | | | | | | | |
| | | TATGATGCCGTTCCTTTAAAGCAG | | | | | | | |
| | | GTTAAAGAGCGTGAATCCTCTGCA | | | | | | | |
| | | TGGAATGCTGTGACAAAAGCTAAC | | | | | | | |
| | | GAACACGCTGACAGGGCCAATAAG | | | | | | | |
| | | GAAGCAAACCGTTCTCGTGATGAG | | | | | | | |
| | | GCCAACCGTGCCCAACGTGAAGCT | | | | | | | |
| | | GACCGCTCAACGCAACAGGCAGGT | | | | | | | |
| | | GTCTCTACACAACAAGCAGTTGAG | | | | | | | |
| | | GCCAAGAAGCAAGCTGACCGTTCT | | | | | | | |
| | | AACTCTGAGGCTAACCGAGCTAAA | | | | | | | |
| | | GGTTATGCTGATAGTATGACCGCA | | | | | | | |
| | | AGTGTCGCTGAGGCCAAGAAGCAA | | | | | | | |
| | | GCTGACCGTTCCACCTCTGAGGCC | | | | | | | |
| | | AATCGTGCCCGTGATGAGGCCAAT | | | | | | | |
| | | CGTGCTGCCGGAGAAGTGACTAAA | | | | | | | |
| | | GCTGCTGCTGAGGTTGCCAAGGCT | | | | | | | |
| | | GCTGCACACGTTGAGACCGCTAAG | | | | | | | |
| | | AGTCACGCCAATCGTGCTAACACT | | | | | | | |
| | | GAGGCAGAACGCTCTAAGAGTGAA | | | | | | | |
| | | GCCGACCGTGCTAAAACCGAGGCT | | | | | | | |
| | | GACAAGCTGGGTAATATGAATGAA | | | | | | | |
| | | TTAGCTGAGGCCATCGACAGGGTT | | | | | | | |
| | | GATGCTCCTGATGTTTACTTCAAG | | | | | | | |
| | | AGGAATGTTATAGCCGGACACAAA | | | | | | | |
| | | TTGGAGACTTCTGGAGGCTCGCAT | | | | | | | |
| | | CTTCACCCTAATGAGATTAAATTG | | | | | | | |
| | | GTTAACAATAAATCTCCAGACCAG | | | | | | | |
| | | CACTTCATTGTACAAGCGTGGGGA | | | | | | | |
| | | ACTGGAGACCCTAATAATAGGTCG | | | | | | | |
| | | CAGGTGTTTGAGTTAAAGGATAAC | | | | | | | |
| | | TTGGGTCCGTACGCATTCTATATC | | | | | | | |
| | | CAGCGCCAGAAGAAGACAGACCAA | | | | | | | |
| | | ACTTGGCAAATGAGTGTGAACGGT | | | | | | | |
| | | ACTGTTAGCTGCGTATCCATCGAG | | | | | | | |
| | | GCTGGCAAGGGTTTCATCTCTAAG | | | | | | | |
| | | GACAGTACAGGCTCCATTCAGATG | | | | | | | |
| | | AAGACTTGGGGGGGTGGTTGGAAC | | | | | | | |
| | | CAATGGACTTCTAATGCTGTTCCA | | | | | | | |
| | | GACATTCAGCACGATATTGAGGCC | | | | | | | |
| | | CAACATGATGCTGCTTATCTTCTG | | | | | | | |
| | | GAGAGGGCTACCATTTGGGGTAAG | | | | | | | |
| | | GAGCATTTATACGCAAAACAACTG | | | | | | | |
| | | TACAATAATGGCACTGGCGCAAAC | | | | | | | |
| | | TTCTTTATTCGCCACTACCTACGA | | | | | | | |
| | | GGCTCTTACTTCGACTTCCGCTCT | | | | | | | |
| | | GATGGTACTCTTATCAGTTCCACC | | | | | | | |
| | | GGATGGAATATCCCGGCAGATGGA | | | | | | | |
| | | AACCTGTACATCAAGAAATATGGA | | | | | | | |
| | | CTATATATTGATGCGTGGGTTAAC | | | | | | | |
| | | AAGCGCTTGGGCGAACACGCCTAC | | | | | | | |
| | | AACAAGAGCGAGACAAACAACCTA | | | | | | | |
| | | GTGAATGGTCGATTGACACAAGCG | | | | | | | |
| | | CAAGCTGATGGTCGCTACATTCGC | | | | | | | |
| | | AAGAACGCTGGTTGGGCTGAGGTC | | | | | | | |
| | | TGGAGCGGCTCTGCTGGTGGTGGT | | | | | | | |
| | | GTTTCCGTAGGTCTCTCACAGGAT | | | | | | | |
| | | GTTCGTTGGAGAACCATCTGGATT | | | | | | | |
| | | CAAACTCAAGGCAGATGGAACCCT | | | | | | | |
| | | GTTCAGATTGGTGATAATGGTGCT | | | | | | | |
| | | TACTACATCTCGTCTAATGGTGGG | | | | | | | |
| | | TGGTTGAAGTTCACCATCTCTAAT | | | | | | | |
| | | AACGGTAGGACGTTCCGTAACGAT | | | | | | | |
| | | GAGGACAGGACTTCTGTTCCTACG | | | | | | | |
| | | AGAATCTTGATTCAAAACCAATAA | | | | | | | |
| | | (SEQ ID NO: 56) | | | | | | | |
| 39 | gp17.5- | ATGTTGTCATTGGATTTTAACAAC | 466 | − | Holin; | 0.060 | 0.060 | 0.10 | 0.059 |
| | gp18 | GAGGTAGTGAAGGCTGCTCCGATT | | | small | | | | |
| | | GTAGGGACAGGGGTTGCCGATGGT | | | terminase | | | | |
| | | GCTTCCAGACTGTTCTTCGGCCTA | | | | | | | |
| | | TCACTTAACGAGTGGTTTTATGTA | | | | | | | |
| | | GCCGCAATTGCCTACACAGTGGCT | | | | | | | |
| | | CAAATTGGTGCCAAGGTAGTCGAC | | | | | | | |
| | | GTGGTTATCAAATGGAAGAAGGAG | | | | | | | |
| | | GGTAGAGATGTCGGATAAATCTTT | | | | | | | |
| | | GATTCAATTCCTTGAGATGCTGGA | | | | | | | |
| | | CACTGAGATGGCTCAACGTATGCT | | | | | | | |
| | | TGCTGACTTGCAGAACGATGAACG | | | | | | | |
| | | CCGTACACCTCAACTGTACAACGC | | | | | | | |
| | | AATCGGGAAACTGTTAGAACGCCA | | | | | | | |
| | | CAAGTTCCAAGTCTCCAAGTTACA | | | | | | | |
| | | GCCAGACCGTAACATCCTTGGTGG | | | | | | | |
| | | ACTGGCAGATGGACTTCAGGAGTA | | | | | | | |
| | | TCAGAACCTAGTAGGTGACGATGG | | | | | | | |
| | | TCTCACTGAAGATGAGAAGTACAC | | | | | | | |
| | | GCTGAACTGA (SEQ ID NO: | | | | | | | |
| | | 57) | | | | | | | |
| 40 | gp18.5- | ATGCTCAAGAATTTAAAGAGCTAC | 444 | − | Spanin, | 0.046 | 0.054 | 0.16 | 0.077 |
| | gp18.7 | GTCATAATCGCTGCGTTTGCCTTG | | | inner | | | | |
| | | GGGATGACCCTTGCGTACAACCAC | | | membrane | | | | |
| | | GGCTATGACAAGGCTAACACTAAA | | | and outer | | | | |
| | | TGGGAACAGGAGGTGCACAATGAG | | | lipoprotein | | | | |
| | | TACGTTAAGAAAGTTAAGGCGACT | | | subunits | | | | |
| | | GAGGATAAACAGGCTGCTATCAAC | | | | | | | |
| | | GAAGTCTCACGACAATACCAAGAA | | | | | | | |
| | | GACCTTGCGGCGCTGGAAGGCAGC | | | | | | | |
| | | ACTGATAGGGTCATTAATGATTTG | | | | | | | |
| | | CGTAACGATGGTAAGCGGCTGCGT | | | | | | | |
| | | GTCAAACTATCAGCCACCTCACGA | | | | | | | |
| | | GAACTCCAAGCTAACGGTGGATGC | | | | | | | |
| | | CTCGTTGATGGTCGAGCCGAACTT | | | | | | | |
| | | GACGAAGAGTTTAGTAAGCGTCTT | | | | | | | |
| | | ATCGGAGTAACACAAAGAGGGGAT | | | | | | | |
| | | GCGTGGATTAAAGCGCTCCAAAAT | | | | | | | |
| | | ACAATAACTGAAATGCAATCTAAG | | | | | | | |
| | | AAGGAGAAATAA (SEQ ID NO: | | | | | | | |
| | | 58) | | | | | | | |
| 41 | hypo- | ATGGCTACTACAAACTCAAGAACC | 558 | − | Hypothetical | 1.0 | 1.0 | 1.1 | 1.2 |
| | thetical | CGCACACTCTGGACCATCAATGGT | | | | | | | |
| | protein | CGCTCACCGGACAGCCGGGATATG | | | | | | | |
| | | TACGACCGAAACAATGTTGGCCTT | | | | | | | |
| | | CGTGTTGGGCGCATCGACATCCCA | | | | | | | |
| | | GCGTCAGAAATGGTCAAGGTAGAT | | | | | | | |
| | | GGCTACGACAAGGTTGAATACGTG | | | | | | | |
| | | GTGCAACTGAGTGATGGCACAACC | | | | | | | |
| | | GAGCAACATGCTACGTTTGAGACC | | | | | | | |
| | | TTGATGTCCACTTGGGGTGGCTCC | | | | | | | |
| | | GTAGAGTCTAAACCGGGTAGTGGG | | | | | | | |
| | | TTCATGACGGTATTTCTGAATGAG | | | | | | | |
| | | CAAAGCACTGAGCGGCTCGGTATG | | | | | | | |
| | | GTTCGAATCGACGTATACAAAGAG | | | | | | | |
| | | TGGACTGGTGCTGACCCTGAGCCG | | | | | | | |
| | | GAAGGTGGTGTGGCTTCATTAGTT | | | | | | | |
| | | AGTGCAACGCTTTCTGCGTCTGAT | | | | | | | |
| | | AGTGCATTTGGTACACAGTTCGTG | | | | | | | |
| | | GATAGCGAAGGGAAGAGCGCTTCC | | | | | | | |
| | | GTTATGTTCCAACCTAGTAACATC | | | | | | | |
| | | GACGGGGCTGTATCTAAGACCGAC | | | | | | | |
| | | ACTATCAAGGCTCTTGAGAAGCGC | | | | | | | |
| | | ATCGCTGCCTTAGAGAAATCTGCC | | | | | | | |
| | | GAGTAA (SEQ ID NO: 59) | | | | | | | |
| 42 | gp19 | ATGTCACAATCCCAAGAAGCTAAG | 1761 | − | Large | 0.11 | 0.11 | <0.0001 | 0.11 |
| | | AATGCGCTCATCATTGCGCAACTC | | | terminase | | | | |
| | | AAGGGTGACTTCGTGGCCTTCCTG | | | | | | | |
| | | TTCGTCTTATGGAAGGCTCTGAAC | | | | | | | |
| | | TTACCTAAACCAACCAAGTGTCAG | | | | | | | |
| | | ATTGATATGGCACGAACACTCGCT | | | | | | | |
| | | AATGGAGACCACAAGAAGTTTATC | | | | | | | |
| | | CTTCAGGCGTTCCGTGGTATCGGC | | | | | | | |
| | | AAGTCCTTTATCACCTGTGCGTTC | | | | | | | |
| | | GTTGTGTGGGTCTTATGGCGTGAC | | | | | | | |
| | | CCTCAACTTAAGGTGCTCATTGTG | | | | | | | |
| | | TCTGCCTCTAAGGAACGTGCGGAT | | | | | | | |
| | | GCTAACTCCATCTTCATCAAGAAC | | | | | | | |
| | | ATCATCGACCTCTTACCGTTCCTC | | | | | | | |
| | | TCTGAGTTGAAACCTCGACCGGGA | | | | | | | |
| | | CAGCGAGACTCCGTGATTAGCTTT | | | | | | | |
| | | GATGTTGGCTTGGCTAAACCCGAC | | | | | | | |
| | | CACTCACCTTCTGTGAAGTCTGTG | | | | | | | |
| | | GGTATTACTGGTCAGTTGACTGGT | | | | | | | |
| | | AGCCGTGCTGACATCATTATTGCA | | | | | | | |
| | | GATGACGTTGAGGTTCCCGGTAAT | | | | | | | |
| | | AGCTCTACGAGTTCTGCCCGTGAG | | | | | | | |
| | | AAGCTCTGGACGTTGGTAACTGAG | | | | | | | |
| | | TTTGCTGCATTGCTTAAGCCGCTG | | | | | | | |
| | | CCTACCTCTCGTGTTATCTACCTT | | | | | | | |
| | | GGCACACCTCAAACCGAGATGACG | | | | | | | |
| | | CTCTACAAGGAACTTGAGGATAAC | | | | | | | |
| | | AAAGGGTACAGCACAGTAATCTGG | | | | | | | |
| | | CCAGCACAGTATCCTCGTAATGAT | | | | | | | |
| | | GCTGAGGCTCTCTACTATGGGGAC | | | | | | | |
| | | CGCTTGGCTCCGATGCTTAAGGCT | | | | | | | |
| | | GAGTACGACGAGGGCTTTGAGTTA | | | | | | | |
| | | CTCCGTGGTCAACCTACTGACCCT | | | | | | | |
| | | GTTCGATTCGACATGGATGACCTC | | | | | | | |
| | | CGTGAACGTGAACTCGAATATGGC | | | | | | | |
| | | AAAGCTGGCTACACTCTTCAGTTC | | | | | | | |
| | | ATGCTTAACCCGAACCTTAGTGAT | | | | | | | |
| | | GCCGAGAAGTACCCACTACGACTC | | | | | | | |
| | | CGTGACGCTATCGTGTGCGCTGTG | | | | | | | |
| | | GACCCTGAACGTGCCCCATTGTCT | | | | | | | |
| | | TACCAGTGGTTGCCGAACCGTCAG | | | | | | | |
| | | AACCGCAATGAGGAACTCCCTAAC | | | | | | | |
| | | GTTGGTCTGAAGGGTGATGACATC | | | | | | | |
| | | CATGCGTTCCATACGTGCTCTTCT | | | | | | | |
| | | CGTACCGCTGAGTATCAGTCTAAG | | | | | | | |
| | | ATTCTGGTCATTGACCCAAGTGGT | | | | | | | |
| | | CGTGGTAAGGATGAGACCGGGTAT | | | | | | | |
| | | GCAGTCCTCTACTCACTGAACGGT | | | | | | | |
| | | TACATCTACCTGATGGAAGTCGGA | | | | | | | |
| | | GGGTTCCGTGGTGGCTACGATGAT | | | | | | | |
| | | GCTACCCTAGAGAAGCTCGCTAAG | | | | | | | |
| | | AAGGCCAAGCAGTGGAAGGTACAG | | | | | | | |
| | | ACAGTCGTCCACGAGTCCAACTTC | | | | | | | |
| | | GGTGATGGTATGTTCGGTAAGATT | | | | | | | |
| | | TTCTCTCCGATTCTCCTGAAGCAC | | | | | | | |
| | | CATAAGTGTGCCTTAGAGGAAATC | | | | | | | |
| | | CGTGCTAAGGGCATGAAGGAGATG | | | | | | | |
| | | CGAATCTGCGATACTATCGAGCCG | | | | | | | |
| | | TTGATGGGTGCACACAAGCTGGTC | | | | | | | |
| | | ATCCGTGATGAGGTCATCCGTGAG | | | | | | | |
| | | GACTACCAGACTGCCCGTGACCTT | | | | | | | |
| | | GATGGGAAACATGATGTCCGCTAC | | | | | | | |
| | | AGTGCGTTCTATCAGATGACACGT | | | | | | | |
| | | ATGACCCGTGAGCGAGGCGCTGTG | | | | | | | |
| | | GCACACGATGACCGCATTGATGCT | | | | | | | |
| | | ATCGCATTGGGTATCGAATATCTC | | | | | | | |
| | | CGCGAGGGGATGCTTGTGGACAGC | | | | | | | |
| | | CGTGTAGGTGAAGAAGAGATGACC | | | | | | | |
| | | CTTGAGTTCCTTGAACACCACATG | | | | | | | |
| | | GAGAAACAGACCATTGGTGGCGAC | | | | | | | |
| | | CAGATTCATAGCTTTGATGTTGGG | | | | | | | |
| | | GGTGTGGACATCTACTATGAGGAT | | | | | | | |
| | | GAGGACGGAGGTTCTAGCTTCATT | | | | | | | |
| | | GAGTGGTAA (SEQ ID NO: | | | | | | | |
| | | 60) | | | | | | | |

### Avs Proteins are Pattern Recognition Receptors Capable of Recognizing a Diverse Range of Terminase and Portal Proteins

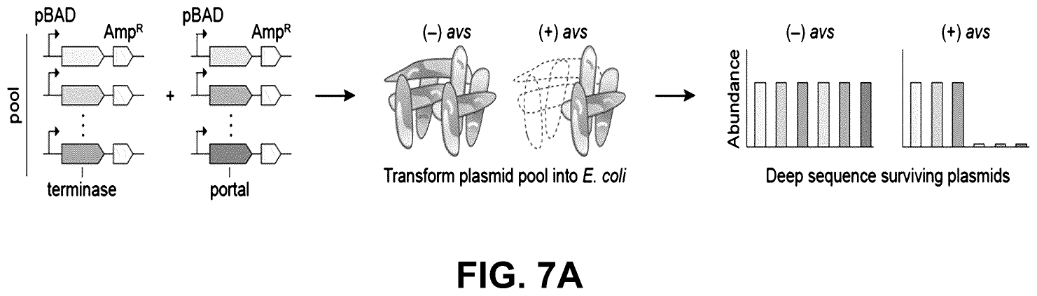

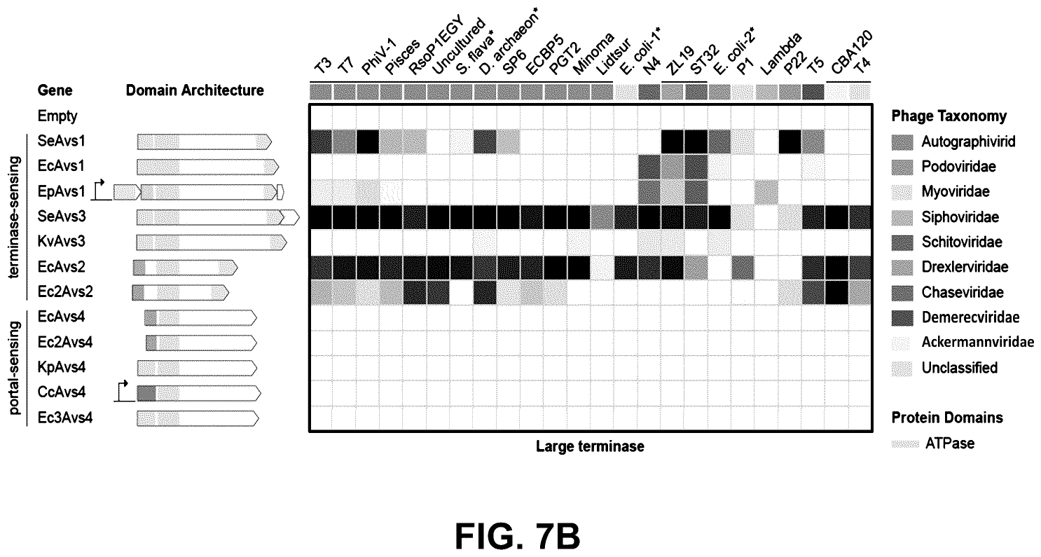

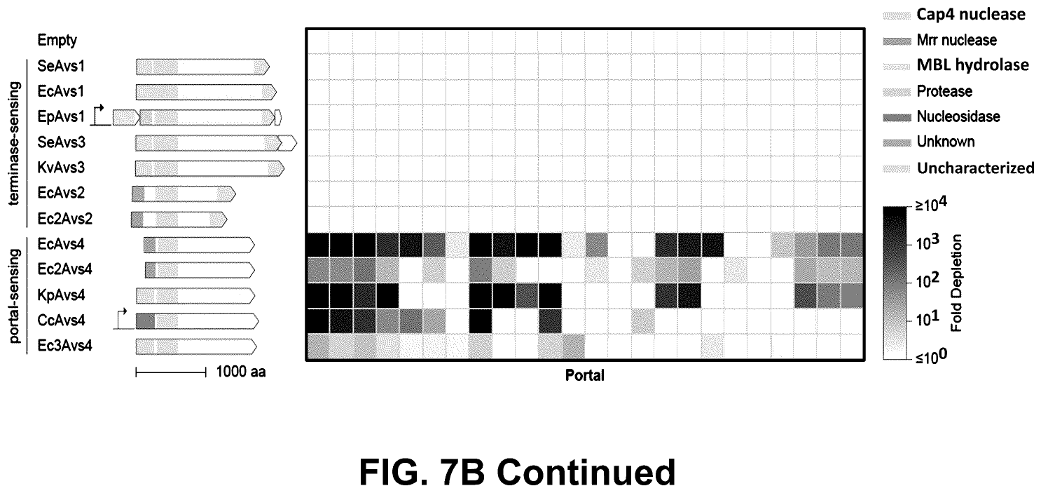

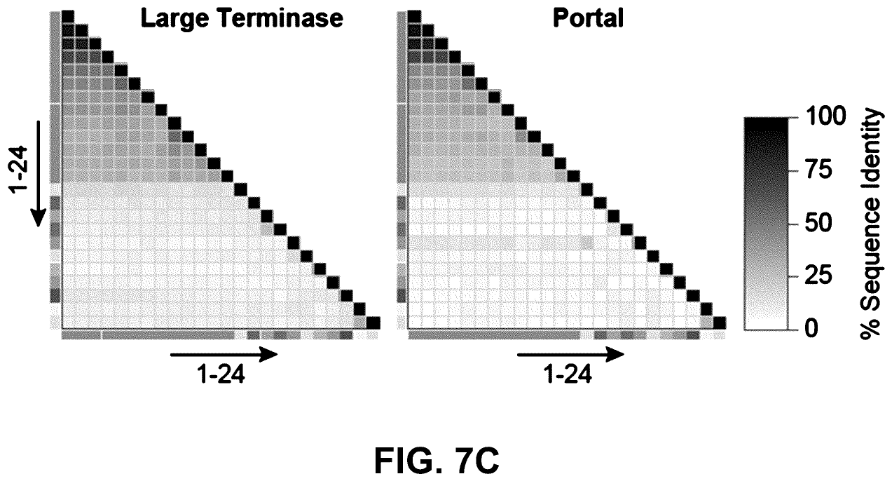

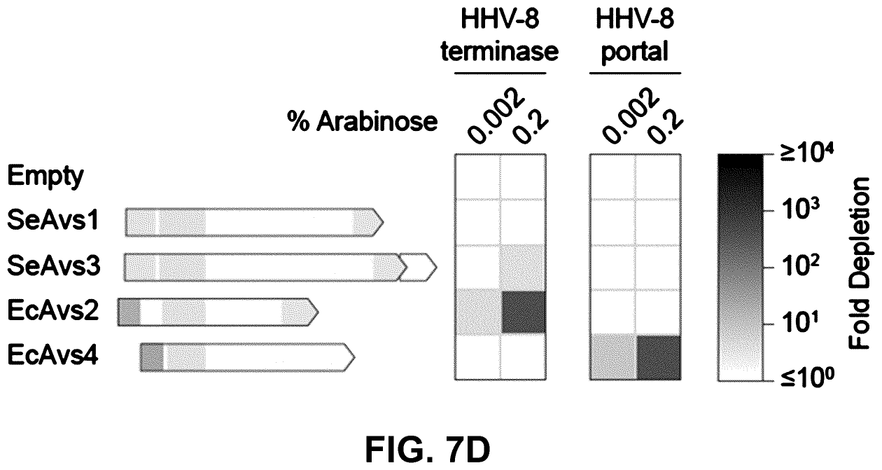

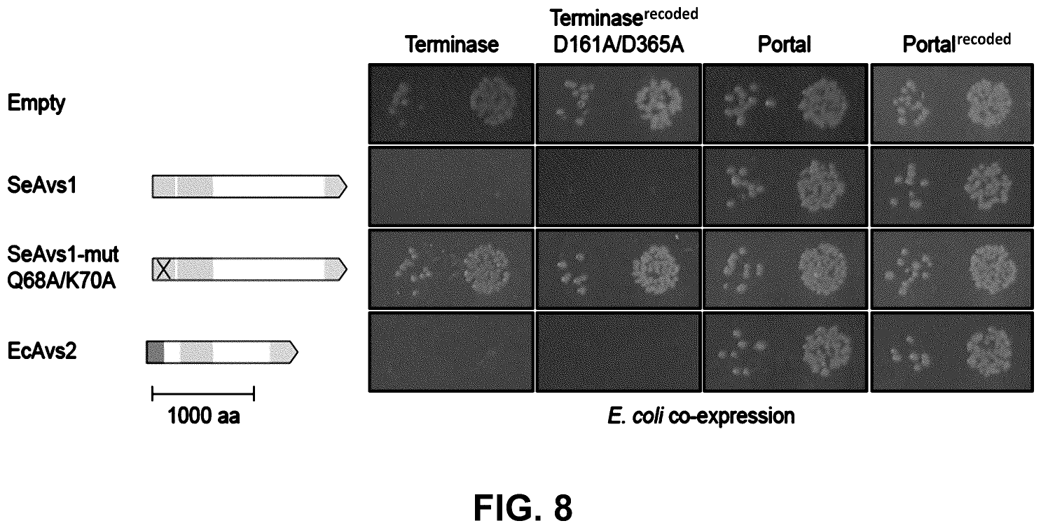

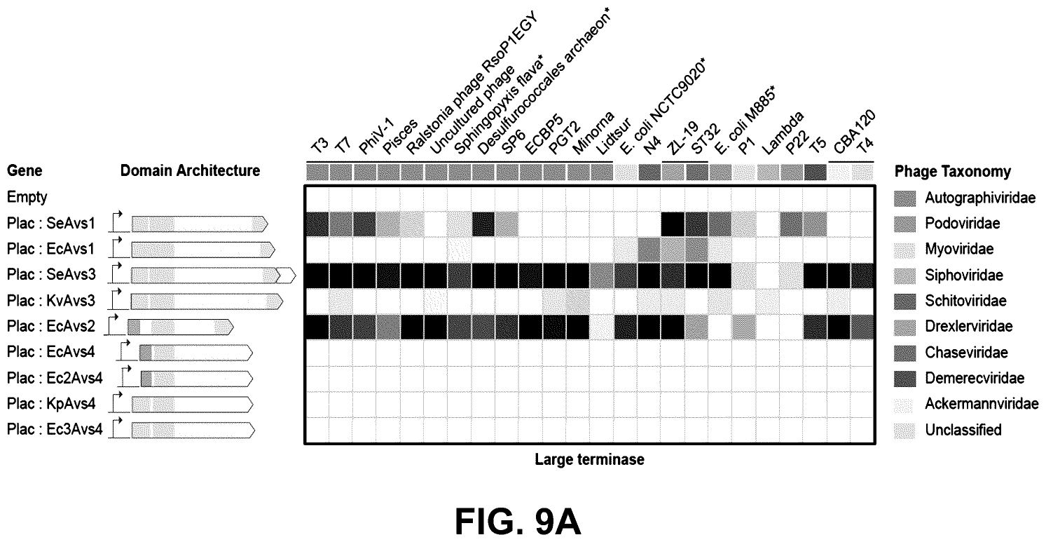

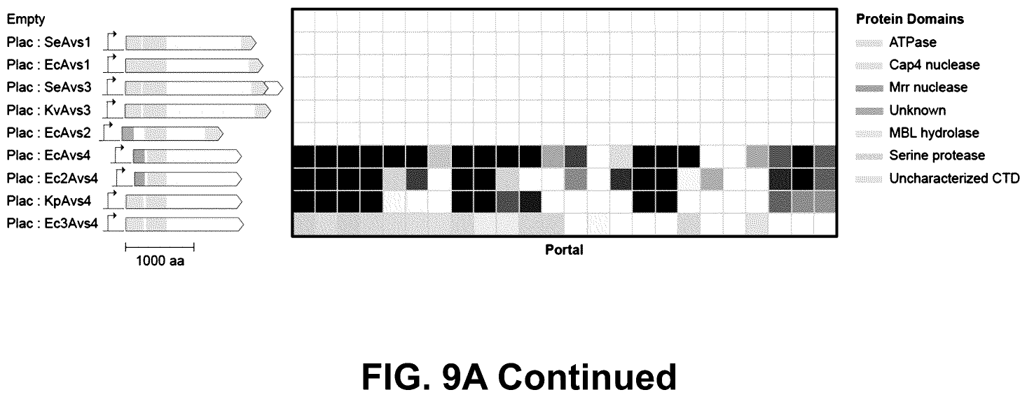

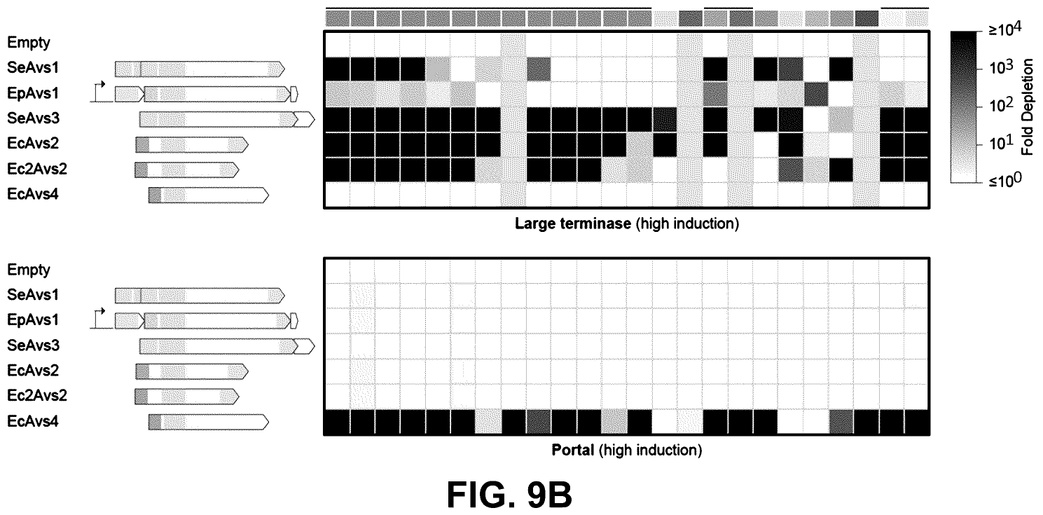

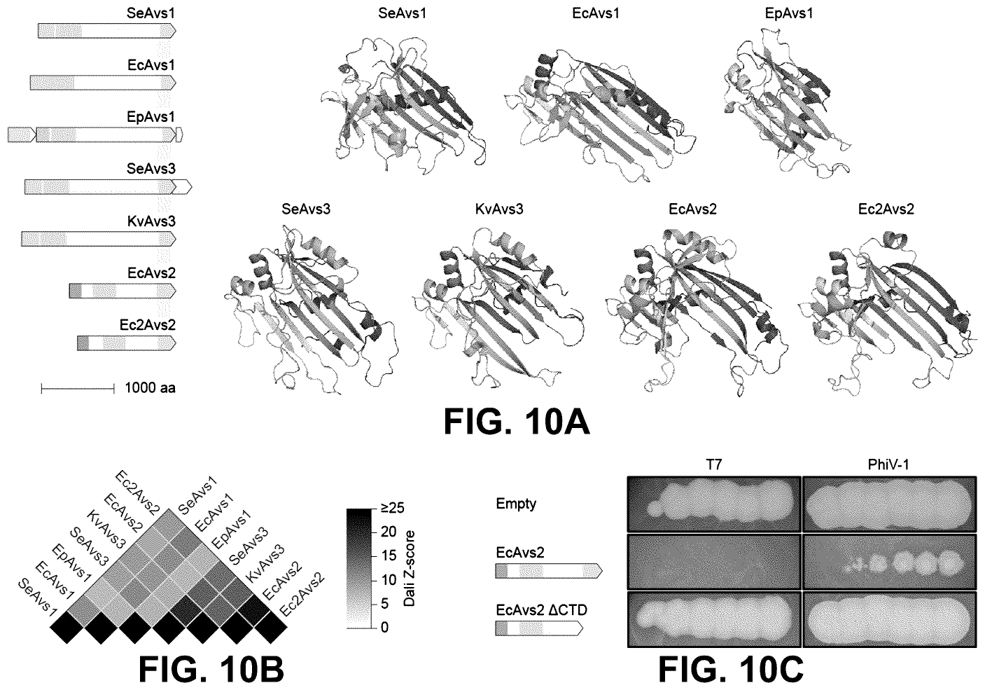

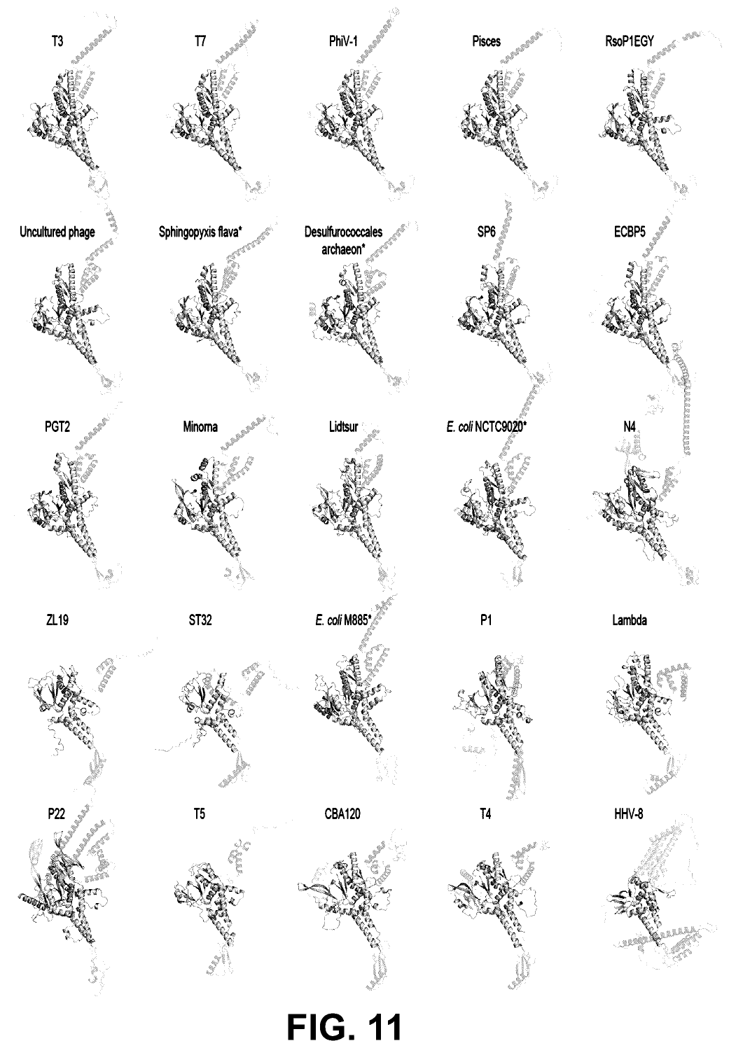

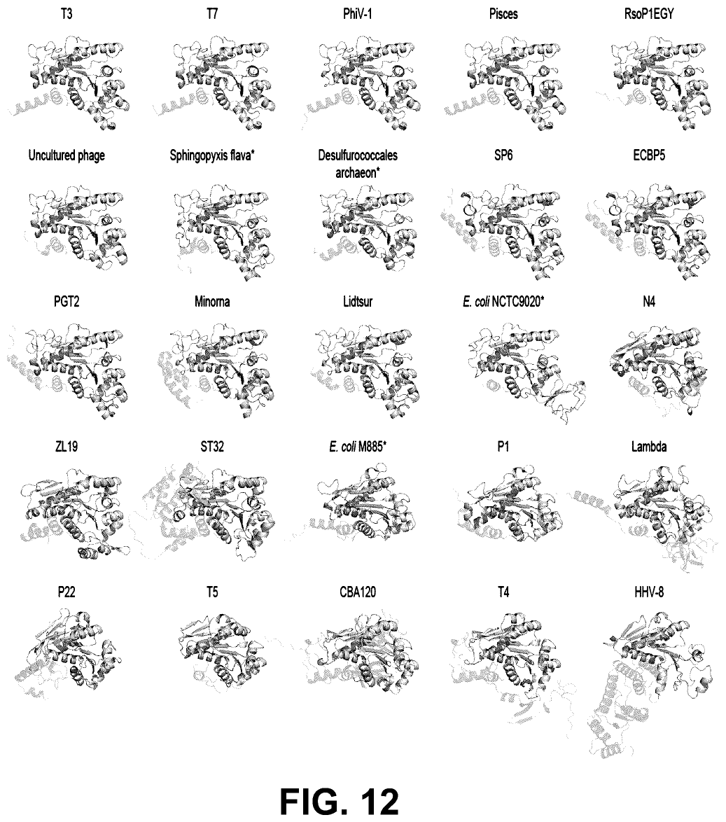

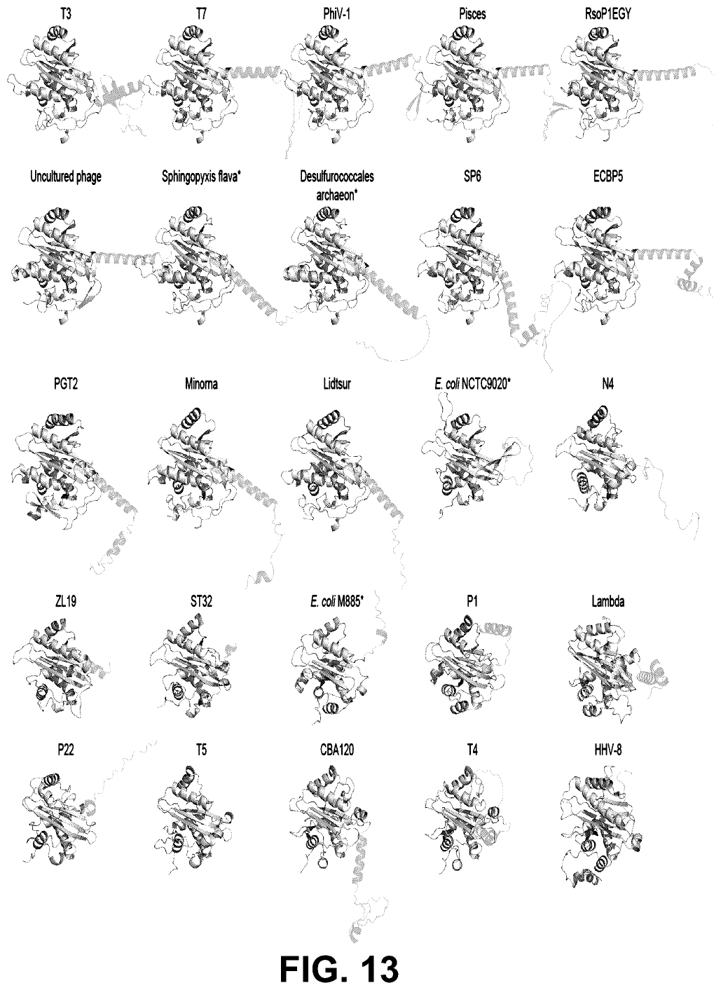

To investigate the specificity of Avs activation, Applicants cloned the portal and large terminase subunit genes from 24 tailed phages, spanning nine major phage families, and co-expressed these genes in E. coli with 12 Avs systems spanning all four Avs families (Tables 1-2 and FIG. 4A-4B). Applicants quantified cellular toxicity and depletion of specific Avs-phage protein pairs for all 576 combinations by deep sequencing (FIG. 7A and Data S2). These experiments revealed precise target specificity: Avs1-3 were activated only by large terminase subunits, whereas Avs4 recognized only the portal protein (FIG. 7B and FIG. 8). To assess the robustness of the assay, Applicant repeated these experiments varying the Avs promoter or the amount of terminase and portal induction, obtaining similar results (FIG. 9A-9B). Surprisingly, Avs1 and Avs2 also recognized terminases despite the lack of substantial sequence similarity among the C-terminal TPRs of Avs1, Avs2, and Avs3, although Applicant detected a structurally similar domain at the end of the TPR arrays in all three proteins (FIG. 10A-10C). These findings demonstrate remarkable conservation of target recognition across Avs families and suggest that the portal and large terminase subunit are key PAMPs recognized by prokaryotic NLR homologs. Moreover, Avs systems recognize PAMPs from diverse phages; for example, SeAvs3 and EcAvs2 are strongly activated by 20 of 24 and 19 of 24 tested terminases, respectively, and EcAvs4 is strongly activated by 15 of 24 tested portals (>100-fold depletion) (FIG. 3F). Because the portals and terminases from different phage families have limited sequence similarity, with less than 5% pairwise sequence identity in some cases (FIG. 7C), but share the same core fold (FIGS. 11-13), this broad range of activity implies that Avs proteins are triggered by conserved structural features rather than by specific peptide sequences. Consistent with this idea, EcAvs2 and EcAvs4 displayed weak but clear recognition of the terminase and portal, respectively, of human herpesvirus 8 (FIG. 7D), which is a highly diverged evolutionary derivative of tailed phages (24) and does not infect prokaryotes.

### SeAvs3 and EcAvs4 are Phage-Activated DNA Endonucleases

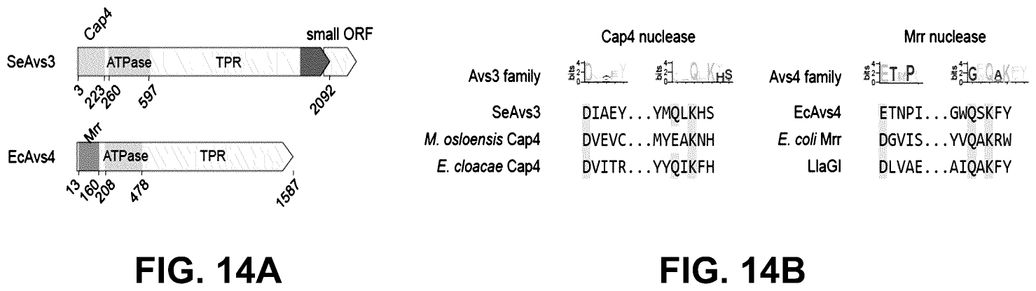

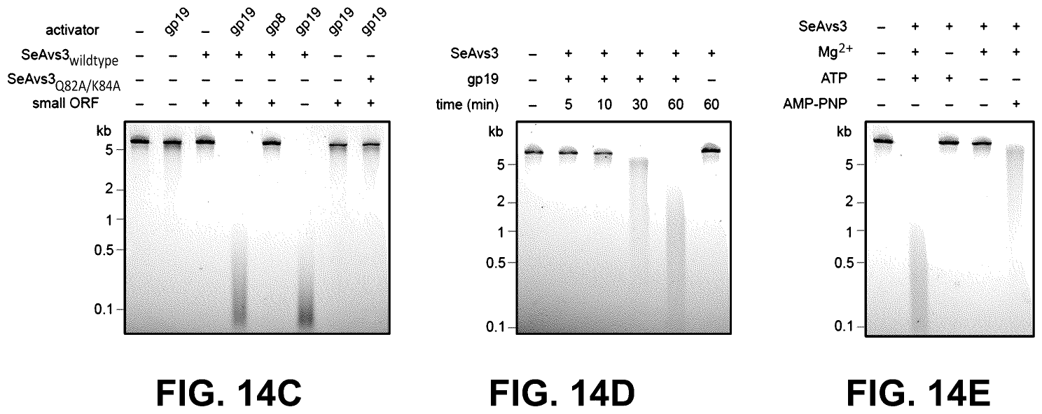

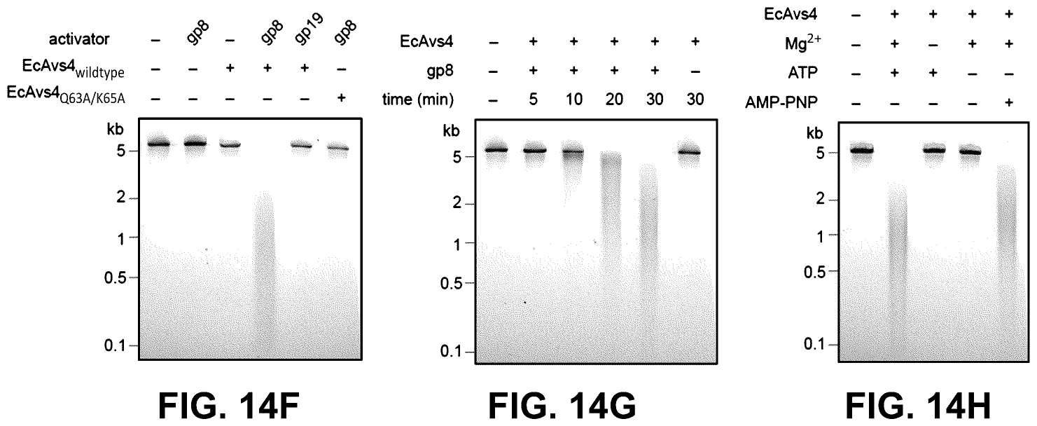

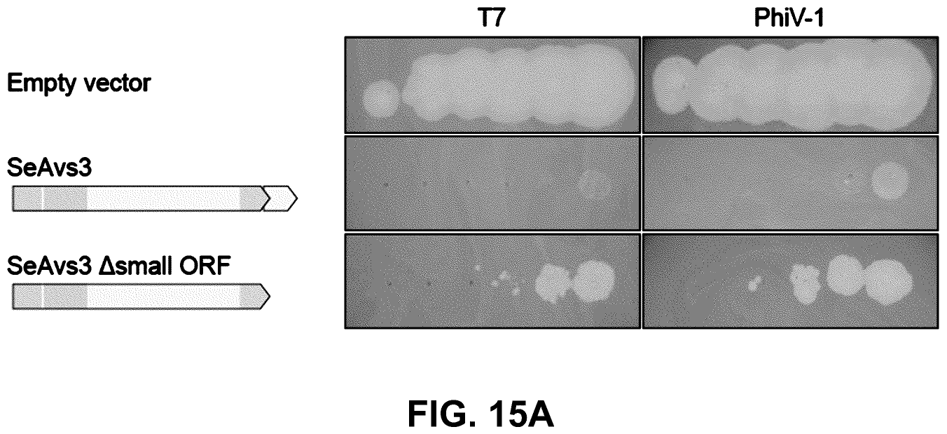

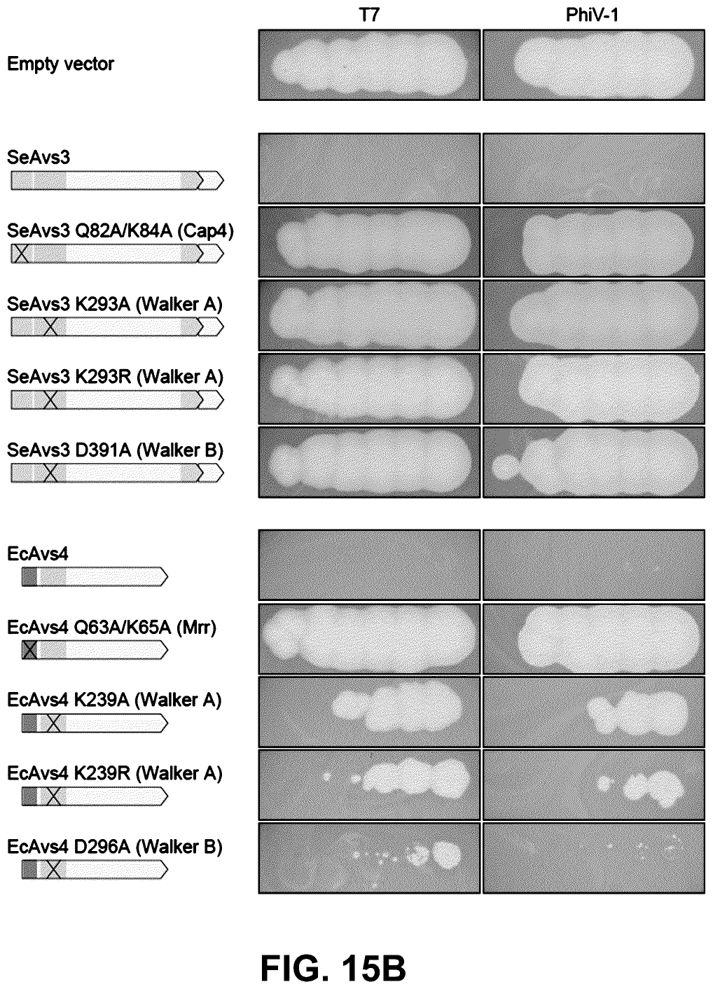

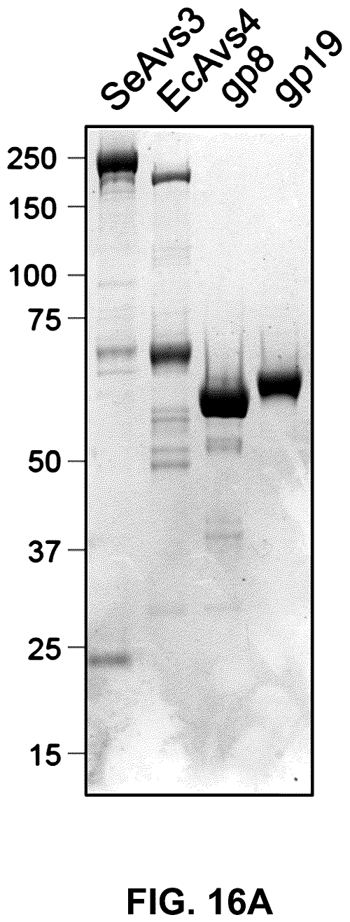

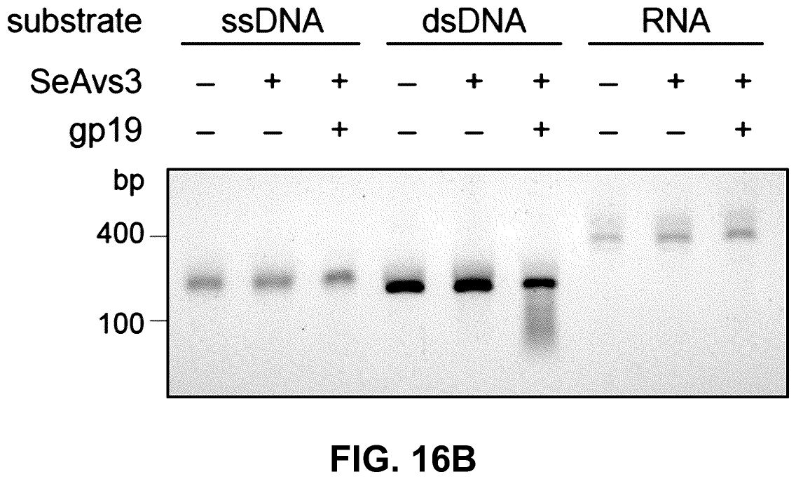

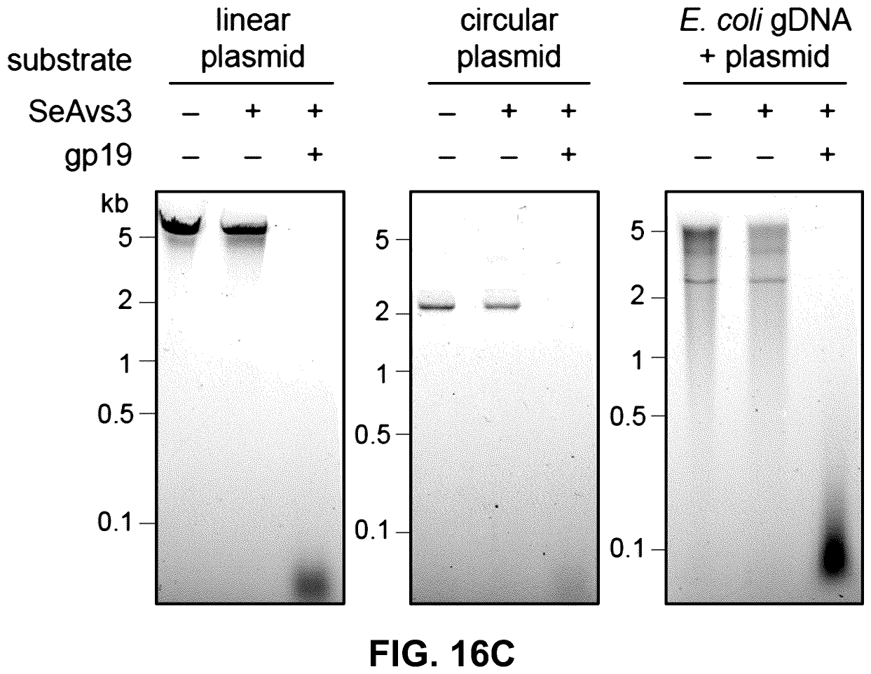

SeAvs3 and EcAvs4 contain predicted N-terminal PD-DExK-family 139 nuclease domains (FIG. 14A), which Applicant hypothesized degrade phage and cellular DNA upon target recognition. The nuclease domain of SeAvs3 is most similar to the recently reported Cap4 effector nucleases of cyclic-oligonucleotide based defense systems (25, 26), whereas the nuclease domain of EcAvs4 is related to Mrr-like restriction endonucleases (27). Both Avs proteins contain conserved D-QxK or E-QxK catalytic motifs (FIG. 14B), and in addition to the STAND NTPase gene, the SeAvs3 system contains a small ORF, the deletion of which reduced antiphage activity in E. coli (FIG. 15A). Applicant sought to biochemically reconstitute Avs activity in vitro, and Applicant purified recombinant SeAvs3, the protein encoded by the small ORF, EcAvs4, and the PhiV-1 portal (gp8) and terminase (gp19) proteins (FIG. 16A). Applicant incubated SeAvs3 and the small ORF product with linear double stranded DNA (dsDNA) and observed progressive degradation of the substrate in the presence of gp19, but not gp8 (FIG. 14C-14D). This nuclease activity was dependent on the catalytic residues of SeAvs3 but did not require the small ORF product (FIG. 14C). Applicant further investigated the substrate specificity of Avs systems and found that the nuclease activity was specific for dsDNA, whereas ssDNA and RNA were not cleaved (FIG. 16B). Moreover, SeAvs3 cleaves both linear and circular dsDNA, including E. coli genomic DNA (FIG. 16C), indicative of endonuclease activity with no specificity for phage DNA, which is consistent with an abortive infection defense mechanism.

Applicant next investigated cofactor requirements of SeAvs3 and found that in vitro activity depends on both Mg²⁺ and adenosine triphosphate (ATP); however, ATP hydrolysis is not strictly required because nuclease activity was observed at a reduced level in the presence of the nonhydrolyzable ATP analogue AMP-PNP (FIG. 14E). Applicant also reconstituted the nuclease activity of EcAvs4 and found that it is activated by gp8, but not by gp19, and was abolished in a Q63A/K65A (Gln⁶³→Ala/Lys⁶⁵→Ala) EcAvs4 mutant (FIG. 14F-14G). Similar to SeAvs3, nuclease activity required the presence, but not the hydrolysis, of ATP (FIG. 14H), consistent with phage plaque assays of SeAvs3 and EcAvs4 ATPase active-site mutants (FIG. 15B). Together, these experiments indicate that SeAvs3 and EcAvs4 are promiscuous DNA endonucleases that are activated by distinct, highly conserved phage proteins in an ATP-dependent manner.

### Structural Basis for Avs Binding and Target Recognition

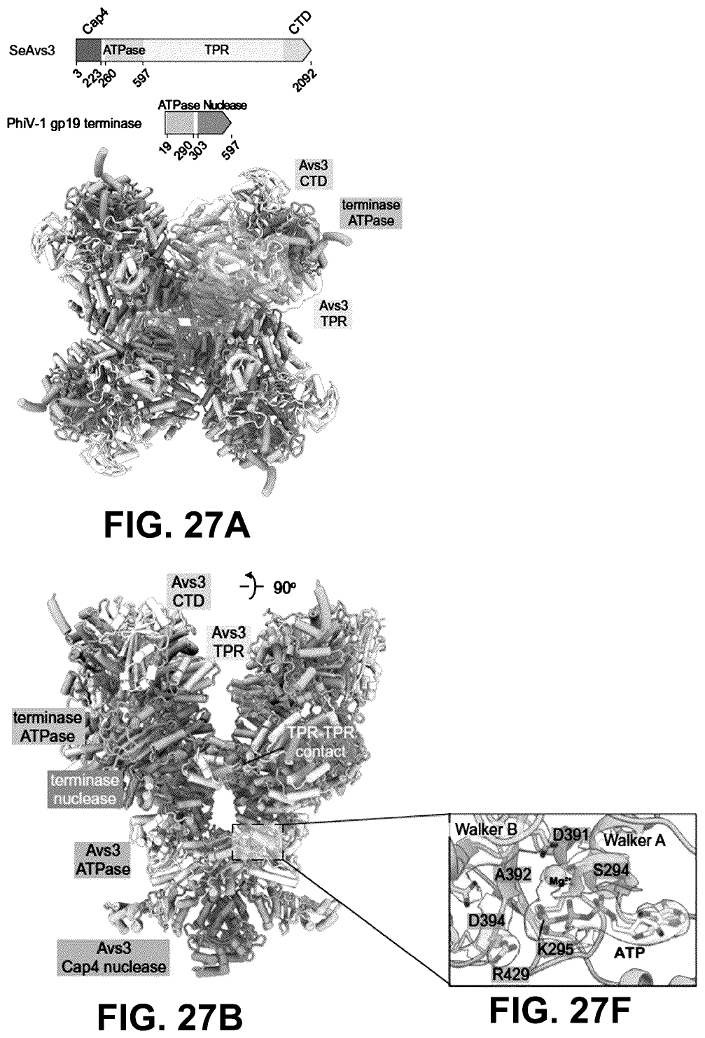

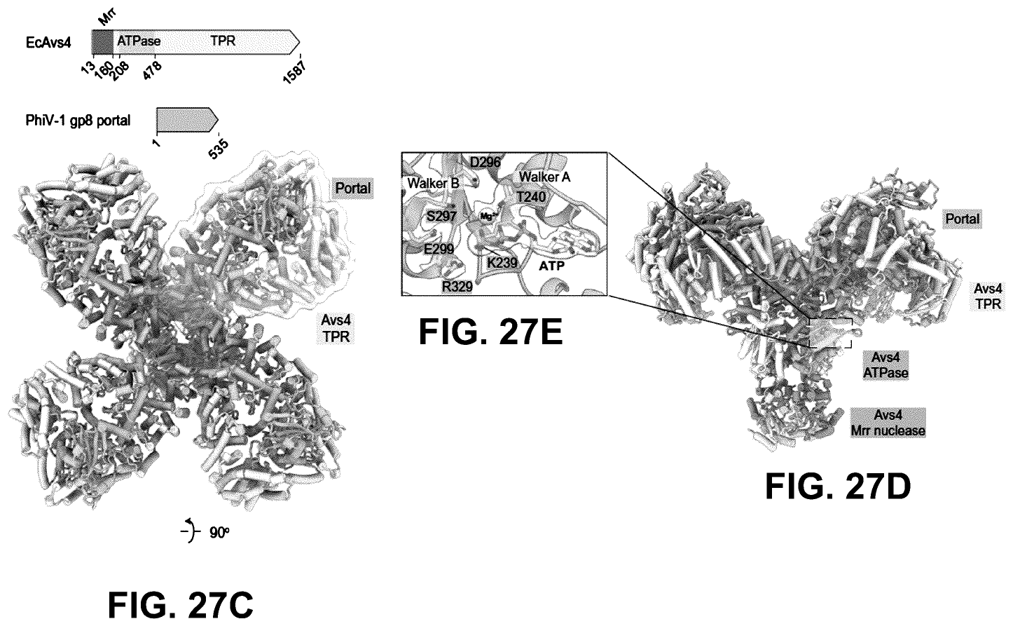

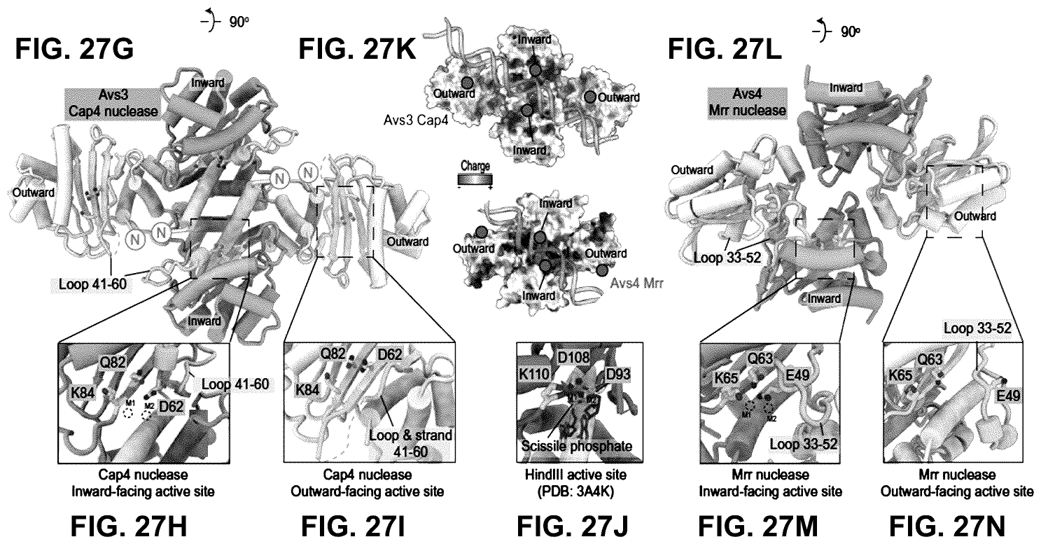

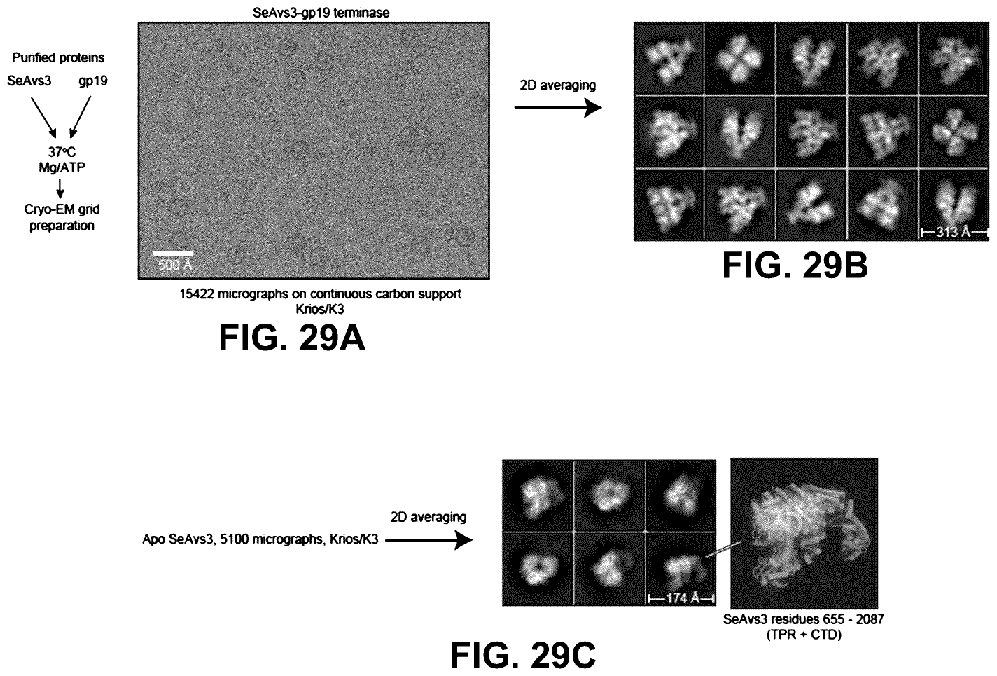

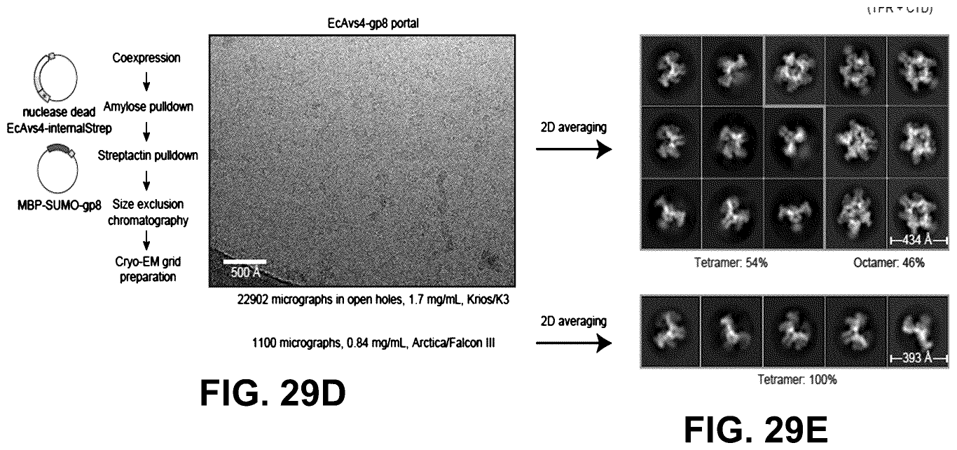

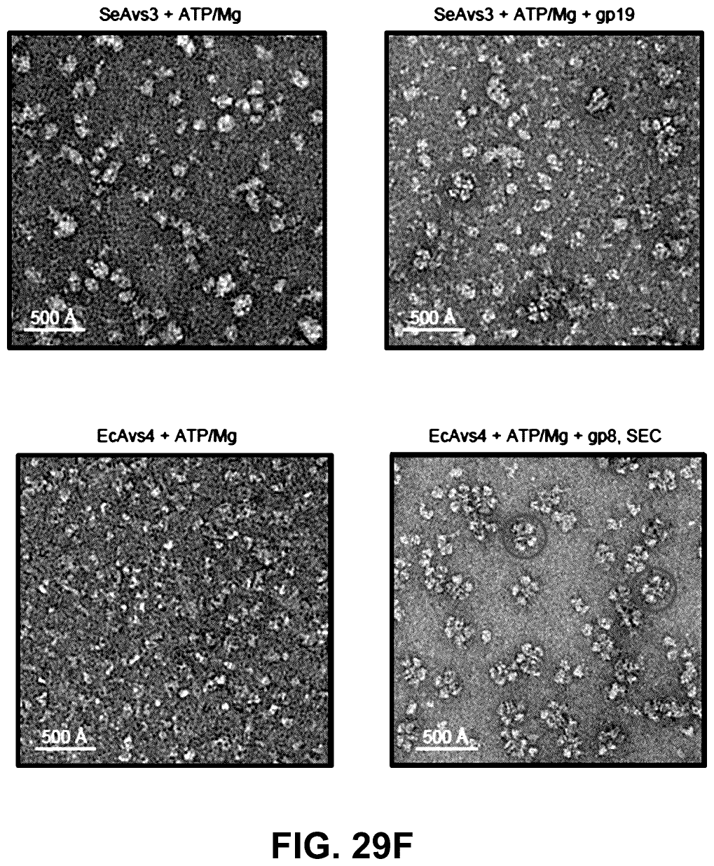

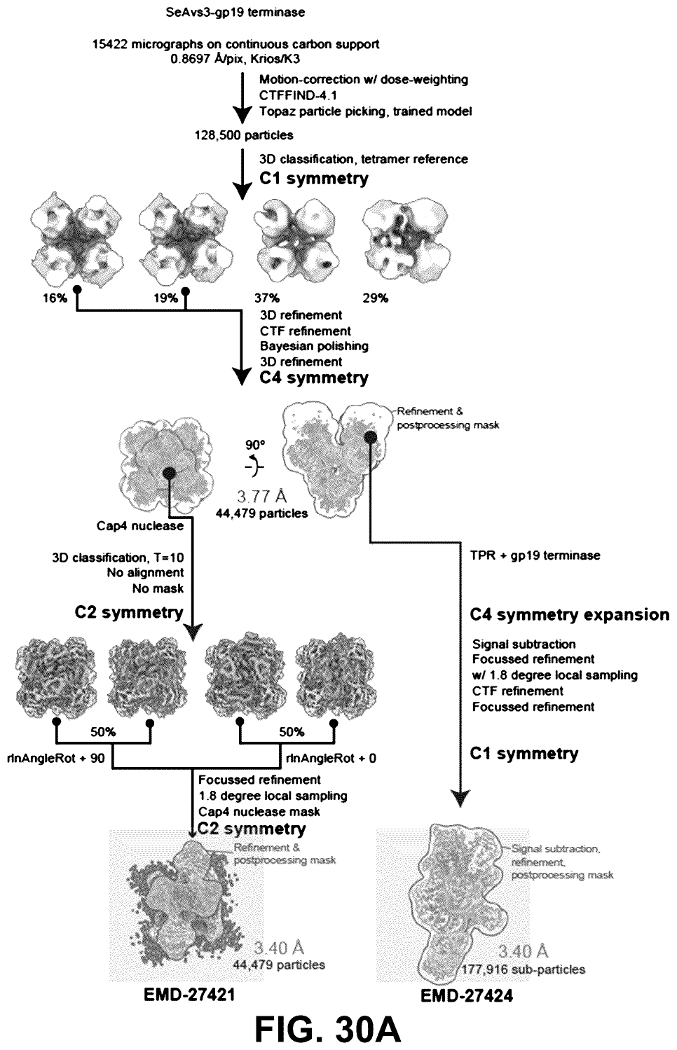

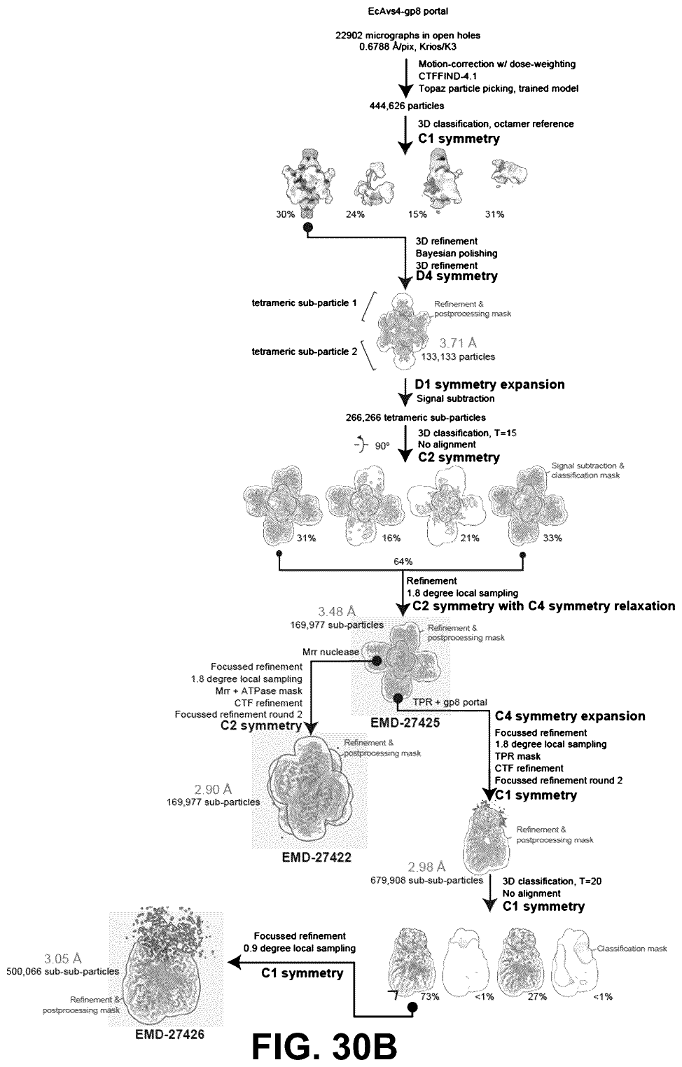

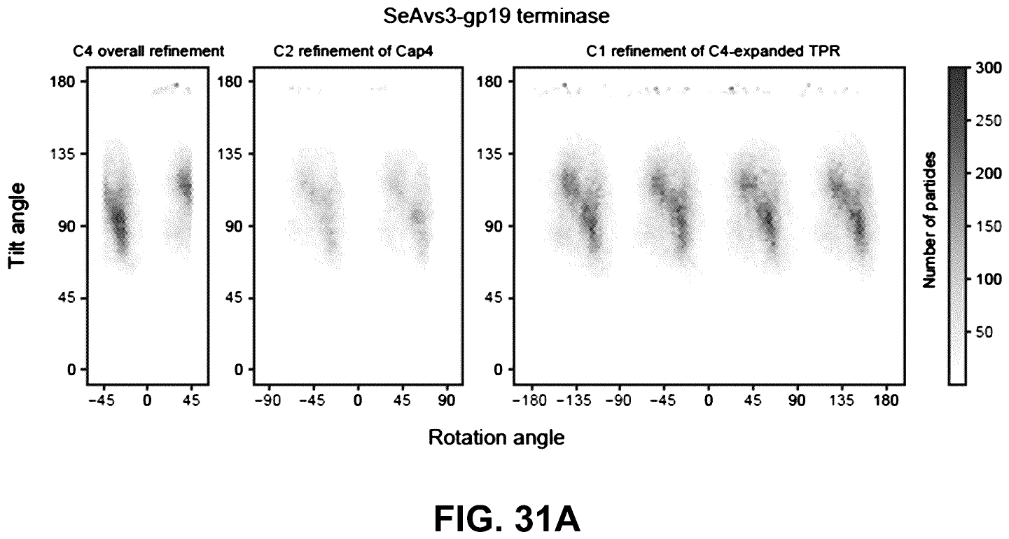

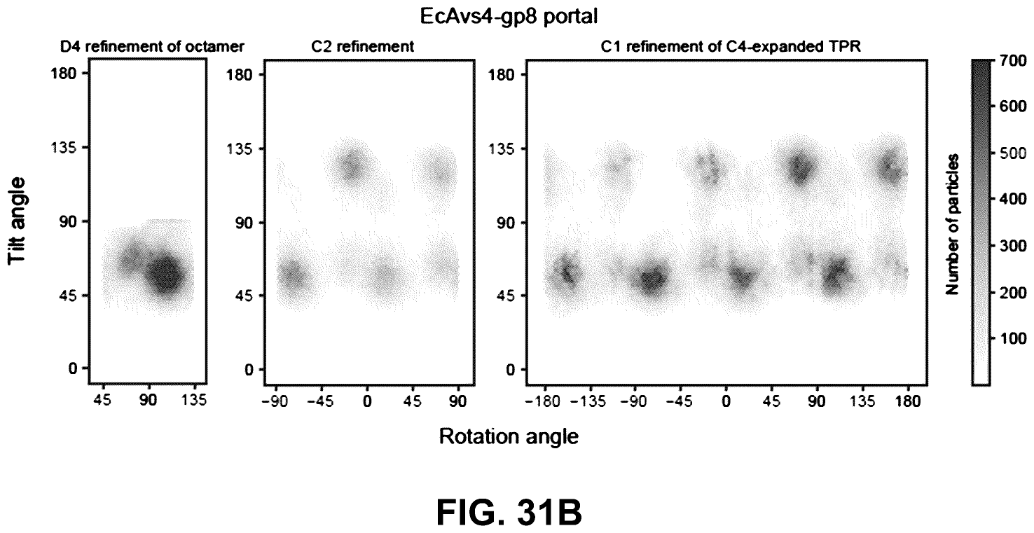

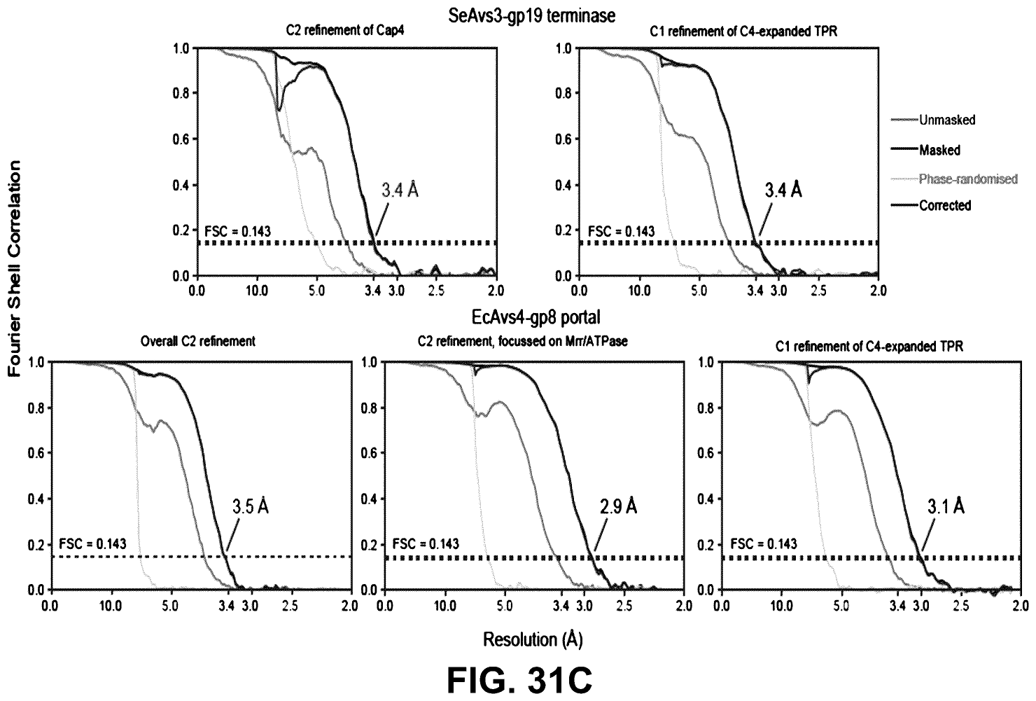

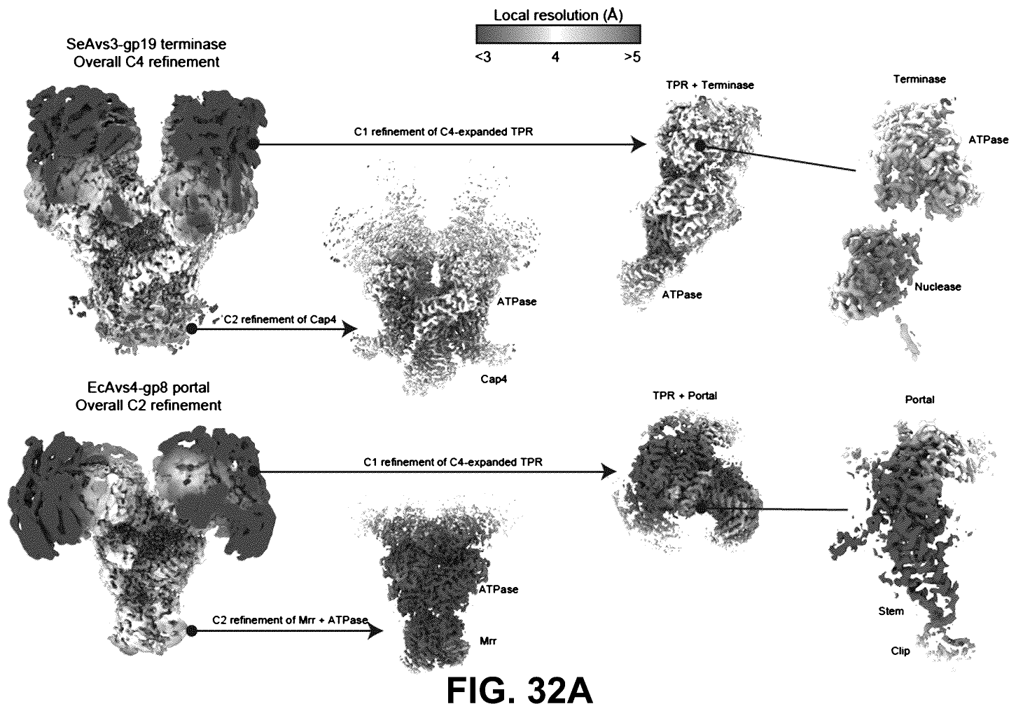

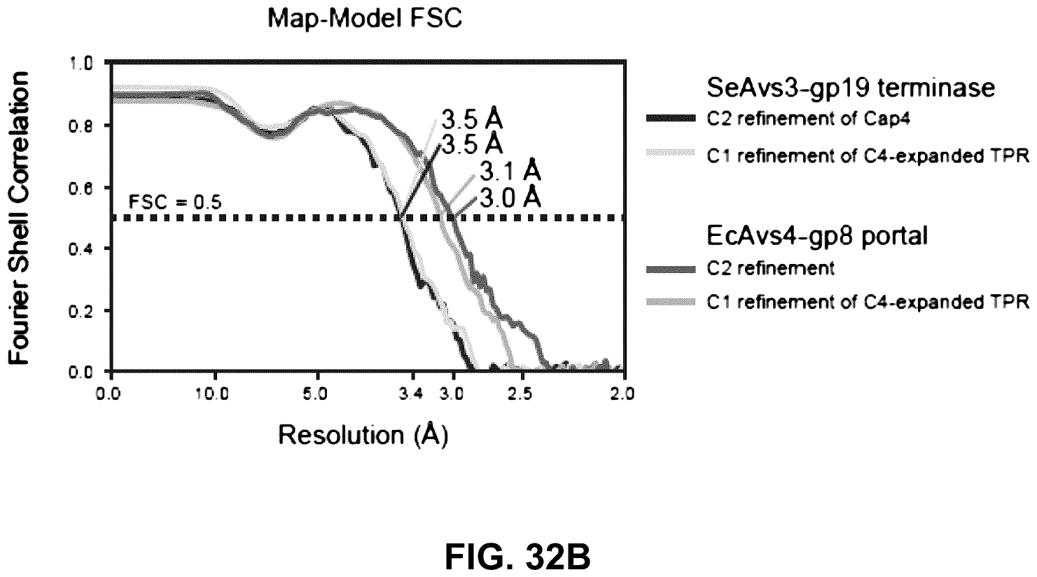

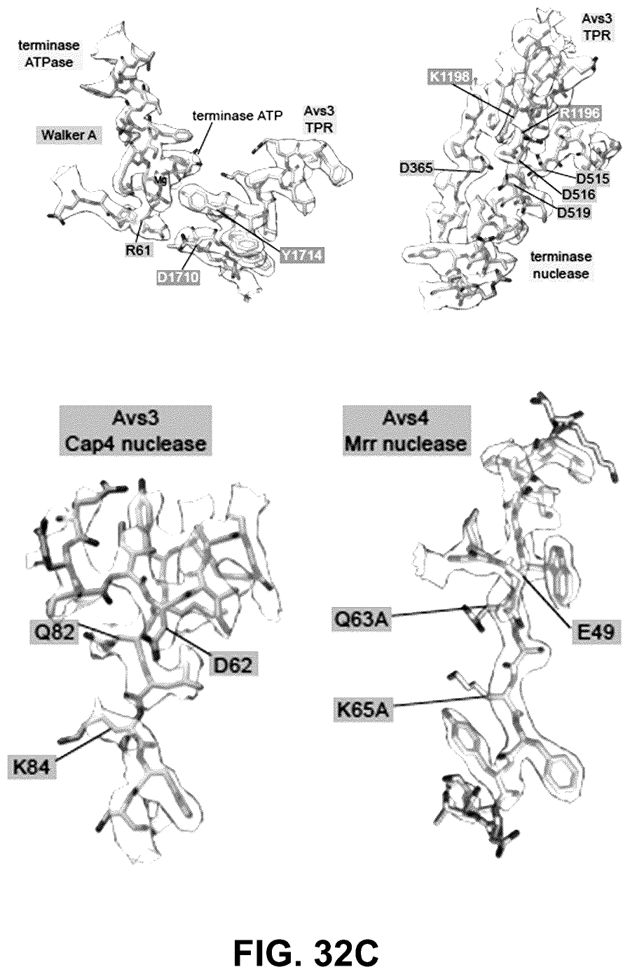

To investigate how Avs systems recognize and bind their cognate phage proteins, Applicant solved cryo-electron microscopy (cryo-EM) structures of the SeAvs3-terminase and EcAvs4-portal complexes in the presence of ATP and Mg²⁺ (FIGS. 29A-29F, 30A-30B, 31A-31C, and 32A-32C and Table S1 of Gao et al., Science, 377, eabm4096 (2022), which is incorporated herein by reference as if expressed in its entirety herein). A reconstruction at 3.4-Å resolution revealed that SeAvs3 forms a tetramer, with each C-terminal TPR domain gripping the ATPase and nuclease domains of the gp19 terminase (FIG. 27A-27B). These TPR lobes are flexible and required symmetry expansion to improve their local resolution to 3.4 Å (see Materials and Methods discussed herein and FIGS. 30A-30B, 31A-31C, and 32A-32C) For EcAvs4 bound to the PhiV-1 gp8 portal (FIG. 27C-27D), image processing revealed equal abundances of a teterameric complex and an octameric complex corresponding to tetramer head-to-head dimerization (FIG. 29A-29F). At lower protein concentrations, however, Applicant observed only the tetramer, indicating that it most likely the functional complex (FIG. 29A-29F). Negative-stain and cryo-EM imaging of SeAvs3 and EcAvs4 in the absence of phage proteins revealed only smaller monomeric particles (FIG. 29A-29F), indicating that phage protein binding is required for the assembly of SeAvs3 and EcAvs4 into tetramers.

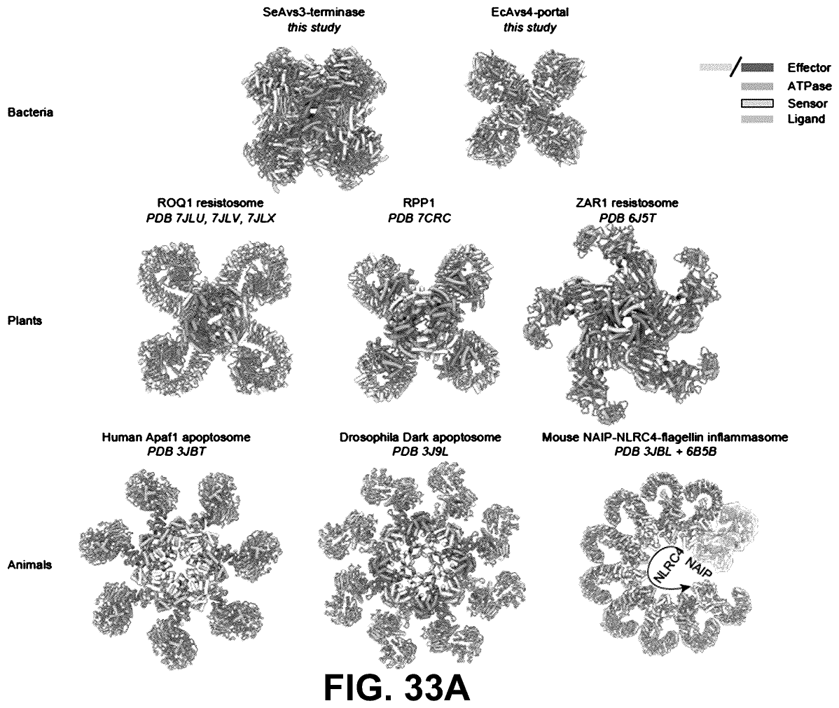

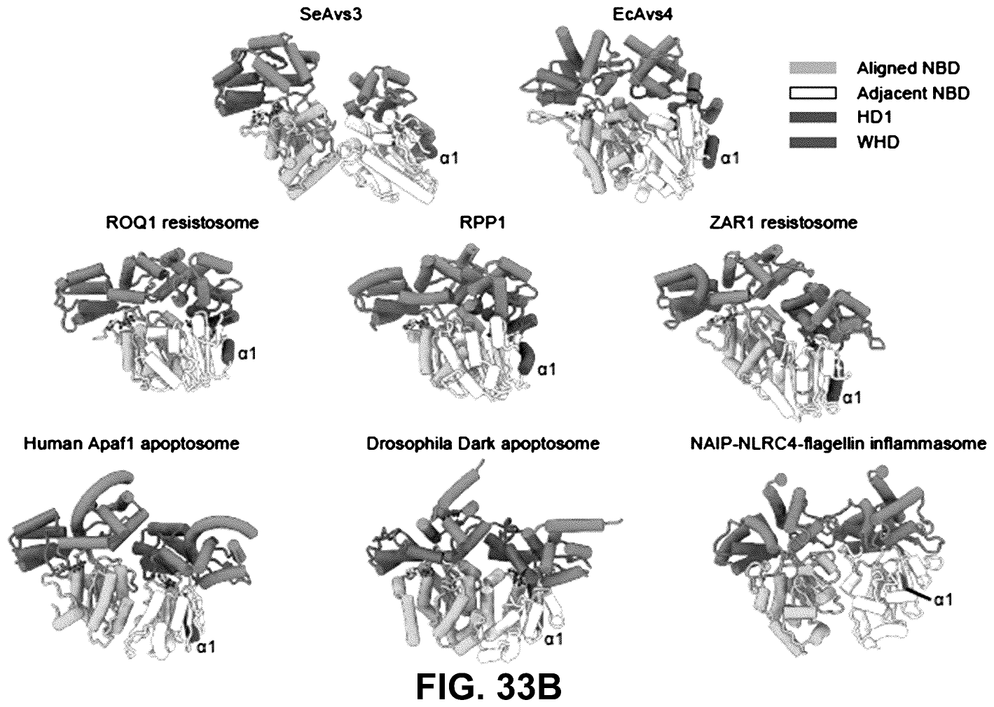

Tetramerization of both SeAvs3 and EcAvs4 is mediated through their STAND ATPase domains, which interact in a manner distinct from each other and from other characterized STAND ATPase oligomers like Roq1 resistosome tetramer (59) or the Apaf1 apoptosome heptamer (60) (FIG. 33A-33B). The SeAvs3 ATPase domain forms a C4-symmetric tetramer by interactions between the nucleotide-binding domain (NBD) and winged-helix domain (WHD) subdomains and the NBD subdomain of the adjacent protomer, whereas the EcAvs4 ATPase domain forms a C2-symmetric dimer of dimers with a tighter interface (1232 Å² of buried surface area, compared to 436 Å² of for SeAvs3), with adjacent WHDs and NBDs both interacting (FIG. 33A-33B). The smaller interface in SeAvs3 is compensated for by additional contacts between its C-terminal TPR domains (FIG. 27A-27B). SeAvs3 and EcAvs4 both maintain ATP in their active sites with and adjacent magnesium ion coordinated by the canonical Walker A and B motifs (FIG. 27E-27F). Notably, in both cases, tetramerization of the STAND ATPAse domains brings adjacent N-terminal nuclease domains close together, forming two nuclease dimers with overall C2 symmetry (FIGS. 27G and 27L).

SeAvs3 and EcAvs4 contain nuclease effectors of the PD-DExK superfamily. Conventional PD-DExK nucleases (e.g., restriction endonucleases) use a pair of acidic residues to coordinate at least one metal ion and a conserved lysine residue to bind the scissile phosphate and stabilize the transition state for nucleolytic cleavage (FIG. 27J) (60, 61). In the SeAvs3 Cap4 tetramer, this arrangement of residues is found in all four promoters; however, in the two “outward-facing” protomers, an extended β strand makes a steric block for DNA binding and/or metal coordination (FIG. 27I). FIG. 27H shows Cap4 nuclease inward-facing active site. Based on the crystal structure of the Hind III restriction endonuclease (62), the inward-facing protomers can be predicted to form a cavity for DNA binding, with each protomer likely cleaving opposite strands of the DNA (FIG. 27K). The EcAvs4 Mrr tetramer shows a similar principle, whereby the two inward-facing protomers contain active sites that resemble canonical PD-DExK nucleases, but in the outward-facing protomers, Glu⁴⁹, which is part of the conserved trio of active-site residues, is displaced (FIG. 27M-27N). Glu⁴⁹ is found in the loop that spans residues 33 to 52, and interactions between this loop on an “inward” protomer and an adjacent “outward” protomer likely stabilize its position in the inward protomer. Like the SeAvs3 Cap4 tetramer, these two inward protomers form a cavity that accommodates DNA in a manner similar to HindIII (FIG. 27K).

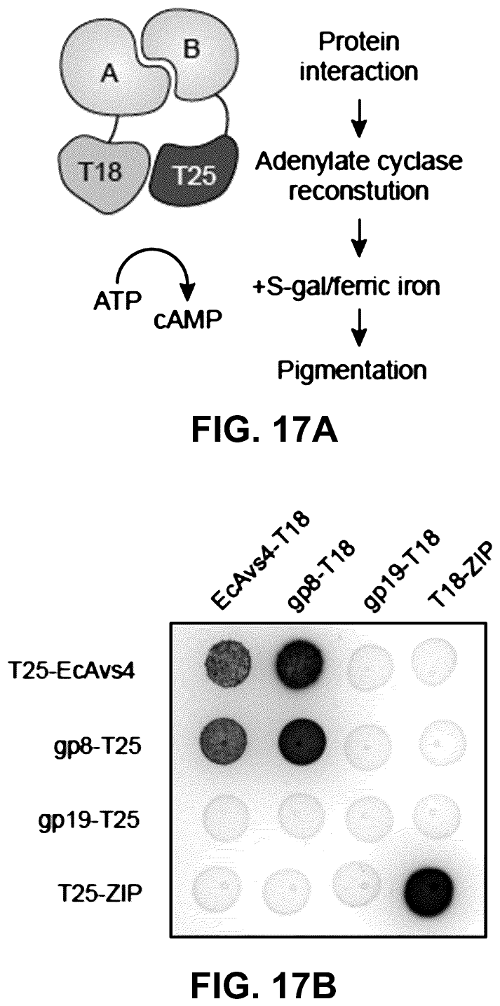

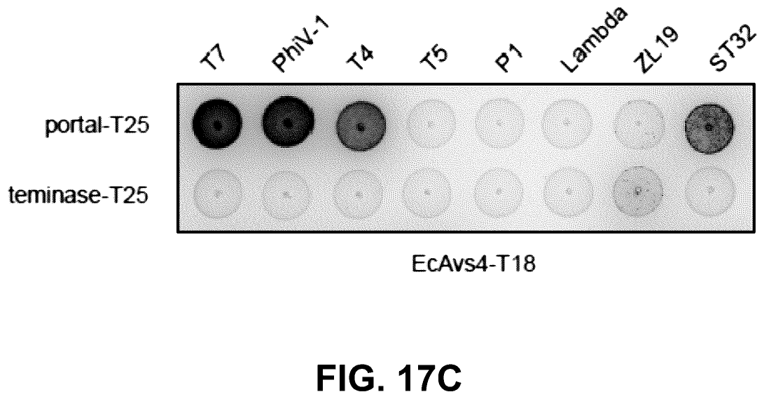

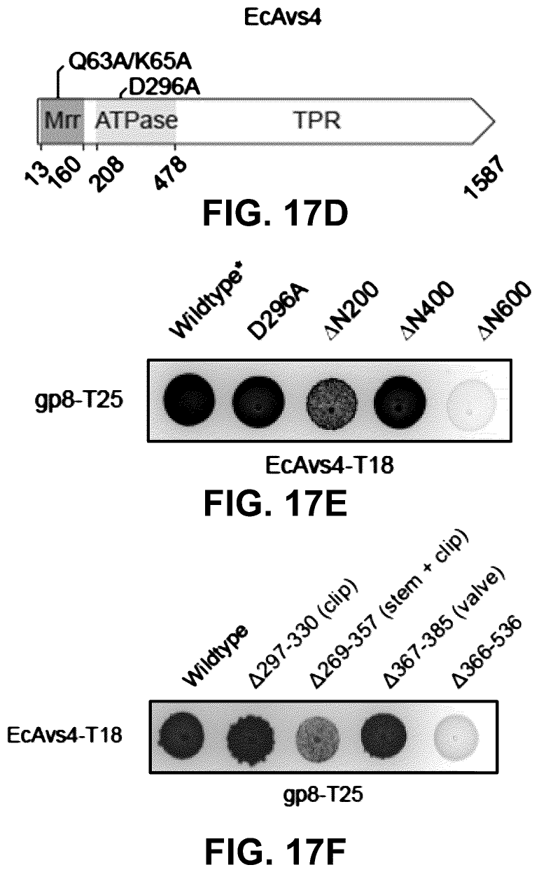

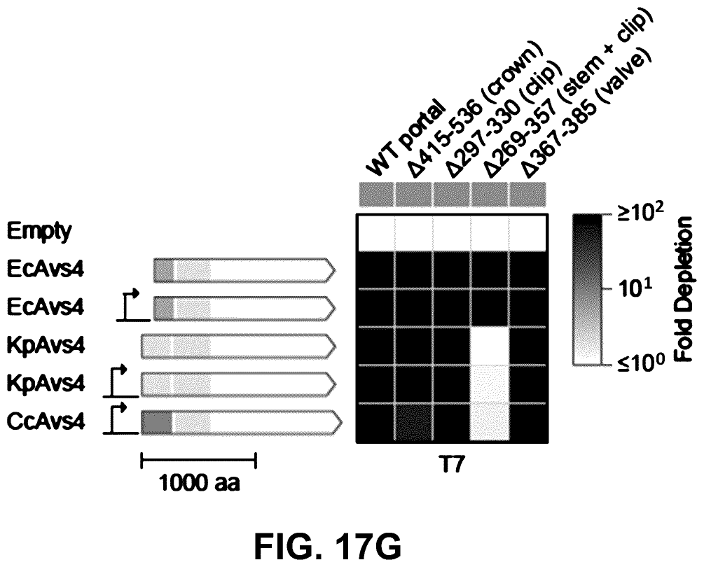

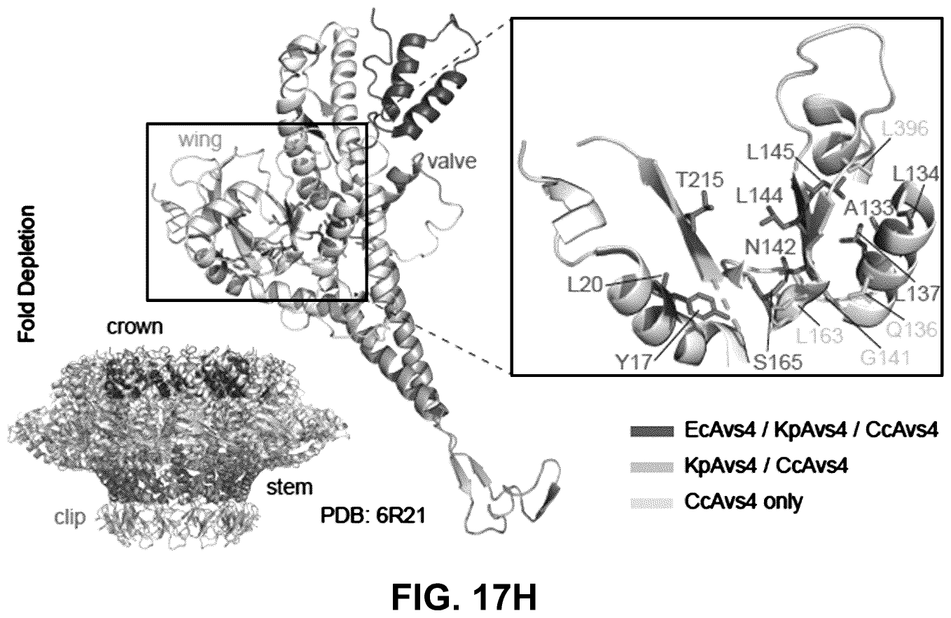

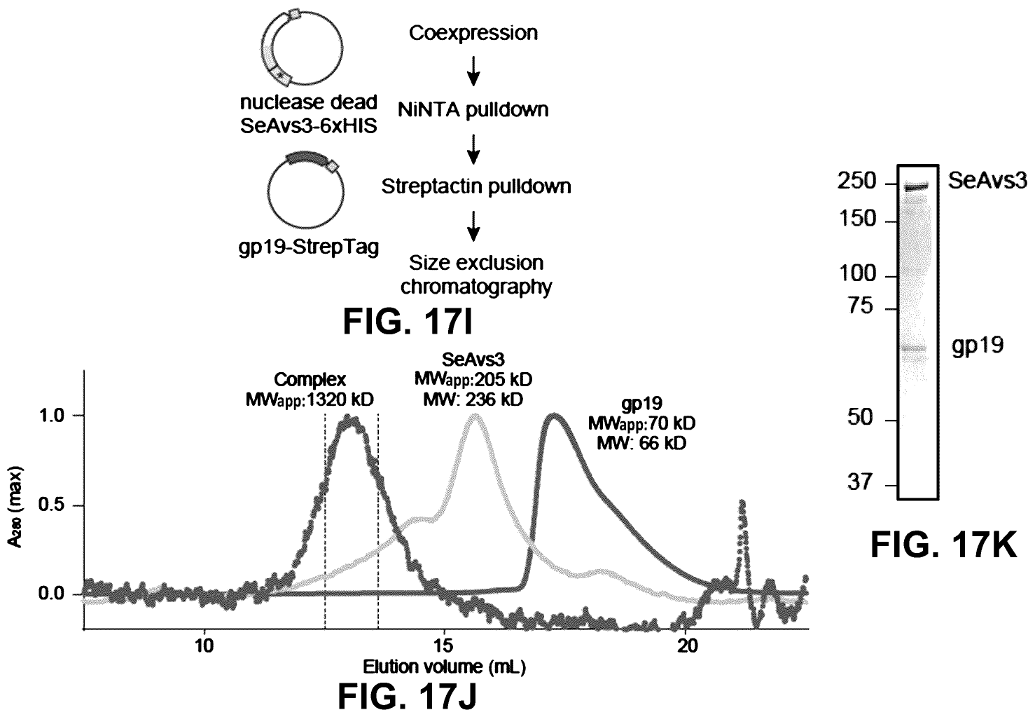

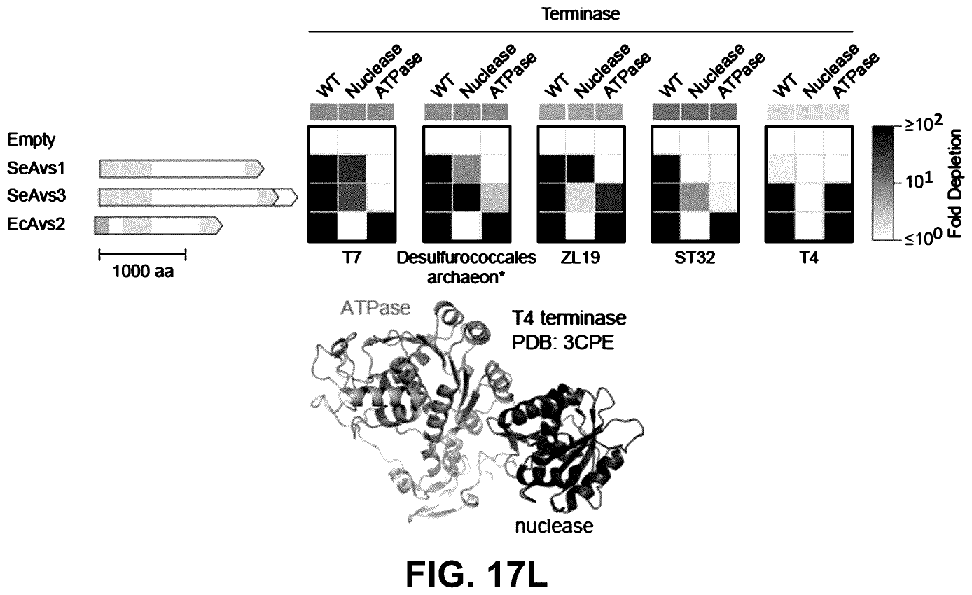

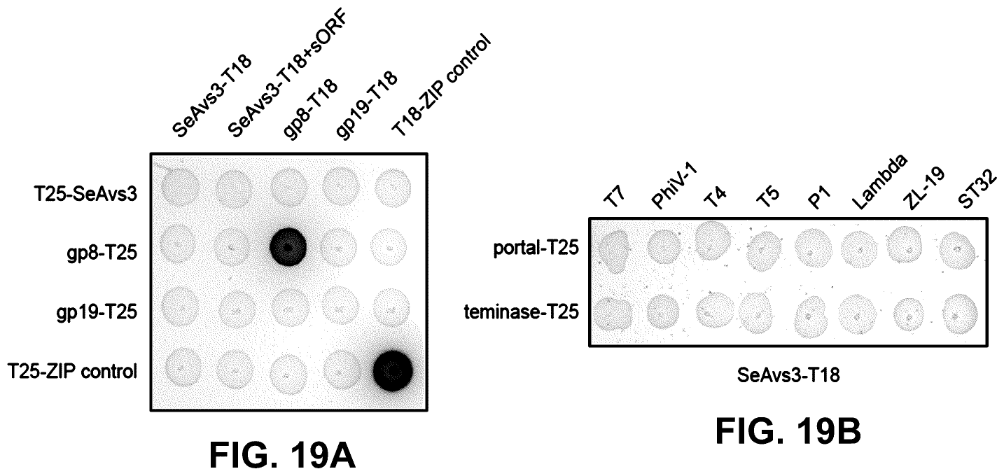

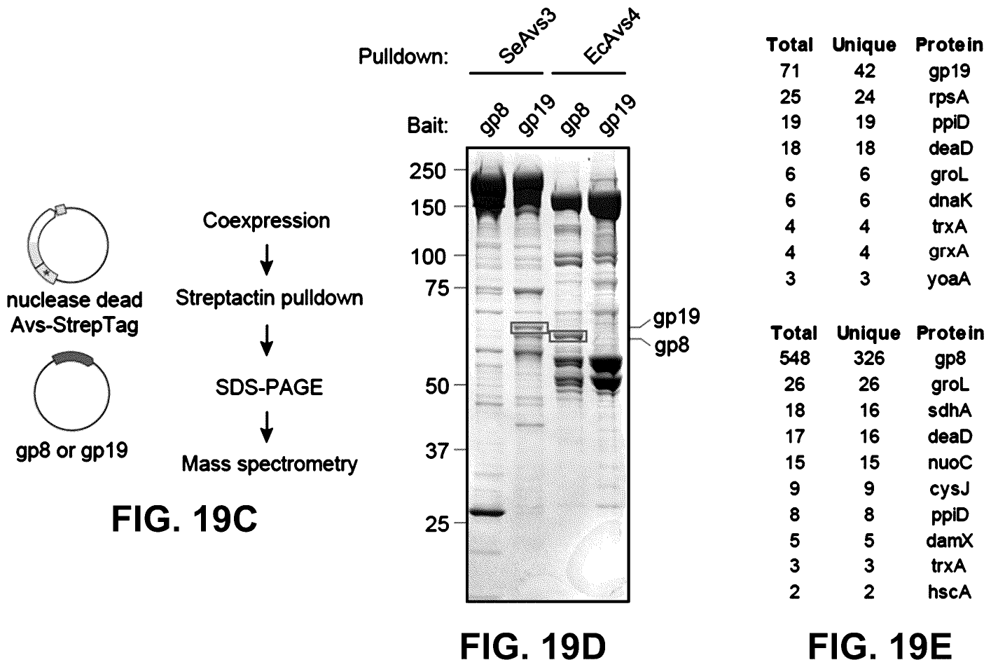

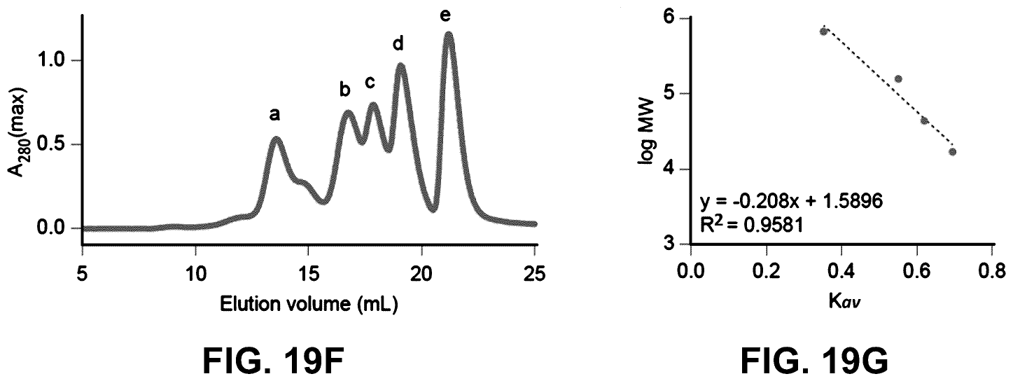

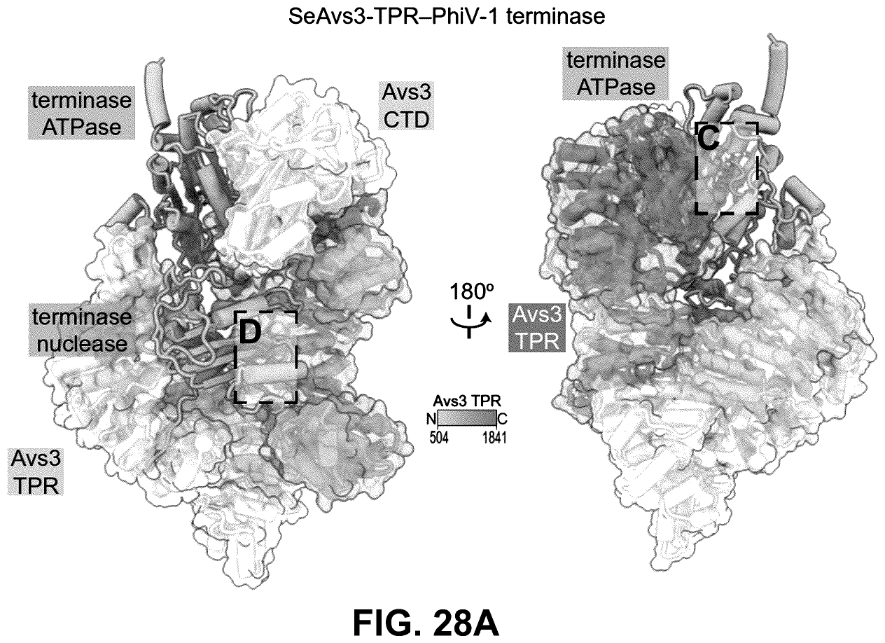

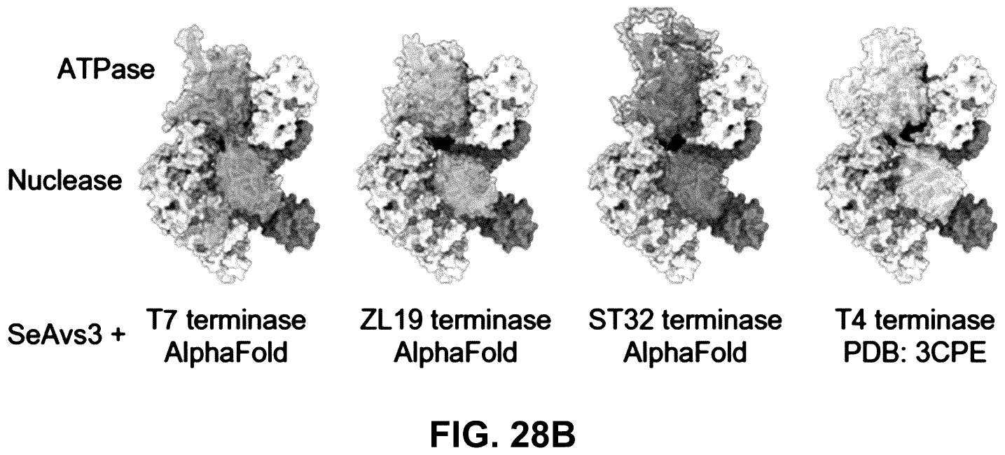

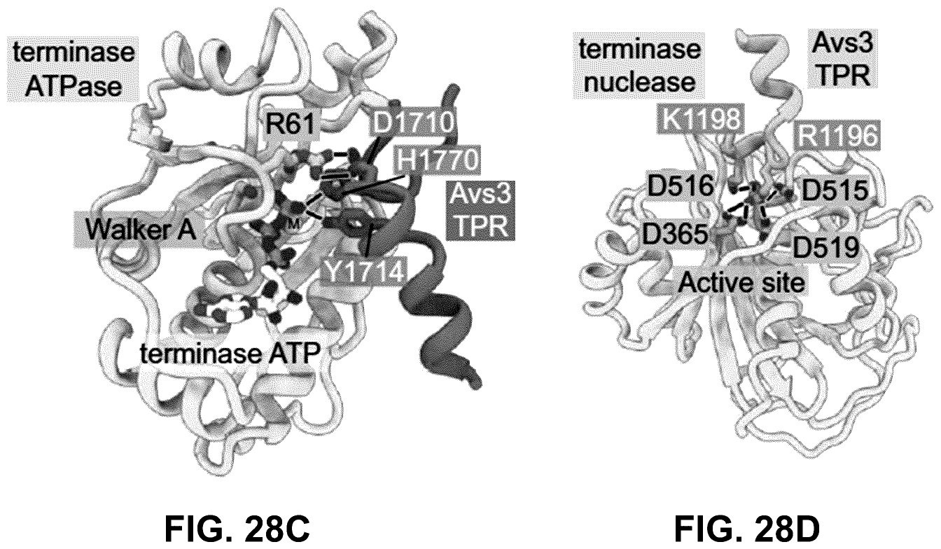

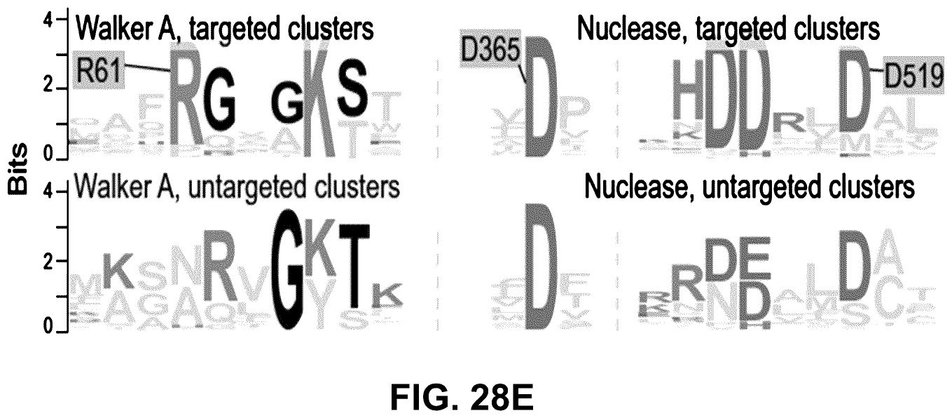

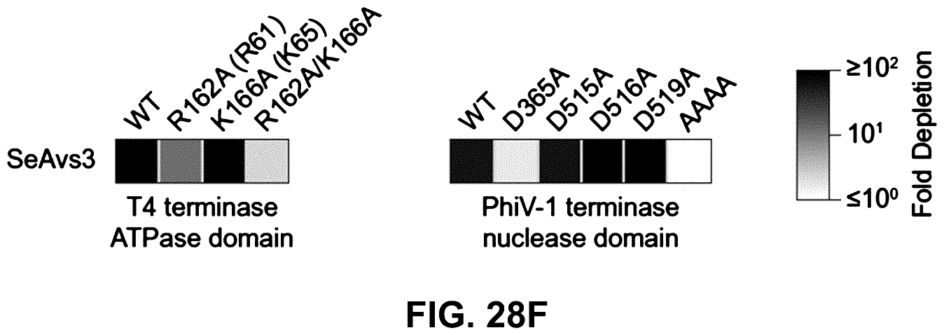

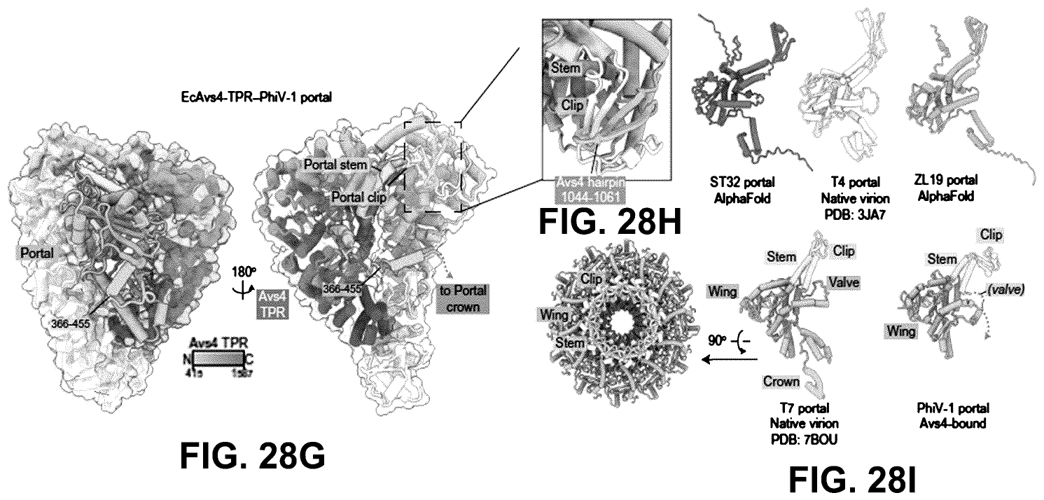

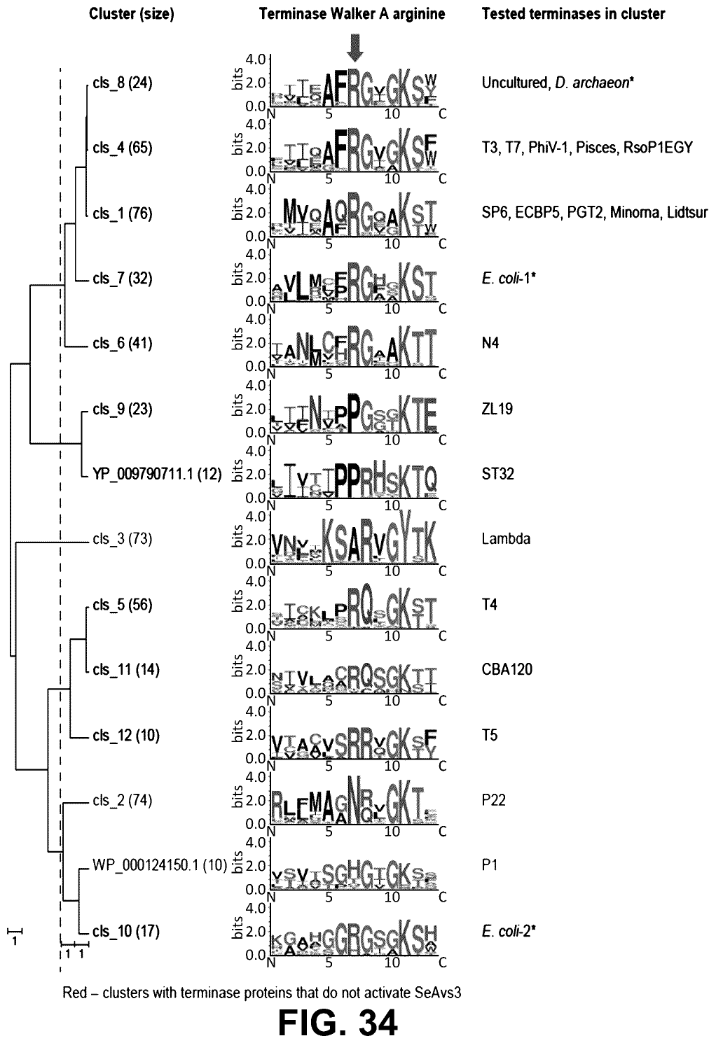

SeAvs3 and EcAvs4 both contain extensive TPR domains for binding their cognate phage proteins, which we confirmed using a bacterial two-hybrid system and protein copurification (FIGS. 17A-17F, 17I-17K, and 19A-19G). The SeAvs3 TPR domain forms a left-hand-like structure capped by a b sheet-rich C-terminal domain (FIG. 28A). This domain has two cavities in which the terminase ATPase and nuclease domains are nestled. Consistent with the ability of SeAvs3 to bind terminases with little sequence similarity (FIG. 7C), there are few specific residue-residue pair contacts between SeAvs3 and the PhiV-1 terminase. Instead, binding is deter-mined by shape and charge complementarity between the two proteins, burying more than 3700 Å2 of solvent-accessible surface area. This complementarity is maintained across a diverse range of experimental structures and AlphaFold models of phage terminases (FIG. 28B). Additionally, SeAvs3 directly recognizes residues within the two terminase active sites. In particular, Asp¹⁷¹⁰ in SeAvs3 forms a salt bridge with the highly conserved Arg⁶¹ within the Walker A motif of the terminase ATPase (FIGS. 28C and 28E). An arginine in this position is found in most terminase ATPases that activate SeAvs3 but not in nonactivating terminases (FIGS. 28E and 34). These observations suggest that Arg⁶¹ within the Walker A motif is a determinant of recognition specificity, and indeed, mutation of the cognate arginine in the T4 terminase ATPase domain substantially reduced SeAvs3 activation by the ATPase domain (FIG. 28F). Notably, an arginine is not typically found in this position in endogenous cellular ATPases (62), suggesting a possible mechanism for avoiding off-target activation. Furthermore, Arg¹¹⁹⁶ and Lys¹¹⁹⁸ in SeAvs3 form salt bridges to the four conserved aspartates that make up the active site of the terminase nuclease (FIG. 28D-28E), and mutation of Asp³⁶⁵ in the PhiV-1 terminase nuclease notably reduced SeAvs3 activation by the nuclease domain (FIG. 28F). Thus, SeAvs3 directly reads the active-site residues of both domains of the terminase. Furthermore, the ATP ligand bound by the terminase is detected by interactions between the gamma phosphate and His¹⁷⁷⁰ and Tyr¹⁷¹⁴ of SeAvs3 (FIG. 28C). Targeting this ligand presumably helps avoid phage escape mutations, because ATP binding is required for the function of the terminase.

Because SeAvs3 detects both domains of the terminase, Applicant hypothesized that there might be some functional redundancy in these interactions. Indeed, SeAvs3 was activated by the nuclease domain alone from some phages, including T7, but was also activated by the ATPase domain alone from T4 and ZL19, a T1 family phage (FIG. 17L). Likewise, SeAvs1 was activated by the nuclease domain from T7, but in the case of ST32, both the nuclease and ATPase domains were required. These results suggest that Avs1 and Avs3 recognize both the nuclease and ATPase domains but differ in the extent of activation by either domain, depending on the terminase. By contrast, deletion of the nuclease domain had no impact on Avs2 activity for any of the five tested terminases, suggesting that Avs2 recognizes the ATPase domain only (FIG. 17L). This pattern of recognition is consistent with the larger size of Avs1 and Avs3 compared with Avs2.

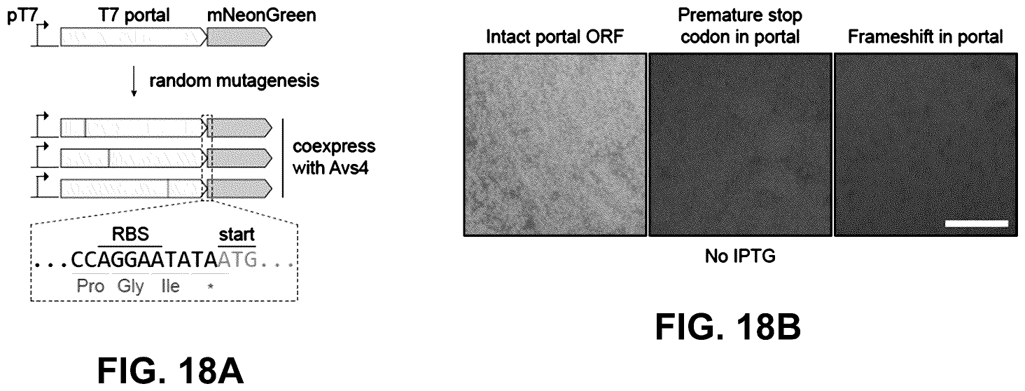

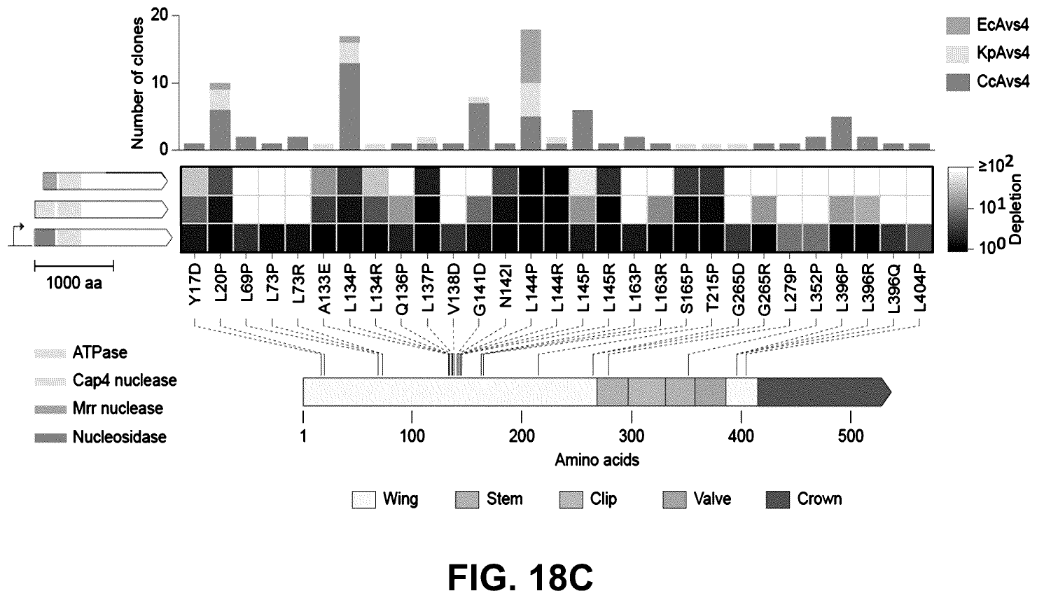

The TPR domain of EcAvs4 also binds the PhiV-1 portal with a large interface, burying 5800 Å2 of solvent-accessible surface area, that includes notably few residue-residue contacts (FIG. 28G). The portal protein is recognized through its stem, clip, and part of its wing domain. In an assembled dodecameric portal complex, these regions are found toward the interior and are therefore more constrained in their fold requirements (FIG. 28I). Consistent with this observation, Applicant performed random mutagenesis by polymerase chain reaction (PCR) to screen for portal mutations that abrogate Avs4 activation and found that all 29 identified mutations were nonconservative and located in the core wing or stem regions (FIGS. 17G-17H, 18A-18C), possibly disrupting the core portal fold. The clip domain, which contains a conserved antiparallel b sheet with an intervening a helix, is recognized by β-sheet augmentation with a hairpin of EcAvs4 (FIG. 28H), a mode of fold recognition that does not depend on the amino acid sequence of the target. Because portal proteins are not enzymes, there are no active-site residues to target as in the SeAvs3-terminase complex. Finally, portal oligomerization is not compatible with the Avs4-bound state (FIG. 28H),suggesting that Avs4 recognizes portal monomers before they assemble into the procapsid.

### Avs Proteins are Widespread in Prokaryotes and Possess Diverse, Modular N-Terminal Effector Domains

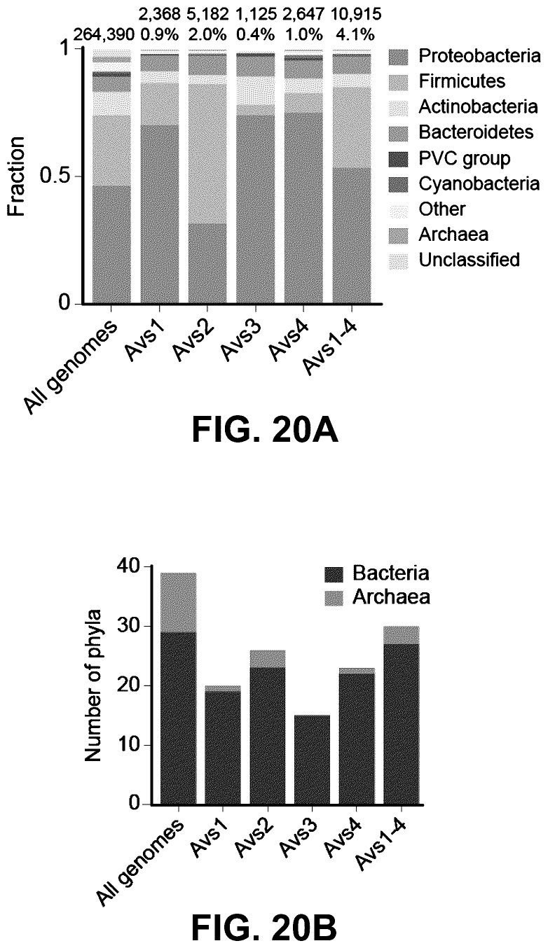

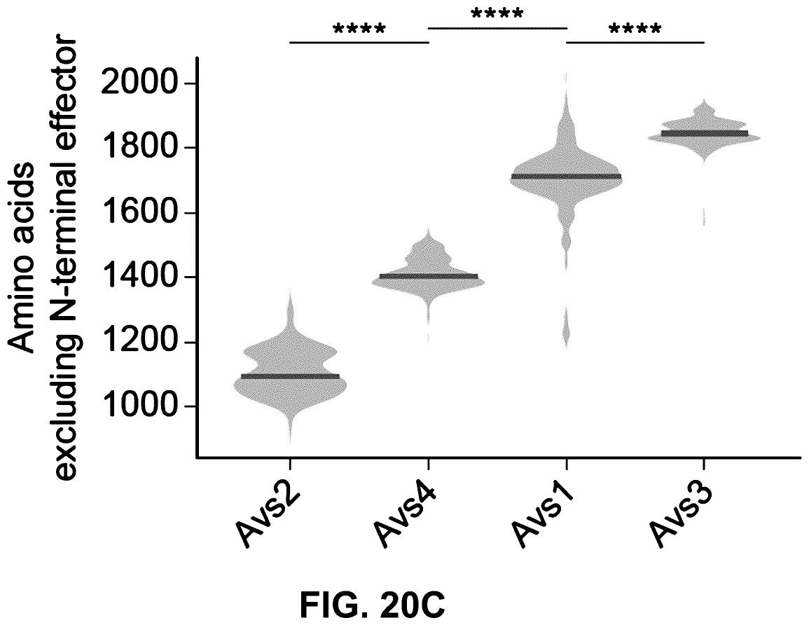

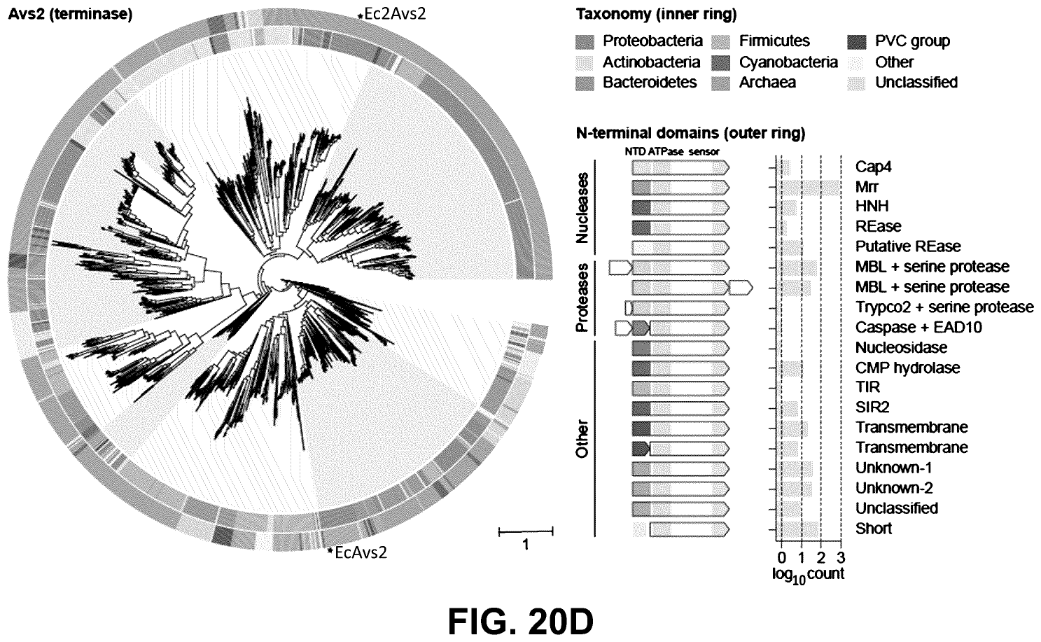

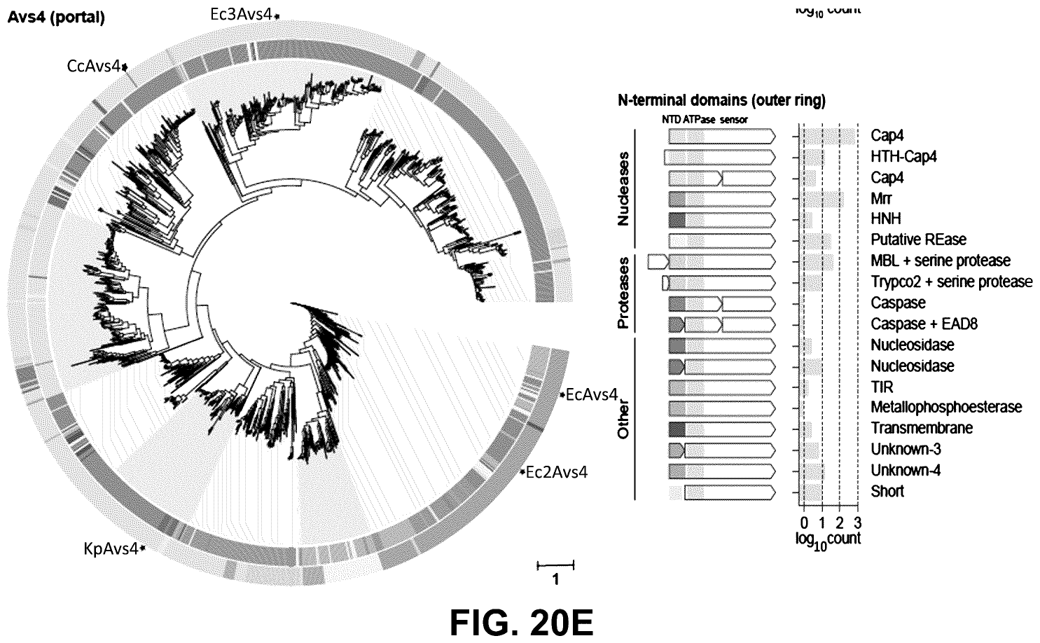

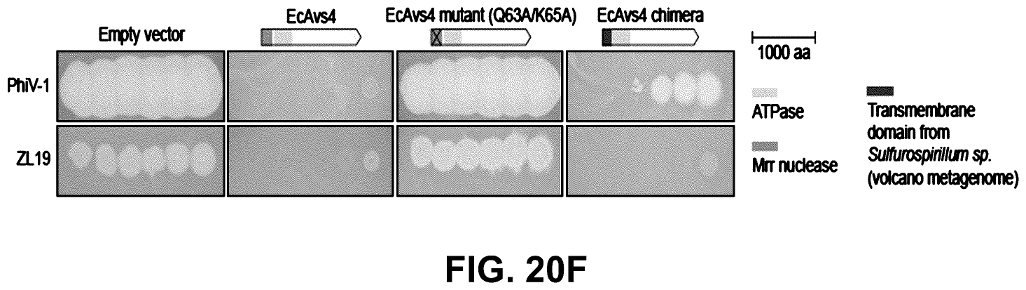

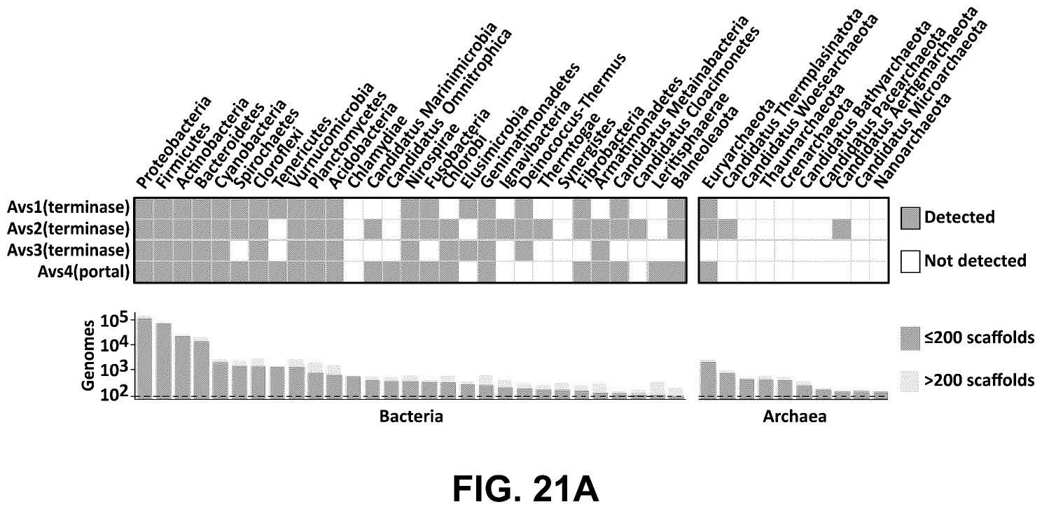

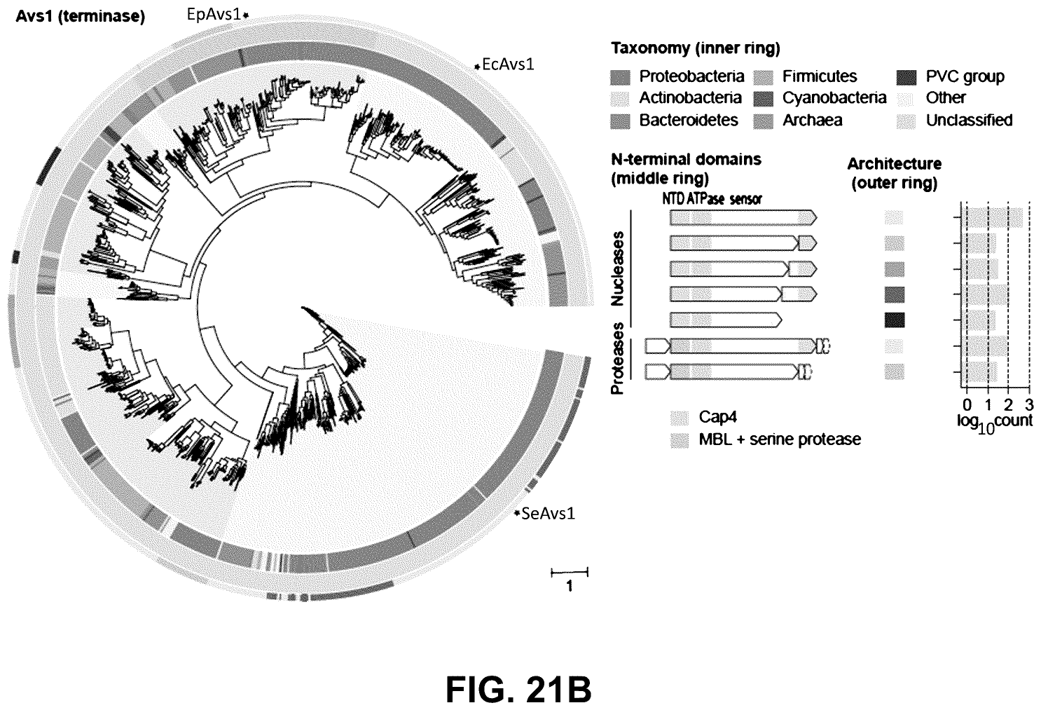

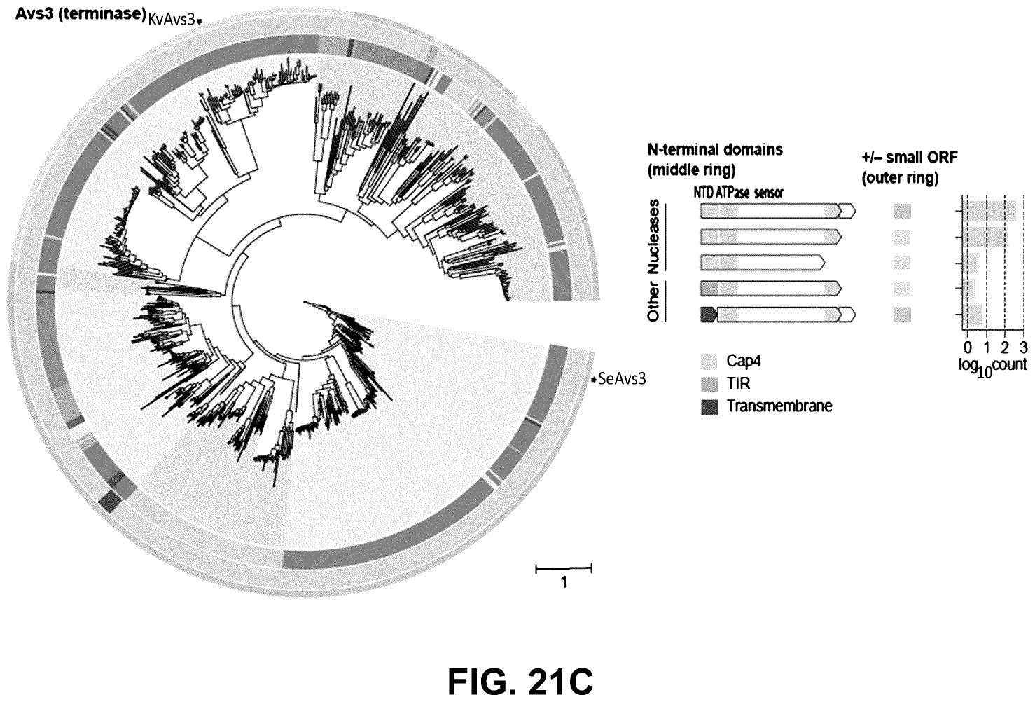

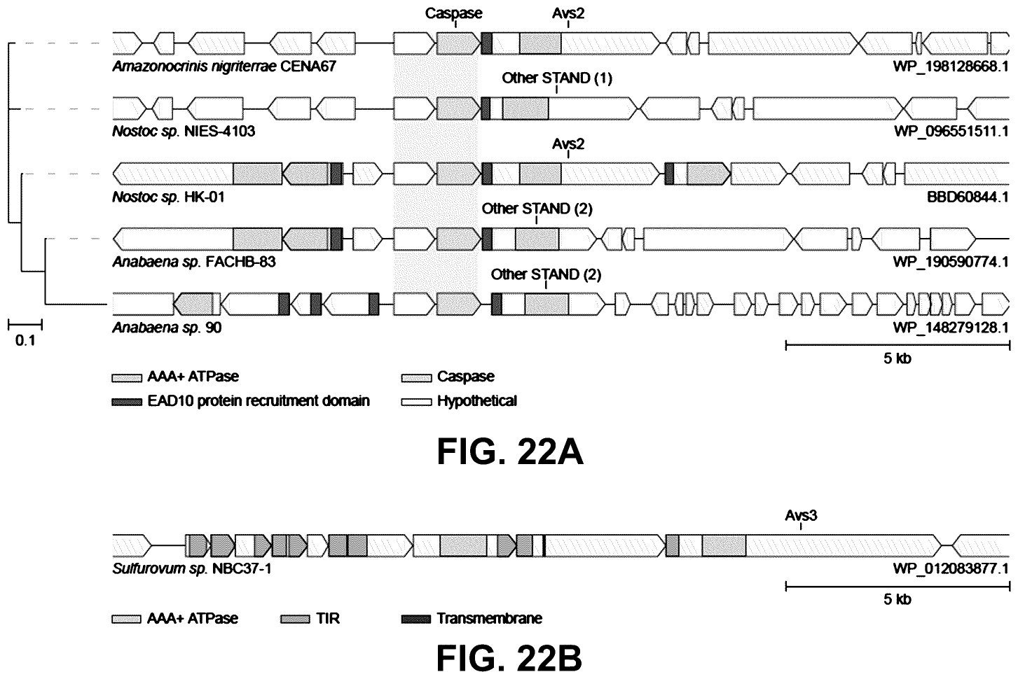

To assess the diversity of avs genes in prokaryotes, Applicant collected all intact homologs from each of the four families present in the National Center for Biotechnology Information (NCBI) nonredundant protein sequence database (Tables 3-6 herein and Data S8 of Gao et al., 2022. “Prokaryotic innate immunity via pattern recognition of conserved viral proteins” Science 377, eabm4096 (2022), which is incorporated by reference as if expressed in its entirety herein. The avs genes were identified in approximately 4-5% of sequenced prokaryotic genomes and are broadly distributed across phyla (FIG. 20A), with at least one avs gene detected in 27 of 29 and 3 of 10 well represented bacterial and archaeal phyla, respectively (FIG. 20B and FIG. 21A). Each Avs family has a characteristic protein size (FIG. 20C), consistent with their distinct mechanisms of target recognition. Applicant next constructed phylogenetic trees of each of the four families (FIG. 20D-20E, and FIG. 21B-21C), and found that these trees did not follow bacterial and archaeal phylogenies, suggesting extensive horizontal gene transfer, particularly for avs2 and avs4, in agreement with previous analyses 226 of STAND NTPases (13, 14). Furthermore, Applicant detected at least 18 distinct types of N-terminal effector domains present in Avs proteins, including non-nuclease domains such as proteases, nucleosidases, sirtuins (SIR2), Toll/interleukin-1 receptor homology (TIR) domains, cytidine monophosphate (CMP) hydrolases, transmembrane helices, and domains with unknown functions (Tables 3-6 herein and Data S8 of Gao et al., 2022. “Prokaryotic innate immunity via pattern recognition of conserved viral proteins” Science 377, eabm4096 (2022), which is incorporated by reference as if expressed in its entirety herein). Some less common variants are predicted to participate in intracellular signaling networks via effector-associated domains (EADs) that recruit a caspase-like enzyme by protein-protein interaction (29,30)(FIG. 22A-22B), reminiscent of animal NLRs.

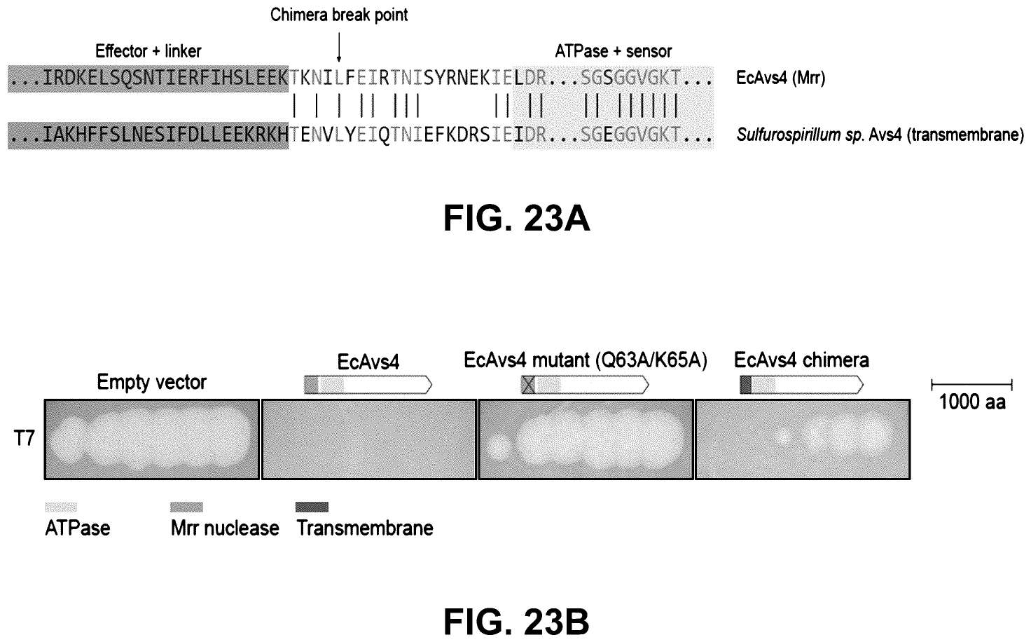

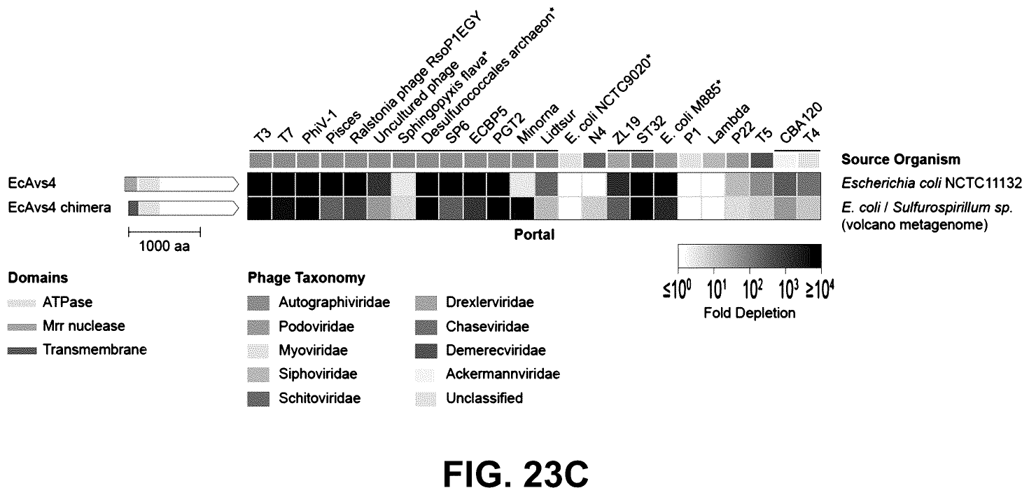

The apparent frequent exchange of N-terminal domains in the evolution of the Avs families emphasizes the modular organization characteristic of STAND NTPases (14) and implies that closely related ATPase and TPR domains can activate a wide range of effector functions beyond DNA cleavage. To test this hypothesis, Applicant chose an Avs4 homolog from Sulfurospirillum sp. that contains ATPase and TPR domains similar to those of EcAvs4, with 44% overall amino acid identity, but encompasses an N-terminal region with predicted transmembrane helices instead of a nuclease (FIG. 23A). Applicant generated a chimeric Avs4 protein by transplanting the transmembrane domain to EcAvs4 and found that the chimera conferred protection against T7, PhiV-1, and ZL19 (FIG. 20F and FIG. 23B) while retaining the ability to recognize the portal proteins from diverse phages (FIG. 23C).

### Phage Inhibit Avs Systems Through Diverse Anti-Defense Proteins.

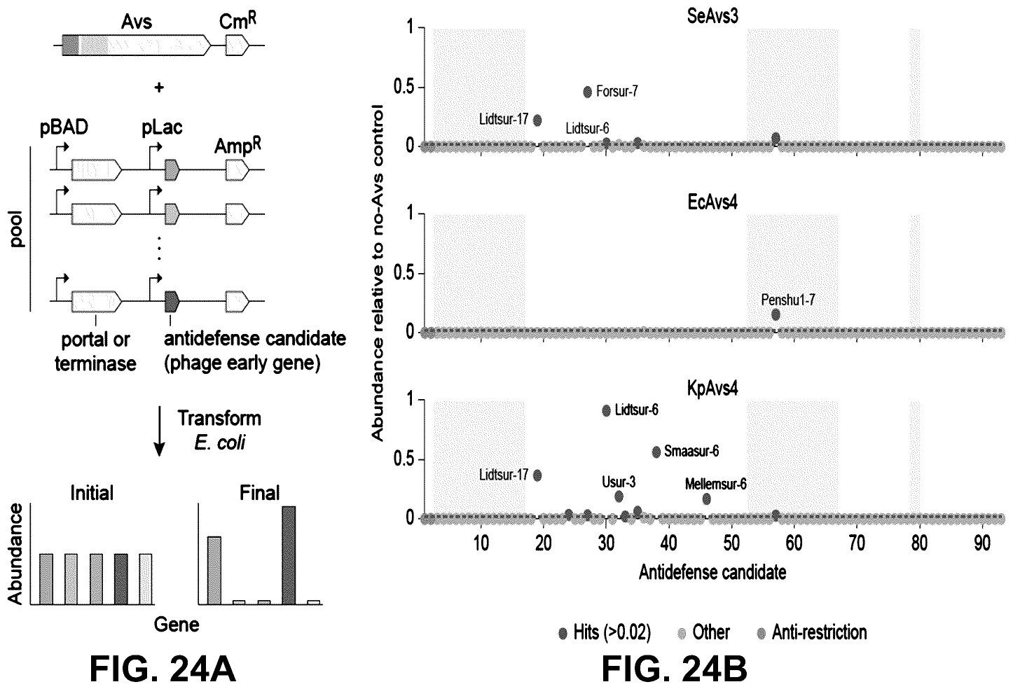

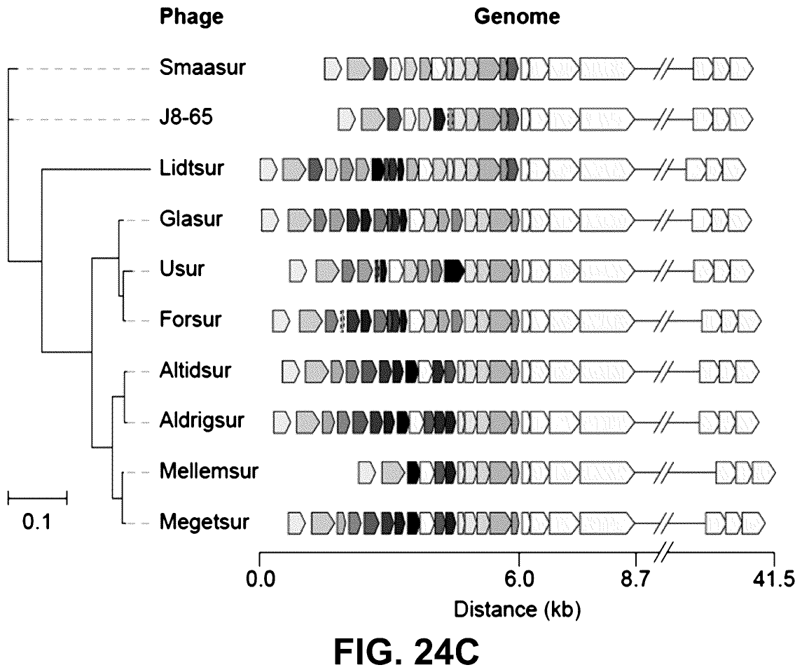

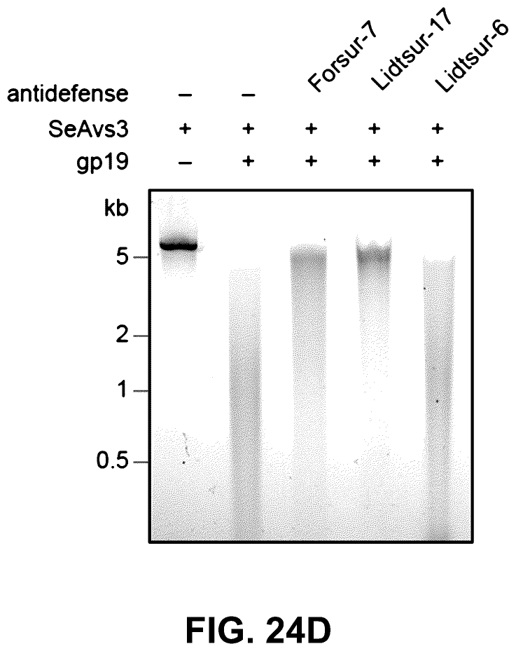

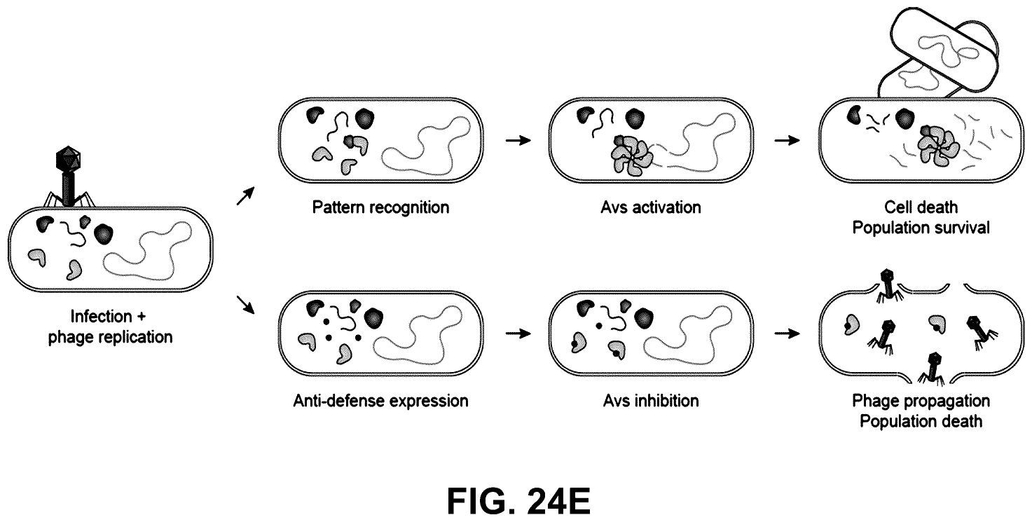

Bacterial and archaeal viruses have evolved diverse mechanisms to counteract defense systems (31), including numerous anti-restriction and anti-CRISPR proteins (32, 33). Applicant hypothesized that Avs inhibitors might be found among phage early genes, which are expressed before the portal and terminase genes during the phage life cycle. Focusing on the Autographiviridae family of T7-like coliphages, which have readily identifiable early genes, as well as portals and terminases that strongly activate Avs proteins, Applicant identified a set of 122 representative early genes that typically encode small proteins (median length 77 amino acids), characteristic of anti-defense (Table 7). Applicant performed a genetic screen for suppressors of Avs toxicity by co-expressing these early genes with SeAvs3, EcAvs4, or KpAvs4 and their cognate phage trigger (FIG. 24A). Applicant identified several early genes which rescued cell growth (FIG. 24B), most of which originate from a hypervariable region within a group of closely related phages isolated from wastewater (FIG. 24C) (34, 35).

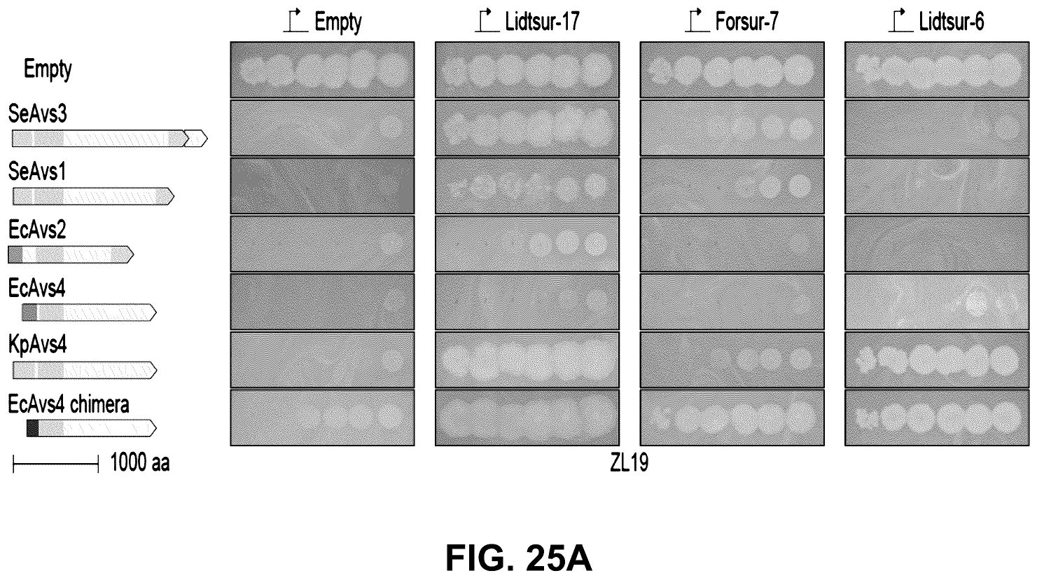

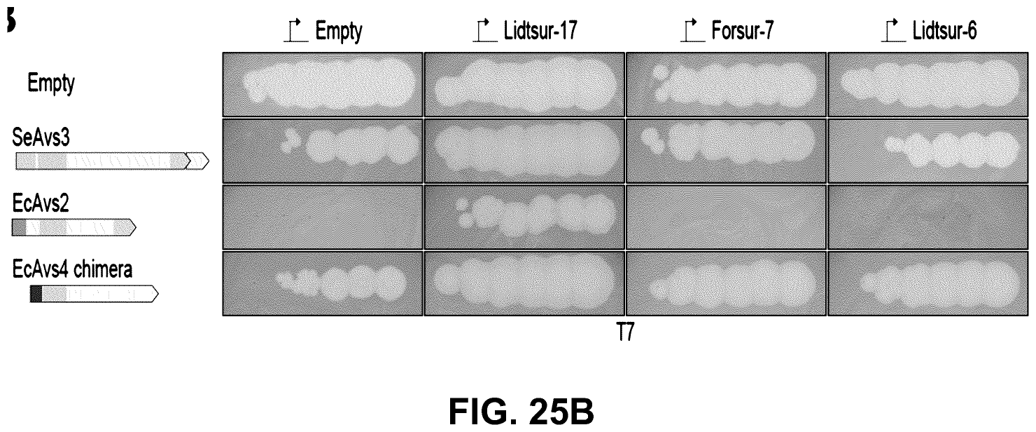

To validate these observations, Applicant produced three of the Avs inhibitors by cell-free translation and observed inhibition of SeAvs3 nuclease activity in vitro by Lidtsur-17, and to a lesser degree by Forsur-7 (FIG. 24D). Lidtsur-17, Forsur-7, and Lidtsur-6 were also active in phage plaque assays and restored phage propagation on Avs-containing E. coli (FIG. 25A-25B). Surprisingly, these inhibitors were active against different Avs families, including the chimeric EcAvs4, where the effector nuclease was replaced with a transmembrane domain. Furthermore, the lack of detectable sequence similarity between these inhibitors suggests distinct modes of action, which resembles the case of the highly diverse anti-CRISPRs (32, 33). Further studies will be required to elucidate the mechanism of how these phage proteins block Avs activity.

TABLE 7
Anti-defense candidates tested.

| | | | | | Abundance relative to no-Avs control | | | |
|---|---|---|---|---|---|---|---|---|
| | Access | Amino | | | EcAvs4 + | KpAvs4 + | SeAvs3 + | |
| # | ion | Acids | Phage | Group | portal | portal | terminase | Note |
| 1 | QLF86333.1 | 168 | LS3 | — | <0.001 | <0.001 | <0.001 | Anti-restriction Ocr-like |
| 2 | NP_041954.1 | 117 | T7 | — | <0.001 | <0.001 | <0.001 | Anti-restriction Ocr |
| 3 | YP_007500976.1 | 95 | UAB | 1 | <0.001 | <0.001 | <0.001 | |
| | | | Phi78 | | | | | |
| 4 | YP_424974.1 | 75 | K1E | 1 | <0.001 | <0.001 | <0.001 | |
| 5 | AQN32039.1 | 75 | vB_EcoP_R | 1 | <0.001 | <0.001 | <0.001 | |
| 6 | YP_654100.1 | 74 | K1-5 | 1 | <0.001 | <0.001 | <0.001 | |
| 7 | YP_009812367.1 | 69 | LL11 | 1 | <0.001 | <0.001 | <0.001 | |
| 8 | YP_007500980.1 | 59 | UAB | 1 | <0.001 | 0.0022 | <0.001 | |
| | | | Phi78 | | | | | |
| 9 | YP_009812365.1 | 64 | LL11 | 1 | <0.001 | <0.001 | <0.001 | |
| 10 | YP_009795380.1 | 61 | mutPK1A2 | 1 | 0.0021 | <0.001 | <0.001 | |
| 11 | YP_007500978.1 | 59 | UAB | 1 | <0.001 | <0.001 | 0.0029 | |
| | | | Phi78 | | | | | |
| 12 | QBQ76569.1 | 58 | vB_EcoP | 1 | <0.001 | <0.001 | <0.001 | |
| | | | R4596 | | | | | |
| 13 | YP_006987767.1 | 55 | vB_EcoP | 1 | <0.001 | <0.001 | <0.001 | |
| | | | ACG- | | | | | |
| | | | C91 | | | | | |
| 14 | YP_424971.1 | 54 | K1E | 1 | <0.001 | <0.001 | <0.001 | |
| 15 | YP_424968.1 | 51 | K1E | 1 | 0.0063 | <0.001 | <0.001 | |
| 16 | YP_009796071.1 | 41 | VEc3 | 1 | <0.001 | <0.001 | <0.001 | |
| 17 | YP_009796066.1 | 32 | Vec3 | 1 | <0.001 | <0.001 | 0.0013 | |
| 18 | QHR73015.1 | 175 | Smaasur | 2 | <0.001 | <0.001 | <0.001 | |
| 19 | YP_009821615.1 | 171 | Lidtsur | 2 | 0.0013 | 0.37 | 0.22 | Lidtsur-17 |
| 20 | QHR65291.1 | 170 | Altidsur | 2 | <0.001 | 0.0019 | 0.0015 | |
| 21 | YP_009821618.1 | 150 | Lidtsur | 2 | <0.001 | <0.001 | <0.001 | |
| 22 | QHR70507.1 | 146 | Megetsur | 2 | <0.001 | <0.001 | <0.001 | |
| 23 | QHR65282.1 | 113 | Altidsur | 2 | <0.001 | <0.001 | <0.001 | |
| 24 | QHR67393.1 | 112 | Glasur | 2 | <0.001 | 0.034 | 0.0016 | |
| 25 | QHR65405.1 | 111 | Mellemsur | 2 | <0.001 | <0.001 | <0.001 | |
| 26 | QHR73014.1 | 103 | Smaasur | 2 | 0.0059 | <0.001 | 0.0064 | |
| 27 | QHR71835.1 | 102 | Forsur | 2 | <0.001 | 0.031 | 0.46 | Forsur-7 |
| 28 | YP_009821614.1 | 99 | Lidtsur | 2 | <0.001 | <0.001 | 0.0016 | |
| 29 | YP_009821603.1 | 98 | Lidtsur | 2 | <0.001 | <0.001 | 0.0044 | |
| 30 | YP_009821604.1 | 97 | Lidtsur | 2 | <0.001 | 0.91 | 0.028 | Lidtsur-6 |
| 31 | QHR73008.1 | 94 | Smaasur | 2 | <0.001 | <0.001 | <0.001 | |
| 32 | QHR72976.1 | 94 | Usur | 2 | <0.001 | 0.19 | 0.016 | Usur-3 |
| 33 | QHR68511.1 | 94 | Aldrigsur | 2 | <0.001 | 0.02 | 0.0015 | |
| 34 | QHR65280.1 | 92 | Altidsur | 2 | <0.001 | <0.001 | <0.001 | |
| 35 | QHR70495.1 | 91 | Megetsur | 2 | <0.001 | 0.06 | 0.031 | |
| 36 | QHR73012.1 | 91 | Smaasur | 2 | 0.0078 | 0.011 | 0.0065 | |
| 37 | QHR73013.1 | 89 | Smaasur | 2 | <0.001 | <0.001 | <0.001 | |
| 38 | QHR73011.1 | 87 | Smaasur | 2 | <0.001 | 0.56 | <0.001 | Smaasur-6 |
| 39 | QHR72971.1 | 83 | Usur | 2 | <0.001 | 0.0023 | <0.001 | |
| 40 | QHR65404.1 | 81 | Mellemsur | 2 | <0.001 | <0.001 | <0.001 | |
| 41 | QHR67402.1 | 81 | Glasur | 2 | <0.001 | <0.001 | <0.001 | |
| 42 | QHR65287.1 | 81 | Altidsur | 2 | 0.0011 | 0.0011 | 0.0039 | |
| 43 | QHR73005.1 | 80 | Smaasur | 2 | <0.001 | <0.001 | <0.001 | |
| 44 | YP_009821616.1 | 79 | Lidtsur | 2 | <0.001 | <0.001 | <0.001 | |
| 45 | QHR67395.1 | 76 | Glasur | 2 | <0.001 | <0.001 | <0.001 | |
| 46 | QHR65403.1 | 76 | Mellemsur | 2 | <0.001 | 0.17 | <0.001 | Mellemsur-6 |
| 47 | QHR68502.1 | 67 | Aldrigsur | 2 | <0.001 | <0.001 | <0.001 | |
| 48 | QHR70492.1 | 64 | Megetsur | 2 | <0.001 | <0.001 | <0.001 | |
| 49 | YP_009821607.1 | 58 | Lidtsur | 2 | <0.001 | <0.001 | <0.001 | |
| 50 | YP_009821608.1 | 57 | Lidtsur | 2 | <0.001 | <0.001 | <0.001 | |
| 51 | QHR73009.1 | 50 | Smaasur | 2 | <0.001 | 0.0014 | <0.001 | |
| 52 | QHR71836.1 | 37 | Forsur | 2 | <0.001 | <0.001 | <0.001 | |
| 53 | QEG09774.1 | 174 | Penshu1 | 3 | <0.001 | <0.001 | <0.001 | |
| 54 | YP_009812836.1 | 140 | IMM-002 | 3 | 0.0012 | <0.001 | 0.0031 | |
| 55 | | 77 | CLB_P1 | 3 | <0.001 | <0.001 | <0.001 | |
| 56 | QEG09666.1 | 92 | Peacock | 3 | <0.001 | <0.001 | <0.001 | |
| 57 | QEG09778.1 | 86 | Penshul | 3 | 0.15 | 0.027 | 0.071 | Penshu1-7 |
| 58 | QOI58465.1 | 81 | vB_EcoP-U8 | 3 | 0.0011 | <0.001 | 0.0029 | |
| 59 | QLF86388.1 | 75 | LS2 | 3 | <0.001 | <0.001 | <0.001 | |
| 60 | QNH91713.1 | 63 | DY1 | 3 | <0.001 | <0.001 | <0.001 | |
| 61 | QEG09773.1 | 62 | Penshu1 | 3 | <0.001 | <0.001 | <0.001 | |
| 62 | YP_009324935.1 | 58 | GA2A | 3 | <0.001 | <0.001 | 0.0016 | |
| 63 | YP_009787259.1 | 55 | ZG49 | 3 | <0.001 | <0.001 | <0.001 | |
| 64 | YP_009789078.1 | 52 | vB_EcoPF | 3 | <0.001 | <0.001 | <0.001 | |
| 65 | YP_002003738.1 | 47 | EcoDS1 | 3 | <0.001 | <0.001 | <0.001 | |
| 66 | YP_009818255.1 | 41 | Ro451w | 3 | <0.001 | <0.001 | <0.001 | |
| 67 | QEG09670.1 | 29 | Peacock | 3 | <0.001 | <0.001 | <0.001 | |
| 68 | YP_009786124.1 | 94 | ECA2 | 4 | <0.001 | <0.001 | <0.001 | |
| 69 | YP_004678726.1 | 77 | K30 | 4 | <0.001 | <0.001 | <0.001 | |
| 70 | YP_009200622.1 | 66 | P483 | 4 | <0.001 | <0.001 | <0.001 | |
| 71 | NP_523299.1 | 55 | T3 | 4 | <0.001 | <0.001 | <0.001 | |
| 72 | YP_009204329.1 | 54 | P694 | 4 | <0.001 | <0.001 | <0.001 | |
| 73 | YP_009804640.1 | 53 | SRT7 | 4 | <0.001 | <0.001 | <0.001 | |
| 74 | YP_009795801.1 | 42 | EG1 | 4 | <0.001 | <0.001 | <0.001 | |
| 75 | YP_009804639.1 | 40 | SRT7 | 4 | <0.001 | 0.0011 | <0.001 | |
| 76 | YP_009146374.1 | 86 | ECBP5 | 5 | <0.001 | <0.001 | <0.001 | |
| 77 | | 78 | ECBP5 | 5 | 0.0025 | 0.0014 | 0.0016 | |
| 78 | YP_009146376.1 | 61 | ECBP5 | 5 | <0.001 | <0.001 | <0.001 | |
| 79 | YP_009821042.1 | 191 | Minorna | 6 | <0.001 | <0.001 | <0.001 | |
| 80 | YP_009821041.1 | 73 | Minorna | 6 | <0.001 | <0.001 | 0.0074 | |
| 81 | YP_009795443.1 | 169 | PGT2 | 7 | <0.001 | <0.001 | <0.001 | |
| 82 | YP_009795442.1 | 111 | PGT2 | 7 | 0.0019 | <0.001 | 0.0031 | |
| 83 | YP_009795446.1 | 101 | PGT2 | 7 | <0.001 | <0.001 | <0.001 | |
| 84 | YP_009795445.1 | 96 | PGT2 | 7 | <0.001 | <0.001 | <0.001 | |
| 85 | YP_009795447.1 | 91 | PGT2 | 7 | <0.001 | <0.001 | <0.001 | |
| 86 | YP_009795436.1 | 88 | PGT2 | 7 | <0.001 | <0.001 | <0.001 | |
| 87 | YP_009795441.1 | 82 | PGT2 | 7 | <0.001 | <0.001 | 0.0014 | |
| 88 | | 80 | phiKT | 7 | <0.001 | <0.001 | <0.001 | |
| 89 | YP_009795435.1 | 79 | PGT2 | 7 | <0.001 | <0.001 | <0.001 | |
| 90 | | 77 | phiKT | 7 | <0.001 | 0.0012 | <0.001 | |
| 91 | YP_009795440.1 | 59 | PGT2 | 7 | <0.001 | <0.001 | <0.001 | |
| 92 | | 56 | phiKT | 7 | <0.001 | <0.001 | <0.001 | |
| 93 | YP_009795439.1 | 37 | PGT2 | 7 | <0.001 | <0.001 | <0.001 | |
| 94 | YP_009821043.1 | 71 | Minorna | 6 | | — | <0.001 | |

| | | | | | |
|---|---|---|---|---|---|
| 95 | YP_424970.1 | 70 | K1E | 1 | Excluded due to toxicity |
| 96 | YP_654099.1 | 59 | K1-5 | 1 | Excluded due to toxicity |
| 97 | YP_007500983.1 | 51 | UAB | 1 | Excluded due to toxicity |

Phi78

| | | | | | |
|---|---|---|---|---|---|
| 98 | YP_009812370.1 | 37 | LL11 | 1 | Excluded due to toxicity |
| 99 | QHR72969.1 | 154 | Usur | 2 | Excluded due to toxicity |
| 100 | YP_009821599.1 | 130 | Lidtsur | 2 | Excluded due to toxicity |
| 101 | QHR72972.1 | 104 | Usur | 2 | Excluded due to toxicity |
| 102 | QHR71845.1 | 56 | Forsur | 2 | Excluded due to toxicity |
| 103 | QEG09776.1 | 115 | Penshu1 | 3 | Excluded due to toxicity |
| 104 | QEG09672.1 | 108 | Peacock | 3 | Excluded due to toxicity |
| 105 | QEG09556.1 | 106 | Pisces | 3 | Excluded due to toxicity |
| 106 | QEG09664.1 | 71 | Peacock | 3 | Excluded due to toxicity |
| 107 | QLF86337.1 | 65 | LS3 | 3 | Excluded due to toxicity |
| 108 | QEG09668.1 | 51 | Peacock | 3 | Excluded due to toxicity |
| 109 | NP_523296.1 | 152 | T3 | 4 | Excluded due to toxicity |
| 110 | YP_009820202.1 | 147 | HZP2 | 4 | Excluded due to toxicity |
| 111 | YP_009804637.1 | 118 | SRT7 | 4 | Excluded due to toxicity |
| 112 | YP_009813769.1 | 72 | N30 | 4 | Excluded due to toxicity |
| 113 | | 60 | K30 | 4 | Excluded due to toxicity |
| 114 | NP_523298.1 | 65 | T3 | 4 | Excluded due to toxicity |
| 115 | QOV06375.1 | 46 | JB01 | 4 | Excluded due to toxicity |
| 116 | YP_009152463.1 | 45 | CICC80001 | 4 | Excluded due to toxicity |
| 117 | YP_009146373.1 | 46 | ECBP5 | 5 | Excluded due to toxicity |
| 118 | YP_009821040.1 | 87 | Minorna | 6 | Excluded due to toxicity |
| 119 | YP_009821038.1 | 67 | Minorna | 6 | Excluded due to toxicity |
| 120 | YP_009821039.1 | 59 | Minorna | 6 | Excluded due to toxicity |
| 121 | YP_009795437.1 | 164 | PGT2 | 7 | Excluded due to toxicity |
| 122 | YP_009795444.1 | 103 | PGT2 | 7 | Excluded due to toxicity |

See also Data S9 of Gao et al., Science 377, eabm4096 (2022), which is incorporated herein by reference as if expressed in its entirety herein.

### Multiple Sequence Alignments

Multiple sequence alignments of Avs1, Avs2, Avs3, Avs4, 24 tested terminases, and 24 tested portals were generated and are shown in Data S10, Data S12, Data S14, Data S16, Data S18, and Data S19, respectively, of Gao et al. Science 377, eabm4096 (2022), which are incorporated by reference as if expressed in their entireties herein.

### Maximum Likelihood Phylogenetic Trees

Avs1, Avs2, Avs3, and Avs4 maximum likelihood phylogenetic trees were generated and are shown in Data S11, Data S13, Data S15, and Data S17, respectively, of Gao et al. Science 377, eabm4096 (2022), which are incorporated by reference as if expressed in their entireties herein.

### DISCUSSION

Here Applicant characterize four families of prokaryotic STAND NTPases and demonstrate that they act as pattern recognition receptors against two hallmark phage proteins, the large terminase subunit and the portal. These proteins, along with the major capsid protein, are the signature proteins of the virus realm Duplodnaviria, which uintes taild phages and tailed archaeal virus (24). Members of this realm, particularly tailed phages, are the most abundant among known viruses (63, 64). The portal protein nucleates virion assembly, occupying the unique pentameric vertex of capsids and providing the attachment site for the phage tail, and serves as the channel for genome entry into and exit from the capsid (36). The terminase is the motor that packages the phage genome into the capsid at high density and pressure, using the energy of ATP hydrolysis, and cleaves DNA concatemers into genome-size units (37). The universal, complex molecular functions of these proteins engender strong selective constraints and hence evolutionary conservation. It is therefore no surprise that these particular phage proteins were selected as the targets for pattern recognition during the coevolution of prokaryotes with viruses. Furthermore, the three groups (Avs1-3) that recognize terminases do not form a clade in the phylogeny of the STAND domain (FIG. 4A-4B), suggesting that defense based on the recognition of this phage protein evolved independently on multiple occasions.

The reconstitution of two Avs systems in vitro described here provides insight into their mechanism of defense, including promiscuous DNA endonuclease activity (FIG. 24E). Although many Avs proteins contain predicted nucleases, Applicant identified diverse N-terminal effector domains throughout Avs families, indicating unique mechanisms of defense that remain to be characterized. The demonstration that at least some of these effectors can be swapped without compromising the defense function of the Avs highlights the modular functionality of these proteins, which appears important for the diversification of defense mechanisms. The effectors of SeAvs3 and EcAvs4 are both activated by tetramerization suggesting that diverse Avs effectors are unified by oligomerization for activity, a common mechanism for signal transduction by STAND NTPases (38). Indeed, oligomerization is also involved in the activation of Cap4 nucleases in cyclic oligonucleotide-based antiphage signaling systems (25).

Notably, Applicant demonstrated that Avs proteinsrecognize conserved structural features of their cognate targets across an extreme variety of amino acid sequences, including those originating from both tailed phages and archael viruses, as well as eukaryotic herpesviruses, which are only distantly related and do not infect prokaryotes. Structural analysis of Avs3 revealed that it directly detects the active-site residues and ATP ligand of the terminase, thereby targeting the moieties that are the most difficult for phages to mutate without abrogating function.

Notable similarities, but also several differences, exist between eukaryotic NLRs and prokaryotic Avs proteins. Both are intracellular receptors of the STAND superfamily that detect PAMPs through C-terminal repetitive structures. Both exhibit triggered oligomerization, but with distinct interfaces between the central ATPase domains (FIG. 33A-33B). Similar to plant NLRs like RPP1 and the Roq1 resistosome, both Avs3 and Avs4 form tetramers, with the effector domains activated by forming a twofold symmetric dimer of dimers (59, 65). In the absence of their ligands, animal and plant NLRs have autoinhibited states that prevent oligomerization and effector activation (18). In these complexes, the effector (e.g., caspase-1) is a separate protein rather than a domain of the NLR. By contrast, Avs effectors are usually the N-terminal domain of the STAND NTPase. This simpler organization might be advantageous because counteracting phage replication requires a rapid, direct cellular response. This contrast parallels the distinction between the mechanisms of prokaryotic and eukaryotic STING proteins, whereby bacterial STING homologs directly activate TIR domain effectors rather than regulate transcription, as mammalian STINGs do (18, 39).

Bacteria and archaea encode numerous diverse STAND NTPases beyond the four families characterized in this study (14). Although some are not involved in defense, such as the well-characterized transcriptional regulators MalT (21), AfsR (22), and GutR (23), several are confirmed defense genes or are predicted to have a defense function based on their enrichment in genome regions adjacent to known defense systems (4, 40). Applicant investigated several of these other defense-associated systems (Table 2) but observed no detectable toxicity when co-expressed with any of the 48 tested terminases or portals. In light of the high success rate of this assay for Avs homologs, these results suggest that they are triggered by other pathogen-related patterns that remain to be identified. Further investigation will shed light on whether these triggers are proteins, and whether they are phage-specific or endogenous to the host. For instance, most characterized plant and fungal NLRs sense the state of host pathways rather than pathogen-specific proteins, and it remains a possibility that other groups of prokaryotic STAND NTPases function similarly.

Given the extensive sequence divergence among the STAND NTPases, it is unclear whether Avs proteins are direct evolutionary ancestors of their eukaryotic NLR homologs, although this remains a possibility. Alternatively, or additionally, the characteristic tripartite domain architectures of diverse STAND NTPases could have evolved convergently, suggesting that this modular organization is a facile way to create allosterically activated enzymes that can respond to PAMPs and could inspire the design of engineered molecular sensors. Overall, the results of this work advance understanding of host-virus interactions in diverse microbes and extend the paradigm of pattern recognition of pathogen-specific proteins to all domains of life.

### Materials and Methods

### Phylogenetic Analysis of STAND NTPases

For STAND phylogenetic analysis (FIG. 4A-4B), PSI-BLAST searches (41) against the database of complete bacterial and archaeal genomes (extracted from Genbank, March 2019) were performed for three iterations using ATPase domains of seven Avs1-3 homologs (WP_126523998.1, WP_115407481.1, WP_084007836.1, WP_060615938.1, WP_139964370.1, WP_063118745.1, WP_001017806.1) investigated experimentally. The 2000 best hits from each run were taken and combined with 949 Avs4 homologs found in the NCBI nonredundant(nr) database in 2021. A non-redundant set of sequences (4843) was used for phylogenetic reconstruction using a hybrid UPGMA/FastTree approach as follows. At the first step, sequence clusters were obtained using MMseqs2 (42) with a sequence similarity threshold of 0.5, and the sequences within each cluster were aligned using MUSCLE (43). At the second step, cluster-to-cluster similarity scores were obtained using HHSEARCH (44) (including trivial clusters consisting of a single sequence each) and normalized by the minimum of the self-scores. Relative similarity scores (s) were converted to distances (d) defined as d=−ln s, and a UPGMA (unweighted pair group method with arithmetic mean) dendrogram was constructed from the distance matrix. At the third step, sequence-based trees were constructed from the cluster alignments using FastTree (45) (WAG evolutionary model, gamma-distributed site rates) and rooted by midpoint; these trees were grafted onto the tips of the profile similarity-based UPGMA dendrograms. FastTree was also used to calculate support values. Only the second step of the above procedure was applied to reconstruct the UPGMA dendrogram using multiple alignments of selected well-supported branches identified by the first procedure.

### Construction of Avs Phylogenetic Trees

Homologs of each of the four clades of avs genes were identified using PSI-BLAST searches against the non-redundant protein sequence database (nr) in June 2021 using position-specific scoring matrices for each clade derived from manually curated multiple sequence alignments (MSAs) of conserved regions. After a round of curation to remove false positives hits and partial proteins, referencing the corresponding genome assemblies to correct misannotated start codons, a list of 1584, 2342, 1018, and 1813 non-redundant full-length proteins were obtained for Avs1, Avs2, Avs3, and Avs4, respectively. To reduce sampling bias, sequences were then clustered at 95% sequence identity (minimum 80% coverage) using MMseqs2 with parameters—min-seq-id 0.95-c 0.8-cov-mode 1. One representative from each cluster was selected for subsequent analyses, resulting in 843, 1255, 630, and 1089 sequences for Avs1, Avs2, Avs3, and Avs4, respectively.

MSAs of each Avs clade, excluding the variable N-terminal domains, were generated using MAFFT v7.450 (46) with global pairwise alignment (parameters—maxiterate 1000—globalpair). Alignments were trimmed using trimA1 1.2 with a gap threshold of 0.25 (−gt 0.25). Phylogenetic trees were built from the trimmed MSAs using IQ-TREE 1.6.12 (47) with the LG+G4 model and 2000 ultrafast bootstrap replicates (parameters-nstop 500-bb 2000-m LG+G4). To categorize the N-terminal domains, the N-terminal sequences were clustered using MMseqs2 with parameters—min-seq-id 0.4-c 0.8, and a representative sequence from each cluster was analyzed using HHpred (48). Phyla classification was determined from the NCBI taxonomy database, and trees were rooted by midpoint and visualized using iTOL (49).

The phylogenetic tree comparing the ATPase domains of NLR-like genes across model organisms (FIG. 3A) was constructed in a similar manner, incorporating the set of 23 human NLRs and the best characterized NLRs from Arabidopsis thaliana (50) and Neurospora crassa (19, 51).

### Taxonomic Distribution of Avs Genes

To determine the taxonomic distribution of avs genes, genome assemblies containing one or more full-length Avs homologs were identified via the NCBI Identical Protein Groups (IPG) database (Data S8 of Gao et al., 2022. “Prokaryotic innate immunity via pattern recognition of conserved viral proteins” Science 377 eabm4096, which is incorporated by reference as if expressed in its entirety herein). Redundant assemblies were removed on the basis of their nine-digit accession numbers. To determine the percentage of genomes containing avs genes, the list of all available prokaryotic assemblies was downloaded from ftp.ncbi.nlm.nih.gov/genomes/GENOME_REPORTS/prokaryotes.txt.

### Construction of Terminase and Portal Alignments

Structures of all tested terminase and portal proteins were predicted using AlphaFold2 (52), and structures were aligned and visualized using PyMOL 2.3.4. Representatives of the predicted structures were used as input for MSA construction using PROMALS3D (53). Prior to computing pairwise sequence identity, MSAs were trimmed to retain only the regions corresponding to the core terminase or portal fold.

### Cloning

Genes were chemically synthesized or amplified with Q5 (New England Biolabs) or Phusion Flash (Thermo Scientific) polymerase. Plasmids were assembled using the Gibson Assembly or NEBuilder HiFi DNA Assembly mix (New England Biolabs). Plasmid sequences were verified by Tn5 tagmentation and high-throughput sequencing, as previously described (4, 54).

### Bacterial Strains

E. coli NovaBlue and NovaBlue(DE3) were obtained from Millipore Sigma. E. coli K-12 (ATCC 25404) and strain C (ATCC 13706) were obtained from the American Type Culture Collection. All genetic assays were performed with E. coli NovaBlue(DE3) unless indicated otherwise.

### Competent Cell Production

E. coli strains were cultured in ZymoBroth with 25 μg/mL chloramphenicol and made competent using Mix & Go buffers (Zymo) according to the manufacturer's recommended protocol.

### PhiV-1 Fragment Screen

DNA fragments consisting of intact open reading frames (ORFs) were amplified from phage PhiV-1 and cloned into expression plasmids after a LacI-repressed T7 promoter. Plasmids were pooled with an mNeonGreen-expressing control plasmid and transformed into E. coli NovaBlue(DE3) containing either SeAvs3, EcAvs4, or an Avs-free pACYC184 empty vector. An additional sample consisting of the plasmidpool transformed into empty vector-containing E. coli NovaBlue, which lacks the ability to express from T7 promoters, was also included to assess the basal toxicity of the phage genes. After 1 hr outgrowth in S.O.C. (super optimal broth with catabolite repression) media (at 37 degrees C., cells were plated on LB agar plates containing 25 μg/mL chloramphenicol and 100 μg/mL ampicillin in the absence of IPTG. Plates were incubated for an additional 12 h at 37 degrees C., after which surviving plasmids were isolated by miniprep (Qiagen). A total of 200 ng of plasmid for each condition was tagmented with Tn5 to yield an average fragment size of about 500 bp. Following addition of 0.5 volumes of 0.1% SDS and column purification, tagmented fragments were amplified over 8 cycles by Q5 DNA polymerase (NEB) with unique i5 and i7 index primers. Amplicons were gel extracted and sequenced on a NextSeq (Illumina) using 150 cycles for the forward read. Reads were mapped to reference sequences using Geneious Prime. The read coverage of each sample was then normalized to the read coverage of the mNeonGreen control within the same sample. Finally, for each sample, the read coverage per base was divided by the corresponding read coverage per base for the empty vector NovaBlue(DE3) control (FIG. 3D), or by that of the empty vector NovaBlue control (FIG. 5A-5B).

### Terminase and Portal Depletion Screens

Terminase and portal genes were amplified directly from phage samples or chemically synthesized (Twist Bioscience) with codon optimization for E. coli. Genes were expressed under the control of a pBAD promoter. Plasmids were pooled with an mCherry-expressing control plasmid and transformed into E. coli NovaBlue (DE3) containing an Avs homolog or a pACYC184 empty vector. After 1 hr outgrowth in S.O.C. at 37 degrees C., cells were plated on LB agar plates containing 25 μg/mL chloramphenicol and 100 μg/mL ampicillin with 0.002% arabinose, or in some cases with 0.2% arabinose as detailed in the figures. After an additional 12 h at 37 degrees C., plasmids were isolated and sequenced, and depletion values were computed as described for the PhiV-1 depletion screen (FIG. 7A-7D, FIG. 9A-9B, and FIG. 23C).

### Portal and Terminase Mutant Screens

Two synonymous versions of the T7 portal gene were randomly mutagenized by PCR using KAPA HiFi HotStart ReadyMix DNA polymerase (Roche) and cloned via Gibson assembly into a plasmid backbone containing a LacI-repressed T7 promoter. Plasmids were column purified and transformed into E. coli NovaBlue(DE3) containing EcAvs4, KpAvs4, or CcAvs4. Cells were plated on LB agar plates containing 25 μg/mL chloramphenicol and 100 μg/mL ampicillin in the absence of IPTG. After overnight growth, surviving colonies were sampled at random, cultured, and sequenced, and those containing single amino acid substitutions in the portal were retained for subsequent analysis. To reduce the number of stop codons and frameshift mutants sampled, a fluorescent protein (mNeonGreen) was included in the plasmid backbone immediately after the portal ORF with a single nucleotide overlap, such that mNeonGreen was translated only if the portal ORF remained intact (55) (FIG. 18A-18B). Both portal and mNeonGreen were translated as separate polypeptides. To quantitatively assess the effect of each mutant on Avs4-mediated toxicity (FIG. 18C), mutant plasmids were pooled and re-transformed into Avs4-containing E. coli as described for the PhiV-1 depletion screen. Fold depletion was also quantified as described, with the exception that only reads containing 20-mer sequences unique to one mutant (i.e., mapping to the mutation site) were counted in the analysis. A similar procedure was followed to quantify the effect of truncation of the terminase or portal (FIGS. 17G and 17L), as well as terminase-domain mutations (FIG. 28F).

### Antidefense Screen

Putative early genes from Autographiviridae coliphages were tabulated clustered at 50% sequence identity and 50% coverage using MMseqs2 (—min-seq-id 0.5-c 0.5), resulting in 120 clusters. One representative was selected from each cluster, along with two additional sequences, for a total of 122 initial candidates (Table 7). Genes were synthesized by Twist Bioscience and cloned via Gibson assembly into expression vectors containing either the portal or terminase from phage PhiV-1 driven by a pBAD promoter. Antidefense candidates were expressed under the control of a lac promoter. Plasmids were pooled and transformed into E. coli containing SeAvs3, EcAvs4, KpAvs4, or an empty vector. Cells were grown at 37 degrees C. for 16h on LB agar plates containing 25 μg/mL chloramphenicol and 100 μg/mL ampicillin with no added arabinose. Following plasmid isolation, anti-defense candidates were amplified over two rounds of PCR to attach 8-nucleotide i7 and i5 index barcodes and sequenced with a 600 cycle MiSeq kit to ensure maximal coverage of each ORF. Reads containing mutations were discarded in the subsequent analysis.

### Phage Plaque Assays

E. coli host strains were grown to saturation at 37 degrees C. in Luria Broth (LB) or terrific broth (TB). To 10 mL top agar (10 g/L tryptone, 5 g/L yeast extract, 10 g/L NaCl, 7 g/L agar) was added chloramphenicol to a final concentration 25 μg/mL and, if needed, ampicillin to a final concentration of 100 μg/mL. Ten-fold dilutions of phage in phosphate-buffered saline were spotted on the plates. After overnight incubation at 37 degrees C., plates were photographed in a dark room with a white backlight.

### Construction of Mutant Phages

PhiV-1 gp8 and gp19 knockout phages were constructed as previously described (56, 57) using plasmid donors with homology arms to gp8 or gp19 in an E. coli trxA— strain (JW5856 from the Keio collection (58)). The sequence encoding TrxA was inserted into the PhiV-1 genome as a selection marker.

### Protein Purification

Avs NTPases were cloned into pCDF-Duet expression plasmids containing a C-terminal 6xHis tag. PhiV-1 gp8 and gp19 genes were cloned into TwinStrep-SUMO expression plasmids. Proteins were expressed in BL21(DE3) cells (NEB #C2527H). Cells were grown in Terrific Broth to mid-log phase and the temperature lowered to 18 degrees C. Expression was induced at OD₆₀₀ with 0.25 mM IPTG for 16-20 h before harvesting and freezing cells at −80 degrees C. Cell paste was resuspended in lysis buffer (50 mM Tris pH 7.5, 500 mM NaCl, 5% glycerol) supplemented with EDTA-free cOmplete protease inhibitor (Roche). Cells were lysed using a LM20 microfluidizer device (Microfluidics) and cleared lysate was bound to either Strep-Tactin Superflow Plus (Qiagen) or Ni-NTA Superflow resin (Qiagen). For TwinStrep-SUMO phage proteins, the resin was washed with lysis buffer and proteins eluted with lysis buffer supplemented with 5 mM desthiobiotin. The TwinStrep-SUMO tag was removed by overnight digest at 4 degrees C. with homemade SUMO protease Ulp1 at a 1:100 weight ratio of protease to target. Cleaved proteins were run on a Superose 6 Increase column (GE Healthcare Life Sciences) with a final storage buffer of 25 mM Tris pH 7.5, 500 mM NaCl, 10% glycerol, 1 mM DTT. Avs proteins containing 6xHis tags were bound to Ni-NTA resin in the presence of 25 mM imidazole, washed with lysis buffer containing 50 mM imidazole, and eluted with lysis buffer containing 300 mM imidazole. SeAvs3 was diluted to a final concentration of 100 mM NaCl and purified using a Resource Q column on an AKTA Pure 25 L (GE Healthcare Life Sciences) with a 100 mM-1M NaCl gradient. EcAvs4 was further purified by diluting to a final concentration 100 mM NaCl and absorbing contaminants by flowing the protein over a Resource Q and Heparin HP column. SeAvs3 and EcAvs4 were concentrated and loaded onto a Superose 6 Increase column with a final storage buffer of 25 mM Tris pH 7.5, 500 mM NaCl, 10% glycerol, 1 mM dithiothreitol (DTT).

Avs proteins containing 6xHis tags were bound to Ni-NTA resin in the presence of 25 mM imidazole, washed with lysis buffer containing 50 mM imidazole, and eluted with lysis buffer containing 300 mM imidazole. SeAvs3 was diluted to a final concentration of 100 mM NaCl and purified using a Resource Q column on an AKTA pure 25 L (GE Health-care Life Sciences) with a 100 mM-1M NaCl gradient. EcAvs4 was further purified by di-luting to a final concentration of 100 mM NaCl and absorbing contaminants by flowing the protein over a Resource Q and Heparin HP column. SeAvs3 and EcAvs4 were concentrated and loaded onto a Superose 6 Increase column with a final storage buffer of 25 mM Tris-HCl pH 7.5, 500 mM NaCl, 10% glycerol, and 1 mM DTT. SeAvs3 for cryo-EM analysis was purified in the same buffer without glycerol and only 300 mM NaCl, then concentrated to 1.4 mg/ml in a 500-μl 100,000 molecular weight cutoff (MWCO) Amicon spin concentrator.

### Avs Complex Purification

Avs-TwinStrep constructs were co-transformed with plasmids expressing either gp8 or gp19 into electrocompetent BL21 cells (Sigma Aldrich CMC0016) and grown and induced as before. Avs pulldowns using Strep-Tactin resin were run on SDS-PAGE gels and bound gp8 and gp19 bands excised and sent for confirmation by mass spectrometry (Taplin Biological Mass Spectrometry Facility, Harvard Medical School). For tandem affinity purification, plasmids containing SeAvs3-6xHis and gp19-StrepTag were cotransformed into electrocompetent E. coli BL21(DE3) and grown and induced as before. An SeAvs3-gp19 complex was purified using Ni-NTA followed by Strep-Tactin SuperFlow Plus resin. The final elution was run on a Superose 6 Increase column and yielded a peak elution at 13 ml containing a 1:1 ratio of SeAvs3 and gp19, as determined by SDS-PAGE band intensity analysis. A standard curve was generated using the Bio-Rad Gel Filtration Standard (1511901), and the gel-phase distribution coefficient (Km) was calculated as (elution volume−void column)/(column volume−void volume).

SeAvs3-Terminase Complex Formation for cryoEM

A total of 20 mg of SeAvs3 was mixed with 8.3 mg of PhiV-1 gp19 terminase in a total volume of 24 ml in the presence of 17 mM Tris-HCl pH 7.5, 280 mM NaCl, 0.8 mM DTT, 2% glycerol, 5 mM MgCl₂,and 1 mMATP. The reaction was incubated at 37° C. for 30 min and placed on ice for about 1 hour before cryo-EM grid preparation. Cryo-EM grids were prepared on a Thermo Scientific Vitrobot Mark IV at 4° C. and 100% humidity. A total of 3 mlofreaction was applied to a freshly glow-discharged (12 s at 15 mA) Cu 300 R2/2 holey carbon grid with a 2-nm layer of amorphous carbon (Quantifoil). After 30 s, the grid was manually blotted with Whatman Grade 1 filter paper and plunged into liquid ethane.

EcAvs4-Portal Complex Formation for cryoEM

PhiV-1 gp8 was cloned into an MBP-bdSUMO expression plasmid, and EcAvs4 was cloned into a pCDF-Duet plasmid with an internal TwinStrep tag added between residues 114 and 115. The EcAvs4 Mrr-like nuclease active site was mutated (Q63A/K65A) to allow co-expression with the portal. These two plasmids were cotransformed into E. coli BL21(DE3). A total of 6 liters of culture was grown in Terrific Broth to mid-log phase, and the temperature was lowered to 18° C. IPTG (0.25 mM) was added to induce expression, and growth was continued overnight. Cell paste was re-suspended in lysis buffer (50 mM Tris-HCl pH 7.4, 250 mM NaCl, 5% glycerol, 5 mM β-mercaptoethanol, 2 mM MgCl₂,0.1 mM ATP) supplemented with EDTA-free cOmplete protease inhibitor (Roche). Cells were lysed using a LM20 microfluidizer (Microfluidics) and cleared lysate was bound to Amylose Resin High Flow (NEB). After extensive washing with lysis buffer, the resin was eluted overnight at 4° C. by addition of 10 mg of homemade bdSENP1 protease. Eluted protein was incubated with Strep-Tactin Superflow Plus resin, washed with lysis buffer, then eluted with lysis buffer supplemented with 5 mM desthiobiotin. The eluate was concentrated in a 6-ml Vivaspin spin concentrator (30,000 MWCO) and run on a Superose 6 Increase column using 20 mM Tris-HCl pH 7.4, 200 mM NaCl, 2 mM MgCl₂,and 0.1 mMATP.Peak fractions containing EcAvs4 and gp8 were concentrated to 1.7 mg/ml using a 0.5 ml Amicon spin concentrator (100,000 MWCO) then immediately used for cryo-EM grid preparation. Cryo-EM grids were prepared on an Thermo Scientific Vitrobot Mark IV at 4° C. and 100% humidity. A total of 3 ml of sample was applied to a freshly glow-discharged (60 s at 15 mA) Cu 300 R1.2/1.3 holey carbon grid (Quantifoil). The grid was blotted for 4 s with blot force +5 and drain time 1 s, then plunged into liquid ethane.

### Cryo-EM Data Collection

All data were collected using the Thermo Scientific Titan Krios G3i cryo TEM at MIT.nano using a K3 direct detector (Gatan) operated in super-resolution mode with twofold binning and an energy filter with slit width of 20 eV.

For SeAvs3-gp19, 15,422 movies were collected at 105,000× magnification giving a real pixel size of 0.8697 Å, with defocus ranging from 1 to 3.5 mm with an exposure time of 1.15 s, fractionated into 30 frames and a flux of 19.7 e-/pix/s giving a total fluence per micro-graph of 30 e-/A2. For EcAvs4-gp8, 22,902 movies were collected at 130,000× magnification giving a real pixel size of 0.6788 Å, with defocus ranging from 1 mm to 2.5 mm with an exposure time of 0.6 s, fractionated into 24 frames and a flux of 23.6 e-/pix/s giving a total fluence per micro-graph of 30.8 e-/A2.

### Cryo-EM Data Processing

All cryo-EM data were processed using RELION-4.0 (66). Movies were corrected for motion using the RELION implementation of MotionCor2, with 5-by-5 patches and dose-weighting. Contrast transfer function (CTF) parameters were estimated using CTFFIND-4.1. All reported resolutions use the gold-standard Fourier shell correlation with a cutoff of 0.143. For the SeAvs3-gp19 dataset, particle picking was first carried out on 800 micrographs using the Topaz general model (67). A good subset of these particles, as determined by three-dimensional (3D) classification, was used to train Topaz, and this trained model was used to pick 128,500 particles from the entire dataset. Extracted particles, downscaled fourfold, were subjected to 3D classification without imposing symmetry using a reference derived from a preliminary dataset. A total of 44,489 particles, corresponding to 34.5% of picked particles, showed sharp features and apparent C4 symmetry and were reextracted without binning and refined with C4 symmetry imposed. After refining per-particle defocus and global magnification, beamtilt, trefoil, and performing Bayesian polishing, a reconstruction was yielded at 3.8-Å resolution with clear density for the SeAvs3 ATPase domain but blurred density for both the N-terminal nuclease and C-terminal TPR+ terminase domains.

To improve density for the N-terminal nuclease domains, 3D classification without alignment was performed while imposing C2 symmetry. This revealed two equal populations of particles each with clear density for the nuclease domains, related by a 90° rotation in the z axis. In the refinement STAR file, the parameter rinAngleRot was therefore incremented by 90° for one of these populations, before focused refinement starting at 1.8° local angular searches with a soft mask around the nuclease domains. This produced a reconstruction at 3.4 Å, measured using the same soft mask.

To improve density for the C-terminal TPR+ terminase domains, C4 symmetry expansion was performed on the C4 refinement data.star file, followed by particle subtraction with recentering using a mask around one of the four asymmetric units. This generated four subparticles for each original particle. Refinement starting at 1.8° local angular searches with a soft mask, followed by CTF refinement and another round of refinement, produced a reconstruction at 3.4-Å resolution.

For the EcAvs4-gp8 dataset, 1825 particles from 80 micrographs were manually picked and used to train Topaz. The trained Topaz model then picked 444,626 particles from the entire dataset, which were extracted with fourfold binning and subjected with 3D classification without imposing symmetry using the octameric (pseudo-D4) EcAvs4-gp8 reference derived from a preliminary dataset. A total of 133,133 particles (29.9%) showing the same pseudo-D4 symmetry were reextracted at 1.034 Å/pix and refined with D4 symmetry imposed. After Bayesian polishing, this yielded a 3.7-Å resolution reconstruction. D1 symmetry expansion followed by particle subtraction was then used to convert these particles to 266,266 subparticles that correspond to the tetrameric complex. Like SeAvs3-gp19, these also had blurry density for the N-terminal nuclease and C-terminal TPR+ terminase domains but additionally had poor density for the ATPase domains, suggesting a C2 reconstruction might be suitable for the whole tetramer.

To improve overall density, a 3D classification without alignment was first performed with C2 symmetry imposed. This produced two equally occupied classes, collectively rep-resenting a 169,977-particle subset (63.8%), that appeared identical but for a 900 rotation, but less clearly distinguished than the same analysis on SeAvs3-gp19. Therefore, they were refined together with local 1.8° angular searches and C2 symmetry but with “Relax symmetry: C4” to account for the pseudo-C4 symmetry. This produced a consensus C2 refinement but still with relatively blurred densities for the nuclease and C-terminal TPR domains. The nuclease domain density was improved by focused refinement with a soft mask, followed by refining anisotropic magnification, per-(sub)particle defocus, and beamtilt, trefoil and fourth-order aberrations, and second refinement, yielding a 2.9-Å resolution reconstruction. The C-terminal TPR domains were improved by C4 symmetry expansion, followed by C1 focused refinement with a soft mask and CTF refinement, but still had unclear density at the periphery at the site of an important EcAvs4-portal contact. Therefore, a final 3D classification was performed with a soft mask just around this contact and a regularization parameter (T) of 20. A total of 500,066 selected subparticles (73%) were then focus-refined with the same mask to yield a reconstruction at 3.0-Å resolution with better density for this region.

### Model Building

Initial models for SeAvs3, EcAvs4, PhiV-1 gp8, and PhiV-1 gp19 were generated using AlphaFold and fit into the cryo-EM maps using ISOLDE (68) with adaptive distance restraints, followed by manual rebuilding in Coot (69) and further refinement in ISOLDE. Coordinates were refined in real space using PHENIX (70), performing one macrocycle of global minimization and atomic displacement parameter (ADP) refinement and skipping local grid searches.

### In Vitro Cleavage Reactions

Purified Avs proteins were incubated with nucleic acid substrates in reaction buffer (20 mM HEPES pH 7.5, 100 mM NaCl, 1 mM DTT, 5% glycerol final concentration). Typical reactions contained approximately 100 ng of DNA substrate, 100 ng Avs protein, and 100 ng gp8 or gp19 in a 10 μL reaction volume. MgCl₂ was added at 5 mM where indicated, ATP and AMP-PNP at 1 mM. Reactions were carried out at 37 C for the indicated time and products purified using a PCR Purification column (Qiagen) before agarose gel analysis on a 1% E-Gel EX (Thermo Fisher Scientific).

### Bacterial Two-Hybrid Assays

Expression plasmids were cloned by fusing either the T18 or T25 fragments of CyaA from Bordetella pertussis to nuclease-deficient Avs proteins as well as PhiV-1 gp8 portal and gp19 terminase. BTH101 cells (F—, cya-99, araD139, galE15, galK16, rpsL1 (Strr), hsdR2, mcrA1, mcrB1) were co-transformed with pairs of T18 and T25 containing plasmids. Overnight cultures were diluted 1:20 and plated on indicator plates containing 50 mg/mL ampicillin, 25 mg/mL kanamycin, 500 μg/mL ammonium iron (III) citrate, 300 μg/mL S-gal, and 0.5 mM IPTG. Cells were grown at 30 C overnight before imaging.

### Southern Blot Analysis

E. coli K-12 (ATCC 25404) cultures were grown to mid-log phase (OD600 0.5), and for each sample, 6 mL of culture was infected with wild type or mutant PhiV-1 at a multiplicity of infection of 1. After 20 min at 37 degrees C., prior to cell lysis, infected cells were pelleted and resuspended in 200 μL of media. After further incubation at 37 degrees C., for a total of 90 min, samples were frozen in liquid nitrogen. DNA was extracted from 200 μL cultures by adding 200 μL lysis buffer (10 mM Tris-Cl, pH 8.0, 1 mM EDTA, 100 mM NaCl, 1% SDS, 2% Triton X-100), 100 μL of glass beads, and 200 μL phenol-chloroform (1:1) followed by brief vortexing. Samples were centrifuged at 4 degrees C. and DNA from the upper layer, extracted with chloroform, and precipitated with the addition of 1 mL ice-cold 100% ethanol and centrifugation at 4 degrees C. DNA pellets were resuspended in 200 μL TE with 300 μg RNAse A (Sigma-Aldrich) and incubated at 37 degrees C. for 1 hour. DNA was precipitated with the addition of 1 mL ice-cold 100% ethanol and 20 μL of 4 M ammonium acetate, centrifuged, dried, and resuspended in TE. DNA was digested with Eco47III and run on a 1% agarose gel in 1×TBE at 100 V. The gel was denatured with 0.5 M NaOH and 1.5 M NaCl for 30 min, and neutralized with 1.5 M NaCl and 0.5 M Tris-Cl pH 7.5 for 30 min. DNA was transferred to Hybond N+ membrane (GE Healthcare Life Sciences) using overnight capillary flow and 10× SSC buffer (1.5 M NaCl, 150 mM sodium citrate, pH 7). Membranes were UV-crosslinked (Stratalinker 1800, Agilent) and blocked at 61 degrees C. with Church hybridization buffer (250 mM NaPO4 pH 7.2, 1 mM EDTA, 7% SDS). Radiolabeled probes complementary to the gp13 gene were generated from purified PCR products using the Prime-It Random labeling kit (Agilent) and a32-dCTP. Membranes were probed overnight, washed three times with 61 degrees C. Church hybridization buffer and exposed overnight with X-ray film (GE Healthcare Life Science) before developing. Quantification of phage DNA bands was performed in Fiji with background signal subtracted.

### REFERENCES FOR EXAMPLE 1

1. K. S. Makarova, Y. I. Wolf, S. Snir, E. V. Koonin, Defense islands in bacterial and archaeal genomes and prediction of novel defense systems. J. Bacteriol. 193, 6039-6056 (2011).
2. E. V. Koonin, K. S. Makarova, Y. I. Wolf, Evolutionary Genomics of Defense Systems in Archaea and Bacteria. Annu. Rev. Microbiol. 71, 233-261 (2017).
3. S. Doron, S. Melamed, G. Ofir, A. Leavitt, A. Lopatina, M. Keren, G. Amitai, R. Sorek, Systematic discovery of antiphage defense systems in the microbial pangenome. Science. 359 (2018), doi:10.1126/science.aar4120.
4. L. Gao, H. Altae-Tran, F. B hning, K. S. Makarova, M. Segel, J. L. Schmid-Burgk, J. Koob, Y. I. Wolf, E. V. Koonin, F. Zhang, Diverse enzymatic activities mediate antiviral immunity in prokaryotes. Science. 369, 1077-1084 (2020).
5. D. Klaiman, E. Steinfels-Kohn, G. Kaufmann, A DNA break inducer activates the anticodon nuclease RloC and the adaptive immunity in Acinetobacter baylyi ADP1. Nucleic Acids Res. 42, 360 328-339 (2014).
6. C. K. Guegler, M. T. Laub, Shutoff of host transcription triggers a toxin-antitoxin system to cleave phage RNA and abort infection. Mol. Cell. 81, 2361-2373.e9 (2021).
7. R. Cheng, F. Huang, H. Wu, X. Lu, Y. Yan, B. Yu, X. Wang, B. Zhu, A nucleotide-sensing endonuclease from the Gabija bacterial defense system. Nucleic Acids Res. 49, 5216-5229 (2021).
8. R. Bingham, S. I. Ekunwe, S. Falk, L. Snyder, C. Kleanthous, The major head protein of bacteriophage T4 binds specifically to elongation factor Tu. J. Biol. Chem. 275, 23219-23226 (2000).
9. A. Millman, A. Bernheim, A. Stokar-Avihail, T. Fedorenko, M. Voichek, A. Leavitt, Y. Oppenheimer-Shaanan, R. Sorek, Bacterial Retrons Function In Anti-Phage Defense. Cell. 183, 1551-1561.e12 (2020).
10. S. Kronheim, M. Daniel-Ivad, Z. Duan, S. Hwang, A. I. Wong, I. Mantel, J. R. Nodwell, K. L. Maxwell, A chemical defence against phage infection. Nature. 564 (2018), pp. 283-286.
11. A. Bernheim, A. Millman, G. Ofir, G. Meitav, C. Avraham, H. Shomar, M. M. Rosenberg, N. Tal, S. Melamed, G. Amitai, R. Sorek, Prokaryotic viperins produce diverse antiviral molecules. Nature. 589, 120-124 (2021).
12. K. S. Makarova, Y. I. Wolf, E. V. Koonin, Comparative genomics of defense systems in archaea and bacteria. Nucleic Acids Res. 41, 4360-4377 (2013). 13. E. V. Koonin, L. Aravind, Origin and evolution of eukaryotic apoptosis: the bacterial connection. Cell Death Differ. 9, 394-404 (2002).
13. Koonin et al., Origin and evolution of eukaryotic apoptosis: The bacterial connection. Cell Death Differ. 9, 394-404 (2002).
14. D. D. Leipe, E. V. Koonin, L. Aravind, STAND, a class of P-loop NTPases including animal and plant regulators of programmed cell death: multiple, complex domain architectures, unusual phyletic patterns, and evolution by horizontal gene transfer. J. Mol. Biol. 343, 1-28 (2004).
15. Y. Zhao, J. Yang, J. Shi, Y.-N. Gong, Q. Lu, H. Xu, L. Liu, F. Shao, The NLRC4 inflammasome receptors for bacterial flagellin and type III secretion apparatus. Nature. 477 (2011), pp. 596-600.
16. E. M. Kofoed, R. E. Vance, Innate immune recognition of bacterial ligands by NAIPs determines inflammasome specificity. Nature. 477, 592-595 (2011).
17. R. Caruso, N. Warner, N. Inohara, G. N ez, NOD1 and NOD2: signaling, host defense, and inflammatory disease. Immunity. 41, 898-908 (2014).
18. J. D. G. Jones, R. E. Vance, J. L. Dangl, Intracellular innate immune surveillance devices in plants and animals. Science. 354 (2016), doi:10.1126/science.aaf6395.
19. J. Heller, C. Clav, P. Gladieux, S. J. Saupe, N. L. Glass, NLR surveillance of essential SEC-9 SNARE proteins induces programmed cell death upon allorecognition in filamentous fungi. Proc. Natl. Acad. Sci. U.S.A 115, E2292-E2301 (2018).
20. S. Bauernfried, M. J. Scherr, A. Pichlmair, K. E. Duderstadt, 394 V. Hornung, Human NLRP1 is a sensor for double-stranded RNA. Science. 371 (2021), doi:10.1126/science.abd0811.
21. O. Danot, A complex signaling module governs the activity of MalT, the prototype of an emerging transactivator family. Proc. Natl. Acad. Sci. U.S.A 98, 435-440 (2001).
22. S. Horinouchi, M. Kito, M. Nishiyama, K. Furuya, S. K. Hong, K. Miyake, T. Beppu, Primary structure of AfsR, a global regulatory protein for secondary metabolite formation in Streptomyces coelicolor A3(2). Gene. 95, 49-56 (1990).
23. R. Ye, S. N. Rehemtulla, S. L. Wong, Glucitol induction in Bacillus subtilis is mediated by a regulatory factor, GutR. J. Bacteriol. 176, 3321-3327 (1994).
24. E. V. Koonin, V. V. Dolja, M. Krupovic, A. Varsani, Y. I. Wolf, N. Yutin, F. M. Zerbini, J. H. Kuhn, Global Organization and Proposed Megataxonomy of the Virus World. Microbiol. Mol. Biol. Rev. 84 (2020), doi:10.1128/MMBR.00061-19.
25. B. Lowey, A. T. Whiteley, A. F. A. Keszei, B. R. Morehouse, I. T. Mathews, S. P. Antine, V. J. Cabrera, D. Kashin, P. Niemann, M. Jain, F. Schwede, J. J. Mekalanos, S. Shao, A. S. Y. Lee, P. J. Kranzusch, CBASS Immunity Uses CARF-Related Effectors to Sense 3′-5′- and 2′-5′-Linked Cyclic Oligonucleotide Signals and Protect Bacteria from Phage Infection. Cell. 182, 38-49.e17 (2020).
26. K. S. Makarova, A. Timinskas, Y. I. Wolf, A. B. Gussow, V. Siksnys, C.
Venclovas, E. V. Koonin, Evolutionary and functional classification of the CARF domain superfamily, key sensors in prokaryotic antivirus defense. Nucleic Acids Res. 48, 8828-8847 (2020).
27. J. Heitman, P. Model, Site-specific methylases induce the SOS DNA repair response in Escherichia coli. J. Bacteriol. 169, 3243-3250 (1987).
28. G. Karimova, J. Pidoux, A. Ullmann, D. Ladant, A bacterial two-hybrid system based on a reconstituted signal transduction pathway. Proc. Natl. Acad. Sci. U.S.A 95, 5752-5756 (1998).
29. G. Kaur, L. M. Iyer, A. M. Burroughs, L. Aravind, Bacterial death and TRADD-N domains help define novel apoptosis and immunity mechanisms shared by prokaryotes and metazoans. Elife. 10 (2021), doi:10.7554/eLife.70394.
30. G. Kaur, A. M. Burroughs, L. M. Iyer, L. Aravind, Highly regulated, diversifying NTP-dependent biological conflict systems with implications for the emergence of multicellularity. Elife. 9 (2020), doi:10.7554/eLife.52696.
31. J. E. Samson, A. H. Magad n, M. Sabri, S. Moineau, Revenge of the phages: defeating bacterial defenses. Nat. Rev. Microbiol. 11, 675-687 (2013).
32. A. Pawluk, A. R. Davidson, K. L. Maxwell, Anti-CRISPR: discovery, mechanism and function. Nat. Rev. Microbiol. 16, 12-17 (2018).
33. J. Bondy-Denomy, A. Pawluk, K. L. Maxwell, A. R. Davidson, Bacteriophage genes that inactivate the CRISPR/Cas bacterial immune system. Nature. 493, 429-432 (2013).
34. N. S. Olsen, L. Forero-Junco, W. Kot, L. H. Hansen, Exploring the Remarkable Diversity of Culturable Phages in the Danish Wastewater Environment. Viruses. 12 (2020), doi:10.3390/v12090986. 35. M. Schmerer, I. J. Molineux, J. J. Bull, Synergy 432 as a rationale for phage therapy using phage cocktails. PeerJ. 2, e590 (2014).
35. M. Schmerer, I. J. Molineux, J. J. Bull, Synergy as a rationale for phage therapy using phage cocktails. PeerJ 2, e590 (2014).
36. C. L. Dedeo, G. Cingolani, C. M. Teschke, Portal Protein: The Orchestrator of Capsid Assembly for the dsDNA Tailed Bacteriophages and Herpesviruses. Annu Rev Virol. 6, 141-160 (2019).
37. S. R. Casjens, The DNA-packaging nanomotor of tailed bacteriophages. Nat. Rev. Microbiol. 9, 647-657 (2011).
38. O. Danot, E. Marquenet, D. Vidal-Ingigliardi, E. Richet, Wheel of Life, Wheel of Death: A Mechanistic Insight into Signaling by STAND Proteins. Structure. 17 (2009), pp. 172-182.
39. B. R. Morehouse, A. A. Govande, A. Millman, A. F. A. Keszei, B. Lowey, G. Ofir, S. Shao, R. Sorek, P. J. Kranzusch, STING cyclic dinucleotide sensing originated in bacteria. Nature. 586, 429-433 (2020).
40. S. A. Shmakov, K. S. Makarova, Y. I. Wolf, K. V. Severinov, E. V. Koonin, Systematic prediction of genes functionally linked to CRISPR-Cas systems by gene neighborhood analysis. Proc. Natl. Acad. Sci. U.S.A 115, E5307-E5316 (2018).
41. S. F. Altschul, T. L. Madden, A. A. Sch ffer, J. Zhang, Z. Zhang, W. Miller, D. J. Lipman, Gapped BLAST and PSI-BLAST: a new generation of protein database search programs. Nucleic Acids Res. 25, 3389-3402 (1997).
42. M. Steinegger, J. S ding, MMseqs2 enables sensitive protein sequence searching for the analysis of massive data sets. Nat. Biotechnol. 35, 1026-1028 (2017).
43. R. C. Edgar, MUSCLE: a multiple sequence alignment method with reduced time and space complexity. BMC Bioinformatics. 5, 113 (2004).
44. M. Steinegger, M. Meier, M. Mirdita, H. V hringer, S. J. Haunsberger, J. S ding, HH-suite3 for fast remote homology detection and deep protein annotation, doi:10.1101/560029.
45. M. N. Price, P. S. Dehal, A. P. Arkin, FastTree 2—approximately maximum-likelihood trees for large alignments. PLoS One. 5, e9490 (2010).
46. K. Katoh, K. Misawa, K.-I. Kuma, T. Miyata, MAFFT: a novel method for rapid multiple sequence alignment based on fast Fourier transform. Nucleic Acids Res. 30, 3059-3066 (2002).
47. L.-T. Nguyen, H. A. Schmidt, A. von Haeseler, B. Q. Minh, IQ-TREE: a fast and effective stochastic algorithm for estimating maximum-likelihood phylogenies. Mol. Biol. Evol. 32, 268-274 (2015).
48. L. Zimmermann, A. Stephens, S.-Z. Nam, D. Rau, J. Kubler, M. Lozajic, F.
Gabler, J. S ding, A. N. Lupas, V. Alva, A Completely Reimplemented MPI Bioinformatics Toolkit with a New HHpred Server at its Core. J. Mol. Biol. 430, 2237-2243 (2018).
49. I. Letunic, P. Bork, Interactive Tree Of Life (iTOL) v5: an online tool for phylogenetic tree display and annotation. Nucleic Acids Res. 49, W293-W296 (2021).
50. A.-L. Van de Weyer, F. Monteiro, O. J. Furzer, M. T. Nishimura, V. Cevik, K. Witek, J. D. G. Jones, J. L. Dangl, D. Weigel, F. Bemm, A Species-Wide Inventory of NLR Genes and Alleles in Arabidopsis thaliana. 469 Cell. 178, 1260-1272.e14 (2019).
51. W. Dyrka, M. Lamacchia, P. Durrens, B. Kobe, A. Daskalov, M. Paoletti, D. J. Sherman, S. J. Saupe, Diversity and variability of NOD-like receptors in fungi. Genome Biol. Evol. 6, 3137-3158 (2014).
52. J. Jumper, R. Evans, A. Pritzel, T. Green, M. Figurnov, O. Ronneberger, K. Tunyasuvunakool, R. Bates, A. Ž dek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature. 596, 583-589 (2021).
53. J. Pei, B. H. Kim, N. V. Grishin, PROMALS3D: a tool for multiple protein sequence and structure alignments. Nucleic Acids Res. 36 (2008), doi:10.1093/nar/gkn072.
54. S. Picelli, A. K. Bj rklund, B. Reinius, S. Sagasser, G. Winberg, R. Sandberg, Tn5 transposase and tagmentation procedures for massively scaled sequencing projects. Genome Res. 24, 2033-2040 (2014).
55. M. Huber, G. Faure, S. Laass, E. Kolbe, K. Seitz, C. Wehrheim, Y. I. Wolf, E. V. Koonin, J. Soppa, Translational coupling via termination-reinitiation in archaea and bacteria. Nat. Commun. 10, 4006 (2019).
56. U. Qimron, B. Marintcheva, S. Tabor, C. C. Richardson, Genomewide screens for Escherichia coli genes affecting growth of T7 bacteriophage. Proc. Natl. Acad. Sci. U.S.A. 103, 19039-19044 (2006).
57. A. M. Grigonyte, C. Harrison, P. R. MacDonald, A. Montero-Blay, M. Tridgett, J. Duncan, A. P. Sagona, C. Constantinidou, A. Jaramillo, A. Millard, Comparison of CRISPR and Marker-Based Methods for the Engineering of Phage T7. Viruses. 12 (2020), doi:10.3390/v12020193.
58. T. Baba, T. Ara, M. Hasegawa, Y. Takai, Y. Okumura, M. Baba, K. A. Datsenko, M. Tomita, B. L. Wanner, H. Mori, Construction of Escherichia coli K-12 in-frame, single-gene knockout mutants: the Keio collection. Mol. Syst. Biol. 2, 2006.0008 (2006).
59. R. Martin et al., Structure of the activated ROQ1 resistosome directly recognizing the pathogen effector XopQ. Science 370, eabd9993 (2020).
60. Steczkiewicz et al., Sequence, structure and functional diversity of PD-(D/E)XK phosphodiesterase superfamily. Nucleic Acids Res. 40, 7016-7045 (2012).
61. Pingoud et al., Type II restriction endonucleases: Structure and mechanism. Cell. Mol. Life Sci. 62, 685-707 (2005).
62. Burroughs et al., Comparative genomics and evolutionary trajectories of viral ATP dependent DNA-packaging systems. Genome Dyn. 3,48-65 (2007).
63. Hatfull et al., Bacteriophages and their genomes. Curr. Opin. Virol. 1, 298-303 (2011).
64. Ackermann et al., 5500 Phages examined in the electron microscope. Arch. Virol. 152, 227-243 (2007).
65. Ma et al., Direct pathogen-induced assembly of an NLR immune receptor complex to form a holoenzyme. Science 370, eabe3069 (2020).
66. Kimanius et al., New tools for automated cryo-EM single-particle analysis in RELION-4.0. Biochem. J. 478, 4169-4185 (2021).
67. Bepler et al., Positive-unlabeled convolutional neural networks for particle picking in cryo-electron micrographs. Nat. Methods 16, 1153-1160 (2019).
68. Croll, T. I., ISOLDE: A physically realistic environment for model building into low-resolution electron-density maps. Acta Crystallogr. D Struct. Biol. 74, 519-530 (2018).
69. Casañal et al., Current developments in Coot for macromolecular model building of electron cryo-microscopy and crystallographic data. Protein Sci. 29, 1069-1078 (2020).
70. Liebschner et al., Macromolecular structure determination using x-rays, neutrons and electrons: Recent developments in Phenix. Acta Crystallogr. D Struct. Biol. 75, 861-877 (2019).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An engineered protein comprising an effector domain, an effector activation domain, and a recognition domain, wherein binding of a target polypeptide to the recognition domain leads to activation of the effector domain via the effector activation domain, and wherein at least one of the effector domain, effector activation domain, and/or recognition domain is derived from a STAND NTPase protein.

2. The engineered protein of aspect 1, wherein the STAND NTPase protein is an antiviral STAND (Avs).

3. The engineered protein of aspect 1 or 2, wherein the Avs is an Avs1, Avs2, Avs3, or Avs4.

4. The engineered protein of any one of aspects 1-3, wherein the effector domain is an endonuclease, a protease, a nucleosidase, hydrolase, or caspase-like domain.

5. The engineered protein of any one of aspects 1-4, wherein the effector activation domain is an NTPase.

6. The engineered protein of any one of aspects 1-5, wherein the recognition domain is engineered to recognize a target polypeptide other than a target polypeptide of a wild-type STAND NTPase protein.

7. The engineered protein of aspect 6, wherein the recognition domain comprises one or more tetratricopeptide repeat (TPR) domains.

8. The engineered protein of any one of aspects 1-7, wherein a microbe comprises the target polypeptide.

9. The engineered protein of aspect 8, wherein the microbe is part of a microbiome.

10. The engineered protein of any one of aspects 1-9, wherein the target polypeptide is a phage polypeptide.

11. An oligomer comprising two or more engineered proteins of any one of aspects 1-10.

12. The oligomer of aspect 11, wherein the oligomer is a tetramer, a trimer, or a dimer.

13. The oligomer of any one of aspects 11-12, wherein each of the two or more engineered proteins are the same.

14. The oligomer of any one of aspects 11-12, wherein at least two of the two or more engineered proteins are different.

15. The oligomer of any one of aspects 11-12, wherein each of the two or more engineered proteins are different.

16. A detection composition comprising:
a. an engineered protein of any one of aspects 1-10 or an oligomer thereof,
b. a detection construct,
wherein binding of a target polypeptide to the recognition domain activates the effector domain and mediates effector domain modification of the detection construct resulting in generation of a detectable signal.

17. A polynucleotide encoding the engineered protein of any one of aspects 1-10.

18. A polynucleotide encoding component (a), component (b), or both of the detection composition of aspect 16.

19. A vector or vector system comprising the polynucleotide of any one of aspects 17-18.

20. A cell or cell population comprising an engineered protein of any one of aspects 1-10, an oligomer of any one of aspects 11-15, a detection composition of aspect 16, a polynucleotide of any one of aspects 17-18, a vector or vector system of aspect 19, or any combination thereof.

21. A formulation comprising an engineered protein of any one of aspects 1-10, an oligomer of any one of aspects 11-15, a detection composition of aspect 16, a polynucleotide of any one of aspects 17-18, a vector or vector system of aspect 19, a cell or cell population of aspect 20, or any combination thereof; and optionally a pharmaceutically acceptable carrier.

22. A method of modifying a target molecule and/or cell comprising:
delivering an engineered protein of any one of aspects 1-10, an oligomer of any one of aspects 11-15, a polynucleotide of aspect 17, a vector or vector system of aspect 19, a formulation thereof, or any combination thereof to the target molecule and/or cell, wherein the target molecule and/or cell is or comprises a target polypeptide; and
activating an effector domain of the engineered protein by allowing binding of the target polypeptide to the recognition domain thereby activating the effector domain via the effector activation domain, wherein effector domain activity modifies the target molecule and/or cell.

23. The method of aspect 22, wherein delivering comprises in vitro, ex vivo, or in vivo delivery.

24. A method of detecting a target molecule and/or cell, the method comprising:
combining a detection composition of aspect 10 or a formulation thereof and a sample or component thereof; and
activating an effector domain of the engineered protein via binding of a target polypeptide in the sample to the recognition domain thereby mediating effector domain modification of the detection construct and generation of a detectable signal.

25. The method of aspect 18, wherein the method is performed in whole or in part in vitro, ex vivo, or in vivo.

26. A method of modifying a microbiome structure comprising:
introducing an engineered protein of any one of aspects 8-10 into a microbiome, wherein activation of the effector domain via binding of a target polypeptide of one or more microbes in the microbiome to the recognition domain results in modification of the one or more microbes thereby modifying the microbiome structure.

27. A method of engineering phage-resistant bacteria comprising:
expressing an engineered protein of aspect 10 or an oligomer comprising one or more engineered proteins of aspect 10 in a bacterium or a bacteria population.

28. A method of cargo delivery comprising:
delivering to a cell
   (a) an engineered protein of any one of aspects 1-10; and
   (b) a cargo,
   (c) a detection composition; or
   (d) any combination thereof,
wherein the engineered protein comprises the cargo or wherein the cargo comprises the target polypeptide, and
wherein activation of the effector domain by binding of the target polypeptide to the recognition domain results in delivery of the cargo and optionally activation of the detection construct thereby monitoring cargo delivery.

29. The method of cargo delivery of aspect 28, wherein the cell comprises the target polypeptide.

LENGTHY TABLES
The patent application contains a lengthy table section. A copy of the table is available in electronic form from the USPTO web site (https://seqdata.uspto.gov/?pageRequest=docDetail&DocID=US20250243471A1). An electronic copy of the table will also be available from the USPTO upon request and payment of the fee set forth in 37 CFR 1.19(b)(3).

1. An engineered protein comprising an effector domain, an effector activation domain, and a recognition domain, wherein binding of a target polypeptide to the recognition domain leads to activation of the effector domain via the effector activation domain, and wherein at least one of the effector domain, effector activation domain, and/or recognition domain is derived from a STAND NTPase protein.

2. The engineered protein of claim 1, wherein the STAND NTPase protein is an antiviral STAND (Avs),
wherein the Avs comprises an Avs1, Avs2, Avs3, or Avs4, and
optionally, wherein
the effector domain is an endonuclease, a protease, a nucleosidase, hydrolase, or caspase-like domain,
the recognition domain is engineered to recognize a target polypeptide other than a target polypeptide of a wild-type STAND NTPase protein, or
the recognition domain comprises one or more tetratricopeptide repeat (TPR) domains.

3-7. (canceled)

8. The engineered protein of claim 1, wherein a microbe comprises the target polypeptide, and wherein the microbe comprises part of a microbiome.

9. (canceled)

10. The engineered protein of claim 8, wherein the target polypeptide is a phage polypeptide.

11. An oligomer comprising the two or more engineered proteins of claim 1,
optionally, wherein
the oligomer is a tetramer, a trimer, or a dimer,
at least two of the two or more engineered proteins are different, or
each of the two or more engineered proteins is different.

12-15. (canceled)

16. A detection composition comprising:
an engineered protein of claim 1 or an oligomer thereof;
a detection construct,
wherein binding of a target polypeptide to the recognition domain activates the effector domain and mediates effector domain modification of the detection construct resulting in generation of a detectable signal.

17. A polynucleotide encoding the engineered protein of claim 1.

18. A polynucleotide encoding component (a), component (b), or both of the detection composition of claim 16.

19. A vector or vector system comprising the polynucleotide of claim 17.

20. A cell or cell population comprising
an engineered protein of claim 1,
an oligomer of claim 11,
a detection composition of claim 16,
a polynucleotide of claim 17,
a vector or vector system of claim 19, or
any combination thereof.

21. A formulation comprising
an engineered protein of claim 1,
an oligomer of claim 11,
a detection composition of claim 16,
a polynucleotide of claim 17,
a vector or vector system of claim 19,
a cell or cell population of claim 20, or
any combination thereof; and
optionally a pharmaceutically acceptable carrier.

22. A method of modifying a target molecule and/or cell comprising:
delivering an engineered protein of claim 1,
an oligomer of claim 11,
a polynucleotide of claim 17,
a vector or vector system of claim 19,
a formulation thereof,
or any combination thereof to the target molecule and/or cell,
wherein the target molecule and/or cell is or comprises a target polypeptide; and
activating an effector domain of the engineered protein by allowing binding of the target polypeptide to the recognition domain thereby activating the effector domain via the effector activation domain,
wherein effector domain activity modifies the target molecule and/or cell.

23. The method of claim 22, wherein delivering comprises in vitro, ex vivo, or in vivo delivery.

24. A method of detecting a target molecule and/or cell, the method comprising:
combining a detection composition of claim 16 or a formulation thereof and a sample or component thereof; and
activating an effector domain of the engineered protein via binding of a target polypeptide in the sample to the recognition domain, thereby mediating effector domain modification of the detection construct and generation of a detectable signal.

25. The method of claim 24, wherein the method is performed in whole or in part in vitro, ex vivo, or in vivo.

26. A method of modifying a microbiome structure comprising:
introducing an engineered protein of claim 8 into a microbiome,
wherein activation of the effector domain via binding of a target polypeptide of one or more microbes in the microbiome to the recognition domain results in modification of the one or more microbes thereby modifying the microbiome structure.

27. A method of engineering phage-resistant bacteria comprising:
expressing an engineered protein of claim 8 or an oligomer comprising one or more engineered proteins of claim 8 in a bacterium or a bacterial population.

28. A method of cargo delivery comprising delivering to a cell
a. an engineered protein of claim 1; and
b. a cargo;
c. a detection composition, or
d. any combination thereof;
wherein the engineered protein comprises the cargo or wherein the cargo comprises the target polypeptide, and
wherein activation of the effector domain by binding of the target polypeptide to the recognition domain results in delivery of the cargo and optionally
activation of the detection construct, thereby monitoring cargo delivery.

29. The method of cargo delivery of claim 28, wherein the cell comprises the target polypeptide.


SEQUENCE LISTING (65 sequences)

SEQ ID NO: 1 - protein, 12 aa, synthetic construct
DIAEYYMQLKHS

SEQ ID NO: 2 - protein, 12 aa, Moraxella osloensis
DVEVCMYEAKNH

SEQ ID NO: 3 - protein, 12 aa, Enterobacter cloacae
DVITRYYQIKFH

SEQ ID NO: 4 - protein, 12 aa, synthetic construct
ETNPIGWQSKFY

SEQ ID NO: 5 - protein, 12 aa, Escherichia coli
DGVISYVQAKRW

SEQ ID NO: 6 - protein, 12 aa, synthetic construct
DLVAEAIQAKFY

SEQ ID NO: 7 - DNA, 14 bp, synthetic construct
  source 1..14: mol_type = other DNA
ccaggaatataatg

SEQ ID NO: 8 - protein, 54 aa, synthetic construct
IRDKELSQSNTIERFIHSLEEKTKNILFEIRTNISYRNEKIELDRSGSGGVGKT

SEQ ID NO: 9 - protein, 54 aa, Sulfurospirillum sp.
IAKHFFSLNESIFDLLEEKRKHTENVLYEIQTNIEFKDRSIEIDRSGEGGVGKT

SEQ ID NO: 10 - protein, 10 aa, synthetic construct
LXXXLXXLXL

SEQ ID NO: 11 - protein, 15 aa, synthetic construct
GGGGGGGGGGGGGGG

SEQ ID NO: 12 - protein, 15 aa, synthetic construct
GGGGSGGGGSGGGGS

SEQ ID NO: 13 - protein, 5 aa, synthetic construct
TEFCA

SEQ ID NO: 14 - protein, 5 aa, synthetic construct
APRPG

SEQ ID NO: 15 - protein, 4 aa, synthetic construct
GALA

SEQ ID NO: 16 - protein, 7 aa, synthetic construct
GPLGIGQ

SEQ ID NO: 17 - DNA, 10 bp, synthetic construct
  source 1..10: mol_type = other DNA
tatatatata

SEQ ID NO: 18 - DNA, 15 bp, synthetic construct
  source 1..15: mol_type = other DNA
gtcgtcgtcgtcgtc

SEQ ID NO: 19 - DNA, 456 bp, synthetic construct
  misc_feature 1..456: note = Synthetic
  source 1..456: mol_type = other DNA
atgattcacactaaagagcaagctaacgttttctatgtactggtaagcgcattccgttctaacctgcaccacttggataacattagccgtcatcgtcgcttagttaacagcttacgcaatgcacctctggaatacggtagcgttgagtctactgatttaactggctgttatcatgaggctggcatggagtcaccctctgaagagcaaaccatccgagtacgctgcacgaccaaggcgcaagctataagtgtcgcacggttggcttgtaacgactatgagcaagactgtgtactggtctataagtcacagactcacagcgctgcactaatctttgctggtggcttggacggttggaagactgagcgtatcgggtcgttccagatggtcgcacctgaagttgaactgacaggcaactacacggtcgatgagttcggacgtggctggaaggtaatctaa

SEQ ID NO: 20 - DNA, 201 bp, synthetic construct
  misc_feature 1..201: note = Synthetic
  source 1..201: mol_type = other DNA
atggtcatggcactctttgtaatcacgatgtacgctctgattggggcgtacttcctaagagacttccgcaagggtctcaaggtacacaaggctaacttctcgcacttgaagtatggctttctaccacgattcacagtccgtgaacgtaacgggcgctttaaggctaacaaggttggtatcttctatgttgcaacgcactga

SEQ ID NO: 21 - DNA, 1104 bp, synthetic construct
  misc_feature 1..1104: note = Synthetic
  source 1..1104: mol_type = other DNA
atgaactacaccgacattcaggcacgtctggcaatcatcaagtcactcccgattagcgaactggacaagcgccagcctcttctggttgcactggctgctgacatcgtgaatggtgagacaagcgatggcaacgacacagacgggtcgcagggtcttgagtatcaggactggtggcacaccttgggtgcactcatgcgtgatgcaggtttccgcatgctgggtaatggtcacttcagtgcggcctactcgcacgaaatgttaccgggtcgagtcatcaaggttggctttaagaaagaggattcaggcgctgcatacactgcattctgtaggatgcaccaaggccgtgctggtatccctaacgtctatcacgtagcacgtcatgcaggttgctatacggtcgtactggaccgcttaaaaagctgtgaccgctacgctaacgatgttcacgcaaagtatgcaagcgctgcgcaggagttcattgagtgctctggaggtgatgccgattggtacgcacagtatgttagcaaggagttcattgagacgtgcaagatgattcgtgagttcttccacggtattgcatctttcgacatgcacagtgggaacattatgttcgatgacaacgatgtcccgtacatcactgacccggttagtttctcacatgaccgtgaacgtgaggatggtttccctctggaccctgatgcgttgattgccgaggtcgaggcgatagctaacgagcgcatcattgaacggtgccgcaaccgcaaggccaaatgtgaccctaacggaacctttcaggttaaccgcaaggccgcaatgaaacgccgtaagcgcaaccgcaagttacgtgctaaacatgcagcggaaggaatgcaccagtttgtcaaagagcgccttaagcgtaaagctattgaccgaaatgaacgccgtgctgagatgctcatggggtctgcatggcattacgcatggttgcataatggtaactcaacgttccgcaaggttgaacaagtgaacggcctcaagtggcaactaggggaccgcctagcaatccaagcgggtctacctctgaacattgacaagttacttgacgcacaactgcaaggctga

SEQ ID NO: 22 - DNA, 2652 bp, synthetic construct
  misc_feature 1..2652: note = Synthetic
  source 1..2652: mol_type = other DNA
atgactaacgtaatcaacgcaccaaagaacgacttctctgacatcgctaacgcaatccagccgtacaacattctggctgaccattacggcgcacagcttgcagctacgcagttacagcttgagcacgaagcacacaccgagggcgagaagcgttttcttaaggcaatggaacgtcaaatcaaagcgggtgagttcggtgataacgcagtggctaaaccgctcctgtcatctctcgcaccgaagttcatcgaggcgtggaacacttggttcaccgaggtggaagcgaagcgtggcaagcgtccagtagcctacaatctggtacagaaggttgcacctgaagcggccgctttcatcacactgaaggtcacactcgcttgcttgacgaaagaagagttcaccaacctgcaatcggtcgccactaagattggtcgcagtattgaggatgaactgcgttttggtcgcatccgtgacgaagaagcgaagcactttaagaaccacgtacaggaggcactgaacaagcgcgttggtatcgtctacaagaaagcattcatgcaagccgtagaaggcaagatgttagacgctggtcaactgcaaaccaagtggactacatggacacctgaagaatcaattcacgttggtgtgcgcatgttggaactgctcatcgggtctactggtctcgttgagttacaccgaccgtttgctggtaacgttgagaaggatggcgagtacatccagttaacagaacaatatgtagacctgctatccaaacgtgctggcgcactggctgcaatcgctccgatgtatcagccttgcgtggtccctccgaagccttggacttctccggttggtggtggctattgggctgctggtcgcaagcctctctctctggttcgcactggtagcaagaagggacttgagcgttacaacgatgtgtatatgcctgaagtctacaaggcggtgaacatcgcacagaatactccttggaagattaacaagaaggttctggcagtggtcaacgagattgtgaactggaagcattgcccggttgaagatgttcctgctctggaacgtggtgaactgcctgtcaaacctgatgacattgacaccaacgaggcatcgctcaaagcgtggaagaaagcggcctccgcaatctaccgcaaagagaaggcacgagtgtctcgtcgtatgagcatggagttcatgttaggtcaggccaataagttcgcacagtttaaagctatctggttcccgatgaacatggactggcgcggccgagtgtatgcggtcccaatgtttaacccgcaaggcaacgacatgaccaaaggcttgctgacgctcgctaaaggtaaaccaatcggtgtcgatgggtactactggctgaagattcacggtgctaacactgctggtgtggacaaggtggacttcgctgaacgcatcaagttcattgatgacaaccacgagaacatcatgagcgttgcggctgacccgattgctaacacttggtgggctgaacaagactctccgttctgcttcctcgcgttctgctttgagtatgcgggagtccagcatcacgggatgaactacaactgctctttaccgctggcgttcgatggctcttgctctggtattcagcacttctccgcaatgctccgcgatgaggtcggtggtcgtgcggttaacctgcttccaagcaaagaggtacaagacatctaccgcatcgttgctgaacgtgtgaacgagattatgcaacaggacgtaatcaacggaactgataacgaagtggaaactgttatcaacaaggacactggagaaatcactgagaagctcaagctgggtacgaaggaactggcgggtcaatggttggcctacggtgtgactcgcaaggtcactaagcgttcggtcatgacgttggcttacggctccaaagagtatggcttccgtgaccaagttcttgaggacacaattcagccagctatcgacgatggcaagggcttgatgttcactcaaccgaaccaagcggctggctacatggctaaactgatttggaatgctgtaacggtcacagttgtcgcagcggttgaagcgatgaactggttgaagtctgctgctaaacttctggctgctgaagtcaaggacaagaagaccaaagaggttctccgcaagcgttgcgcagtacattgggtaactcctgatggcttccctgtgtggcaggaatacaagaagccagtacagacccgcttgaatctgatgttcttaggccagattcgtctacagcctaccgtgaacaccaacaaggatagcgggattgatgctcgtaaacaggagtcaggcatcgcacctaactttgtccactcaatggatggaagccacctgcgcatgaccgtggttcgctccaatgaggtctacggtgtggaatcgttcgcactgattcacgactcctttggtactatccctgctgatgctggcaacctgtttaaagcagtgcgtgaaacgatggtcaacacttacgaagagaacgatgtacttgcagacttctatgagcaatttgctgaccagttgcacgaatcacagcttgacaagatgcctgagatgccagctaaaggttctctggacctgcaagaaatccttaagtcagactttgcgtttgcctaa

SEQ ID NO: 23 - DNA, 558 bp, synthetic construct
  misc_feature 1..558: note = Synthetic
  source 1..558: mol_type = other DNA
atgtttactttcatcacttcagtaggcgctctcgtatcaacaattcttgcagcaacatactataatcgttcaaaggtatttctcactaagctaatcaaagaagagaagaactctcatgcactcaaagagagacttgaagatggatggcgagagaagcaggaactagctggtcgaatccgtaccttagagttcgaacaacaatacgacatgtcgaaccaacgagaactcattgagcgtctacgcgctgaggcagtggagtaccgcaaggaacttaaagaggcaaagagtaccatccgtgagtacactcacagtgacatcacgtctatcaagtttaccgctcacatcccggatgctgggtggaagcgcactgagttcaagctgggtatagggtcttgtggcaaagaggttacatgtttgcactggaaagaacagtcggaccgctatctgctgacacagacgtgcactgacggtgagcgcaaagagttcacctattataagaaagatgtagctggtcgcattgagattgcacgtaaggttgcagacaatacgactcactaa

SEQ ID NO: 24 - DNA, 150 bp, synthetic construct
  misc_feature 1..150: note = Synthetic
  source 1..150: mol_type = other DNA
atgcgtaccaactttgaaccaaccactaagcgcaacaacgtaatcaacgaacacggtgctgaatggcaggaacgtaaagaccgcatgaagaaacgccacaagacacaacgtggcaacgctgagaaacgtaactggaaggaggcactctaa

SEQ ID NO: 25 - DNA, 273 bp, synthetic construct
  misc_feature 1..273: note = Synthetic
  source 1..273: mol_type = other DNA
atgggtcgcttatatagtggtaacttaatggcgttaaaggatgtcatcgagagacttaaagaagaccacgatgtcagtgttgtaactgagactttccgctatgagaactacgcacagcgccgatgggtctccggtgagactctccgcattgttctacgcaatggcctcgtactgacatccaagtgtttcgaacagtcagacgaagatgtccggtgcaatgcacagactgagtggctccgcaaggtacacgcagacctgaagcactggaagtaa

SEQ ID NO: 26 - DNA, 1032 bp, synthetic construct
  misc_feature 1..1032: note = Synthetic
  source 1..1032: mol_type = other DNA
atggaaatcacattcaagactaacccttttaaagctgtatcctacgttgagtctgccattaagaaagcactcgacaaagctggctacctgattgctgacactaagcatgatggtgtacgtggtaacatcgtggttgacaatacggctgactgcctgtggctatcccgtgtctccaagaccatccccgcgcttgagcacctcaatgggttcgaccaacgctgggctaatgttctcaaagatgaccgctggattttccctgatggcttcatgctggatggtgaactgatggtcaaaggcgtagacttcaacacagggtctggcctactgcgtaccaagtggctcaagaagacaaactgtggatttgatattggtggagtagagcctctcaaggggagtaaagtagacttctgcctagaccctaagaaacttaaggttgtcctgtatgccgctatgccgtatcacgatatggtagaaggcaaagagtacggcgttatgtccctcctgatgcaagagcatgtgcgttccctcattccggttcttagtgaacacttcccggaaatcacatgggaactctctgaggccaaagaagtatacgacatggtagaacttcaggaactctacgagaaagcccgtgccgatggtcatgaaggtctggtcgtcaaagacccgctggaagtctacaagcgtggcaagaagtctggctggtggaagatgaagcctgagaatgaagctgatggtattatcgttggtatcaattgggggactaaaggtctcgctaatgaaggcaaagttattggcttcgaggttctcttggagtccggtcgtgtagtctctgccaacaacattagtcaagcattgatggatgagtttaccgctgccgtcaaacatgctgagtctgtgggtatgggtatgccgtacactggcttcgcctgtcaggtagcctacatggaggaaactcctgatggttctcttcgtcacccatcgttcgttatgttccgtggtgtcgagtcagacccaacggtcaagatgtaa

SEQ ID NO: 27 - DNA, 81 bp, synthetic construct
  misc_feature 1..81: note = Synthetic
  source 1..81: mol_type = other DNA
atgctatttcttattgctgctgttatctttgccacgtttgccttcgttatcattgacgacaactgctggcctgattgctaa

SEQ ID NO: 28 - DNA, 575 bp, synthetic construct
  misc_feature 1..575: note = Synthetic
  source 1..575: mol_type = other DNA
atgttacgactacacttcaacaaatcaaatggcatcttctctgcccgtggcgctgaccgttcaaccgttctggcctctgagaaacacgcagtcatccctaaagtgcttggctcgaccattgagttagcacctcgtgttcacgctgtaatcaccactggcttatataaagaagcgaccactggctcccggcctttcattccggttctcgtcacccgtttccctaaagtccgtctggtaattgcgcgtatcaaggaggtgttctaatggtctgcatggcatgtttagagaatgatgaccgctacccacatacatgtgaacctgtagctaaaccacaagatatagagggtgttcgaactccgagccactatatgttgtttgatgacattgaggctatcgaagtgattgcccggtcgatgacccgtgaacagttcaaagggtactgcttagggaacatccttaagtatcgcctacgtgctggtaagaaatccgaactggcattcttagagaaggacatggcgaaagcttggttctacggtgaactgtacgagaaacataagggtaagtgctatgcttaa

SEQ ID NO: 29 - DNA, 98 bp, synthetic construct
  misc_feature 1..98: note = Synthetic
  source 1..98: mol_type = other DNA
atggaaaagcctagcgaatggtgtgagcgtatgtttgaaaagacaggcaacaccgactaccttgaattatataatcactggaaggagagaggacaatg

SEQ ID NO: 30 - DNA, 192 bp, synthetic construct
  misc_feature 1..192: note = Synthetic
  source 1..192: mol_type = other DNA
atgagcactctgaataaagggtcactcgtaaacggtgacaagaagttcttcgcaacggtcgaaggccaaggccaatcgttcgaagtccctgtgttcgctgcgtctcttgaagaggcaaacgagttagctgaatggcagtatgttccggctggcttcgcagtgacacgcatccgtccagaccgaaaggtttaa

SEQ ID NO: 31 - DNA, 699 bp, synthetic construct
  misc_feature 1..699: note = Synthetic
  source 1..699: mol_type = other DNA
atggctttcaacaaacgtaaagtattcacttccgctcgtggcgttgctgaaccgtattgctatctggctaaaccggacttcggtagcggtgacttcaagaacgaacgtggtgtctacaaggtgtcacttactgtatccaacgacgacccccgctgtcagaaaatgatttacgagattgtcgaagcccatgaagcggactatgctgctcgtctcgaagagtacgaagcgaacccgccgaaagttgtcaagggtaagaaaccgctgaagccgtatgttggcgacatgcctttcgtggataacgaagacggaaccactacgttcaacttcaagtgctacggctcgttcactgacaagaagactggtgagaacaaaccaatcgacttggctatcgtggactccaagggcaagcgcattcgtggtgaacgtccagctatctctggtggctctgaactgaaaatcaaatacaccctgttcccttatggctggtctgctgtggctggtgcatccgttaaactacaactggattctgtgatgctgattaagctggtcgagttcggtggtggtgatgacgattggggtgatgaagtcgaagaagacggttatgtagctgatgaagcacagacccgcaagccgcagaacgactctggctgggacgaagaaccagaagaggacgatgaagatgaagacggtgacttctaa

SEQ ID NO: 32 - DNA, 447 bp, synthetic construct
  misc_feature 1..447: note = Synthetic
  source 1..447: mol_type = other DNA
atggctaagggttatgcaggtcgaggcactcgtgtgggtgcctttcgctctggcttggaggataaggtaagcaagcaactcgaagggaagaacattaagttcgactacgagcgctggcgtgtaccatatgttgttcctgcaagcaaccatacctattgcccggacttcctattacctaatggcatctttgtggaaacaaagggtctgtgggatagtgacgaccgcaagaaacacttactgattcgtgaacagtatcctgaacttgatgtgcgcatcgtgttctcttcaagccgctctaagctatacaaagggtctccgaccagttatggtgagtggtgcgagaagcatggcattctgtttgctgacaaattaattccagttgattggctgaaagagccttctaaggatgttccgttcgataaatttaaagaggtgaagaagaaatga

SEQ ID NO: 33 - DNA, 456 bp, synthetic construct
  misc_feature 1..456: note = Synthetic
  source 1..456: mol_type = other DNA
atgagtaaggtacaattcaaaccacgcgctgtgacagaagcaatctttgtccactgtagcgcaaccaaggcgtccatgaatgttggactgcgtgaaatccgtcagtggcataaagaacagggctggcttgatgtaggctaccacttcattattcgccgtgatggtacaatcgaagaaggccgtccggtcgatgtagttgggtctcacgttaaggactggaacagtaagtcagtcggtgtgtgcctcgttggtggtatcgacgataagggcagacacgaagctaactttacgccagcacagatgcactctcttaaagagaaactggcagacctgaaagacctgtacccggatgcagaaatcaaagcacaccacgatgtggctcctaaagcgtgtccaagttttaacttgagtcgctggctgaagactggtgaattagtaacaagtgattggggttaa

SEQ ID NO: 34 - DNA, 90 bp, synthetic construct
  misc_feature 1..90: note = Synthetic
  source 1..90: mol_type = other DNA
atgatgattattcatgagatgggaatgtacgatatgatttaccgctgtgtattcgttctggcattgacctatatcttaactcgtaagtaa

SEQ ID NO: 35 - DNA, 87 bp, synthetic construct
  misc_feature 1..87: note = Synthetic
  source 1..87: mol_type = other DNA
atgcgtaagctatttggtgaagcacttccggtgacacttttggtcatcatcattatcgtttgtggcgcactgcgtccgttcctgtag

SEQ ID NO: 36 - DNA, 1707 bp, synthetic construct
  misc_feature 1..1707: note = Synthetic
  source 1..1707: mol_type = other DNA
atggatatggaagaacaacaagagagtatattcctctatcatttaccttgtgagaactgtgggtcgtcagatggtaatggcatgtattctgatggacaccagtattgttttgtgtgtcagaaccatgtgcgtggaaccgaggaaactcgtgagaccgtggcgaagactcgcagaagctcatacaacactggaggtgacaaaatgtctaacctgttaaactttggggattcagacggtcgctacacgaacctcaaagctcgtggccttatggaagctatctgccgtaaatatggctattgggtagcgaaggtcaacggtgagatgctgcaagttgcaaactactatgacgttgagggaaatctcgtaggtcagaaggtacgcgacaagcacaaagagttctccgcaaaggggaaacttaaagctgacctattgtttggtaagcagttatggaatggtggcaagaagattgtggtgactgagggtgagattgattgtctgaccgtagcgcaacttcaagaagggaagtatcccgttgtgagtctccctatgggtgctcaagccgcaaagaaaacatgtgctgcaaactatgaatacttcgaccagttcgatgagattatcctgatgttcgacatggatgagccgggccgcaaggccatcgaagagtgtgctcctgtcctgcctagcgggaaagtccgagtcgctgtactgcctcttaaggatgccaatgagtgtttgctcaatggacaagccaaagcagtaaccgaccagatttggaatgcgcaaccttgggtgcctgatggtgttgtctctgctgtgtcacttaaagaccgtgtgcgtgaagcgatggtcaaagaggaaacaacaggtctactcttcacgggtcaaccgaagctcaacgacatgacacttggtgctcgtggtggtgaagtcatcatggtaacttccggttccggtatgggtaagtcaacattcgttcgtcaacagatgctcatgtggggcaagggtggtgcgaaggtaggcttggcgatgcttgaggaagccgtagaggaaactgttcaggacttgatgggcttgaacaaccatgtccgtctacgtcaagacaaagaactcaagatgcaaatcttagaggatggacgcttcgatgagtggtacgatgtactgttcaacacagatatgttccacctgtatgattcctttgcggagtcacaggaagaccgtctgttcgcaaagctggcctacatggtcgatggcttagactgtaacgttatcctgcttgaccatatctctattgtggtctccgggatggaagataactccgatgagcgtaagacgattgaccgactgatgaccaagctgaaagcctttgcgaagactaaaggtgtggtcgtggtggtaatctgtcacctcaagaatcctgagaaggggaaagcacatgaagaaggtcgtcctgttagtattactgacctgcgtggttctggcgcattacgccaacttagcgatactatcattgccttggaacgtaaccagcaaggtgactatcctaatcttgtccagttgcgtgttctcaagtgtcgttttactggcgatactggcgtggctggacacatggcctacaacaaagaaacgggctggcttgaaccgactgtctcacctgaagaagatggaggtggagattgggaaccggaagaagacggtcaagacttctga

SEQ ID NO: 37 - DNA, 357 bp, synthetic construct
  misc_feature 1..357: note = Synthetic
  source 1..357: mol_type = other DNA
atgaagaaggtcgtcctgttagtattactgacctgcgtggttctggcgcattacgccaacttagcgatactatcattgccttggaacgtaaccagcaaggtgactatcctaatcttgtccagttgcgtgttctcaagtgtcgttttactggcgatactggcgtggctggacacatggcctacaacaaagaaacgggctggcttgaaccgactgtctcacctgaagaagatggaggtggagattgggaaccggaagaagacggtcaagacttctgatagtaaatcaatagaactcactaatgggacagacactctgttcccacttatcaatccacttaaggagaatcacaatgtttaa

SEQ ID NO: 38 - DNA, 213 bp, synthetic construct
  misc_feature 1..213: note = Synthetic
  source 1..213: mol_type = other DNA
atgtttaaattcatcaacactcttggtaagctggtagttaaaatgtacttccgtgaagctaaacgtctgaacgacaaggcacgtaaggatgcagaacaggctcaacgcttggcgaaacaggcgcgtctactatctgaagatgcaagtgctggcgttacgtctgctgctaagattgcagcgaaagcaaccgacctgaacaagttcttcctgtga

SEQ ID NO: 39 - DNA, 180 bp, synthetic construct
  misc_feature 1..180: note = Synthetic
  source 1..180: mol_type = other DNA
atggttacgataacccttgtgctcactctgataactgtgagcggacaaacaataagtcaagtccctatcacggcaccgaaggctgaggcatacgagatatgcgaacagcaagggcgctcatggtctaaggacttgatgaagaacggcagggttttcactgccaactacaactgtatctaa

SEQ ID NO: 40 - DNA, 303 bp, synthetic construct
  misc_feature 1..303: note = Synthetic
  source 1..303: mol_type = other DNA
atggctattaaattccccggcaacacaatccgtctgtccgacaccatcgaccagtatgcccgtcgagtgcacatcaacgtccgcaacggaaaggtaacactggtctaccgttggaaggacagtaagtccaccaaggcgcacacacagcgtgtgactctggatgacacacaggcaggtcgtctgctggcctccgttgctgtagccgctactgtggctgtaggtgaagacaatgttcgtcaggttctgctgggtaaagccgtggagtctgagggcaacatgcttgctgacaagtgtgaagagtaa

SEQ ID NO: 41 - DNA, 417 bp, synthetic construct
  misc_feature 1..417: note = Synthetic
  source 1..417: mol_type = other DNA
atgcgtaatcgtaatgtaatcgaagctgaaatcgctaaactggaagctgaactggcagatgttaaagagtatgactgtgctcgtgactccgctgtccacatcctgaagaacttagggtggacccgcaagtatggcaagtgggtcaaaccagtagccaactgtggtagcgttggtatgcagactttcgacgcagacaagatgacccacattaaggctggggacttctgcacctccaccagcaacgcagacatttggtatgtccgtaaggtcgaagggacagacatttggtgctcccgtgtgactcgcattgatgcaatgggtacagtcgctgggattcagtactacaagatgaaccacaagaacctgcgtgtggttgaccctaagaacttcatgggataccgcaaatctacattttaa

SEQ ID NO: 42 - DNA, 2115 bp, synthetic construct
  misc_feature 1..2115: note = Synthetic
  source 1..2115: mol_type = other DNA
atgttactatccgacatcgaaacaaacgggttgctgcacaacgttagtcaatttcactgtgcggtgacttacgactacacaactgaccagtacatccgttaccgacctgatgactttggggcgtacattgatgcactcgaagccgaggttgctcgtggtggtctcattgtgttccataacggtcacaagtatgacatcccggtaatcgagttgctcgccaagaaactgttaggccgcgacgttaagttcccgaaagagaacgtactggataccctcgtgatgtcccgcttgatttactctaacatcaaagacaccgatgctggtctgctgcgttctggcaagctgccgggtaaacgttttggttcccacgctctggaggcttggggttatcgcttaggcgaaatgaagggtgagtacaaggacgacttcaaggcttcactggctgagtctggtgaagaatatgttgatggtatggaatggctcctgttcaacgaagatatgatggaatataacgttcaagacgttgtagtcaccaaggctctcttcgagaaactttgtagcaacacattctatttccctaacgagacacccgctggaagcacagaagcagaccgtttctggaatggtagccttgaggctgttaaactggaacatgatgctgcatggttgttagccaagatggaacgtaacggtttcccgattgacattaagtctcttgagaacctgtacgcagaactcgctggtagacgtggtgagctactcgtagagttaaccaatacgtttggctcatggtactccgctaagggcggtactgaagcattcagacaccctcgtaccggaaagccgctcaccaagtatcctcgtgttaagtacccgaaacagggtggcatctttaagaagcctaagaacaagaagcaacgtgagggattagagccgtgcgagttggatacccgcgattacgttgagggtgctccgtatactccggttgagcacgttgtgtttaaccctgcgagccgagaccacatcacccgtgttctcaccaaggctggttgggttcccgttgagttcaccccaagtggtgcaccaaaggtagacgacgaggtcttagagcacgttaaggtagacgaccctgatgcacagcgttgcattgagttgattaaagaataccttatgattcagaaacgtatcggtcaggttgctgagggtgataacggctggctccgaatgattggagaagatggtcgtattcacggaagcgttaacccgaacggtgcggtaactggtcgtgcaactcatagtttccctaacgtggcacaggtgccttcaatccgtgctgcatatggcgaaccttgccgtgctgctttcggtgccgagcacaacatgaaagacggtaagccagacccttggattcaagttggtgtggatgcgtccggccttgagctacgatgcttaggtcacttcatgtatcgctatgatgagggagagtacgttgagactatcctcactggtgacatccatacgaagaaccaattggctgctggtctgccaactcgtgacaacgctaagacgtttatctatgggttcctctatggtgctggcgctgcgaagataggacagattgttggtggtactgccgaagacggtaagcgactcatcaagaacttccttgagcaaactccggctatcgctgcattacgtgaggccattacaggtactctcgtcaaggactctaagtgggttggcggtgaacagaaagtaacttggaaacgccgttgggttcgtggattggatggtcgtaaagtacacgtccgtagtcctcacgctgcgttgaacaccttgttgcagtctgctggtgctctgatttgtaaactgtggattgtcgagactgaacgactgttactggaagctggcctgaagcatggctgggatggtgactttgcgtacatggcatgggttcacgacgaaatacaggtcgctgcaaggactcaagagattgccgataagattgtcgagttggcacagcaagcaatgcgtaatgtgggagaccactttaactttagatgtcagttagataccgaaggtaagactggcgcaaactggaaggattgtcactaa

SEQ ID NO: 43 - DNA, 228 bp, synthetic construct
  misc_feature 1..228: note = Synthetic
  source 1..228: mol_type = other DNA
atggcttggttatatagaatacgtgaagttagacttactacaggagagacactctactaccctcaattccgccacaagtggttaccgttttggtgccacttccgggattattcaaatgagaacaaagtgtcattcacttattactctgaagctgtcaactttgttaacgagtcgaaagcaaatgacgaacctgaagttaaacaggttgtccgtaatcactatctttaa

SEQ ID NO: 44 - DNA, 285 bp, synthetic construct
  misc_feature 1..285: note = Synthetic
  source 1..285: mol_type = other DNA
atggctattactaaacgttttaaagtatcatttgaagttaccgctgtaatcaactctgagatggagaaaggcttcaccgaggaactggttcatttggctaagaaggctgcggcaggtgagaagattagtcctcgtgaccgtgaacttctggtacaagcgctgactcatggtgtcggtggtgctatctcctttgccgccaaacagggtatgcgagagttcatccgtaccgagttaccggaagatggtctgaaggtttctccagcaactatccgtgaggtgaagtaa

SEQ ID NO: 45 - DNA, 210 bp, synthetic construct
  misc_feature 1..210: note = Synthetic
  source 1..210: mol_type = other DNA
atgggtgaatatctacgtgtactggcggctatcaaaagctgccctaagacctttcagtcgaactatgtgcgcaacaatgcgtcactggtggctgaggctgcgagtcgaggtcatatctcttgcctatcagtcgatgggcgtaatgctggcgcttgggaagtaacaggcgctggtgtccgattcctgaagaagatgggaggttgcgtatga

SEQ ID NO: 46 - DNA, 186 bp, synthetic construct
  misc_feature 1..186: note = Synthetic
  source 1..186: mol_type = other DNA
atgagtctgcacacagacaacgttacagtgacccgtgaggcgtggaacgacatgcagtcgtacatccagtcactggagaagaaagctgagaagctggacgctctggaggctggaggtgtggacaataggtccggctactggttctctctggaagaggctggtctcacaggggaggacgacttatga

SEQ ID NO: 47 - DNA, 915 bp, synthetic construct
  misc_feature 1..915: note = Synthetic
  source 1..915: mol_type = other DNA
atgagtgcaattacactgaaggagttctacgagttacgccaagggaaatccgataagggtgttctcgtaatggatggtgactggttggtattccaagcgatggcagcaagtgaagtcgagacagattggggtgacgatgtgtggaccttagagtgtgaccacgggaaagcgtggaacattctggttgactctatcaagtcctatgcgagccgtaagaaggcttggcgtgatgcacctatcgtattggccttcactgacgctgtgaactggcgtaaggaactggtggacccaacctataaggagaaccgtaaggcatcccgtaagcctgtaggctaccgtgcgttcatcgagcgtgtccaagcctgtgaggactggacgtctatccttgaggaacgacttgagggtgatgacgtaatgggaatcatcgggtctggtgctaaacattttggtttcaagaaggctgttcttgtctcctgtgataaggattttaagaccatccctgattgtgacttcttgtggtgcacgactggtaacatcttgactcaagaccaagcgactgctgactactggcatatctatcagaccattaaaggtgacttgacagatggttatggtggcattccgggctggggcgagactaccgctgagtggctggagaacccatatgcctttgaacaggttgagaaaactttcaagtctggtgcccgtaagggtcagacagttctcgaatggaagaaggttgacatgggtgagcgaaccttatgggaagccattgtgacacttggtgcgaaagctgggatgaccgaagaggaaatcattaagcaaggccggatggctcgcatccttcgattcgaagagtacaactttatcgacaaggagatttacctatggtcgccggaacagttgcgttcgtaa

SEQ ID NO: 48 - DNA, 246 bp, synthetic construct
  misc_feature 1..246: note = Synthetic
  source 1..246: mol_type = other DNA
atgttaacccctatcaagaaatacatggagaacccacaagatattcctagcgttccacgtagtgttatggagtatttacaggttcaatataatgctggatatgccttgcagtctggtttagttaaccgattgaaacaggctggttggtctgaaagctacattgctgggttcctcgcgggtctcaactatgcgtctcaaaccttagacgacatggaggcaatccgtaaggaacaggctgagtcctaa

SEQ ID NO: 49 - DNA, 273 bp, synthetic construct
  misc_feature 1..273: note = Synthetic
  source 1..273: mol_type = other DNA
atgtgcttttcaccaaagattaagacacctaaagtaaagaccgatactgtccgagctattgacccagcgccactgactgaggaaccgaaaggtattctctttggaggcgatgaggacacagatagcggaacctcttctgaggtgccaacgggtggcaagaagtcccttaaggtgaaactggatgactccgttgagaagtccaagaaggctgatgatgcagccaagaagaagtctaaatctggtatccgtaccagtatctttaagaagaaataa

SEQ ID NO: 50 - DNA, 315 bp, synthetic construct
  misc_feature 1..315: note = Synthetic
  source 1..315: mol_type = other DNA
atgggttggggtaaaaagattcgaaaatcagttaagaaggctgtcaagaaagtaaccaagcctatcgagaaggtagtcaaggaaactggcaaggcagttgagaaggttggtaaggaagctgggaaagtagttggtggtgttgctggtactgttcaagctgcacctcctgctgcacagattattgagccaccgagtaaagacgatgtagactccgatgatgatgcacagactgaatccggtaagaagaaagcccgtgctggcggtaagaaatctctgagtgtcgctcgcagttctggcggtggtctgaatatctaa

SEQ ID NO: 51 - DNA, 1608 bp, synthetic construct
  misc_feature 1..1608: note = Synthetic
  source 1..1608: mol_type = other DNA
atggcaagctcacagaaacgtgaaggcttcgcggagaatggtgccaaggcggtgtatgacgcattgaagaacgaccggaactcctacgagacccgtgcggaaaactgtgcgaagtacaccatcccttcgttgttccctaaagactccgacaacgcctctactgactacaccactccgtggcaagcagttggcgctcgtggcttgaacaacttggcctctaagctcatgctcgctctgttccctatgcagacttggatgaagctgaccatctctgagttcgaagcgaaacaattggttgcacagccagccgaactagcgaaggtcgaagaaggtctctcaatggtcgagcgaatcttgatgaactacatcgagtccaactcgtaccgtgtgacactctttgagaccctcaagcagttggtcgttgcaggtaacgctctgctctacattcctgaacctgaaggtgcttacaatcccatgaagctataccgcctgtcttcttatgttgtccaacgagacgcattcggcacggtgctacagattgtaaccttagataagactgcctatgcagcacttccagaagacgtgcgaaacgctatggactccggtcaggaacataaaggcgacgaaatgattgatgtgtacactcacatttatcttgacgaagaatcaggagaatacctcaagtacgaagagattgatggtgttgaagtcgatggtacagatgcttcttatccggtggacgcttgtccttatatcccggttcgcatggtacgcattgatggagaatcatatggtcgttcttactgtgaggaatacttaggtgacttacggtcccttgagaaccttcaggaagctatcgttaagatgtcaatgattagtgcaaaagttatcggcttggttaacccggctggtatcacacaggttcgtcgtttgaccaaagcacagacaggtgacttcgtgtccggtcgtccagaagacatctcgttccttcagcttgaaaaggccgctgacttctctgtagcgaaagctgtgagtgaacagattgagggccgactgtcctatgcctttatgctgaactccgctgtgcaacgtactggtgagcgtgtgactgctgaagagattcgatatgtcgcatctgaactcgaagatacccttggtggtgtctattcgattctctcacaggaacttcagttgccaatggttcgtgtcctgctgaagcaacttcaagcaaccaatcagattcctgagttaccgaaagaggccgttgagcctactatcagtaccgggatggaagcattgggtcgtggtcaagacctcgataagctggaacgttgtattgctgcgtggtctgcgctggctcctatgcagaatgatcctgacatcaacattgcgaccattaagttgcgcattgcgaacgctatcggcatcgacacttctggtatcctcaagacacctgaagagaaacaacaggagatggctgaggctgctcaaggcactgctctggagaacgctgctgcgtctgctggtgctggtgctggtgcactggctactgcaagtcctgagaacatggaagctgcggctgcacaggctggtatggttcctaattaa

SEQ ID NO: 52 - DNA, 912 bp, synthetic construct
  misc_feature 1..912: note = Synthetic
  source 1..912: mol_type = other DNA
atggctggtgaatctaacgctgacgtatacgcatctttcggtgtgaactctgcggttgttggtgggtctactcatactgaacacgaacaggctatgcttgaactcgatgtttctgcccgtgatggcgatgatgcaatcgagctagttagcaacgatgacccttatggcaatccagacccattcggtgaagaggatgagagtcgtactgagattcgcatctccactgatggtgaacctgaagtaaccgaagaaggtgaaatcgactatgaaggaaccgaagatgaaggctccgaagagtttgaaccagttggtgacgttccagatgaactgaacgaagcctcaagtcaactcgaagagcacgaagctggtttcgaagagatggtcaatcaggcggctgaacgtggtctgtcagaagaaaccatcgtgcgtatccaacaggaatacggagaggacggtctgtctgaagagtcttacgaagagttagccaaagctggttactccaagtcgtttgttgactcctacatccgtggtcaggaagcactggtagaacagtatgttcagtccgttatggaatacgctggcggtgaagcacagttccaagcaatctacaatcatctcgaagtgtccaacccggatgctgctgagtctctgattagtgccttggagaaccgtgacctcgcaacagttaaggctatcgtcaatctcgctggtgcaagccgtgctaagactttcggtaagcctgctgcccgtagtatctctaaacgtgccgtacctgctgcacctgtacgcaccaaacagtctggctttgagtcacaggctgagatgattgctgcaatgtccgaccctcgctatcgccacgacagtaagttccgtgcggaagttgagcgcaagatgatgtacagtcagttctaa

SEQ ID NO: 53 - DNA, 1178 bp, synthetic construct
  misc_feature 1..1178: note = Synthetic
  source 1..1178: mol_type = other DNA
atggctaacatgaatggtggacagcagatgggtaaagaccaaggtaaaggcatgagcgctggtgacaaactggctctgttcctgaaggtcttcggtggtgaagttctgactgcatttacccgtacctccgtaactatgaacaaacaccttgtgcgttctattcagtccggtaagtccgcgcagttccctgtgctgggtcgtaccaaagctgcttacctgcaaccgggtgagaacctcgatgacaaacgtaaagacatgaagcacactgagaaggtcatcaacattgatggtctgctgactgctgacgttctgatttacgacatcgaagacgcaatgaaccactatgacgtgcgctccgagtataccgcacagttgggtgaatctctcgcgatggctgctgatggcgcggttctggctgaaatggctaaactgtgtaacctgccgtctgcgtctgatgagaacatcgctggtctcggtaaggctcatgtactggaagttggcaagcagtctgatcttcagggagaccaagttaagctgggtcaggctattatcgcacagttaactctggctcgtgccaagctgactagcaactatgttccgtctgctgaccgtgtgttctacaccactccagacaactactctgctattctggctgctctgatgcctaacgctgcaaactatcaggctctgattgacccgtctactggctctatccgcaacgttatgggcttcgaagttattgaggttccgcatctgactgctggtggtgctggcgaagaccgtccagaagagggtgtaaatccgactggtcagaaacatgcgttccctgagacttcctctggtgacacccgtgttgctctggacaacgttgtcggtctgttcaatcaccgctctgccgttggtactgttaagctgaaagatatggcactggaacgtgctcgtcgagctaacttccaagcagaccagattatcgctaagtacgctatgggtcacggtggtctgcgccctgaagcatgtggcgcgctggttttcaacaaggcttaacttctcgcaaactcgtgggggacattgagtcccttactgaagttgagcaagagctaacgccacaacagaaagctgcccgtacccgtgcggcaaacaaggctcgaaaggaagctgagttagctgctactgaatga

SEQ ID NO: 54 - DNA, 2995 bp, synthetic construct
  misc_feature 1..2995: note = Synthetic
  source 1..2995: mol_type = other DNA
atgcgttcctatgagactaccctagagactggcgaagagttggctgcggtaaatgacatcttggcctctatcggtgaaccacctgtgtccactttggaaggtgatgctaacgccgatgttgccaatgctcgtcgagttctcaataagattaaccgacagattcagtccaagggttggacgttcaacattgaggaagaccaacagttgctacctgatgtgttcaacggattgattccttatatgtccgactacctgtctgtgatgacaccgggaggcgctactgcctatgtcaaccggggtggttacgtgtttgaccggactactggaactgacgtgtttaaagaaccaatcactgtgaccattatcaagctgagagagttctacgagatgcctgaatgtttccgcttctggattgtcaccaaagctgcccgtcagtttaacaacaggttctttggtgctcccgagattgacgcagtgttagctgaagaagaacaagaagcaaagatgcagtgtcacgagtatgaactggactttggtaacttcaacatgcttgatggtgatgccttcactggcggtctgctatctcgctaatgataacaacttaaggaggtctctaatggctcttatttcacaatccgtcaagaacctgaagggcggtatcagtcaacagccggacatcttaaggttccccgaacagggttccgaacagattaacggctggtcttcggagactgagggtcttcagaagcgtccaccttttgtcttcactaagaccattggagaccagaatgcccttggtgcaaaaccgctcatccatttaatcaaccgtgatagcgtagaacagtattacgttgtgttcacaggacagtcagtccgagtgttcgacctcaatggcaaagagtacacagtgaaaggtgacttgagctacgtgaaggtagggaacccgcgtgatgacctgcgaatggtaacggttgctgactatacgtttatcgtcaaccgcaacatggtggttcgtgccgataagacaccactctatactctgaaagagaacggagactgtttgattaacgttcgtggtggacagtatgggagaacgcttgcatttacaataaacaacgttcgaattgagtacaagattgcacaaggagataagcctgaacatgtacaagagaccgatgcacagtggctcgttaagaaactggcaggtcttgctcgtgctcacggttccttcaaggactggaggttcgacgaagggtctggcttcatccacgtaatcgctccgggtaacagtcagattaaccagctgtccactgaggatggctatgctaaccagttgatgaacgcggtaatgcacacaagccagtcattcagtaagttgccaatcgaggctcctaatgggtacactgtgaagattgttggtgacacctctaagacttccgacatgttctatgtccagtatgacaacgtgaagaaagtctggaaggaagtcgccggatggggagttgagaagggtctcaatggaggcactatgccacacgcattggttcgtcagtctgatggttccttccagatgcaggtgctaccgtgggcgcaacgaacttgtggtgacatggatactaacccgactccatccatcgtggaccagaagattaacgatgtgttcttcttccgtaaccgcttagggttcctcgctggtgagaacattgtgatgtctcgtacctctaagtatttctcactgttccctgcgtcagtagctaacctgtccgacgatgacccgattgatgtggctgtatctcacaaccgtatctcaatcctgaagtatgccgtaccgttctctgaagagttgctgctatggtctgaccaagcacagttcgtcctgagcgcttccggtatcctatcaccgaagacagttgagttgaaccttacgaccgagttcgatgtgtccgaccgagcgagaccttttggcgttgggcgtggtgtgtactttgcgtcacctcgtgcttcctacacgtcactgaaccgttactatgcggtacaggatgttagctccgtgaagtctgctgaggatatgagtgctcacgttccaagttacattccgaacggtgtgttctccattcgcggctccggtactgagaactttatctccgtgctctctgcgaacgccccaagtaaaatcttcctatataagttcctctacttgaatgaagagattgcccaacagtcgtggtctcattgggaacttggtagtaacgtaacggttctggcttgtgactctatcggctcaacgatgtaccttgtgttgcgcaatcagtcacacacttggatgtgccgagcacactttacgaagaactccattgacttcaacgatgaaccatatcggctgtacatcgacaacaaggtcaagcatgtgattcctaaaggtgcgtacaacgatgacacctacacgaccactatcaagcctgtggacatctacggtatgaaatattggactggtaggttctacatcgtggcctctgatggtatggtctcgtggttcgaccctccgcgtggtggttggcctaatggtgtccctgtgttgtcaatgagtgggaaccgtgaggatgagacaatctacgtcggcttggctatcaacttccgttatgtgttctctaagttcctcatcaagaagactgctgatgatggctcgacagctaccgaggacattgggcgcttgcaattacgccgagcgtgggtgaactatgaggactctggtgcattcactgtagaagtggagaacacctctcgactgttcagctatgacatggcgggtgctcgcttgggttccaatgcgttacgcactggtggacttaacgttggtactggtcagttccgattcccggttgctggcaacgcacagttgaacgaagtaaggattatctctgaccacactacaccacttaacgtgattggctgtggatgggaaggtaactacctacgacgctcttctagtatctaa

SEQ ID NO: 55 - DNA, 7290 bp, synthetic construct
  misc_feature 1..7290: note = Synthetic
  source 1..7290: mol_type = other DNA
atgctaataatcagacctactaaagaatcagactttgctaggttcactccgtcacctgaagacatcgctgaggcaaaagcctatggcattgaaccttcatttccaccagctaacgagtgcgtcactatgtcacttcatggtatgcccgtggctatcggtggtaattgtggcgaccaagtgtggttcgtaacgtccgctcgtgtttggaaactaagtatgaagacccgaagagagttccgaaagctcattctggagtatcgtgataccatgctcaaacagtatccgattatctggaactacgtttgggtcggcaacaagtcccacattaggttcttgaagtctattggtgcggtgttccataatgagtttactggtgacagtaatcaattccaactattcactataggaggttaactatgtgctggatggctgcaatcccaatcgccatgcaaggtgcgtctatggtaatgggcggtatgcagggtgaacaggcaaaggccgcacagattgaccaaggtcgtcgacaaagctggcagatgattaaggagatgaactacaacgatgctaaccttaagttagaatctcgtgacctcatcgactcaactgtccaagagatgacccaagcgaatatgaaccgtgtgcgaaacatggggactatccgtgcggcaattggtgaagggatgcttgaaggtaactcaatggaacgtgtagctcgcgttactgagggtgacttccttcgagagtctcaaggtctcaccgagaactaccaacgtgactacagtgtcatcttaggtaagcgtttagctaaccgtgagaataccgtaagtcaaatcaatgaaatcaacaagtccgaaccacgtctgaaaggcaagctggaacaaatcattgacccattaggcttaggcttaacgaagctggtcgatgtggcaactgctggtagccctctgactaacaagctgggtaagaaggcagctgctaaagttaaggcttcggatgctaagtctaccggacaaggcaagtaaacacaggaggtaactgtatgagtaagttagcacaagcattgggtaacatgggtgctcctgctgcaagccgactccgtggtactggtcgtatggacgttaaggctgcgactgtctatgaagaccctagctatgccaataagtccgcactgattgggactgtaggtaaacttgctgagatgggtgctgatgcgtacatgaagtatgaccagaagcagaaggacaaggcagatgagcgctccaacgagattatccgcaagttgactcctgagcaacgacgtgaggccattaagaatggaacacttctgtatcaggacgacccttacgcgatggaagcattgaagattaagactggtcgcaacgcggcctacttggttgacgatgaggttgcacagaaggttaagaacggtgagttccgtactcgtcaagagttggaagagttccgtcacagccgactgcaagaagccgctaagaactatgcggaacagttcggtatcaatgagtcagatgagttttatcagaagggtttcaactccgacatcactgaacgtaacatcgccatctatggtgctcatgataacttcctgagtgaccaagctaagaagggtgccgttatcaacagccgtgtcgaactgaactctgtactcaacgaccctgagacccttcgttccccatatgctggtgagttctttgagaactacttcagtgctggattgacgactggtagtatccctagtgatgaccaagcgttcacaatgattagccaaggtctgtctgacgtggttaaccgtgaaggtggcggtcagttcctacaacagattgagaaccgtaaggtgaaactccacggaaaagagacaaccttcaaggaactgatgggtgccgaacagtggaacaatctgatggttaaggctcaacataatgagttccagttgaatgctaagaagaccgaggcgttccaacttaatgtgaactctgcgttgaaccaagagaacgtgaacactggctgggaacagttgcaatccatcaaggctgaactggacacactgcaaccgggcgaagagatgacacctgaacgtcaggcgttaatcaatgcacagacgcagatgcaggacaggatgaagcgggagactgctgagttagcaaagcagatggacaagcaacagaagtcgatgaacaagatgaacgtcattgacgcacagttccagaagcgactcaatggtcagtatgtgtctactgcgtatggcgatatgcctaccaacgagaacactggtgagttcacccatagtgacatggttaactatgcgaacaagaagttagccgacattgacgctatggacatccctgctgaacagaaggaccgaatgaagctggactacctcaaggctgactccgagaagggtgctttccgtactgcggtgggtgaactgattggtgacgctgagaaggaatggacgtccgctgtgattaatgggaagatgcctgaagatggcggtgttgcccttaacgctcttcgtcgtgtccgtaatgctgaccctgagttatttgctgcgctgtatcctgataaagccgagatgttcctcactatggacatgatggacaaccaagggattgacccgcagattctcttagatgcagacaaggctcgacaatctctgaccaaggatatgcagtatgaggacgacaaggcatgggctgctctcaagaacaactcacagtctcctgaactgtcccgtatgccagccaccttggatgctggtgctcgtaagatttatgactctgtgaagtaccgtactggtaacgctgacatggcaatgcaacaggtcgataagtatctcaaagagaacacaacgactctcactggtgatgatgttgatggcgataccattggtgtcttgacgagaaactctctgcgtgtgaccgatgacccggactcttggaaacaaggtaaggacatcatcgaccaagccgctaagaaactcgctgagacgaacccttgggtgactaataagcaactcactgtgttcgagcgtggtgactctatctacctgatggacactaccggacaggttaacgtaaggtatgacaagcagttgctctcgaagatgtaccaagagaatcaggccaagttggatgcggaagcacgcgacaaggcactcaaagatgccaacaagcgaaccctgcatacacgagctatgaaccgtaagcgtgaacgtgaagccaagaaacctaagcgttctggtagcatgtatgattcggtgagtggtaaaggtattctggacacgctgactggtaaagactaacagaccacgataggaggttccaagtggataagtataaccagaacgaaccgcatgaatatgatgcgttatttcaacaagctgctgacacacatggagtctcctatggccttcttcggaaggtcggttgggtagagtcccgctttaagcctacggctcaatcaccaacagggccgagaggtgtcatgcagttcactaaggcaactggtcaggcttatggtctccagaacgatgaggacttcgctgacccggctaagtccattgatgcaggtgctcgttaccttgcagacctcgttaagaaatacaatggggatgaacttaaagctgccctagcgtacaaccaaggtgagggacgtaatggtaaaccccaattggaagcctacgactctggtaactttgctggcatcggtgatgaggggcgcaactatctccgctcacttctggatgtcgctaagtctcctaagagtggtgacattgagtcattcgggggtatcaccccaaagggtaaagggattccgttcgatactgctatgagtggtatcgggaagaaaggaaaggtaactacagaactccctgagtctcactcaatgtcctttcaaggtaaagaacagtccgctcccaatcagccgttcggaaaagactattgggaagcaaaaggaactacactcgacgaagctaatgagcgttcaaccttcttcggattcggtaatgctactgaagcagaactctccaactcaaccttgggtgtggccttccgtgctggtaagcgtgataatggtttcgatgttgttactgatgtacttcaaccgacacggtttaacagccacatttggtcgcctgaagaactcgaaaggattcgcaacgaagtgaagaaccctgcgtacatgaacgtagtgcttggaggttcctctgagaatctggatgaactcatcaagttagccaatgagaacttcgagttggactctaaggccgctgatgctggtattggtgccaaactgtctgctggccttattggtgctggtgttgacccattgacctacgttcctatcgctgggactgcggctaaagggttcaagctggttaacaaggcattgattgtaggtgctcaagctggcgctcttaacgtggcctctgaaggactccgcacatcggttgctggcggtgatgctcactatgctgaagctgcccttgctggtatgctgttcgctggtggccttactgctatcgctgatggtgtggctgctggtctccgtaagtccggtgctgaacagattgagaacccattcgctgctgcacagatgcgctttgaggctcgtgagactgcacgtaacactggtggttatgacgcaagccgtatgcctcctagtgaagaccgagtgttctcgaatcacaatggcgttgagtattcacctcttgaaactgaaccgggtgccgtagtgttgcgtgatggttccattatcagtgatacaaacttggctaacccgatgacagccaaagagttcgcagaagtagaccctgaacgtgctgcgtggggcttgcctatgcgtggactgagtgaaatcggcttgaggactttgcgttctgaacacgctgaaatccgtggtctcgctaaggacttagtgcgttcacctacagggatggagtctggctcacatggtaagttcggtgctacggcctctgacatcaaagagcgtctgcacagtaccaaccagcgaacctacaatgacctctatgatgcaatgaaggaagcaatggctgaccctgagtggtcagtgggtatgttcaagcgtggtgctcaaggtgcccgtcaggaaatctatcgtcgtgctgctatcgctattgagcgtccagaacttcaagccaacttaacgaaggctgaacgtaaggtaatggacatcatgaaggaacatttcgacctcaagcgtgagatgatggagaacccggctatgttcggtaacaaggcaacttccatcttccctaacagtcgtcacaaagggacttacgttcctcatgtgtactcacgggaagccaagcagttgtacgcacaggctcttggtggctctgacggtttacaggaagctatcgctgcaagctggatgacctcttaccgttcacgccctgaagttaaggctcgtgtggatgagcatttagcggagacattgggtatcgacccgaaggctgtaaccgaagagatggtgatgaaacatgccagcgacaaggcttatggtattgcgaagactgatgagttcaactcttcgagtgtcatcgacgataacattgagggtctcgttgggattgagaacaactcgttccttgaagcccgtaacctgtttgactctgacatgccagtcactctacctaatggacagcaattctctgtgaatgacctccgtgacttcgatatgaagcatgtgatgccagcctatgaccgccgtgttgatggtgacattgctatcatgggtggtacaggaaagactactgctgaactcaaggattccattatggctctcgataagaagtcagaagggaagggtacgatgaaaggtgaagttgaagcactcaaggataccgttaagattctcacaggtcgcgctcgacgcaatcaggatactatgggtgacactatggttcgcgctctgtccgatatgtccttctttacgaagaacgcatacatgggcttgcagaacctaactgaaatctctggtctcttggcgaagggtaacacccgtgcgatgctgcatggcattcctgctctgcgtgacctagcattccgtaacaagccagtgtcaggtaaggaacttaaggaactacactctatggtgttcggtaaagagttcgatgacttaatcagaccgacacgtcaggacatcattcagcgactccgtgaatctacggatacacctgatgttgccgctaaggttgttggtacggttaagcacaccactcaagagtttgctgctcgctctccgttcactaagttcctgaatggtacatcaaactacattctcgacatggctcgacagggtgtgatgggtgatgtggtcactcatgctatcactggcaagggtgccaacaagtggattaaaggcgacatgctgaagtctgcgagtatctcaaaggaacagtgggaaggcattcagaacctcatccgtgagaacgtcactcaaggtgctgacggtaagtacaccttcaaggacaaacgcaagctggctaatgaccctcgtgctatggacttgtggcgcttagctgataaggttgccgatgagacaatgctgagacctcacaaggtttcacttcaggattcccatgcgtttggtgctgccgctaagttggtccttcagttcaagtctttcgtaatcaagtcgatgaactctaagtttatccgctctggtcacgaggctatgaagaatcaccgagctatggacatggcattaacctatgcaatctctggaggcatcgctggtgcttactatgttgcacaggcacacatgaaggctgctggtctacctaaagagcaacagaaggattacttgaagaaagctctggaccctaagatgattgcgtatgctgctgcatcccgtagttcacatttaggttccccgttgagtatcgccaacttcgctatgggtgctgctggttacgaccaaggtctcatggtgcgctccacgattctaccgaaaggagaagacaagcgtgaccggaataaagcagtgacctctcgtgatatgggagaatccatcatgggcgctattggtgaacagattcctgctcttggtttcgctggggcaactcttgctgctggtcgaaatgcttatggtgtcctaacggctcctaacaaagtgaccgagcgtgagatgatgactggcttgatgaatgctcatcgtgagatggttcctaacgaccctatcactcaacagatgctcattaagttctatgaggctaacggtgttcaccttaagtctgacaagaagtaa

SEQ ID NO: 56 - DNA, 1968 bp, synthetic construct
  misc_feature 1..1968: note = Synthetic
  source 1..1968: mol_type = other DNA
atggctaacaagattcgtactgtgatgacttacccacttaatggtgccgtgaacttcacgattacctttgagtatctggcacgtaagttcatccaagtgacactcattggtaaagaccgtaaagaactggttctcaatcaggactaccgttttaccagtaagactcaaattactactacccgtgcgtggactgctgctgatggctaccagatgattgagattcgtcgattcacttcggctactgaccgtctggttgactttgccgatggctcaatcctccgtgcgtatgacctgaacatctcgcagattcaaacactgcatgttgccgaagaagcacgagacctcactgcggataccattggtgtcaacaatgatggtcacttagatgctcgtggtcgtaagattgttaacttggcgtatgccacaagtgactatgatgccgttcctttaaagcaggttaaagagcgtgaatcctctgcatggaatgctgtgacaaaagctaacgaacacgctgacagggccaataaggaagcaaaccgttctcgtgatgaggccaaccgtgcccaacgtgaagctgaccgctcaacgcaacaggcaggtgtctctacacaacaagcagttgaggccaagaagcaagctgaccgttctaactctgaggctaaccgagctaaaggttatgctgatagtatgaccgcaagtgtcgctgaggccaagaagcaagctgaccgttccacctctgaggccaatcgtgcccgtgatgaggccaatcgtgctgccggagaagtgactaaagctgctgctgaggttgccaaggctgctgcacacgttgagaccgctaagagtcacgccaatcgtgctaacactgaggcagaacgctctaagagtgaagccgaccgtgctaaaaccgaggctgacaagctgggtaatatgaatgaattagctgaggccatcgacagggttgatgctcctgatgtttacttcaagaggaatgttatagccggacacaaattggagacttctggaggctcgcatcttcaccctaatgagattaaattggttaacaataaatctccagaccagcacttcattgtacaagcgtggggaactggagaccctaataataggtcgcaggtgtttgagttaaaggataacttgggtccgtacgcattctatatccagcgccagaagaagacagaccaaacttggcaaatgagtgtgaacggtactgttagctgcgtatccatcgaggctggcaagggtttcatctctaaggacagtacaggctccattcagatgaagacttgggggggtggttggaaccaatggacttctaatgctgttccagacattcagcacgatattgaggcccaacatgatgctgcttatcttctggagagggctaccatttggggtaaggagcatttatacgcaaaacaactgtacaataatggcactggcgcaaacttctttattcgccactacctacgaggctcttacttcgacttccgctctgatggtactcttatcagttccaccggatggaatatcccggcagatggaaacctgtacatcaagaaatatggactatatattgatgcgtgggttaacaagcgcttgggcgaacacgcctacaacaagagcgagacaaacaacctagtgaatggtcgattgacacaagcgcaagctgatggtcgctacattcgcaagaacgctggttgggctgaggtctggagcggctctgctggtggtggtgtttccgtaggtctctcacaggatgttcgttggagaaccatctggattcaaactcaaggcagatggaaccctgttcagattggtgataatggtgcttactacatctcgtctaatggtgggtggttgaagttcaccatctctaataacggtaggacgttccgtaacgatgaggacaggacttctgttcctacgagaatcttgattcaaaaccaataa

SEQ ID NO: 57 - DNA, 466 bp, synthetic construct
  misc_feature 1..466: note = Synthetic
  source 1..466: mol_type = other DNA
atgttgtcattggattttaacaacgaggtagtgaaggctgctccgattgtagggacaggggttgccgatggtgcttccagactgttcttcggcctatcacttaacgagtggttttatgtagccgcaattgcctacacagtggctcaaattggtgccaaggtagtcgacgtggttatcaaatggaagaaggagggtagagatgtcggataaatctttgattcaattccttgagatgctggacactgagatggctcaacgtatgcttgctgacttgcagaacgatgaacgccgtacacctcaactgtacaacgcaatcgggaaactgttagaacgccacaagttccaagtctccaagttacagccagaccgtaacatccttggtggactggcagatggacttcaggagtatcagaacctagtaggtgacgatggtctcactgaagatgagaagtacacgctgaactga

SEQ ID NO: 58 - DNA, 444 bp, synthetic construct
  misc_feature 1..444: note = Synthetic
  source 1..444: mol_type = other DNA
atgctcaagaatttaaagagctacgtcataatcgctgcgtttgccttggggatgacccttgcgtacaaccacggctatgacaaggctaacactaaatgggaacaggaggtgcacaatgagtacgttaagaaagttaaggcgactgaggataaacaggctgctatcaacgaagtctcacgacaataccaagaagaccttgcggcgctggaaggcagcactgatagggtcattaatgatttgcgtaacgatggtaagcggctgcgtgtcaaactatcagccacctcacgagaactccaagctaacggtggatgcctcgttgatggtcgagccgaacttgacgaagagtttagtaagcgtcttatcggagtaacacaaagaggggatgcgtggattaaagcgctccaaaatacaataactgaaatgcaatctaagaaggagaaataa

SEQ ID NO: 59 - DNA, 558 bp, synthetic construct
  misc_feature 1..558: note = Synthetic
  source 1..558: mol_type = other DNA
atggctactacaaactcaagaacccgcacactctggaccatcaatggtcgctcaccggacagccgggatatgtacgaccgaaacaatgttggccttcgtgttgggcgcatcgacatcccagcgtcagaaatggtcaaggtagatggctacgacaaggttgaatacgtggtgcaactgagtgatggcacaaccgagcaacatgctacgtttgagaccttgatgtccacttggggtggctccgtagagtctaaaccgggtagtgggttcatgacggtatttctgaatgagcaaagcactgagcggctcggtatggttcgaatcgacgtatacaaagagtggactggtgctgaccctgagccggaaggtggtgtggcttcattagttagtgcaacgctttctgcgtctgatagtgcatttggtacacagttcgtggatagcgaagggaagagcgcttccgttatgttccaacctagtaacatcgacggggctgtatctaagaccgacactatcaaggctcttgagaagcgcatcgctgccttagagaaatctgccgagtaa

SEQ ID NO: 60 - DNA, 1761 bp, synthetic construct
  misc_feature 1..1761: note = Synthetic
  source 1..1761: mol_type = other DNA
atgtcacaatcccaagaagctaagaatgcgctcatcattgcgcaactcaagggtgacttcgtggccttcctgttcgtcttatggaaggctctgaacttacctaaaccaaccaagtgtcagattgatatggcacgaacactcgctaatggagaccacaagaagtttatccttcaggcgttccgtggtatcggcaagtcctttatcacctgtgcgttcgttgtgtgggtcttatggcgtgaccctcaacttaaggtgctcattgtgtctgcctctaaggaacgtgcggatgctaactccatcttcatcaagaacatcatcgacctcttaccgttcctctctgagttgaaacctcgaccgggacagcgagactccgtgattagctttgatgttggcttggctaaacccgaccactcaccttctgtgaagtctgtgggtattactggtcagttgactggtagccgtgctgacatcattattgcagatgacgttgaggttcccggtaatagctctacgagttctgcccgtgagaagctctggacgttggtaactgagtttgctgcattgcttaagccgctgcctacctctcgtgttatctaccttggcacacctcaaaccgagatgacgctctacaaggaacttgaggataacaaagggtacagcacagtaatctggccagcacagtatcctcgtaatgatgctgaggctctctactatggggaccgcttggctccgatgcttaaggctgagtacgacgagggctttgagttactccgtggtcaacctactgaccctgttcgattcgacatggatgacctccgtgaacgtgaactcgaatatggcaaagctggctacactcttcagttcatgcttaacccgaaccttagtgatgccgagaagtacccactacgactccgtgacgctatcgtgtgcgctgtggaccctgaacgtgccccattgtcttaccagtggttgccgaaccgtcagaaccgcaatgaggaactccctaacgttggtctgaagggtgatgacatccatgcgttccatacgtgctcttctcgtaccgctgagtatcagtctaagattctggtcattgacccaagtggtcgtggtaaggatgagaccgggtatgcagtcctctactcactgaacggttacatctacctgatggaagtcggagggttccgtggtggctacgatgatgctaccctagagaagctcgctaagaaggccaagcagtggaaggtacagacagtcgtccacgagtccaacttcggtgatggtatgttcggtaagattttctctccgattctcctgaagcaccataagtgtgccttagaggaaatccgtgctaagggcatgaaggagatgcgaatctgcgatactatcgagccgttgatgggtgcacacaagctggtcatccgtgatgaggtcatccgtgaggactaccagactgcccgtgaccttgatgggaaacatgatgtccgctacagtgcgttctatcagatgacacgtatgacccgtgagcgaggcgctgtggcacacgatgaccgcattgatgctatcgcattgggtatcgaatatctccgcgaggggatgcttgtggacagccgtgtaggtgaagaagagatgacccttgagttccttgaacaccacatggagaaacagaccattggtggcgaccagattcatagctttgatgttgggggtgtggacatctactatgaggatgaggacggaggttctagcttcattgagtggtaa

SEQ ID NO: 61 - protein, 4 aa, synthetic construct
KDEL

SEQ ID NO: 62 - protein, 5 aa, synthetic construct
RGDYC

SEQ ID NO: 63 - protein, 5 aa, synthetic construct
ASSHN

SEQ ID NO: 64 - protein, 6 aa, synthetic construct
KCCYSL

SEQ ID NO: 65 - protein, 7 aa, synthetic construct
YEVGHRC